(19) United States
(12) Patent Application Publication
(10) Pub. No.: US 2025/0057981 A1
(43) Pub. Date: Feb. 20, 2025

(54) COMPOSITIONS AND METHODS FOR REDUCING PAIN AND INFLAMMATION THROUGH EPIGENETIC MODULATION

(71) Applicant: Navega Therapeutics, Inc., San Diego, CA (US)

(72) Inventors: Ana Maria MORENO COLLADO, San Diego, CA (US); Fernando ALEMAN GUILLEN, San Diego, CA (US)

(21) Appl. No.: 18/754,071

(22) Filed: Jun. 25, 2024

Related U.S. Application Data
(60) Provisional application No. 63/523,353, filed on Jun. 26, 2023.

(51) Int. Cl. A61K 48/00; A61P 29/00; C12N 9/22; C12N 15/11; C12N 15/86

(52) U.S. Cl. CPC A61K 48/005; A61P 29/00; C12N 9/22; C12N 15/111; C12N 15/86; C12N 2310/20; C12N 2740/15043; C12N 2750/14143

(57) ABSTRACT

Described herein are epigenetic modulators that modulate expression of voltage gated sodium channels, such as Naᵥ1.7 and Naᵥ1.8, without editing the genetic sequence. An epigenetic modulator may include a nucleic acid-binding agent that binds to a target sequence within a gene encoding the voltage gated sodium channel and an expression modulating agent that modulates expression of the gene. Also described herein are methods of treating inflammation, pain, or both using an epigenetic modulator of Naᵥ1.7 or Naᵥ1.8.

### CROSS-REFERENCE

This application claims the benefit of U.S. Provisional Application No. 63/523,353, entitled “COMPOSITIONS AND METHODS FOR REDUCING PAIN AND INFLAMMATION THROUGH EPIGENETIC MODULATION,” filed Jun. 26, 2023, which application is incorporated herein by reference in its entirety.

### GOVERNMENT SUPPORT

This invention was made with government support under R44CA239940 awarded by the National Institute of Health. The government has certain rights in the invention.

### SEQUENCE LISTING

The instant application contains a Sequence Listing which has been submitted electronically in eXtensible Markup Language (XML file) format and is hereby incorporated by reference in its entirety. Said XML copy, created on Aug. 8, 2024, is named 423157-707031_SL.xml and is 349,058 bytes in size.

### BACKGROUND

Chronic pain affects between 19% to 50% of the world population, with more than 100 million people affected in the U.S. alone. Despite their side-effects and limited efficacy, opioids have been a preferred treatment for chronic pain among both private and VA prescribers in recent years. Opioids, however, are highly addictive, and over 130 Americans die each day due to an overdose. Thus, opioid overdose represents a threat that significantly impacts public health. Even though chronic pain is more prevalent than cancer, diabetes and cardiovascular disease combined, drug development for chronic pain has not undergone the remarkable progress seen in these other therapeutic areas. Despite decades of research, broad-acting, long-lasting, non-addictive and effective therapeutics for chronic pain remain elusive.

### SUMMARY

In various aspects, the present disclosure provides a composition comprising: a Naᵥ1.7 epigenetic modulator comprising a SCN9A-binding agent and a first repressor domain, or a polynucleotide encoding the Naᵥ1.7 epigenetic modulator; and a Naᵥ1.8 epigenetic modulator comprising a SCN10A-binding agent and a second repressor domain, or a polynucleotide encoding the Naᵥ1.8 epigenetic modulator.

In some aspects, the SCN9A-binding agent comprises a SCN9A-binding protein. In some aspects, the SCN9A-binding protein comprises a SCN9A-binding zinc finger protein or a SCN9A-binding transcription activator-like effector (TALE). In some aspects, the SCN9A-binding zinc finger protein comprises a sequence having at least 90% sequence identity to any one of SEQ ID NO: 134-SEQ ID NO: 148 or SEQ ID NO: 278-SEQ ID NO: 290; optionally, wherein the SCN9A-binding zinc finger protein comprises a sequence of SEQ ID NO: 140 or SEQ ID NO: 142.

In some aspects, the SCN9A-binding agent comprises a SCN9A-binding polynucleotide. In some aspects, the SCN9A-binding polynucleotide comprises a SCN9A-binding guide RNA. In some aspects, the SCN9A-binding guide RNA comprises a sequence having at least 90% sequence identity to any one of SEQ ID NO: 55-SEQ ID NO: 98. In some aspects, the composition further comprises a Cas protein or a polynucleotide encoding the Cas protein. In some aspects, the Cas protein comprises a sequence having at least 90% sequence identity to any one of SEQ ID NO: 1-SEQ ID NO: 43.

In some aspects, the SCN9A-binding agent has affinity for a SCNA9 polynucleotide sequence. In some aspects, the SCN9A-binding agent has affinity for a sequence having at least 90% sequence identity to any one of SEQ ID NO: 206-SEQ ID NO: 220 or SEQ ID NO: 291-SEQ ID NO: 302.

In some aspects, the first repressor domain comprises ZIM3, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof. In some aspects, the first repressor domain comprises a sequence having at least 90% sequence identity to any one of SEQ ID NO: 193-SEQ ID NO: 205. In some aspects, the SCN9A-binding agent is linked to the first repressor domain.

In some aspects, the SCN10A-binding agent comprises a SCN10A-binding protein. In some aspects, the SCN10A-binding protein comprises a SCN10A-binding zinc finger protein or a SCN10A-binding transcription activator-like effector (TALE). In some aspects, the SCN10A-binding zinc finger protein comprises a sequence having at least 90% sequence identity to any one of SEQ ID NO: 149-SEQ ID NO: 192.

In some aspects, the SCN10A-binding agent comprises a SCN10A-binding polynucleotide. In some aspects, the SCN10A-binding polynucleotide comprises a SCN10A-binding guide RNA. In some aspects, the SCN10A-binding guide RNA comprises a sequence having at least 90% sequence identity to any one of SEQ ID NO: 99-SEQ ID NO: 108 or SEQ ID NO: 268-SEQ ID NO: 277. In some aspects, the composition further comprises a Cas protein or a polynucleotide encoding the Cas protein. In some aspects, the Cas protein comprises a sequence having at least 90% sequence identity to any one of SEQ ID NO: 1-SEQ ID NO: 43.

In some aspects, the SCN10A-binding agent has affinity for a SCN10A polynucleotide sequence. In some aspects, the SCN10A-binding agent has affinity for a sequence having at least 90% sequence identity to any one of SEQ ID NO: 221-SEQ ID NO: 263.

In some aspects, the second repressor domain comprises ZIM3, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof. In some aspects, the second repressor domain comprises a sequence having at least 90% sequence identity to any one of SEQ ID NO: 193-SEQ ID NO: 205. In some aspects, the SCN10A-binding agent is linked to the second repressor domain.

In some aspects, the polynucleotide Naᵥ1.7 epigenetic modulator, the polynucleotide encoding the Naᵥ1.8 epigenetic modulator, or both are under transcriptional control of a promoter. In some aspects, the promoter comprises a sequence having at least 90% sequence identity to any one of SEQ ID NO: 44-SEQ ID NO: 54.

In some aspects, the Naᵥ1.7 epigenetic modulator, the polynucleotide encoding the Naᵥ1.7 epigenetic modulator, the Naᵥ1.8 epigenetic modulator, the polynucleotide encoding the Naᵥ1.8 epigenetic modulator, or a combination thereof are encapsulated in a delivery vector. In some aspects, the delivery comprises a viral vector. In some aspects, the viral vector comprises a delivery-enhancing peptide. In some aspects, the delivery-enhancing peptide comprises a protoxin, a jingzhaotoxina, a theraphotoxin, a phlotoxin, Grammostola porter toxin, a huwentoxin, a Ceratogyrus cornuatus toxin, a heteropodatoxin, a heteroscodratoxin, or a penetration enhancing peptide. In some aspects, the delivery-enhancing peptide comprises a sequence having at least 90% sequence identity to any one of SEQ ID NO: 109-SEQ ID NO: 133. In some aspects, the viral vector comprises an AAV vector or a lentiviral vector. In some aspects, the delivery comprises a lipid nanoparticle.

In various aspects, the present disclosure provides a method of downregulating a Naᵥ1.7 and a Naᵥ1.8 in a cell, the method comprising delivering to the cell a Naᵥ1.7 epigenetic modulator comprising a SCN9A-binding agent and a first repressor domain and a Naᵥ1.8 epigenetic modulator comprising a SCN10A-binding agent and a second repressor domain.

In some aspects, the Naᵥ1.7 epigenetic modulator, the Naᵥ1.8 epigenetic modulator, or both to the cell comprises expressing the Naᵥ1.7 epigenetic modulator, the Naᵥ1.8 epigenetic modulator, or both in the cell.

In various aspects, the present disclosure provides a method of downregulating a Naᵥ1.7 and a Naᵥ1.8 in a cell, the method comprising contacting the cell with a composition as described herein.

In various aspects, the present disclosure provides a method of treating inflammation in a subject in need thereof, the method comprising repressing transcription of a Naᵥ1.7 and a Naᵥ1.8 in a cell of the subject, wherein the cell expresses Naᵥ1.7, Naᵥ1.8, or both.

In various aspects, the present disclosure provides a method of treating inflammation in a subject in need thereof, the method comprising administering to the subject a composition comprising: a Naᵥ1.7 epigenetic modulator comprising a SCN9A-binding agent and a first repressor domain; a polynucleotide encoding the Naᵥ1.7 epigenetic modulator; a Naᵥ1.8 epigenetic modulator comprising a SCN10A-binding agent and a second repressor domain; a polynucleotide encoding the Naᵥ1.8 epigenetic modulator vector; or combinations thereof.

In some aspects, the SCN9A-binding agent comprises a SCN9A-binding protein. In some aspects, the SCN9A-binding protein comprises a SCN9A-binding zinc finger protein or a SCN9A-binding transcription activator-like effector (TALE). In some aspects, the SCN9A-binding zinc finger protein comprises a sequence having at least 90% sequence identity to any one of SEQ ID NO: 134-SEQ ID NO: 148 or SEQ ID NO: 278-SEQ ID NO: 290; optionally, wherein the SCN9A-binding zinc finger protein comprises a sequence of SEQ ID NO: 140 or SEQ ID NO: 142.

In some aspects, the SCN9A-binding agent comprises a SCN9A-binding polynucleotide. In some aspects, the SCN9A-binding polynucleotide comprises a SCN9A-binding guide RNA. In some aspects, the SCN9A-binding guide RNA comprises a sequence having at least 90% sequence identity to any one of SEQ ID NO: 55-SEQ ID NO: 98. In some aspects, the composition further comprises a Cas protein or a polynucleotide encoding the Cas protein. In some aspects, the Cas protein comprises a sequence having at least 90% sequence identity to any one of SEQ ID NO: 1-SEQ ID NO: 43.

In some aspects, the SCN9A-binding agent has affinity for a SCNA9 polynucleotide sequence. In some aspects, the SCN9A-binding agent has affinity for a sequence having at least 90% sequence identity to any one of SEQ ID NO: 206-SEQ ID NO: 220 or SEQ ID NO: 291-SEQ ID NO: 302.

In some aspects, the first repressor domain comprises ZIM3, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof. In some aspects, the first repressor domain comprises a sequence having at least 90% sequence identity to any one of SEQ ID NO: 193-SEQ ID NO: 205. In some aspects, the SCN9A-binding agent is linked to the first repressor domain.

In some aspects, the SCN10A-binding agent comprises a SCN10A-binding protein. In some aspects, the SCN10A-binding protein comprises a SCN10A-binding zinc finger protein or a SCN10A-binding transcription activator-like effector (TALE). In some aspects, the SCN10A-binding zinc finger protein comprises a sequence having at least 90% sequence identity to any one of SEQ ID NO: 149-SEQ ID NO: 192.

In some aspects, the SCN10A-binding agent comprises a SCN10A-binding polynucleotide. In some aspects, the SCN10A-binding polynucleotide comprises a SCN10A-binding guide RNA. In some aspects, the SCN10A-binding guide RNA comprises a sequence having at least 90% sequence identity to any one of SEQ ID NO: 99-SEQ ID NO: 108 or SEQ ID NO: 268-SEQ ID NO: 277. In some aspects, the composition further comprises a Cas protein or a polynucleotide encoding the Cas protein. In some aspects, the Cas protein comprises a sequence having at least 90% sequence identity to any one of SEQ ID NO: 1-SEQ ID NO: 43.

In some aspects, the SCN10A-binding agent has affinity for a SCN10A polynucleotide sequence. In some aspects, the SCN10A-binding agent has affinity for a sequence having at least 90% sequence identity to any one of SEQ ID NO: 221-SEQ ID NO: 263.

In some aspects, the second repressor domain comprises ZIM3, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof. In some aspects, the second repressor domain comprises a sequence having at least 90% sequence identity to any one of SEQ ID NO: 193-SEQ ID NO: 205. In some aspects, the SCN10A-binding agent is linked to the second repressor domain.

In some aspects, the polynucleotide Naᵥ1.7 epigenetic modulator, the polynucleotide encoding the Naᵥ1.8 epigenetic modulator, or both are under transcriptional control of a promoter. In some aspects, the promoter comprises a sequence having at least 90% sequence identity to any one of SEQ ID NO: 44-SEQ ID NO: 54.

In some aspects, the Naᵥ1.7 epigenetic modulator, the polynucleotide encoding the Naᵥ1.7 epigenetic modulator, the Naᵥ1.8 epigenetic modulator, the polynucleotide encoding the Naᵥ1.8 epigenetic modulator, or a combination thereof are encapsulated in a delivery vector. In some aspects, the delivery comprises a viral vector. In some aspects, the viral vector comprises a delivery-enhancing peptide. In some aspects, the delivery-enhancing peptide comprises a protoxin, a jingzhaotoxina, a theraphotoxin, a phlotoxin, Grammostola porter toxin, a huwentoxin, a Ceratogyrus cornuatus toxin, a heteropodatoxin, a heteroscodratoxin, or a penetration enhancing peptide. In some aspects, the delivery-enhancing peptide comprises a sequence having at least 90% sequence identity to any one of SEQ ID NO: 109-SEQ ID NO: 133. In some aspects, the viral vector comprises an AAV vector or a lentiviral vector. In some aspects, the delivery comprises a lipid nanoparticle. In some aspects, the composition comprises a composition as described herein.

In some aspects, the inflammation is associated with arthritis. In some aspects, the arthritis is rheumatoid arthritis or osteoarthritis. In some aspects, treating inflammation comprises preventing inflammation. In some aspects, treating inflammation comprises reducing inflammation.

In some aspects, the method further comprises treating pain in the subject. In some aspects, treating pain comprises preventing pain. In some aspects, treating pain comprises reducing pain. In some aspects, the pain is associated with a condition or with a treatment of a condition. In some aspects, the pain is associated with neuropathy, chemotherapy, or inflammation.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a plot of withdrawal threshold in mice treated with K/BxN serum with or without a Naᵥ1.7 epigenetic modulator or naïve mice in a pain and inflammation prevention model.

FIG. 1B shows a plot of arthritis score in mice treated with K/BxN serum with or without a Naᵥ1.7 epigenetic modulator or naïve mice in a pain and inflammation prevention model.

FIG. 1C shows a plot of the area under the curve (AUC) of the data plotted in FIG. 1A.

FIG. 1D shows a plot of the area under the curve (AUC) of the data plotted in FIG. 1B.

FIG. 2A shows photographs of hind feet of mice treated with K/BxN serum with or without a Naᵥ1.7 epigenetic modulator and a naïve mouse.

FIG. 2B shows photographs of mice treated with K/BxN serum with or without a Naᵥ1.7 epigenetic modulator.

FIG. 3A shows a plot of withdrawal threshold in mice treated with K/BxN serum with or without a Naᵥ1.7 epigenetic modulator or naïve mice in a pain and inflammation reversion model.

FIG. 3B shows a plot of arthritis score in mice treated with K/BxN serum with or without a Naᵥ1.7 epigenetic modulator or naïve mice in a pain and inflammation reversion model.

FIG. 3C shows a plot of the area under the curve (AUC) of the data plotted in FIG. 3A.

FIG. 3D shows a plot of the area under the curve (AUC) of the data plotted in FIG. 3B.

FIG. 4A shows confocal images of sections collected from mice treated with K/BxN serum with or without a Naᵥ1.7 epigenetic modulator or naïve mice. Tissues were stained for PGP and GAP43.

FIG. 4B shows a plot of the PGP staining in FIG. 4A.

FIG. 4C shows a plot of the GAP43 staining in FIG. 4A.

FIG. 5A shows confocal images of sections collected from mice treated with K/BxN serum with or without a Naᵥ1.7 epigenetic modulator or naïve mice. Tissues were stained for CGRP and TH.

FIG. 5B shows a plot of the CGRP staining in FIG. 5A.

FIG. 5C shows a plot of the TH staining in FIG. 5A.

FIG. 6A shows a plot of ankle width in mice treated with complete Freud's adjuvant (CFA) with a Naᵥ1.7 epigenetic modulator, a Naᵥ1.8 epigenetic modulator, no epigenetic modulator, or naïve mice.

FIG. 6B shows a plot of the area under the curve (AUC) of the data plotted in FIG. 6A.

FIG. 6C shows a plot of withdrawal threshold in mice treated with complete Freud's adjuvant (CFA) with a Naᵥ1.7 epigenetic modulator, a Naᵥ1.8 epigenetic modulator, no epigenetic modulator, or naïve mice.

FIG. 6D shows a plot of the area under the curve (AUC) of the data plotted in FIG. 6C.

FIG. 7A shows a plot of withdrawal threshold in mice treated with complete Freud's adjuvant (CFA) with a low concentration of Naᵥ1.7 epigenetic modulator, a low concentration of Naᵥ1.8 epigenetic modulator, no epigenetic modulator, or naïve mice.

FIG. 7B shows a plot of the area under the curve (AUC) of the data plotted in FIG. 7A.

FIG. 7C shows a plot of ankle width in mice treated with complete Freud's adjuvant (CFA) with a low concentration of Naᵥ1.7 epigenetic modulator, a low concentration of Naᵥ1.8 epigenetic modulator, no epigenetic modulator, or naïve mice.

FIG. 7D shows a plot of the area under the curve (AUC) of the data plotted in FIG. 7C.

FIG. 8A and FIG. 8B show mouse tissue sections stained with hematoxylin and eosin (“H&E”).

FIG. 9 shows confocal images of mouse tissue sections stained for endomucin (top) or CD68 and DAPI (bottom).

FIG. 10A shows a plot of withdrawal threshold in mice treated with complete Freud's adjuvant (CFA) with varying concentrations of both an Naᵥ1.7 epigenetic modulator and a Naᵥ1.8 epigenetic modulator or no epigenetic modulator.

FIG. 10B shows a plot of hyperalgesic index in mice treated with complete Freud's adjuvant (CFA) with varying concentrations of both an Naᵥ1.7 epigenetic modulator and a Naᵥ1.8 epigenetic modulator or no epigenetic modulator.

FIG. 10C shows a plot of hyperalgesic index in mice treated with complete Freud's adjuvant (CFA) at two concentrations of a Naᵥ1.7 epigenetic modulator, a Naᵥ1.8 epigenetic modulator, both a Naᵥ1.7 epigenetic modulator and a Naᵥ1.8 epigenetic modulator, or no epigenetic modulator.

FIG. 11A shows in situ repression of Naᵥ1.7, Naᵥ1.8, and of simultaneous Naᵥ1.7+Naᵥ1.8 via KRAB-dCas9 reverses paclitaxel-induced tactile allodynia in male mice.

FIG. 11B shows Relative mechanical allodynia threshold relative to AAV9-KRAB-dCas9-no-gRNA injected male mice.

FIG. 11C shows in situ repression of Naᵥ1.7, Naᵥ1.8, and of simultaneous Naᵥ1.7+Naᵥ1.8 via KRAB-dCas9 reverses paclitaxel-induced tactile allodynia in female mice.

FIG. 11D shows relative mechanical allodynia threshold relative to AAV9-KRAB-dCas9-no-gRNA injected female mice.

FIG. 11E shows in vivo Naᵥ1.7 repression levels: mice DRG (L4-L6) were harvested and Naᵥ1.7 repression efficacy was determined by qPCR.

FIG. 11F shows in vivo Naᵥ1.8 repression levels: mice DRG (L4-L6) were harvested and Naᵥ1.8 repression efficacy was determined by qPCR.

FIG. 12A shows in vivo 1L-10 gene regulation in mice treated with complete Freud's adjuvant (CFA) with a Naᵥ1.7 epigenetic modulator: the IL-10 gene expression level was determined by qPCR.

FIG. 12B shows in vivo NGFR gene regulation in mice treated with complete Freud's adjuvant (CFA) with a Naᵥ1.7 epigenetic modulator: the NGFR gene expression level was determined by qPCR.

### DETAILED DESCRIPTION

Described herein are compositions and methods to epigenetically modify gene expression without editing the genome. Also described herein are methods of treating pain, inflammation, or both using epigenetic modulation of gene expression. A composition for epigenetic modulation may comprise a nucleic acid-binding agent (e.g., a nucleic acid-binding protein, a guide RNA, or combinations thereof), or a polynucleotide encoding the nucleic acid-binding agent, that binds to a target sequence within a genome. The target sequence may be a region (e.g., a coding region or a regulatory region) of a gene, such as a gene associated with pain or inflammation. In some embodiments, binding of the nucleic acid-binding agent to the target sequence may modulate (e.g., downregulate or upregulate) expression of the gene. In some embodiments, binding of the nucleic acid-binding agent to the target sequence may deliver an expression modulating agent (e.g., a transcriptional repressor, a transcriptional activator, or an epigenetic editor) to the gene, thereby modulating expression of the gene. The gene may encode a voltage-gated sodium channel (e.g., Naᵥ1.7 or Naᵥ1.8) associated with pain. For example, the gene may be SCN9A or SCN10A. Nucleic acid-binding agents (e.g., SCN9A-binding agents, SCN10A-binding agents, or combinations thereof) may include nucleic acid-binding proteins, such as zinc finger proteins or transcription activator-like effectors (TALEs), or nucleic acid-binding polynucleotides, such as guide RNAs. In some embodiments, the nucleic acid-binding agent may be part of a CRISPR-Cas system, such as a nuclease-inactivated CRISPR-Cas systems (e.g., for CRISPRi or CRISPRa). A dead Cas system may comprise a nuclease-inactivated Cas (also referred to as “dead Cas” or “dCas”) protein and a guide polynucleotide (e.g., a guide RNA) that binds to the target sequence. The nucleic acid-binding agent may be expressed with or linked to an expression modulating agent (e.g., ZIM3, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof) that modulates expression of the gene.

Epigenetic modulation of gene expression using a composition of the present disclosure may (e.g., a composition modulating expression of Naᵥ1.7, Naᵥ1.8, or a combination thereof) may be used to treat pain, inflammation, or both in a subject. The pain or inflammation may be associated with a disorder (e.g., arthritis or a neurological disorder) or with a treatment of a disorder (e.g., chemotherapy for treatment of cancer). In some embodiments, a method of modulating gene expression may comprise delivering a polynucleotide encoding a nucleic acid-binding agent targeting a gene of interest and an expression modulating agent to a cell of the subject and expressing the nucleic acid-binding agent and the expression modulating agent in the cell, thereby modulating gene expression. Various methods may be used to deliver the polynucleotide to the cell of the subject. For example, the polynucleotide may be delivered using a viral vector (e.g., an adeno-associated virus (AAV) or a lentivirus vector) or a plasmid. In some embodiments, a method of modulating gene expression may comprise delivering the nucleic acid-binding agent and the expression modulating agent to a cell of the subject, thereby modulating gene expression. For example, the nucleic acid-binding agent and the expression modulating agent may be delivered on or in a nanoparticle, such as a lipid nanoparticle.

### Epigenetic Modulators

A composition of the present disclosure may comprise or encode an epigenetic modulator. An epigenetic modulator may modulate expression of a gene of interest without editing the genomic sequence. In some embodiments, an epigenetic modulator may comprise a nucleic acid-binding agent, an expression modulating agent, or both. The nucleic acid-binding agent may be linked to the expression modulating agent (e.g., expressed as a fusion protein), such that binding of the nucleic acid-binding agent to a target sequence delivers the expression modulating agent to the target sequence. Once in proximity to the target sequence, the expression modulating agent may modulate expression of a gene containing the target sequence.

### Nucleic Acid-Binding Agents

Provided herein are nucleic acid-binding (e.g., nucleic acid-binding proteins or nucleic acid-binding polynucleotides) that may modulate gene expression without permanently editing the genome. In certain embodiments, the nucleic acid-binding agent binds to a target sequence. The target sequence may be a region of the gene. In certain embodiments, the nucleic acid binding domain binds to DNA. In certain embodiments, the nucleic acid binding domain binds to RNA or DNA complementary to the RNA.

### CRISPR/Cas Systems

An example nucleic acid-binding agent is a component of a Clustered Regularly Interspaced Short Palindromic Repeats (CRISPR) system, such as a nuclease dead Cas protein (known as dCas or CRISPRi), a guide RNA, or both. The Cas proteins described herein do not have nuclease activity and therefore do not edit the genome. Non-limiting examples of dCas proteins are provided in TABLE 1.

TABLE 1
Exemplary Dead Cas Proteins

| SEQ | |
|---|---|
| ID | |
| NO | Sequence |
| SEQ | MDKKYSIGLDIGTNSVGWAVITDEYKVPSKKFKVLGNTDRHSIKKNLIGALLF |
| ID | DSGETAEATRLKRTARRRYTRRKNRICYLQEIFSNEMAKVDDSFFHRLEESFL |
| NO: | VEEDKKHERHPIFGNIVDEVAYHEKYPTIYHLRKKLVDSTDKADLRLIYLALA |
| 1 | HMIKFRGHFLIEGDLNPDNSDVDKLFIQLVQTYNQLFEENPINASGVDAKAIL |
| | SARLSKSRRLENLIAQLPGEKKNGLFGNLIALSLGLTPNFKSNFDLAEDAKLQ |
| | LSKDTYDDDLDNLLAQIGDQYADLFLAAKNLSDAILLSDILRVNTEITKAPLS |
| | ASMIKRYDEHHQDLTLLKALVRQQLPEKYKEIFFDQSKNGYAGYIDGGASQE |
| | EFYKFIKPILEKMDGTEELLVKLNREDLLRKQRTFDNGSIPHQIHLGELHAILR |
| | RQEDFYPFLKDNREKIEKILTFRIPYYVGPLARGNSRFAWMTRKSEETITPWN |
| | FEEVVDKGASAQSFIERMTNFDKNLPNEKVLPKHSLLYEYFTVYNELTKVKY |
| | VTEGMRKPAFLSGEQKKAIVDLLFKTNRKVTVKQLKEDYFKKIECFDSVEISG |
| | VEDRFNASLGTYHDLLKIIKDKDFLDNEENEDILEDIVLTLTLFEDREMIEERL |
| | KTYAHLFDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELGS |
| | QILKEHPVENTQLQNEKLYLYYLQNGRDMYVDQELDINRLSDYDVDHIVPQS |
| | FLKDDSIDNKVLTRSDKNRGKSDNVPSEEVVKKMKNYWRQLLNAKLITQRK |
| | FDNLTKAERGGLSELDKAGFIKRQLVETRQITKHVAQILDSRMNTKYDENDK |
| | LIREVKVITLKSKLVSDFRKDFQFYKVREINNYHHAHDAYLNAVVGTALIKK |
| | YPKLESEFVYGDYKVYDVRKMIAKSEQEIGKATAKYFFYSNIMNFFKTEITLA |
| | NGEIRKRPLIETNGETGEIVWDKGRDFATVRKVLSMPQVNIVKKTEVQTGGF |
| | SKESILPKRNSDKLIARKKDWDPKKYGGFDSPTVAYSVLVVAKVEKGKSKKL |
| | KSVKELLGITIMERSSFEKNPIDFLEAKGYKEVKKDLIIKLPKYSLFELENGRK |
| | RMLASAGELQKGNELALPSKYVNFLYLASHYEKLKGSPEDNEQKQLFVEQH |
| | KHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| SEQ | MNNSIKSKPEVTIGLDLGVGSVGWAIVDNETNIIHHLGSRLFSQAKTAEDRRS |
| ID | FRGVRRLIRRRKYKLKRFVNLIWKYNSYFGFKNKEDILNNYQEQQKLHNTVL |
| NO: | NLKSEALNAKIDPKALSWILHDYLKNRGHFYEDNRDFNVYPTKELAKYFDK |
| 2 | YGYYKGIIDSKEDNDNKLEEELTKYKFSNKHWLEEVKKVLSNQTGLPEKFKE |
| | EYESLFSYVRNYSEGPGSINSVSPYGIYHLDEKEGKVVQKYNNIWDKTIGKCN |
| | IFPDEYRAPKNSPIAMIFNEINELSTIRSYSIYLTGWFINQEFKKAYLNKLLDLLI |
| | KTNGEKPIDARQFKKLREETIAESIGKETLKDVENEEKLEKEDHKWKLKGLK |
| | LNTNGKIQYNDLSSLAKFVHKLKQHLKLDFLLEDQYATLDKINFLQSLFVYL |
| | GKHLRYSNRVDSANLKEFSDSNKLFERILQKQKDGLFKLFEQTDKDDEKILA |
| | QTHSLSTKAMLLAITRMTNLDNDEDNQKNNDKGWNFEAIKNFDQKFIDITKK |
| | NNNLSLKQNKRYLDDRFINDAILSPGVKRILREATKVFNAILKQFSEEYDVTK |
| | VVIELARELSEEKELENTKNYKKLIKKNGDKISEGLKALGISEDEIKDILKSPTK |
| | SYKFLLWLQQDHIDPYSLKEIAFDDIFTKTEKFEIDHIIPYSISFDDSSSNKLLVL |
| | AESNQAKSNQTPYEFISSGNAGIKWEDYEAYCRKFKDGDSSLLDSTQRSKKF |
| | AKMMKTDTSSKYDIGFLARNLNDTRYATIVFRDALEDYANNHLVEDKPMFK |
| | VVCINGSVTSFLRKNFDDSSYAKKDRDKNIHHAVDASIISIFSNETKTLFNQLT |
| | QFADYKLFKNTDGSWKKIDPKTGVVTEVTDENWKQIRVRNQVSEIAKVIEKY |
| | IQDSNIERKARYSRKIENKTNISLFNDTVYSAKKVGYEDQIKRKNLKTLDIHES |
| | AKENKNSKVKRQFVYRKLVNVSLLNNDKLADLFAEKEDILMYRANPWVINL |
| | AEQIFNEYTENKKIKSQNVFEKYMLDLTKEFPEKFSEFLVKSMLRNKTAIIYD |
| | DKKNIVHRIKRLKMLSSELKENKLSNVIIRSKNQSGTKLSYQDTINSLALMIM |
| | RSIDPTAKKQYIRVPLNTLNLHLGDHDFDLHNMDAYLKKPKFVKYLKANEIG |
| | DEYKPWRVLTSGTLLIHKKDKKLMYISSFQNLNDVIEIKNLIETEYKENDDSD |
| | SKKKKKANRFLMTLSTILNDYILLDAKDNFDILGLSKNRIDEILNSKLGLDKIV |
| | K |
| SEQ | MGRKPYILSLDIGTGSVGYACMDKGFNVLKYHDKDALGVYLFDGALTAQER |
| ID | RQFRTSRRRKNRRIKRLGLLQELLAPLVQNPNFYQFQRQFAWKNDNMDFKN |
| NO: | KSLSEVLSFLGYESKKYPTIYHLQEALLLKDEKFDPELIYMALYHLVKYRGHF |
| 3 | LFDHLKIENLTNNDNMHDFVELIETYENLNNIKLNLDYEKTKVIYEILKDNEM |
| | TKNDRAKRVKNMEKKLEQFSIMLLGLKFNEGKLFNHADNAEELKGANQSHT |
| | FADNYEENLTPFLTVEQSEFIERANKIYLSLTLQDILKGKKSMAMSKVAAYDK |
| | FRNELKQVKDIVYKADSTRTQFKKIFVSSKKSLKQYDATPNDQTFSSLCLFDQ |
| | YLIRPKKQYSLLIKELKKIIPQDSELYFEAENDTLLKVLNTTDNASIPMQINLYE |
| | AETILRNQQKYHAEITDEMIEKVLSLIQFRIPYYVGPLVNDHTASKFGWMERK |
| | SNESIKPWNFDEVVDRSKSATQFIRRMTNKCSYLINEDVLPKNSLLYQEMEVL |
| | NELNATQIRLQTDPKNRKYRMMPQIKLFAVEHIFKKYKTVSHSKFLEIMLNSN |
| | HRENFMNHGEKLSIFGTQDDKKFASKLSSYQDMTKIFGDIEGKRAQIEEIIQWI |
| | TIFEDKKILVQKLKECYPELTSKQINQLKKLNYSGWGRLSEKLLTHAYQGHSII |
| | ELLRHSDENFMEILTNDVYGFQNFIKEENQVQSNKIQHQDIANLTTSPALKKG |
| | IWSTIKLVRELTSIFGEPEKIIMEFATEDQQKGKKQKSRKQLWDDNIKKNKLK |
| | SVDEYKYIIDVANKLNNEQLQQEKLWLYLSQNGKCMYSGQSIDLDALLSPNA |
| | TKHYEVDHIFPRSFIKDDSIDNKVLVIKKMNQTKGDQVPLQFIQQPYERIAYW |
| | KSLNKAGLISDSKLHKLMKPEFTAMDKEGFIQRQLVETRQISVHVRDFLKEEY |
| | PNTKVIPMKAKMVSEFRKKFDIPKIRQMNDAHHAIDAYLNGVVYHGAQLAY |
| | PNVDLFDFNFKWEKVREKWKALGEFNTKQKSRELFFFKKLEKMEVSQGERLI |
| | SKIKLDMNHFKINYSRKLANIPQQFYNQTAVSPKTAELKYESNKSNEVVYKG |
| | LTPYQTYVVAIKSVNKKGKEKMEYQMIDHYVFDFYKFQNGNEKELALYLAQ |
| | RENKDEVLDAQIVYSLNKGDLLYINNHPCYFVSRKEVINAKQFELTVEQQLSL |
| | YNVMNNKETNVEKLLIEYDFIAEKVINEYHHYLNSKLKEKRVRTFFSESNQT |
| | HEDFIKALDELFKVVTASATRSDKIGSRKNSMTHRAFLGKGKDVKIAYTSISG |
| | LKTTKPKSLFKLAESRNEL |
| SEQ | MKYHVGIDVGTFSVGLAAIEVDDAGMPIKTLSLVSHIHDSGLDPDKIKSAVTR |
| ID | LASSGIARRTRRLYRRKRRRLQQLDKFIQRQGWPVIELEDYSDPLYPWKVRA |
| NO: | ELAASYIADEKERGEKLSVALRHIARHRGWRNPYAKVSSLYLPDEPSDAFKAI |
| 4 | REEIKRASGQPVPETATVGQMVTLCELGTLKLRGEGGVLSARLQQSDHAREI |
| | QEICRMQEIGQELYRKIIDVVFAAESPKGSASSRVGKDPLQPGKNRALKASDA |
| | FQRYRIAALIGNLRVRVDGEKRILSVEEKNLVFDHLVNLAPKKEPEWVTIAEI |
| | LGIDRGQLIGTATMTDDGERAGARPPTHDTNRSIVNSRIAPLVDWWKTASAL |
| | EQHAMVKALSNAEVDDFDSPEGAKVQAFFADLDDDVHAKLDSLHLPVGRA |
| | AYSEDTLVRLTRRMLADGVDLYTARLQEFGIEPSWTPPAPRIGEPVGNPAVD |
| | RVLKTVSRWLESATKTWGAPERVIIEHVREGFVTEKRAREMDGDMRRRAAR |
| | NAKLFQEMQEKLNVQGKPSRADLWRYQSVQRQNCQCAYCGSPITFSNSEMD |
| | HIVPRAGQGSTNTRENLVAVCHRCNQSKGNTPFAIWAKNTSIEGVSVKEAVE |
| | RTRHWVTDTGMRSTDFKKFTKAVVERFQRATMDEEIDARSMESVAWMANE |
| | LRSRVAQHFASHGTTVRVYRGSLTAEARRASGISGKLEFLDGVGKSRLDRRH |
| | HAIDAAVIAFTSDYVAETLAVRSNLKQSQAHRQEAPQWREFTGKDAEHRAA |
| | WRVWCQKMEKLSALLTEDLRDDRVVVMSNVRLRLGNGSAHEETIGKLSKV |
| | KLGSQLSVSDIDKASSEALWCALTREPDFDPKDGLPANPERHIRVNGTHVYA |
| | GDNIGLFPVSAGSIALRGGYAELGSSFHHARVYKITSGKKPAFAMLRVYTIDL |
| | LPYRNQDLFSVELKPQTMSMRQAEKKLRDALATGNAEYLGWLVVDDELVV |
| | DTSKIATDQVKAVEAELGTIRRWRVDGFFGDTRLRLRPLQMSKEGIKKESAP |
| | ELSKIIDRPGWLPAVNKLFSEGNVTVVRRDSLGRVRLESTAHLPVTWKVQ |
| SEQ | MLGSSRYLRYNLTSFEGKEPFLIMGYYKEYNKELSSKAQKEFNDQISEFNSYY |
| ID | KLGIDLGDKTGIAIVKGNKIILAKTLIDLHSQKLDKRREARRNRRTRLSRKKRL |
| NO: | ARLRSWVMRQKVGNQRLPDPYKIMHDNKYWSIYNKSNSANKKNWIDLLIHS |
| 5 | NSLSADDFVRGLTIIFRKRGYLAFKYLSRLSDKEFEKYIDNLKPPISKYEYDED |
| | LEELSSRVENGEIEEKKFEGLKNKLDKIDKESKDFQVKQREEVKKELEDLVDL |
| | FAKSVDNKIDKARWKRELNNLLDKKVRKIRFDNRFILKCKIKGCNKNTPKKE |
| | KVRDFELKMVLNNARSDYQISDEDLNSFRNEVINIFQKKENLKKGELKGVTIE |
| | DLRKQLNKTFNKAKIKKGIREQIRSIVFEKISGRSKFCKEHLKEFSEKPAPSDRI |
| | NYGVNSAREQHDFRVLNFIDKKIFKDKLIDPSKLRYITIESPEPETEKLEKGQIS |
| | EKSFETLKEKLAKETGGIDIYTGEKLKKDFEIEHIFPRARMGPSIRENEVASNL |
| | ETNKEKADRTPWEWFGQDEKRWSEFEKRVNSLYSKKKISERKREILLNKSNE |
| | YPGLNPTELSRIPSTLSDFVESIRKMFVKYGYEEPQTLVQKGKPIIQVVRGRDT |
| | QALRWRWHALDSNIIPEKDRKSSFNHAEDAVIAACMPPYYLRQKIFREEAKIK |
| | RKVSNKEKEVTRPDMPTKKIAPNWSEFMKTRNEPVIEVIGKVKPSWKNSIMD |
| | QTFYKYLLKPFKDNLIKIPNVKNTYKWIGVNGQTDSLSLPSKVLSISNKKVDS |
| | STVLLVHDKKGGKRNWVPKSIGGLLVYITPKDGPKRIVQVKPATQGLLIYRN |
| | EDGRVDAVREFINPVIEMYNNGKLAFVEKENEEELLKYFNLLEKGQKFERIRR |
| | YDMITYNSKFYYVTKINKNHRVTIQEESKIKAESDKVKSSSGKEYTRKETEEL |
| | SLQKLAELISI |
| SEQ | MRILGFDIGINSIGWAFVENDELKDCGVRIFTKAENPKNKESLALPRRNARSS |
| ID | RRRLKRRKARLIAIKRILAKELKLNYKDYVAADGELPKAYEGSLASVYELRY |
| NO: | KALTQNLETKDLARVILHIAKHRGYMNKNEKKSNDAKKGKILSALKNNALK |
| 6 | LENYQSVGEYFYKEFFQKYKKNTKNFIKIRNTKDNYNNCVLSSDLEKELKLIL |
| | EKQKEFGYNYSEDFINEILKVAFFQRPLKDFSHLVGACTFFEEEKRACKNSYS |
| | AWEFVALTKIINEIKSLEKISGEIVPTQTINEVLNLILDKGSITYKKFRSCINLHE |
| | SISFKSLKYDKENAENAKLIDFRKLVEFKKALGVHSLSRQELDQISTHITLIKD |
| | NVKLKTVLEKYNLSNEQINNLLEIEFNDYINLSFKALGMILPLMREGKRYDEA |
| | CEIANLKPKTVDEKKDFLPAFCDSIFAHELSNPVVNRAISEYRKVLNALLKKY |
| | GKVHKIHLELARDVGLSKKAREKIEKEQKENQAVNAWALKECENIGLKASA |
| | KNILKLKLWKEQKEICIYSGNKISIEHLKDEKALEVDHIYPYSRSFDDSFINKV |
| | LVFTKENQEKLNKTPFEAFGKNIEKWSKIQTLAQNLPYKKKNKILDENFKDK |
| | QQEDFISRNLNDTRYIATLIAKYTKEYLNFLLLSENENANLKSGEKGSKIHVQ |
| | TISGMLTSVLRHTWGFDKKDRNNHLHHALDAIIVAYSTNSIIKAFSDFRKNQE |
| | LLKARFYAKELTSDNYKHQVKFFEPFKSFREKILSKIDEIFVSKPPRKRARRAL |
| | HKDTFHSENKIIDKCSYNSKEGLQIALSCGRVRKIGTKYVENDTIVRVDIFKKQ |
| | NKFYAIPIYAMDFALGILPNKIVITGKDKNNNPKQWQTIDESYEFCFSLYKND |
| | LILLQKKNMQEPEFAYYNDFSISTSSICVEKHDNKFENLTSNQKLLFSNAKEGS |
| | VKVESLGIQNLKVFEKYIITPLGDKIKADFQPRENISLKTSKKYGLR |
| SEQ | MKQEYFLGLDMGTGSLGWAVTDSTYQVMRKHGKALWGTRLFESASTAEER |
| ID | RMFRTARRRLDRRNWRIQVLQEIFSEEISKVDPGFFLRMKESKYYPEDKRDAE |
| NO: | GNCPELPYALFVDDNYTDKNYHKDYPTIYHLRKMLMETTEIPDIRLVYLVLH |
| 7 | HMMKHRGHFLLSGDISQIKEFKSTFEQLIQNIQDEELEWHISLDDAAIQFVEHV |
| | LKDRNLTRSTKKSRLIKQLNAKSACEKAILNLLSGGTVKLSDIFNNKELDESE |
| | RPKVSFADSGYDDYIGIVEAELAEQYYIIASAKAVYDWSVLVEILGNSVSISEA |
| | KIKVYQKHQADLKTLKKIVRQYMTKEDYKRVFVDTEEKLNNYSAYIGMTKK |
| | NGKKVDLKSKQCTQADFYDFLKKNVIKVIDHKEITQEIESEIEKENFLPKQVT |
| | KDNGVIPYQVHDYELKKILDNLGTRMPFIKENAEKIQQLFEFRIPYYVGPLNR |
| | VDDGKDGKFTWSVRKSDARIYPWNFTEVIDVEASAEKFIRRMTNKCTYLVG |
| | EDVLPKDSLVYSKFMVLNELNNLRLNGEKISVELKQRIYEELFCKYRKVTRK |
| | KLERYLVIEGIAKKGVEITGIDGDFKASLTAYHDFKERLTDVQLSQRAKEAIV |
| | LNVVLFGDDKKLLKQRLSKMYPNLTTGQLKGICSLSYQGWGRLSKTFLEEIT |
| | VPAPGTGEVWNIMTALWQTNDNLMQLLSRNYGFTNEVEEFNTLKKETDLSY |
| | KTVDELYVSPAVKRQIWQTLKVVKEIQKVMGNAPKRVFVEMAREKQEGKR |
| | SDSRKKQLVELYRACKNEERDWITELNAQSDQQLRSDKLFLYYIQKGRCMY |
| | SGETIQLDELWDNTKYDIDHIYPQSKTMDDSLNNRVLVKKNYNAIKSDTYPL |
| | SLDIQKKMMSFWKMLQQQGFITKEKYVRLVRSDELSADELAGFIERQIVETR |
| | QSTKAVATILKEALPDTEIVYVKAGNVSNFRQTYELLKVREMNDLHHAKDA |
| | YLNIVVGNAYFVKFTKNAAWFIRNNPGRSYNLKRMFEFDIERSGEIAWKAGN |
| | KGSIVTVKKVMQKNNILVTRKAYEVKGGLFDQQIMKKGKGQVPIKGNDERL |
| | ADIEKYGGYNKAAGTYFMLVKSLDKKGKEIRTIEFVPLYLKNQIEINHESAIQ |
| | YLAQERGLNSPEILLSKIKIDTLFKVDGFKMWLSGRTGNQLIFKGANQLILSH |
| | QEAAILKGVVKYVNRKNENKDAKLSERDGMTEEKLLQLYDTFLDKLSNTVY |
| | SIRLSAQIKTLTEKRAKFIGLSNEDQCIVLNEILHMFQCQSGSANLKLIGGPGSA |
| | GILVMNNNITACKQISVINQSPTGIYEKEIDLIKL |
| SEQ | MAAFKPNPMNYILGLDIGIASVGWAIVEIDEEENPIRLIDLGVRVFERAEVPKT |
| ID | GDSLAAARRLARSVRRLTRRRAHRLLRARRLLKREGVLQAADFDENGLIKSL |
| NO: | PNTPWQLRAAALDRKLTPLEWSAVLLHLIKHRGYLSQRKNEGETADKELGA |
| 8 | LLKGVADNTHALQTGDFRTPAELALNKFEKESGHIRNQRGDYSHTFNRKDLQ |
| | AELNLLFEKQKEFGNPHVSDGLKEGIETLLMTQRPALSGDAVQKMLGHCTFE |
| | PTEPKAAKNTYTAERFVWLTKLNNLRILEQGSERPLTDTERATLMDEPYRKS |
| | KLTYAQARKLLDLDDTAFFKGLRYGKDNAEASTLMEMKAYHAISRALEKEG |
| | LKDKKSPLNLSPELQDEIGTAFSLFKTDEDITGRLKDRVQPEILEALLKHISFDK |
| | FVQISLKALRRIVPLMEQGNRYDEACTEIYGDHYGKKNTEEKIYLPPIPADEIR |
| | NPVVLRALSQARKVINGVVRRYGSPARIHIETAREVGKSFKDRKEIEKRQEEN |
| | RKDREKSAAKFREYFPNFVGEPKSKDILKLRLYEQQHGKCLYSGKEINLGRL |
| | NEKGYVEIDHALPFSRTWDDSFNNKVLALGSENQNKGNQTPYEYFNGKDNS |
| | REWQEFKARVETSRFPRSKKQRILLQKFDEDGFKERNLNDTRYINRFLCQFVA |
| | DHMLLTGKGKRRVFASNGQITNLLRGFWGLRKVRAENDRHHALDAVVVAC |
| | STIAMQQKITRFVRYKEMNAFDGKTIDKETGEVLHQKAHFPQPWEFFAQEV |
| | MIRVFGKPDGKPEFEEADTPEKLRTLLAEKLSSRPEAVHKYVTPLFISRAPNR |
| | KMSGQGHMETVKSAKRLDEGISVLRVPLTQLKLKDLEKMVNREREPKLYEA |
| | LKARLEAHKDDPAKAFAEPFYKYDKAGNRTQQVKAVRVEQVQKTGVWVH |
| | NHNGIADNATIVRVDVFEKGGKYYLVPIYSWQVAKGILPDRAVVQGKDEED |
| | WTVMDDSFEFKFVLYANDLIKLTAKKNEFLGYFVSLNRATGAIDIRTHDTDS |
| | TKGKNGIFQSVGVKTALSFQKYQIDELGKEIRPCRLKKRPPVR |
| SEQ | MYSIGLDLGISSVGWSVIDERTGNVIDLGVRLFSAKNSEKNLERRTNRGGRRL |
| ID | IRRKTNRLKDAKKILAAVGFYEDKSLKNSCPYQLRVKGLTEPLSRGEIYKVTL |
| NO: | HILKKRGISYLDEVDTEAAKESQDYKEQVRKNAQLLTKYTPGQIQLQRLKEN |
| 9 | NRVKTGINAQGNYQLNVFKVSAYANELATILKTQQAFYPNELTDDWIALFVQ |
| | PGIAEEAGLIYRKRPYYHGPGNEANNSPYGRWSDFQKTGEPATNIFDKLIGKD |
| | FQGELRASGLSLSAQQYNLLNDLTNLKIDGEVPLSSEQKEYILTELMTKEFTR |
| | FGVNDVVKLLGVKKERLSGWRLDKKGKPEIHTLKGYRNWRKIFAEAGIDLA |
| | TLPTETIDCLAKVLTLNTEREGIENTLAFELPELSESVKLLVLDRYKELSQSIST |
| | QSWHRFSLKTLHLLIPELMNATSEQNTLLEQFQLKSDVRKRYSEYKKLPTKD |
| | VLAEIYNPTVNKTVSQAFKVIDALLVKYGKEQIRYITIEMPRDDNEEDEKKRI |
| | KELHAKNSQRKNDSQSYFMQKSGWSQEKFQTTIQKNRRFLAKLLYYYEQDG |
| | ICAYTGLPISPELLVSDSTEIDHIIPISISLDDSINNKVLVLSKANQVKGQQTPYD |
| | AWMDGSFKKINGKFSNWDDYQKWVESRHFSHKKENNLLETRNIFDSEQVEK |
| | FLARNLNDTRYASRLVLNTLQSFFTNQETKVRVVNGSFTHTLRKKWGADLD |
| | KTRETHHHHAVDATLCAVTSFVKVSRYHYAVKEETGEKVMREIDFETGEIVN |
| | EMSYWEFKKSKKYERKTYQVKWPNFREQLKPVNLHPRIKFSHQVDRKANRK |
| | LSDATIYSVREKTEVKTLKSGKQKITTDEYTIGKIKDIYTLDGWEAFKKKQDK |
| | LLMKDLDEKTYERLLSIAETTPDFQEVEEKNGKVKRVKRSPFAVYCEENDIPA |
| | IQKYAKKNNGPLIRSLKYYDGKLNKHINITKDSQGRPVEKTKNGRKVTLQSL |
| | KPYRYDIYQDLETKAYYTVQLYYSDLRFVEGKYGITEKEYMKKVAEQTKGQ |
| | VVRFCFSLQKNDGLEIEWKDSQRYDVRFYNFQSANSINFKGLEQEMMPAEN |
| | QFKQKPYNNGAINLNIAKYGKEGKKLRKFNTDILGKKHYLFYEKEPKNIIK |
| SEQ | MEKKRKVTLGFDLGIASVGWAIVDSETNQVYKLGSRLFDAPDTNLERRTQR |
| ID | GTRRLLRRRKYRNQKFYNLVKRTEVFGLSSREAIENRFRELSIKYPNIIELKTK |
| NO: | ALSQEVCPDEIAWILHDYLKNRGYFYDEKETKEDFDQQTVESMPSYKLNEFY |
| 10 | KKYGYFKGALSQPTESEMKDNKDLKEAFFFDFSNKEWLKEINYFFNVQKNIL |
| | SETFIEEFKKIFSFTRDISKGPGSDNMPSPYGIFGEFGDNGQGGRYEHIWDKNI |
| | GKCSIFTNEQRAPKYLPSALIFNFLNELANIRLYSTDKKNIQPLWKLSSVDKLN |
| | ILLNLFNLPISEKKKKLTSTNINDIVKKESIKSIMISVEDIDMIKDEWAGKEPNV |
| | YGVGLSGLNIEESAKENKFKFQDLKILNVLINLLDNVGIKFEFKDRNDIIKNLE |
| | LLDNLYLFLIYQKESNNKDSSIDLFIAKNESLNIENLKLKLKEFLLGAGNEFEN |
| | HNSKTHSLSKKAIDEILPKLLDNNEGWNLEAIKNYDEEIKSQIEDNSSLMAKQ |
| | DKKYLNDNFLKDAILPPNVKVTFQQAILIFNKIIQKFSKDFEIDKVVIELAREM |
| | TQDQENDALKGIAKAQKSKKSLVEERLEANNIDKSVENDKYEKLIYKIFLWIS |
| | QDFKDPYTGAQISVNEIVNNKVEIDHIIPYSLCFDDSSANKVLVHKQSNQEKS |
| | NSLPYEYIKQGHSGWNWDEFTKYVKRVFVNNVDSILSKKERLKKSENLLTAS |
| | YDGYDKLGFLARNLNDTRYATILFRDQLNNYAEHHLIDNKKMFKVIAMNGA |
| | VTSFIRKNMSYDNKLRLKDRSDFSHHAYDAAIIALFSNKTKTLYNLIDPSLNGI |
| | ISKRSEGYWVIEDRYTGEIKELKKEDWTSIKNNVQARKIAKEIEEYLIDLDDEV |
| | FFSRKTKRKTNRQLYNETIYGIATKTDEDGITNYYKKEKFSILDDKDIYLRLLR |
| | EREKFVINQSNPEVIDQIIEIIESYGKENNIPSRDEAINIKYTKNKINYNLYLKQY |
| | MRSLTKSLDQFSEEFINQMIANKTFVLYNPTKNTTRKIKFLRLVNDVKINDIRK |
| | NQVINKENGKNNEPKAFYENINSLGAIVFKNSANNFKTLSINTQIAIFGDKNW |
| | DIEDFKTYNMEKIEKYKEIYGIDKTYNFHSFIFPGTILLDKQNKEFYYISSIQTV |
| | RDIIEIKFLNKIEFKDENKNQDTSKTPKRLMFGIKSIMNNYEQVDISPFGINKKI |
| | FE |
| SEQ | MRYKIGLDIGITSVGWAVMNLDIPRIEDLGVRIFDRAENPQTGESLALPRRLA |
| ID | RSARRRLRRRKHRLERIRRLVIREGILTKEELDKLFEEKHEIDVWQLREALD |
| NO: | RKLNNDELARVLLHLAKRRGFKSNRKSERSNKENSTMLKHIEENRAILSSYRT |
| 11 | VGEMIVKDPKFALHKRNKGENYTNTIARDDLEREIRLIFSKQREFGNMSCTEE |
| | FENEYIAIWASQRPVASKDDIEKKVGFCAFEPKEKRAPKATYTFQSFIAWEHI |
| | NKLRLISPSGARGLTDEERRLLYEQAFQKNKITYHDIRTLLHLPDDTYFKGIVY |
| | DRGESRKQNENIRFLELDAYHQIRKAVDKVYGKEKSSSFLPIDFDTFGYALTL |
| | FKDDADIHSYLRNEYEQNGKRMPNLANKVYDNELIEELLNLSFTKFGHLSLK |
| | ALRSILPYMEQGEVYSSACERAGYTFTGPKKKQKTMLLPNIPPIANPVVMRAL |
| | TQARKVVNAIIKKYGSPVSIHIELARDLSQTFDERRKTKKEQDENRKKNETAI |
| | RQLMEYGLTLNPTGHDIVKFKLWSEQNGRCAYSLQPIEIERLLEPGYVEVDH |
| | VIPYSRSLDDSYTNKVLVLTRENREKGNRIPAEYLGVGTERWQQFETFVLTN |
| | KQFSKKKRDRLLRLHYDENEETEFKNRNLNDTRYISRFFANFIREHLKFAESD |
| | DKQKVYTVNGRVTAHLRSRWEFNKNREESDLHHAVDAVIVACTTPSDIAKV |
| | TAFYQRREQNKELAKKTEPHFPQPWPHFADELRARLSKHPKESIKALNLGNY |
| | DDQKLESLQPVFVSRMPKRSVTGAAHQETLRRYVGIDERSGKIQTVVKTKLS |
| | EIKLDASGHFPMYGKESDPRTYEAIRQRLLEHNNDPKKAFQEPLYKPKKNGE |
| | PGPVIRTVKIIDTKNQVIPLNDGKTVAYNSNIVRVDVFEKDGKYYCVPVYTM |
| | DIMKGILPNKAIEPNKPYSEWKEMTEDYTFRFSLYPNDLIRIELPREKTVKTAA |
| | GEEINVKDVFVYYKTIDSANGGLELISHDHRFSLRGVGSRTLKRFEKYQVDVL |
| | GNIYKVRGEKRVGLASSAHSKPGKTIRPLQSTRD |
| SEQ | MKRNYILGLDIGITSVGYGIIDYETRDVIDAGVRLFKEANVENNEGRRSKRGA |
| ID | RRLKRRRRHRIQRVKKLLFDYNLLTDHSELSGINPYEARVKGLSQKLSEEEFS |
| NO: | AALLHLAKRRGVHNVNEVEEDTGNELSTKEQISRNSKALEEKYVAELQLERL |
| 12 | KKDGEVRGSINRFKTSDYVKEAKQLLKVQKAYHQLDQSFIDTYIDLLETRRT |
| | YYEGPGEGSPFGWKDIKEWYEMLMGHCTYFPEELRSVKYAYNADLYNALN |
| | DLNNLVITRDENEKLEYYEKFQIIENVFKQKKKPTLKQIAKEILVNEEDIKGYR |
| | VTSTGKPEFTNLKVYHDIKDITARKEIIENAELLDQIAKILTIYQSSEDIQEELTN |
| | LNSELTQEEIEQISNLKGYTGTHNLSLKAINLILDELWHTNDNQIAIFNRLKLV |
| | PKKVDLSQQKEIPTTLVDDFILSPVVKRSFIQSIKVINAIIKKYGLPNDIIIELARE |
| | KNSKDAQKMINEMQKRNRQTNERIEEIIRTTGKENAKYLIEKIKLHDMQEGK |
| | CLYSLEAIPLEDLLNNPFNYEVDHIIPRSVSFDNSFNNKVLVKQEENSKKGNRT |
| | PFQYLSSSDSKISYETFKKHILNLAKGKGRISKTKKEYLLEERDINRFSVQKDFI |
| | NRNLVDTRYATRGLMNLLRSYFRVNNLDVKVKSINGGFTSFLRRKWKFKKE |
| | RNKGYKHHAEDALIIANADFIFKEWKKLDKAKKVMENQMFEEKQAESMPEI |
| | ETEQEYKEIFITPHQIKHIKDFKDYKYSHRVDKKPNRELINDTLYSTRKDDKG |
| | NTLIVNNLNGLYDKDNDKLKKLINKSPEKLLMYHHDPQTYQKLKLIMEQYG |
| | DEKNPLYKYYEETGNYLTKYSKKDNGPVIKKIKYYGNKLNAHLDITDDYPNS |
| | RNKVVKLSLKPYRFDVYLDNGVYKFVTVKNLDVIKKENYYEVNSKCYEEAK |
| | KLKKISNQAEFIASFYNNDLIKINGELYRVIGVNNDLLNRIEVNMIDITYREYL |
| | ENMNDKRPPRIIKTIASKTQSIKKYSTDILGNLYEVKSKKHPQIIKKG |
| SEQ | MQTTNLSYILGLDLGIASVGWAVVEINENEDPIGLIDVGVRIFERAEVPKTGES |
| ID | LALSRRLARSTRRLIRRRAHRLLLAKRFLKREGILSTIDLEKGLPNQAWELRV |
| NO: | AGLERRLSAIEWGAVLLHLIKHRGYLSKRKNESQTNNKELGALLSGVAQNH |
| 13 | QLLQSDDYRTPAELALKKFAKEEGHIRNQRGAYTHTFNRLDLLAELNLLFAQ |
| | QHQFGNPHCKEHIQQYMTELLMWQKPALSGEAILKMLGKCTHEKNEFKAAK |
| | HTYSAERFVWLTKLNNLRILEDGAERALNEEERQLLINHPYEKSKLTYAQVR |
| | KLLGLSEQAIFKHLRYSKENAESATFMELKAWHAIRKALENQGLKDTWQDL |
| | AKKPDLLDEIGTAFSLYKTDEDIQQYLTNKVPNSVINALLVSLNFDKFIELSLK |
| | SLRKILPLMEQGKRYDQACREIYGHHYGEANQKTSQLLPAIPAQEIRNPVVLR |
| | TLSQARKVINAIIRQYGSPARVHIETGRELGKSFKERREIQKQQEDNRTKRESA |
| | VQKFKELFSDFSSEPKSKDILKFRLYEQQHGKCLYSGKEINIHRLNEKGYVEID |
| | HALPFSRTWDDSFNNKVLVLASENQNKGNQTPYEWLQGKINSERWKNFVAL |
| | VLGSQCSAAKKQRLLTQVIDDNKFIDRNLNDTRYIARFLSNYIQENLLLVGKN |
| | KKNVFTPNGQITALLRSRWGLIKARENNNRHHALDAIVVACATPSMQQKITR |
| | FIRFKEVHPYKIENRYEMVDQESGEIISPHFPEPWAYFRQEVNIRVFDNHPDTV |
| | LKEMLPDRPQANHQFVQPLFVSRAPTRKMSGQGHMETIKSAKRLAEGISVLR |
| | IPLTQLKPNLLENMVNKEREPALYAGLKARLAEFNQDPAKAFATPFYKQGGQ |
| | QVKAIRVEQVQKSGVLVRENNGVADNASIVRTDVFIKNNKFFLVPIYTWQVA |
| | KGILPNKAIVAHKNEDEWEEMDEGAKFKFSLFPNDLVELKTKKEYFFGYYIG |
| | LDRATGNISLKEHDGEISKGKDGVYRVGVKLALSFEKYQVDELGKNRQICRP |
| | QQRQPVR |
| SEQ | MKKPYSIGLDIGTNSVGWAVVTDDYKVPAKKMKVLGNTDKSHIEKNLLGAL |
| ID | LFDSGNTAEDRRLKRTARRRYTRRRNRILYLQEIFSEEMGKVDDSFFHRLEDS |
| NO: | FLVTEDKRGERHPIFGNLEEEVKYHENFPTIYHLRQYLADNPEKVDLRLVYLA |
| 14 | LAHIIKFRGHFLIEGKFDTRNNDVQRLFQEFLAVYDNTFENSSLQEQNVQVEEI |
| | LTDKISKSAKKDRVLKLFPNEKSNGRFAEFLKLIVGNQADFKKHFELEEKAPL |
| | QFSKDTYEEELEVLLAQIGDNYAELFLSAKKLYDSILLSGILTVTDVGTKAPLS |
| | ASMIQRYNEHQMDLAQLKQFIRQKLSDKYNEVESDVSKDGYAGYIDGKTNQ |
| | EAFYKYLKGLLNKIEGSGYFLDKIEREDFLRKQRTFDNGSIPHQIHLQEMRAII |
| | RRQAEFYPFLADNQDRIEKLLTFRIPYYVGPLARGKSDFAWLSRKSADKITPW |
| | NFDEIVDKESSAEAFINRMTNYDLYLPNQKVLPKHSLLYEKFTVYNELTKVK |
| | YKTEQGKTAFFDANMKQEIFDGVFKVYRKVTKDKLMDFLEKEFDEFRIVDLT |
| | GLDKENKVFNASYGTYHDLCKILDKDFLDNSKNEKILEDIVLTLTLFEDREMI |
| | RKRLENYSDLLTKEQVKKLERRHYTGWGRLSAELIHGIRNKESRKTILDYLID |
| | DGNSNRNFMQLINDDALSFKEEIAKAQVIGETDNLNQVVSDIAGSPAIKKGIL |
| | QSLKIVDELVKIMGHQPENIVVEMARENQFTNQGRRNSQQRLKGLTDSIKEF |
| | GSQILKEHPVENSQLQNDRLFLYYLQNGRDMYTGEELDIDYLSQYDIDHIIPQ |
| | AFIKDNSIDNRVLTSSKENRGKSDDVPSKDVVRKMKSYWSKLLSAKLITQRK |
| | FDNLTKAERGGLTDDDKAGFIKRQLVETRQITKHVARILDERENTETDENNK |
| | KIRQVKIVTLKSNLVSNFRKEFELYKVREINDYHHAHDAYLNAVIGKALLGV |
| | YPQLEPEFVYGDYPHFHGHKENKATAKKFFYSNIMNFFKKDDVRTDKNGEII |
| | WKKDEHISNIKKVLSYPQVNIVKKVEEQTGGFSKESILPKGNSDKLIPRKTKK |
| | FYWDTKKYGGFDSPIVAYSILVIADIEKGKSKKLKTVKALVGVTIMEKMTFE |
| | RDPVAFLERKGYRNVQEENIIKLPKYSLFKLENGRKRLLASARELQKGNEIVL |
| | PNHLGTLLYHAKNIHKVDEPKHLDYVDKHKDEFKELLDVVSNFSKKYTLAE |
| | GNLEKIKELYAQNNGEDLKELASSFINLLTFTAIGAPATFKFFDKNIDRKRYTS |
| | TTEILNATLIHQSITGLYETRIDLNKLGGD |
| SEQ | MNKPYSIGLDIGTNSVGWSIITDDYKVPAKKMRVLGNTDKEYIKKNLIGALLF |
| ID | DGGNTAADRRLKRTARRRYTRRRNRILYLQEIFAEEMSKVDDSFFHRLEDSF |
| NO: | LVEEDKRGSKYPIFATMQEEKYYHEKFPTIYHLRKELADKKEKADLRLVYLA |
| 15 | LAHIIKFRGHFLIEDDRFDVRNTDIQKQYQAFLEIFDTTFENNHLLSQNVDVEA |
| | ILTDKISKSAKKDRILAQYPNQKSTGIFAEFLKLIVGNQADFKKHFNLEDKTPL |
| | QFAKDSYDEDLENLLGQIGDEFADLFSVAKKLYDSVLLSGILTVTDLSTKAPL |
| | SASMIQRYDEHHEDLKHLKQFVKASLPENYREVFADSSKDGYAGYIEGKTNQ |
| | EAFYKYLLKLLTKQEGSEYFLEKIKNEDFLRKQRTFDNGSIPHQVHLTELRAII |
| | RRQSEYYPFLKENQDRIEKILTFRIPYYVGPLAREKSDFAWMTRKTDDSIRPW |
| | NFEDLVDKEKSAEAFIHRMTNNDLYLPEEKVLPKHSLIYEKFTVYNELTKVRF |
| | LAEGFKDFQFLNRKQKETIFNSLFKEKRKVTEKDIISFLNKVDGYEGIAIKGIE |
| | KQFNASLSTYHDLKKILGKDFLDNTDNELILEDIVQTLTLFEDREMIKKCLDIY |
| | KDFFTESQLKKLYRRHYTGWGRLSAKLINGIRNKENQKTILDYLIDDGSANR |
| | NFMQLINDDDLSFKPIIDKARTGSHSDNLKEVVGELAGSPAIKKGILQSLKIVD |
| | ELVKVMGYEPEQIVVEMARENQTTAKGLSRSRQRLTTLRESLANLKSNILEE |
| | KKPKYVKDQVENHHLSDDRLFLYYLQNGRDMYTKKALDIDNLSQYDIDHIIP |
| | QAFIKDDSIDNRVLVSSAKNRGKSDDVPSIEIVKARKMFWKNLLDAKLMSQR |
| | KYDNLTKAERGGLTSDDKARFIQRQLVETRQITKHVARILDERENNEVDNGK |
| | KICKVKIVTLKSNLVSNFRKEFGFYKIREVNDYHHAHDAYLNAVVAKAILTK |
| | YPQLEPEFVYGMYRQKKLSKIVHEDKEEKYSEATRKMFFYSNLMNMFKRVV |
| | RLADGSIVVRPVIETGRYMRKTAWDKKKHFATVRKVLSYPQNNIVKKTEIQT |
| | GGFSKESILAHGNSDKLIPRKTKDIYLDPKKYGGFDSPIVAYSVLVVADIKKG |
| | KAQKLKTVTELLGITIMERSRFEKNPSAFLESKGYLNIRDDKLMILPKYSLFEL |
| | ENGRRRLLASAGELQKGNELALPTQFMKFLYLASRYNESKGKPEEIEKKQEF |
| | VNQHVSYFDDILQLINDFSKRVILADANLEKINKLYQDNKENIPVDELANNIIN |
| | LFTFTSLGAPAAFKFFDKIVDRKRYTSTKEVLNSTLIHQSITGLYETRIDLGKL |
| | GED |
| SEQ | MTNGKILGLDIGIASVGVGIIEAKTGKVVHANSRLFSAANAENNAERRGFRGS |
| ID | RRLNRRKKHRVKRVRDLFEKYGIVTDFRNLNLNPYELRVKGLTEQLKNEELF |
| NO: | AALRTISKRRGISYLDDAEDDSTGSTDYAKSIDENRRLLKNKTPGQIQLERLE |
| 16 | KYGQLRGNFTVYDENGEAHRLINVESTSDYEKEARKILETQADYNKKITAEFI |
| | DDYVEILTQKRKYYHGPGNEKSRTDYGRFRTDGTTLENIFGILIGKCNFYPDE |
| | YRASKASYTAQEYNFLNDLNNLKVSTETGKLSTEQKESLVEFAKNTATLGPA |
| | KLLKEIAKILDCKVDEIKGYREDDKGKPDLHTFEPYRKLKFNLESINIDDLSRE |
| | VIDKLADILTLNTEREGIEDAIKRNLPNQFTEEQISEIIKVRKSQSTAFNKGWHS |
| | FSAKLMNELIPELYATSDEQMTILTRLEKFKVNKKSSKNTKTIDEKEVTDEIY |
| | NPVVAKSVRQTIKIINAAVKKYGDFDKIVIEMPRDKNADDEKKFIDKRNKEN |
| | KKEKDDALKRAAYLYNSSDKLPDEVFHGNKQLETKIRLWYQQGERCLYSGK |
| | PISIQELVHNSNNFEIDHILPLSLSFDDSLANKVLVYAWTNQEKGQKTPYQVID |
| | SMDAAWSFREMKDYVLKQKGLGKKKRDYLLTTENIDKIEVKKKFIERNLVD |
| | TRYASRVVLNSLQSALRELGKDTKVSVVRGQFTSQLRRKWKIDKSRETYHH |
| | HAVDALIIAASSQLKLWEKQDNPMFVDYGKNQVVDKQTGEILSVSDDEYKE |
| | LVFQPPYQGFVNTISSKGFEDEILFSYQVDSKYNRKVSDATIYSTRKAKIGKD |
| | KKEETYVLGKIKDIYSQNGFDTFIKKYNKDKTQFLMYQKDSLTWENVIEVILR |
| | DYPTTKKSEDGKNDVKCNPFEEYRRENGLICKYSKKGKGTPIKSLKYYDKKL |
| | GNCIDITPEESRNKVILQSINPWRADVYFNPETLKYELMGLKYSDLSFEKGTG |
| | NYHISQEKYDAIKEKEGIGKKSEFKFTLYRNDLILIKDIASGEQEIYRFLSRTMP |
| | NVNHYVELKPYDKEKFDNVQELVEALGEADKVGRCIKGLNKPNISIYKVRTD |
| | VLGNKYFVKKKGDKPKLDFKNNKK |
| SEQ | MAAFKPNPMNYILGLDIGIASVGWAMVEVDEEENPIRLIDLGVRVFERAEVP |
| ID | KTGDSLAMARRLARSVRRLTRRRAHRLLRARRLLKREGVLQDADFDENGLV |
| NO: | KSLPNTPWQLRAAALDRKLTCLEWSAVLLHLVKHRGYLSQRKNEGETADKE |
| 17 | LGALLKGVADNAHALQTGDFRTPAELALNKFEKESGHIRNQRGDYSHTFSRK |
| | DLQAELNLLFEKQKEFGNPHVSDGLKEDIETLLMAQRPALSGDAVQKMLGH |
| | CTFEPAEPKAAKNTYTAERFIWLTKLNNLRILEQGSERPLTDTERATLMDEPY |
| | RKSKLTYAQARKLLGLEDTAFFKGLRYGKDNAEASTLMEMKAYHAISRALE |
| | KEGLKDKKSPLNLSTELQDEIGTAFSLFKTDKDITGRLKDRVQPEILEALLKHI |
| | SFDKFVQISLKALRRIVPLMEQGKRYDEACAEIYGDHYCKKNAEEKIYLPPIP |
| | ADEIRNPVVLRALSQARKVINCVVRRYGSPARIHIETAREVGKSFKDRKEIEK |
| | RQEENRKDREKAAAKFREYFPNFVGEPKSKDILKLRLYEQQHGKCLYSGKEI |
| | NLVRLNEKGYVEIDHALPFSRTWDDSFNNKVLVLGSENQNKGNQTPYEYFN |
| | GKDNSREWQEFKARVETSRFPRSKKQRILLQKFDEEGFKERNLNDTRYVNRF |
| | LCQFVADHILLTGKGKRRVFASNGQITNLLRGFWGLRKVRTENDRHHALDA |
| | VVVACSTVAMQQKITRFVRYKEMNAFDGKTIDKETGEVLHQKAHFPQPWEF |
| | FAQEVMIRVFGKPDGKPEFEEADTPEKLRTLLAEKLSSRPEAVHEYVTPLFVS |
| | RAPNRKMSGQGHMETVKSAKRLDEGISVLRVPLTQLKLKGLEKMVNREREP |
| | KLYDALKAQLETHKDDPAKAFAEPFYKYDKAGSRTQQVKAVRIEQVQKTGV |
| | WVRNHNGIADNATMVRVDVFEKGGKYYLVPIYSWQVAKGILPDRAVVAFK |
| | DEEDWTVMDDSFEFRFVLYANDLIKLTAKKNEFLGYFVSLNRATGAIDIRTH |
| | DTDSTKGKNGIFQSVGVKTALSFQKNQIDELGKEIRPCRLKKRPPVR |
| SEQ | MNVKILPIAIDLDVKNTGVFSAFYQKGTSLEKLDNKNGKVYELSKDSYTLLM |
| ID | NNRTARRHQRRGIDRKQLVKRLFKLVWTEQLNLEWDKDTQQAISFLFNRRG |
| NO: | FSFITDGYSTEYLNIVPEQVKAILMDIFDDYNGEDDLDSYLKLATEQESKISEIY |
| 18 | NKLMQKILEFKLRKLCTDIKDDKVSTKTLKEITSYEFELLADYLANYSESLKIQ |
| | KFSYTDKQGNLKELSYYHHDKYNIQEFLKRHATINDEILDTLLTDDFDIWNFN |
| | FEKFDFDKNEEKLQNQEDKDHTQAHLHHFVFAVNKIKSEMASGGRHRSQYF |
| | QEITNVLDENNHQEGYLKNFCENLHNKKYSNLSVKNLVNLVGNLSNLELKPL |
| | RKYFNDKIHAKADHWDEQKFTETYCHWILGEWRVGVKDQDKKDGAKYSY |
| | KDLCNELKQKVTKAGLIDFLLELDPCRTIPPYLDNNNRKPPKCQSLILNPKFLD |
| | NQYPNWQQYLQELKKLQSIQDYLDSFETDLKDLKSSKDQPYFVEYKSSNQQ |
| | MASGQRDYKDLDARILQFIFDRVKASDELLLNEIYFQAKKLKQKASSELEKLE |
| | SSKKLDEVIANSQLSQILKSQHTNGIFEQGTFLHLVCKYYKQRQRARDSRLYI |
| | MPEYRYDKKLDKYNNTGRFDDNNQLLTYCNHKPRQKRYQLLNDLAGVLQV |
| | SRNQLLSSVEEWFQQAQRVGEISKSQDEQIFEWLKSFKIASYCKAAVEMQKQ |
| | YRGTLKNAIQTAIFRQSENINKNKNKNTGNQQQALSENSKDVKSLTADEKKL |
| | LKLIENIAKASQKIGESLGLNDKQIKKFNSIYSFAQIQQIAFAERKGNANTCAV |
| | CSADNAHRMQQIKITELVEDNKDNIILSAKAQRLPAIPTRIVDGAVKKMATIL |
| | AKNIVDDNWQNIKQVLSAKHQLHIPIITESNAFEFEPALADVKGKSLKDRRKK |
| | ALERISPENIFKDKNNRIKEFAKGISAYSGANLTDGDFDGAKEELDHIIPRSHK |
| | KYGTLNDEANLICVTRDDNKNIFAIDTSKWFEIETPSDLRDIGVATIQYKIDNN |
| | SRPKVRVKLDYVIDDDSKINYFMNHSLLKSRYPDKVLEILKQSTIIEFESSGEN |
| | KTIKEMLGMTLAGIYNETSNN |
| SEQ | MKRTSLRAYRLGVDLGANSLGWFVVWLDDHGQPEGLGPGGVRIFPDGRNP |
| ID | QSKQSNAAGRRLARSARRRRDRYLQRRGKLMGLLVKHGLMPADEPARKRL |
| NO: | ECLDPYGLRAKALDEVLPLHHVGRALFHLNQRRGLFANRAIEQGDKDASAIK |
| 19 | AAAGRLQTSMQACGARTLGEFLNRRHQLRATVRARSPVGGDVQARYEFYPT |
| | RAMVDAEFEAIWAAQAPHHPTMTAEAHDTIREAIFSQRAMKRPSIGKCSLDP |
| | ATSQDDVDGFRCAWSHPLAQRFRIWQDVRNLAVVETGPTSSRLGKEDQDKV |
| | ARALLQTDQLSFDEIRGLLGLPSDARFNLESDRRDHLKGDATGAILSARRHFG |
| | PAWHDRSLDRQIDIVALLESALDEAAIIASLGTTHSLDEAAAQRALSALLPDG |
| | YCRLGLRAIKRVLPLMEAGRTYAEAASAAGYDHALLPGGKLSPTGYLPYYG |
| | QWLQNDVVGSDDERDTNERRWGRLPNPTVHIGIGQLRRVVNELIRWHGPPA |
| | EITVELTRDLKLSPRRLAELEREQAENQRKNDKRTSLLRKLGLPASTHNLLKL |
| | RLWDEQGDVASECPYTGEAIGLERLVSDDVDIDHLIPFSISWDDSAANKVVC |
| | MRYANREKGNRTPFEAFGHRQGRPYDWADIAERAARLPRGKRWRFGPGAR |
| | AQFEELGDFQARLLNETSWLARVAKQYLAAVTHPHRIHVLPGRLTALLRAT |
| | WELNDLLPGSDDRAAKSRKDHRHHAIDALVAALTDQALLRRMANAHDDTR |
| | RKIEVLLPWPTFRIDLETRLKAMLVSHKPDHGLQARLHEDTAYGTVEHPETE |
| | DGANLVYRKTFVDISEKEIDRIRDRRLRDLVRAHVAGERQQGKTLKAAVLSF |
| | AQRRDIAGHPNGIRHVRLTKSIKPDYLVPIRDKAGRIYKSYNAGENAFVDILQ |
| | AESGRWIARATTVFQANQANESHDAPAAQPIMRVFKGDMLRIDHAGAEKFV |
| | KIVRLSPSNNLLYLVEHHQAGVFQTRHDDPEDSFRWLFASFDKLREWNAELV |
| | RIDTLGQPWRRKRGLETGSEDATRIGWTRPKKWP |
| SEQ | MNGLVLGLDIGIASVGVGILEKDTGKIIHASSRLFPAATADNNVERRSNRQGR |
| ID | RLNRRKKHRSVRLQDLFEGYGLLTDFSKVSMNLNPYQLRVQGMENQLTNEE |
| NO: | LFVALKNIVKRRGISYLDDASEDGGTVSSDYGKAVEENRKLLAEKTPGQIQLE |
| 20 | RFEKYGQLRGDFTVEENGEKHRLINVESTSAYRKEAERILRKQQEFNSKITDE |
| | FIEDYLIILTGKRKYYHGPGNEKSRTDYGRFRTDGTTLDNIFGILIGKCTFYTEE |
| | YRASKASYTAQEFNLLNDLNNLTVPTETKKLSEEQKKLIIEYAKSAKTLGAST |
| | LLKYIAKMIDASVDQIRGYRVDVNNKPEMHTFEVYRKMQSLETIKVEELPRK |
| | VLDELAHILTLNTEREGIEEAINSKLKDIFNRDQVLELVQFRKNNSSLFSKGW |
| | HNFSIKLMMELIPELYETSEEQMTILTRLGKQRSKETSKRTKYIDEKELTEEIY |
| | NPVVAKSVRQAIKIINEATKKYGIFDNIVIEMARENNEEDAKKDYIKRQKANQ |
| | DEKNAAMEKAAFQYNGKKELPDNIFHGHKELTTKIRLWHQQGEKCLYTGKN |
| | IPISDLIHNQYKYEIDHILPLSLSFDDSLSNKVLVLATANQEKGQRTPFQALDS |
| | MDDAWSYREFKSYVKDSKLLSNKKKDYLLTEEDISKIEVKQKFIERNLVDTR |
| | YSSRVVLNALQDFYKSHQLDTTISVVRGQFTSQLRRKWGIEKSRETYHHHAV |
| | DALIIAASSQLRLWKKHSNPLIAYKEGQFVDSETGEIVSLSDEEYKELVFKAPY |
| | DHFVDTLRSKKFEDSILFSYQVDSKYNRKISDATIYATRKAKLDKEKKEYTYT |
| | LGKIKDIYALGTKTPSKTGFYKFLDLYKTDKSQFLMYQKDRKTWDEVIEKIIE |
| | QYRPFKEYDKNGKEVDENPFEKYRIGNGPIRKYSKKGNGPEIKSLKYYDILLG |
| | KHKNITPDGSRNTVALLSLNPWRTDVYYNSETKKYEFLGLKYADLCFEEGGA |
| | YGISEVKYKKIREKEGIGKNSEFKFTLYKNDLILIKDTETNCQQFFRFWSRTGK |
| | DNPKSFEKHKIELKPYEKAKFEKGEELKVLGKVPPSSNQFQKNMQIENLSIYK |
| | VKTDILGNKHFIKKEGDEPKLKFKK |
| SEQ | MARILAFDIGISSIGWAFSENDELKDCGVRIFTKAENPKTGESLALPRRLARSA |
| ID | RKRLARRKARLNHLKHLIANEFKLNYEDYQSFDESLAKAYKGSLISPYELRFR |
| NO: | ALNELLSKQDFARVILHIAKRRGYDDIKNNGDEEKSEILKAIKQNEEKLVNYQ |
| 21 | SVGEYLYKEYFQKFKENSKEFINVRNKKESYERCITQSFLKDELKLIFQKQREF |
| | GFSFSKKFEEEVLSVAFYKRALKDFSHLVGNCSFFTDEKRAPKNSPLAFMFVA |
| | LTRIINILNNLKNTEGILYTKDDLNALLNEVLKNGTLTYKQTKKLLGLSDDYE |
| | FKGEKGTYFIEFKKYKEFIKALGEHNLSQDDLNEIAKDITLIKDEIKLKKALAK |
| | YDLNQNQIDSLSKLEFKDHLNISFKALKLITPLMLEGKKYDEACNELNLKVAI |
| | NEDKKDFLPAFNETYYKDEVTNPVVLRAIKEYRKILNALLKKYGKVHKINIEL |
| | AREVGKNHSQRAKIEKEQNENYKAKKDAEIECEKLGLKINSKNILKLRLFKE |
| | QKEFCAYSGEKIKLSDLQDEKMLEIDHIYPYSRSFDDSYMNKVLVFTKQNQE |
| | KLNKTPFEAFGNDSTKWQKIEVLAKNLPEKKQKRILDKNYKDKEQKDFKDR |
| | NLNDTRYIARLVLNYTKDYLDFLPLSDDENTKLNDTQKGSKVHVEAKSGML |
| | TSALRHTWGFSAKDRNNHLHHAIDAAIIAYANNSIVKAFSDFKKEQESNSVEL |
| | YAKKISELDYKNKRKFFEPFSGFRQKVLDKIDEIFVSKPERKKPSGALHEETFR |
| | KEEEFYQSYGGKEGVLKALELGKIRKVNGKIVKNGDMFRVDIFKHKKTNKF |
| | YAVPIYTMDFALKVLPNKAVARSKKGEIKDWILMDENYEFCFSLYKDSLILIQ |
| | TKDMQEPELVYFNAFTSSTVSLIVSKHDNKFETLSKNQKILFKNANEKEVIAK |
| | SIGIQNLKVFEKYIVSALGEVTKAEFRQREDFKK |
| SEQ | MRLGLDIGTSSIGWWLYETDGAGSDARITGVVDGGVRIFSDGRDPKSGASLA |
| ID | VDRRAARAMRRRRDRYLRRRATLMKVLAETGLMPADPAEAKALEALDPFA |
| NO: | LRAAGLDEPLPLPHLGRALFHLNQRRGFKSNRKTDRGDNESGKIKDATARLD |
| 22 | MEMMANGARTYGEFLHKRRQKATDPRHVPSVRTRLSIANRGGPDGKEEAG |
| | YDFYPDRRHLEEEFHKLWAAQGAHHPELTETLRDLLFEKIFFQRPLKEPEVGL |
| | CLFSGHHGVPPKDPRLPKAHPLTQRRVLYETVNQLRVTADGREARPLTREER |
| | DQVIHALDNKKPTKSLSSMVLKLPALAKVLKLRDGERFTLETGVRDAIACDP |
| | LRASPAHPDRFGPRWSILDADAQWEVISRIRRVQSDAEHAALVDWLTEAHGL |
| | DRAHAEATAHAPLPDGYGRLGLTATTRILYQLTADVVTYADAVKACGWHH |
| | SDGRTGECFDRLPYYGEVLERHVIPGSYHPDDDDITRFGRITNPTVHIGLNQLR |
| | RLVNRIIETHGKPHQIVVELARDLKKSEEQKRADIKRIRDTTEAAKKRSEKLEE |
| | LEIEDNGRNRMLLRLWEDLNPDDAMRRFCPYTGTRISAAMIFDGSCDVDHIL |
| | PYSRTLDDSFPNRTLCLREANRQKRNQTPWQAWGDTPHWHAIAANLKNLPE |
| | NKRWRFAPDAMTRFEGENGFLDRALKDTQYLARISRSYLDTLFTKGGHVWV |
| | VPGRFTEMLRRHWGLNSLLSDAGRGAVKAKNRTDHRHHAIDAAVIAATDPG |
| | LLNRISRAAGQGEAAGQSAELIARDTPPPWEGFRDDLRVRLDRIIVSHRADHG |
| | RIDHAARKQGRDSTAGQLHQETAYSIVDDIHVASRTDLLSLKPAQLLDEPGRS |
| | GQVRDPQLRKALRVATGGKTGKDFENALRYFASKPGPYQAIRRVRIIKPLQA |
| | QARVPVPAQDPIKAYQGGSNHLFEIWRLPDGEIEAQVITSFEAHTLEGEKRPH |
| | PAAKRLLRVHKGDMVALERDGRRVVGHVQKMDIANGLFIVPHNEANADTR |
| | NNDKSDPFKWIQIGARPAIASGIRRVSVDEIGRLRDGGTRPI |
| SEQ | MAAFKPNPINYILGLDIGIASVGWAMVEIDEDENPICLIDLGVRVFERAEVPKT |
| ID | GDSLAMARRLARSVRRLTRRRAHRLLRARRLLKREGVLQAADFDENGLIKSL |
| NO: | PNTPWQLRAAALDRKLTPLEWSAVLLHLIKHRGYLSQRKNEGETADKELGA |
| 23 | LLKGVADNAHALQTGDFRTPAELALNKFEKESGHIRNQRGDYSHTFSRKDLQ |
| | AELNLLFEKQKEFGNPHISDGLKEGIETLLMTQRPALSGDAVQKMLGHCTFEP |
| | AEPKAAKNTYTAERFIWLTKLNNLRILEQGSERPLTDTERTTLIDEPYRKSKLT |
| | YAQARKLLELDNTAFFKGLRYGKDNAEASTLMEMKAYHAISRALEKEGLKD |
| | KKSPLNLSPELQDEIGTAFSLFKTDEDITGRLKDRVQPEILEALLKHISFDKFVQ |
| | ISLKALRRIVPLMEQGKRYDEACAEIYGDHYGKKNTEEKIDLPPIPADEIRNPV |
| | VLRALSQARKVINGVVRRYGSPARIHIETAREVGKSFKDRKEIEKRQEENRKD |
| | REKAATKFREYFPNFVGEPKSKDILKLRLYEQQHGKCLYSGKEINLGRLNEK |
| | GYVEIDHALPFSRTWDDSFNNKVLVLGSENQNKGNQTPYEYFNGKDNSREW |
| | QEFKARVETSRFPRSKKQRILLQKFDEEGFKERNLNDTRYVNRFLCQFVADH |
| | MLLTGKGKRRVFASNGQITNLLRGFWGLRKVRAENDRHHALDAVVVACST |
| | VAMQQKITRFVRYKEMNAFDGKTIDKETGEVLHQKTHFPQPWEFFAQEVMI |
| | RVFGKPDGKPEFEEADTPEKLRTLLAEKLSSRPEAVHEYVTPLFVSRAPNRKM |
| | SGQGHMETVKSAKRLDEGVSVLRVPLTQLKLKDLEKMVNREREPKLYEALK |
| | ARLEAHKDDPAKAFAEPFYKYDKAGNRTQQVKAVRVEQVQKTGVWVRNH |
| | NGIADNATMVRVDVFEKGDKYYLVPIYSWQVAKGILPDRAVVQGKDEEDW |
| | QLIDDSFNFKFSLHPNDLVEVITKKARMFGYFASCHRGTGNINIRIHDLDHKIG |
| | KNGILEGIGVKTALSFQKYQIDELGKEIRPCRLKKRPPVR |
| SEQ | MQKNINTKQNHIYIKQAQKIKEKLGDKPYRIGLDLGVGSIGFAIVSMEENDGN |
| ID | VLLPKEIIMVGSRIFKASAGAADRKLSRGQRNNHRHTRERMRYLWKVLAEQ |
| NO: | KLALPVPADLDRKENSSEGETSAKRFLGDVLQKDIYELRVKSLDERLSLQELG |
| 24 | YVLYHIAGHRGSSAIRTFENDSEEAQKENTENKKIAGNIKRLMAKKNYRTYG |
| | EYLYKEFFENKEKHKREKISNAANNHKFSPTRDLVIKEAEAILKKQAGKDGF |
| | HKELTEEYIEKLTKAIGYESEKLIPESGFCPYLKDEKRLPASHKLNEERRLWET |
| | LNNARYSDPIVDIVTGEITGYYEKQFTKEQKQKLFDYLLTGSELTPAQTKKLL |
| | GLKNTNFEDIILQGRDKKAQKIKGYKLIKLESMPFWARLSEAQQDSFLYDWN |
| | SCPDEKLLTEKLSNEYHLTEEEIDNAFNEIVLSSSYAPLGKSAMLIILEKIKNDL |
| | SYTEAVEEALKEGKLTKEKQAIKDRLPYYGAVLQESTQKIIAKGFSPQFKDKG |
| | YKTPHTNKYELEYGRIANPVVHQTLNELRKLVNEIIDILGKKPCEIGLETAREL |
| | KKSAEDRSKLSREQNDNESNRNRIYEIYIRPQQQVIITRRENPRNYILKFELLEE |
| | QKSQCPFCGGQISPNDIINNQADIEHLFPIAESEDNGRNNLVISHSACNADKAK |
| | RSPWAAFASAAKDSKYDYNRILSNVKENIPHKAWRFNQGAFEKFIENKPMA |
| | ARFKTDNSYISKVAHKYLACLFEKPNIICVKGSLTAQLRMAWGLQGLMIPFA |
| | KQLITEKESESFNKDVNSNKKIRLDNRHHALDAIVIAYASRGYGNLLNKMAG |
| | KDYKINYSERNWLSKILLPPNNIVWENIDADLESFESSVKTALKNAFISVKHD |
| | HSDNGELVKGTMYKIFYSERGYTLTTYKKLSALKLTDPQKKKTPKDFLETAL |
| | LKFKGRESEMKNEKIKSAIENNKRLFDVIQDNLEKAKKLLEEENEKSKAEGK |
| | KEKNINDASIYQKAISLSGDKYVQLSKKEPGKFFAISKPTPTTTGYGYDTGDSL |
| | CVDLYYDNKGKLCGEIIRKIDAQQKNPLKYKEQGFTLFERIYGGDILEVDFDI |
| | HSDKNSFRNNTGSAPENRVFIKVGTFTEITNNNIQIWFGNIIKSTGGQDDSFTIN |
| | SMQQYNPRKLILSSCGFIKYRSPILKNKEG |
| SEQ | MSRSLTFSFDIGYASIGWAVIASASHDDADPSVCGCGTVLFPKDDCQAFKRRE |
| ID | YRRLRRNIRSRRVRIERIGRLLVQAQIITPEMKETSGHPAPFYLASEALKGHRT |
| NO: | LAPIELWHVLRWYAHNRGYDNNASWSNSLSEDGGNGEDTERVKHAQDLMD |
| 25 | KHGTATMAETICRELKLEEGKADAPMEVSTPAYKNLNTAFPRLIVEKEVRRIL |
| | ELSAPLIPGLTAEIIELIAQHHPLTTEQRGVLLQHGIKLARRYRGSLLFGQLIPRF |
| | DNRIISRCPVTWAQVYEAELKKGNSEQSARERAEKLSKVPTANCPEFYEYRM |
| | ARILCNIRADGEPLSAEIRRELMNQARQEGKLTKASLEKAISSRLGKETETNVS |
| | NYFTLHPDSEEALYLNPAVEVLQRSGIGQILSPSVYRIAANRLRRGKSVTPNY |
| | LLNLLKSRGESGEALEKKIEKESKKKEADYADTPLKPKYATGRAPYARTVLK |
| | KVVEEILDGEDPTRPARGEAHPDGELKAHDGCLYCLLDTDSSVNQHQKERRL |
| | DTMTNNHLVRHRMLILDRLLKDLIQDFADGQKDRISRVCVEVGKELTTFSAM |
| | DSKKIQRELTLRQKSHTDAVNRLKRKLPGKALSANLIRKCRIAMDMNWTCPF |
| | TGATYGDHELENLELEHIVPHSFRQSNALSSLVLTWPGVNRMKGQRTGYDFV |
| | EQEQENPVPDKPNLHICSLNNYRELVEKLDDKKGHEDDRRRKKKRKALLMV |
| | RGLSHKHQSQNHEAMKEIGMTEGMMTQSSHLMKLACKSIKTSLPDAHIDMIP |
| | GAVTAEVRKAWDVFGVFKELCPEAADPDSGKILKENLRSLTHLHHALDACV |
| | LGLIPYIIPAHHNGLLRRVLAMRRIPEKLIPQVRPVANQRHYVLNDDGRMML |
| | RDLSASLKENIREQLMEQRVIQHVPADMGGALLKETMQRVLSVDGSGEDAM |
| | VSLSKKKDGKKEKNQVKASKLVGVFPEGPSKLKALKAAIEIDGNYGVALDP |
| | KPVVIRHIKVFKRIMALKEQNGGKPVRILKKGMLIHLTSSKDPKHAGVWRIES |
| | IQDSKGGVKLDLQRAHCAVPKNKTHECNWREVDLISLLKKYQMKRYPTSYT |
| | GTPR |
| SEQ | MDKKYSIGLDIGTNSVGWAVITDDYKVPSKKFKVLGNTDRHSIKKNLIGALL |
| ID | FDSGETAEATRLKRTARRRYTRRKNRICYLQEIFSNEIAKVDDSFFHRLEESFL |
| NO: | VEEDKKHERHPIFGNIVDEVAYHEKYPTIYHLRKKLADSTDKADLRLIYLALA |
| 26 | HMIKFRGHFLIEGDLNPDNSDVDKLFIQLVQTYNQLFEENPINASRVDAKAILS |
| | ARLSKSRRLENLIAQLPGEKRNGLFGNLIALSLGLTPNFKSNFDLAEDAKLQL |
| | SKDTYDDDLDNLLAQIGDQYADLFLAAKNLSDAILLSDILRVNTEITKAPLSA |
| | SMIKRYDEHHQDLTLLKALVRQQLPEKYKEIFFDQSKNGYAGYIDGGASQEE |
| | FYKFIKPILEKMDGTEELLAKLNREDLLRKQRTFDNGSIPHQIHLGELHAILRR |
| | QEDFYPFLKDNREKIEKILTFRIPYYVGPLARGNSRFAWMTRKSEETITPWNFE |
| | EVVDKGASAQSFIERMTNFDKNLPNEKVLPKHSLLYEYFTVYNELTKVKYVT |
| | EGMRKPAFLSGEQKKAIVDLLFKTNRKVTVKQLKEDYFKKIECFDSVEISGVE |
| | DRFNASLGTYHDLLKIIKDKDFLDNEENEDILEDIVLTLTLFEDREMIEERLKT |
| | YAHLFDDKVMKQLKRRRYTGWGRLSRKLINGIRDKQSGKTILDFLKSDGFA |
| | NRNFMQLIHDDSLTFKEDLQKAQVSGQGDSLHEHIANLAGSPAIKKGILQTV |
| | KVVDELVKVMGRHKPENIVIEMARENQTTQKGQKNSRERMKRIEEGIKELGS |
| | DILKEYPVETTQLQNEKLYLYYLQNGRDMYVDQELDINRLSDYDVDHIVPQS |
| | FLKDDSIDNKVLTRSDKNRGKSDNVPSEEVVKKMKNYWKQLLNAKLITQRK |
| | FDNLTKAERGGLSELDKAGFIKRQLVETRQITKHVAQILDSRMNTKYDENDK |
| | LIREVRVITLKSKLVSDFRKDFQFYKVREINNYHHAHDAYLNAVVGTALIKK |
| | YPKLESEFVYGDYKVYDIRKMIAKSEQEIGKATAKYFFYSNIMNFFKTEITLA |
| | NGEIRKRPLIETNGETGEIVWDKGRDFATVRKVLSMPQVNIVKKTEVQTGGF |
| | SKESILPKRNSDKLIARKKDWDPKKYGGFDSPTVAYSVLVVAKVEKGKSKKL |
| | KSVKELVGITIMERSSFEKDPVDFLEAKGYKEVRKDLIIKLPKYSLFELENGRK |
| | RMLASAGELQKGNELALPSKYVNFLYLASHYEKLKGSPEDNEQKQLFVEQH |
| | KHYLDEIIEQISEFSKRVILADANLDKVLSAYNKHRDKPIREQAENIIHLFTLTN |
| | LGAPAAFKCFDTTIGRNRYKSIKEVLDATLIHQSITGLYETRIDLSQLGGD |
| SEQ | MAAFKPNPINYILGLDIGIASVGWAMVEIDEDENPICLIDLGVRVFERAEVPKT |
| ID | GDSLAMARRLARSVRRLTRRRAHRLLRARRLLKREGVLQAADFDENGLIKSL |
| NO: | PNTPWQLRAAALDRKLTPLEWSAVLLHLIKHRGYLSQRKNEGETADKELGA |
| 27 | LLKGVADNAHALQTGDFRTPAELALNKFEKESGHIRNQRGDYSHTFSRKDLQ |
| | AELILLFEKQKEFGNPHVSGGLKEGIETLLMTQRPALSGDAVQKMLGHCTFEP |
| | AEPKAAKNTYTAERFIWLTKLNNLRILEQGSERPLTDTERATLMDEPYRKSKL |
| | TYAQARKLLGLEDTAFFKGLRYGKDNAEASTLMEMKAYHAISRALEKEGLK |
| | DKKSPLNLSPELQDEIGTAFSLFKTDEDITGRLKDRIQPEILEALLKHISFDKFV |
| | QISLKALRRIVPLMEQGKRYDEACAEIYGDHYGKKNTEEKIYLPPIPADEIRNP |
| | VVLRALSQARKVINGVVRRYGSPARIHIETAREVGKSFKDRKEIEKRQEENRK |
| | DREKAAAKFREYFPNFVGEPKSKDILKLRLYEQQHGKCLYSGKEINLGRLNE |
| | KGYVEIDHALPFSRTWDDSFNNKVLVLGSENQNKGNQTPYEYFNGKDNSRE |
| | WQEFKARVETSRFPRSKKQRILLQKFDEDGFKERNLNDTRYVNRFLCQFVAD |
| | RMRLTGKGKKRVFASNGQITNLLRGFWGLRKVRAENDRHHALDAVVVACS |
| | TVAMQQKITRFVRYKEMNAFDGKTIDKETGEVLHQKTHFPQPWEFFAQEVM |
| | IRVFGKPDGKPEFEEADTPEKLRTLLAEKLSSRPEAVHEYVTPLFVSRAPNRK |
| | MSGQGHMETVKSAKRLDEGVSVLRVPLTQLKLKDLEKMVNREREPKLYEAL |
| | KARLEAHKDDPAKAFAEPFYKYDKAGNRTQQVKAVRVEQVQKTGVWVRN |
| | HNGIADNATMVRVDVFEKGDKYYLVPIYSWQVAKGILPDRAVVQGKDEED |
| | WQLIDDSFNFKFSLHPNDLVEVITKKARMFGYFASCHRGTGNINIRIHDLDHK |
| | IGKNGILEGIGVKTALSFQKYQIDELGKEIRPCRLKKRPPVR |
| SEQ | MARILAFDIGISSIGWAFSENDELKDCGVRIFTKVENPKTGESLALPRRLARSA |
| ID | RKRLARRKARLNHLKHLIANEFKLNYEDYQSFDESLAKAYKGSLISPYELRFR |
| NO: | ALNELLSKQDFARVILHIAKRRGYDDIKNSDDKEKGAILKAIKQNEEKLANY |
| 28 | QSVGEYLYKEYFQKFKENSKEFTNVRNKKESYERCIAQSFLKDELKLIFKKQR |
| | EFGFSFSKKFEEEVLSVAFYKRALKDFSHLVGNCSFFTDEKRAPKNSPLAFMF |
| | VALTRIINLLNNLKNTEGILYTKDDLNALLNEVLKNGTLTYKQTKKLLGLSD |
| | DYEFKGEKGTYFIEFKKYKEFIKALGEHNLSQDDLNEIAKDITLIKDEIKLKKA |
| | LAKYDLNQNQIDSLSKLEFKDHLNISFKALKLVTPLMLEGKKYDEACNELNL |
| | KVAINEDKKDFLPAFNETYYKDEVTNPVVLRAIKEYRKVLNALLKKYGKVH |
| | KINIELAREVGKNHSQRAKIEKEQNENYKAKKDAELECEKLGLKINSKNILKL |
| | RLFKEQKEFCAYSGEKIKISDLQDEKMLEIDHIYPYSRSFDDSYMNKVLVFTK |
| | QNQEKLNQTPFEAFGNDSAKWQKIEVLAKNLPTKKQKRILDKNYKDKEQKN |
| | FKDRNLNDTRYIARLVLNYTKDYLDFLPLSDDENTKLNDTQKGSKVHVEAK |
| | SGMLTSALRHTWGFSAKDRNNHLHHAIDAVIIAYANNSIVKAFSDFKKEQES |
| | NSAELYAKKISELDYKNKRKFFEPFSGFRQKVLDKIDEIFVSKPERKKPSGALH |
| | EETFRKEEEFYQSYGGKEGVLKALELGKIRKVNGKIVKNGDMFRVDIFKHKK |
| | TNKFYAVPIYTMDFALKVLPNKAVARSKKGEIKDWILMDENYEFCFSLYKDS |
| | LILIQTKDMQEPEFVYYNAFTSSTVSLIVSKHDNKFETLSKNQKILFKNANEKE |
| | VIAKSIGIQNLKVFEKYIVSALGEVTKAEFRQREDFKK |
| SEQ | MAQHVFGLDIGIASVGWAILGEQRIIDLGVRCFDKAETAKEGDPLNLTRRQA |
| ID | RLLRRRLYRRAWRLTQLSRLLKRKGLIADAKLFAKAPSYGDSAWELRRQGL |
| NO: | DRLLTPLEWARVIYHQCKHRGFHWTSKAEEAKADSDAEGGRVKQGLAHTK |
| 29 | ALMQAKNYRSAAEMVLAEFPDAQRNKRGQYDKALSRVLLGEELALLFATQ |
| | RRLGNPHASDFFEKLILGDGDRKSGLFWQQKPALSGADLLKMLGKCTFEKGE |
| | YRAPKASFSVERHVWLTRLNNLRIVVDGRSRPLNEAERQAALLLPYQTETSK |
| | YKTLKNAFIKAGLWGDGVRFGGLAYPSQAQIDAEKTKDPEDQFLVKLPAWH |
| | ELRKAFKAAGHEALWQQISTPALDGDPTLLDQIATVLSVYKDGAEVVQQLR |
| | QLALPEPAASIAVLEKISFDKFSSLSLKALRRIVPLMQSGLRYDEAVAQIPEYG |
| | HHSQRIEPGAAKHLYLPPFYEAQRKYAGKGDHIGSMQFRDDADIPRNPVVLR |
| | ALNQARKVVNALIREYGSPIAVNIEMARDLSRPLDERNKVKRAQEEFRDRND |
| | RARSEFERDFGYKPKAAAFEKWMLYREQLGQCAYSQQPLDIQRVLDDHNYA |
| | QVDHALPYSRSYDDSKNNKVLVLTHENQNKGNRTAFEYLTSFPDGEDGERW |
| | RTFVAWVQGNKAYRMAKRNRLLRKNYGVDESKGFIDRNLNDTRYICKFFKN |
| | YVEEHLQLAARADGDTARRCVVVNGQLTAFLRARWGLTKVRGDSDRHHAL |
| | DAAVVAACTHGMVKALADYSRRKEISFLQEGFPDPETGEILNPAAFDRARQH |
| | FPEPWTHFAHELKARLFTDDLAALREDMQRLGSYTTEDLGRLRTLFVSRAPQ |
| | RRSGGAVHKETIYAQPESLKQQGGVIEKILLTSLKLQDFDKLLNPESNDHFVE |
| | PHRNERLYAAIRQRLEQFGGRADKAFGPDNLFHKPDKNNQPTGPVVRSIKLV |
| | RGKQTGIPIRGGLAKNDSMLRVDIFTKAGKFHLVPVYVHHRVTGLPNRAIVA |
| | FKDEDEWTLIDESFAFLFSVYPNDYVKVTLKKEQQSGYYSGADRSTGAMNL |
| | WAHDRAASVGKDGLIRGIGVKTALSVEKFNVDVLGRIYLAPPETRSGLA |
| SEQ | MAAFKPNPINYILGLDIGIASVGWAMVEIDEEENPIRLIDLGVRVFERAEVPKT |
| ID | GDSLAMVRRLARSVRRLTRRRAHRLLRARRLLKREGVLQAADFDENGLIKSL |
| NO | PNTPWQLRAAALDRKLTPLEWSAVLLHLIKHRGYLSQRKNEGETADKELGA |
| 30 | LLKGVADNAHALQTGDFRTPAELALNKFEKESGHIRNQRGDYSHTFSRKDLQ |
| | AELILLFEKQKEFGNPHISGGLKEGIETLLMTQRPALSGDAVQKMLGHCTFEP |
| | AEPKAAKNTYTAERFIWLTKLNNLRILEQGSERPLTDTERATLMDEPYRKSKL |
| | TYAQARKLLGLEDTAFFKGLRYGKDNAEASTLMEMKAYHAISRALEKEGLK |
| | DKKSPLNLSPELQDEIGTAFSLFKTDEDITGRLKDRIQPEILEALLKHISFDKFV |
| | QISLKALRRIVPLMEQGKRYDEACAEIYGDHYGKKNTEEKIYLPPIPADEIRNP |
| | VVLRALSQARKVINGVVRRYGSPARIHIETAREVGKSFKDRKEIEKRQEENRK |
| | DREKAAAKFREYFPNFVGEPKSKDILKLRLYEQQHGKCLYSGKEINLGRLNE |
| | KGYVEIDHALPFSRTWDDSFNNKVLVLGSENQNKGNQTPYEYFNGKDNSRE |
| | WQEFKARVETSRFPRSKKQRILLQKFDEDGFKERNLNDTRYVNRFLCQFVAD |
| | RMRLTGKGKKRVFASNGQITNLLRGFWGLRKVRAENDRHHALDAVVVACS |
| | TVAMQQKITRFVRYKEMNAFDGKTIDKETGEVLHQKTHFPQPWEFFAQEVM |
| | IRVFGKPDGKPEFEEADTPEKLRTLLAEKLSSRPEAVHEYVTPLFVSRAPNRK |
| | MSGQGHMETVKSAKRLDEGVSVLRVPLTQLKLKDLEKMVNREREPKLYEAL |
| | KARLEAHKDDPAKAFAEPFYKYDKAGNRTQQVKAVRVEQVQKTGVWVRN |
| | HNGIADNATMVRVDVFEKGDKYYLVPIYSWQVAKGILPDRAVVQGKDEED |
| | WQLIDDSFNFKFSLHPNDLVEVITKKARMFGYFASCHRGTGNINIRIHDLDHK |
| | IGKNGILEGIGVKTALSFQKYQIDELGKEIRPCRLKKRPPVR |
| SEQ | MTKLNQPYGIGLDIGSNSIGFAVVDANSHLLRLKGETAIGARLFREGQSAADR |
| ID | RGSRTTRRRLSRTRWRLSFLRDFFAPHITKIDPDFFLRQKYSEISPKDKDRFKY |
| NO: | EKRLFNDRTDAEFYEDYPSMYHLRLHLMTHTHKADPREIFLAIHHILKSRGHF |
| 31 | LTPGAAKDFNTDKVDLEDIFPALTEAYAQVYPDLELTFDLAKADDFKAKLLD |
| | EQATPSDTQKALVNLLLSSDGEKEIVKKRKQVLTEFAKAITGLKTKFNLALGT |
| | EVDEADASNWQFSMGQLDDKWSNIETSMTDQGTEIFEQIQELYRARLLNGIV |
| | PAGMSLSQAKVADYGQHKEDLELFKTYLKKLNDHELAKTIRGLYDRYINGD |
| | DAKPFLREDFVKALTKEVTAHPNEVSEQLLNRMGQANFMLKQRTKANGAIPI |
| | QLQQRELDQIIANQSKYYDWLAAPNPVEAHRWKMPYQLDELLNFHIPYYVG |
| | PLITPKQQAESGENVFAWMVRKDPSGNITPYNFDEKVDREASANTFIQRMKT |
| | TDTYLIGEDVLPKQSLLYQKYEVLNELNNVRINNECLGTDQKQRLIREVFERH |
| | SSVTIKQVADNLVAHGDFARRPEIRGLADEKRFLSSLSTYHQLKEILHEAIDDP |
| | TKLLDIENIITWSTVFEDHTIFETKLAEIEWLDPKKINELSGIRYRGWGQFSRKL |
| | LDGLKLGNGHTVIQELMLSNHNLMQILADETLKETMTELNQDKLKTDDIEDV |
| | INDAYTSPSNKKALRQVLRVVEDIKHAANGQDPSWLFIETADGTGTAGKRTQ |
| | SRQKQIQTVYANAAQELIDSAVRGELEDKIADKASFTDRLVLYFMQGGRDIY |
| | TGAPLNIDQLSHYDIDHILPQSLIKDDSLDNRVLVNATINREKNNVFASTLFAG |
| | KMKATWRKWHEAGLISGRKLRNLMLRPDEIDKFAKGFVARQLVETRQIIKLT |
| | EQIAAAQYPNTKIIAVKAGLSHQLREELDFPKNRDVNHYHHAFDAFLAARIG |
| | TYLLKRYPKLAPFFTYGEFAKVDVKKFREFNFIGALTHAKKNIIAKDTGEIVW |
| | DKERDIRELDRIYNFKRMLITHEVYFETADLFKQTIYAAKDSKERGGSKQLIP |
| | KKQGYPTQVYGGYTQESGSYNALVRVAEADTTAYQVIKISAQNASKIASANL |
| | KSREKGKQLLNEIVVKQLAKRRKNWKPSANSFKIVIPRFGMGTLFQNAKYGL |
| | FMVNSDTYYRNYQELWLSRENQKLLKKLFSIKYEKTQMNHDALQVYKAIID |
| | QVEKFFKLYDINQFRAKLSDAIERFEKLPINTDGNKIGKTETLRQILIGLQANG |
| | TRSNVKNLGIKTDLGLLQVGSGIKLDKDTQIVYQSPSGLFKRRIPLADL |
| SEQ | MKYILGLDLGVASIGWAVTEINEKGEPVGLIDANSVIFKPLDNDKGKLYNVE |
| ID | RRDKRGSRRILRRKKHRVERTKMLLVNSSFLTNEEIDNLYVGKLDNIYEVRL |
| NO: | KGLKEQLSKNEIARLMIYYCKNRGFKSNRKTEDLEILKSLGEKISEKDSDEKK |
| 32 | LKPIIAKNTELIESKGLTPIEVIYMIRENDNSILGFKNKEGNYKFGFKRNQVIDE |
| | VRKILGTQQILSKEIVDEYIDILSSQRDFSDGPGGDSKYKIDYTKLAGKCKYTG |
| | EVRAVKSAPSYEIFTMLQKLNDIRYVKFTSEDKIEKKKLSKEVIHKLYDLVVE |
| | KNKTLTYDLIEKSIDEENIKLLNIPKLSKSKYVELRKSYFKERNDNENLTEEEI |
| | NAFNEKLNNERMKQELIRNNLKAYKELKSKFKKNNIDENRIKELGGIYFLDL |
| | VAELLTYSKTDEKLEYFVENVERYSLFKEQRDIVEIIKTFPNYTKNGNLSLSLV |
| | RELNKLLMEGHDYESSLSSLNYYIDTDVDWEKFPTISEIEDSLSTKITNPNVKH |
| | ILVILRKLYNTLLFKYGRPEKVHLELSRDFSNDFSTRNKIQKEQLENKVRREV |
| | AAFEMYGANKDIVAGKDRLSNDDFVRIKLWEEQNKVCMYSGRTIEKYQLTS |
| | AEVQIDHILPYSKSFDNSYSNKVLVFSNENQDKKERTPYQWLKGTEKWNEFK |
| | QRVRLNLNISNKKKENLLFEDEVVNNEFLERELHATSYSSRLALNIFQRLIPVS |
| | DEDKYDEHGNEKVKYLYNRNVIAFQGKMTSMLRNLYSLNKYTHSFESDTLD |
| | IDNKAFILKEFEINKDNLKMTAYNVNTGLEITSETKVEKNKSGEFKTIKDELLK |
| | KELDKKENLIEISDYFKDKVLFDLKIVDFEEIDTVEPEIISILYKMITALKEEVYS |
| | KNRENHLHHSLDAFLLTIMNRSMQMKLIKYNQLISVLKNKEIEIFNEEKGEYI |
| | DSKEFAEELQKQKIIDIDGNERGIKFNVNGKTHRLNLVKPYENFLDDIKNKIFD |
| | KRENNKLPYHVVKSKVSGALHAETILGESKGEITKRISVFDINYKNLGKIFDK |
| | DGSQKEIYETLVKWLEAKSKDYPKLKNGNIIKKVKIVDGNKDKLIKLGNKRY |
| | VEMGRTTVKILVLKKEAEEGLKFASIGRYKYNLLKSEKDVNISIWKDAIHFCK |
| | VRYNKLKENGYRLIHELIPGETIELELNKGNISKCLVVGFSGGKIEISSVIGDAL |
| | DLINDKISTRIKERYYITVSTIKSIKKINKNILGD |
| SEQ | MIRTLGIDIGIASIGWAVIEGEYTDKGLENKEIVASGVRVFTKAENPKNKESLA |
| ID | LPRTLARSARRRNARKKGRIQQVKHYLSKALGLDLECFVQGEKLATLFQTSK |
| NO: | DFLSPWELRERALYRVLDKEELARVILHIAKRRGYDDITYGVEDNDSGKIKK |
| 33 | AIAENSKRIKEEQCKTIGEMMYKLYFQKSLNVRNKKESYNRCVGRSELREEL |
| | KTIFQIQQELKSPWVNEELIYKLLGNPDAQSKQEREGLIFYQRPLKGFGDKIGK |
| | CSHIKKGENSPYRACKHAPSAEEFVALTKSINFLKNLTNRHGLCFSQEDMCV |
| | YLGKILQEAQKNEKGLTYSKLKLLLDLPSDFEFLGLDYSGKNPEKAVFLSLPS |
| | TFKLNKITQDRKTQDKIANILGANKDWEAILKELESLQLSKEQIQTIKDAKLNF |
| | SKHINLSLEALYHLLPLMREGKRYDEGVEILQERGIFSKPQPKNRQLLPPLSEL |
| | AKEESYFDIPNPVLRRALSEFRKVVNALLEKYGGFHYFHIELTRDVCKAKSAR |
| | MQLEKINKKNKSENDAASQLLEVLGLPNTYNNRLKCKLWKQQEEYCLYSGE |
| | KITIDHLKDQRALQIDHAFPLSRSLDDSQSNKVLCLTSSNQEKSNKTPYEWLG |
| | SDEKKWDMYVGRVYSSNFSPSKKRKLTQKNFKERNEEDFLARNLVDTGYIG |
| | RVTKEYIKHSLSFLPLPDGKKEHIRIISGSMTSTMRSFWGVQEKNRDHHLHHA |
| | QDAIIIACIEPSMIQKYTTYLKDKETHRLKSHQKAQILREGDHKLSLRWPMSN |
| | FKDKIQESIQNIIPSHHVSHKVTGELHQETVRTKEFYYQAFGGEEGVKKALKF |
| | GKIREINQGIVDNGAMVRVDIFKSKDKGKFYAVPIYTYDFAIGKLPNKAIVQG |
| | KKNGIIKDWLEMDENYEFCFSLFKNDCIKIQTKEMQEAVLAIYKSTNSAKATI |
| | ELEHLSKYALKNEDEEKMFTDTDKEKNKTMTRESCGIQGLKVFQKVKLSVL |
| | GEVLEHKPRNRQNIALKTTPKHV |
| SEQ | MAAFKPNSINYILGLDIGIASVGWAMVEIDEEENPIRLIDLGVRVFERAEVPKT |
| ID | GDSLAMARRLARSVRRLTRRRAHRLLRTRRLLKREGVLQAANFDENGLIKSL |
| NO: | PNTPWQLRAAALDRKLTPLEWSAVLLHLIKHRGYLSQRKNEGETADKELGA |
| 34 | LLKGVAGNAHALQTGDFRTPAELALNKFEKESGHIRNQRSDYSHTFSRKDLQ |
| | AELILLFEKQKEFGNPHVSGGLKEGIETLLMTQRPALSGDAVQKMLGHCTFEP |
| | AEPKAAKNTYTAERFIWLTKLNNLRILEQGSERPLTDTERATLMDEPYRKSKL |
| | TYAQARKLLGLEDTAFFKGLRYGKDNAEASTLMEMKAYHAISRALEKEGLK |
| | DKKSPLNLSPELQDEIGTAFSLFKTDEDITGRLKDRIQPEILEALLKHISFDKFV |
| | QISLKALRRIVPLMEQGKRYDEACAEIYGDHYGKKNTEEKIYLPPIPADEIRNP |
| | VVLRALSQARKVINGVVRRYGSPARIHIETAREVGKSFKDRKEIEKRQEENRK |
| | DREKAAAKFREYFPNFVGEPKSKDILKLRLYEQQHGKCLYSGKEINLGRLNE |
| | KGYVEIDHALPFSRTWDDSFNNKVLVLGSENQNKGNQTPYEYFNGKDNSRE |
| | WQEFKARVETSRFPRSKKQRILLQKFDEDGFKERNLNDTRYVNRFLCQFVAD |
| | RMRLTGKGKKRVFASNGQITNLLRGFWGLRKVRAENDRHHALDAVVVACS |
| | TVAMQQKITRFVRYKEMNAFDGKTIDKETGEVLHQKTHFPQPWEFFAQEVM |
| | IRVFGKPDGKPEFEEADTLEKLRTLLAEKLSSRPEAVHEYVTPLFVSRAPNRK |
| | MSGQGHMETVKSAKRLDEGVSVLRVPLTQLKLKDLEKMVNREREPKLYEAL |
| | KARLEAHKDDPAKAFAEPFYKYDKAGNRTQQVKAVRVEQVQKTGVWVRN |
| | HNGIADNATMVRVDVFEKGDKYYLVPIYSWQVAKGILPDRAVVQGKDEED |
| | WQLIDDSFNFKFSLHPNDLVEVITKKARMFGYFASCHRGTGNINIRIHDLDHK |
| | IGKNGILEGIGVKTALSFQKYQIDELGKEIRPCRLKKRPPVR |
| SEQ | MAAFKPNPINYILGLDIGIASVGWAMVEIDEDENPICLIDLGVRVFERAEVPKT |
| ID | GDSLAMARRLARSVRRLTRRRAHRLLRARRLLKREGVLQAADFDENGLIKSL |
| NO: | PNTPWQLRAAALDRKLTPLEWSAVLLHLIKHRGYLSQRKNEGETADKELGA |
| 35 | LLKGVADNAHALQTGDFRTPAELALNKFEKESGHIRNQRGDYSHTFSRKDLQ |
| | AELILLFEKQKEFGNPHVSGGLKEGIETLLMTQRPALSSDAVQKMLGHCTFEP |
| | AEPKAAKNTYTAERFIWLTKLNNLRILEQGSERPLTDTERATLMDEPYRKSKL |
| | TYAQARKLLGLEDTAFFKGLRYGKDNAEASTLMEMKAYHAISRALEKEGLK |
| | DKKSPLNLSPELQDEIGTAFSLFKTDEDITGRLKDRIQPEILEALLKHISFDKFV |
| | QISLKALRRIVPLMEQGKRYDEACAEIYGDHYGKKNTEEKIYLPPIPADEIRNP |
| | VVLRALSQARKVINGVVRRYGSPARIHIETAREVGKSFKDRKEIEKRQEENRK |
| | DREKAAAKFREYFPNFVGEPKSKDILKLRLYEQQHGKCLYSGKEINLGRLNE |
| | KGYVEIDHALPFSRTWDDSFNNKVLVLGSENQNKGNQTPYEYFNGKDNSRE |
| | WQEFKARVETSRFPRSKKQRILLQKFDEDGFKERNLNDTRYVNRFLCQFVAD |
| | RMRLTGKGKKRVFASNGQITNLLRGFWGLRKVRAENDRHHALDAVVVACS |
| | TVAMQQKITRFVRYKEMNAFDGKTIDKETGEVLHQKTHFPQPWEFFAQEVM |
| | IRVFGKPDGKPEFEEADTPEKLRTLLAEKLSSRPEAVHEYVTPLFVSRAPNRK |
| | MSGQGHMETVKSAKRLDEGVSVLRVPLTQLKLKDLEKMVNREREPKLYEAL |
| | KARLEAHKDDPAKAFAEPFYKYDKAGNRTQQVKAVRVEQVQKTGVWVRN |
| | HNGIADNATMVRVDVFEKGDKYYLVPIYSWQVAKGILPDRAVVQGKDEED |
| | WQLIDDSFNFKFSLHPNDLVEVITKKARMFGYFASCHRGTGNINIRIHDLDHK |
| | IGKNGILEGIGVKTALSFQKYQIDELGKEIRPCRLKKRPPVR |
| SEQ | MARILAFDIGISSIGWAFSENDELKDCGVRIFTKAENPKTGESLALPRRLARSA |
| ID | RKRLARRKARLNHLKHLIANEFKLNYEDYQSFDESLAKAYKGSLISPYELRFR |
| NO: | ALNELLSKQDFARVILHIAKRRGYDDIKNNGDEEKSEILKAIKQNEEKLVNYQ |
| 36 | SVGEYLYKEYFQKFKENSKEFINVRNKKESYERCIAQSFLKDELKLIFQKQRE |
| | FGFSFSKKFEEEVLSVAFYKRALKDFSHLVGNCSFFTDEKRAPKNSPLAFMFV |
| | ALTRIINLLNNLKNTEGILYTKDDLNTLLNEVLKNGTLTYKQTKKLLGLSDDY |
| | EFKGEKGTYFIEFKKYKEFIKALGEHNLSQDNLNEIAKDITLIKDEIKLKKALA |
| | KYDLNQNQIDSLSKLEFKDHLNISFKALKLITPLMLEGKKYDEAYNELNLKV |
| | AINEDKKDFLPAFNETYYKDEVTNPVVLRAIKEYRKVLNALLKKYGKVHKIN |
| | IELAREVGKNHSQRAKIEKEQNENYKAKKDAELECEKLGLKINSKNILKLRLF |
| | KEQKEFCAYSGEKIKISDLQDEKMLEIDHIYPYSRSFDDSYMNKVLVFTKQNQ |
| | EKLNQTPFEAFGNDSAKWQKIEVLAKNLPTKKQKRILDKNYKDKEQKDFKD |
| | RNLNDTRYIARLVLNYTKDYLDFLPLSDDENTKLNDTQKGSKVHVEAKSGM |
| | LTSALRHTWGFSAKDRNNHLHHAIDAVIIAYANNSIVKAFSDFKKEQESNSAE |
| | LYAKKISELDYKNKRKFFEPFSGFRQKVLDKIDEIFVSKPERKKPSGALHEETF |
| | RKEEEFYQSYGGKEGVLKALELGKIRKVNGKIVKNGDMFRVDIFKHKKTNK |
| | FYPVPIYTMDFALKVLPNKAVVQGKDKKSGLIKDWILMDENYEFCFSLYKDS |
| | LILIQTKDMQEPELVYFNAFTSSTVSLIVSKHDNKFETLSKNQKILFKNANEKE |
| | VIAKSIGIQNLKVFEKYIVSALGEVTKAEFRQREDFKK |
| SEQ | MARILAFDIGISSIGWAFSENDELKDCGVRIFTKAENPKTGESLALPRRLARSA |
| ID | RKRLARRKARLNHLKHLIANEFKLNYEDYQSFDESLAKAYKGSLISPYELRFR |
| NO: | ALNELLSKQDFARVILHIAKRRGYDDIKNSDDKEKGAILKAIKQNEEKLANY |
| 37 | QSVGEYLYKEYFQKFKENSKEFTNVRNKKESYERCIAQSFLKDELKLIFKKQR |
| | EFGFSFSKKFEEEVLSVAFYKRALKDFSHLVGNCSFFTDEKRAPKNSPLAFMF |
| | VALTRIINLLNNLKNTEGILYTKDDLNALLNEVLKNGTLTYKQTKKLLGLSD |
| | DYEFKGEKGTYFIEFKKYKEFIKALGDHSLSQDDLNEIAKDITLIKDEIKLKKA |
| | LAKYDLNQNQIDSLSKLEFKDHLNISFKALKLITPLMLEGKKYDEACNELNLK |
| | VAINEDKKDFLPAFNETYYKDEVTNPVVLRAIKEYRKVLNALLKKYGKVHKI |
| | NIELAREVGKNYSQRAKIEKEQNENYKAKKDAELECEKLGLKINSKNILKLRL |
| | FKEQKEFCAYSGEKIKISDLQDEKMLEIDHIYPYSRSFDDSYMNKVLVFTKQN |
| | QEKLNQTPFEAFGNDSAKWQKIEVLAKNLPTKKQKRILDKNYKDKEQKDFK |
| | DRNLNDTRYIARLVLNYTKDYLDFLPLSDDENTKLNDTQKGSKVHVEAKSG |
| | MLTSALRHTWGFSAKDRNNHLHHAIDAVIIAYANNSIVKAFSDFKKEQESNS |
| | AELYAKKISELDYKNKRKFFEPFSGFRQKVLDKIDEIFVSKPERKKPSGALHEE |
| | TFRKEEEFYQSYGGKEGVLKALELGKIRKVNGKIVKNGDMFRVDIFKHKKTN |
| | KFYAVPIYTMDFALKVLPNKAVARSKKGEIKDWILMDENYEFCFSLYKDSLI |
| | LIQTKDMQEPEFVYYNAFTSSTVSLIVSKHDNKFETLSKNQKILFKNANEKEVI |
| | AKSIGIQNLKVFEKYIVSALGEVTKAEFRQREGFKK |
| SEQ | MVRILAFDIGISSIGWAFSENDELKDCGVRIFTKVENPKTGESLALPRRLARSA |
| ID | RKRLARRKARLNHLKHLIANEFKLNYEDYQSFDESLAKAYKGSLISPYELRFR |
| NO: | ALNELLSKQDFARVILHIAKRRGYDDIKNSDDKEKGAILKAIKQNEEKLANY |
| 38 | QSVGEYLYKEYFQKFKENSKEFTNVRNKKESYERCIAQSFLKDELKLIFKKQR |
| | EFGFSFSKKFEEEVLSVAFYKRALKDFSHLVGNCSFFTDEKRAPKNSPLAFMF |
| | VALTRIINLLNNLKNTEGILYTKDDLNALLNEVLKNGTLTYKQTKKLLGLSD |
| | DYEFKGEKGTYFIEFKKYKEFIKALGEHNLSQDDLNEIAKDITLIKDEIKLKKA |
| | LAKYDLNQNQIDSLSKLEFKDHLNISFKALKLITPLMLEGKKYDEACNELNLK |
| | VAINEDKKDFLPAFNETYYKDEVTNPVVLRAIKEYRKVLNALLKKYGKVHKI |
| | NIELAREVGKNHSQRAKIEKEQNENYKAKKDAELECEKLGLKINSKNILKLRL |
| | FKEQKEFCAYSGEKIKISDLQDEKMLEIDHIYPYSRSFDDSYMNKVLVFTKQN |
| | QEKLNKTPFEAFGNDSAKWQKIEVLAKNLPTKKQKRILDKNYKDKEQKDFK |
| | DRNLNDTRYIARLVLNYTKDYLDFLPLSDDENTKLNDTQKGSKVHVEAKSG |
| | MLTSALRHTWGFSTKDRNNHLHHAIDAVIIAYANNSIVKAFSDFKKEQESNS |
| | AELYAKKISELDYKNKRKFFEPFSGFRQKVLDKIDEIFVSKPERKKPSGALHEE |
| | TFRKEEEFYQSYGGKEGVLKALELGKIRKVNGKIVKNGDMFRVDIFKHKKTN |
| | KFYAVPIYTMDFALKVLPNKAVARSKKGEIKDWILMDENYEFCFSLYKDSLI |
| | LIQTKDMQEPEFVYYNAFTSSTVSLIVSKHDNKFETLSKNQKILFKNANEKEVI |
| | AKSIGIQNLKVFEKYIVSALGEVTKAEFRQREDFKK |
| SEQ | MARILAFDIGISSIGWAFSENDELKDCGVRIFTKAENPKTGESLALPRRLARSA |
| ID | RKRLARRKARLNHLKHLIANEFKLNYEDYQSFDESLAKAYKGSLISPYELRFR |
| NO: | ALNELLSKQDFARVILHIAKRRGYDDIKNSDDKEKGAILKAIKQNEEKLANY |
| 39 | QSVGEYLYKEYFQKFKENSKEFTNVRNKKESYERCIAQSFLKDELKLIFKKQR |
| | EFGFSFSKKFEEEVLSVAFYKRALKDFSHLVGNCSFFTDEKRAPKNSPLAFMF |
| | VALTRIINLLNNLKNTEGILYTKDDLNALLNEVLKNGTLTYKQTKKLLGLSD |
| | DYEFKGEKGTYFIEFKKYKEFIKALGDHSLSQDDLNEIAKDITLIKDEIKLKKA |
| | LAKYDLNQNQIDSLSKLEFKDHLNISFKALKLITPLMLEGKKYDEACNELNLK |
| | VAINEDKKDFLPAFNETYYKDEVTNPVVLRAIKEYRKILNALLKKYGKVHKI |
| | NIELAREVGKNHSQRAKIEKEQNENYKAKKDAEIECEKLGLKINSKNILKLRL |
| | FKEQKEFCAYSGEKIKLSDLQDEKMLEIDHIYPYSRSFDDSYMNKVLVFTKQ |
| | NQEKLNKTPFEAFGNDSTKWQKIEVLAKNLPEKKQKRILDKNYKDKEQKDF |
| | KDRNLNDTRYIARLVLNYTKDYLDFLPLSDDENTKLNDTQKGSKVHVEAKS |
| | GMLTSALRHTWGFSAKDRNNHLHHAIDAAIIAYANNSIVKAFSDFKKEQESN |
| | SVELYAKKISELDYKNKRKFFEPFSGFRQKVLDKIDEIFVSKPERKKPSGALHE |
| | ETFRKEEEFYQSYGGKEGVLKALELGKIRKVNGKIVKNGDMFRVDIFKHKKT |
| | NKFYAVPIYTMDFALKVLPNKAVARSKKGEIKDWILMDENYEFCFSLYKDSL |
| | ILIQTKDMQEPELVYFNAFTSSTVSLIVSKHDNKFETLSKNQKILFKNANEKEV |
| | IAKSIGIQNLKVFEKYIVSALGEVTKAEFRQREDFKK |
| SEQ | MSIYQEFVNKYSLSKTLRFELIPQGKTLENIKARGLILDDEKRAKDYKKAKQII |
| ID | DKYHQFFIEEILSSVCISEDLLQNYSDVYFKLKKSDDDNLQKDFKSAKDTIKK |
| NO: | QISEYIKDSEKFKNLFNQNLIDAKKGQESDLILWLKQSKDNGIELFKANSDITD |
| 40 | IDEALEIIKSFKGWTTYFKGFHENRKNVYSSNDIPTSIIYRIVDDNLPKFLENKA |
| | KYESLKDKAPEAINYEQIKKDLAEELTFDIDYKTSEVNQRVFSLDEVFEIANFN |
| | NYLNQSGITKFNTIIGGKFVNGENTKRKGINEYINLYSQQINDKTLKKYKMSV |
| | LFKQILSDTESKSFVIDKLEDDSDVVTTMQSFYEQIAAFKTVEEKSIKETLSLLF |
| | DDLKAQKLDLSKIYFKNDKSLTDLSQQVEDDYSVIGTAVLEYITQQIAPKNLD |
| | NPSKKEQELIAKKTEKAKYLSLETIKLALEEFNKHRDIDKQCRFEEILANFAAI |
| | PMIFDEIAQNKDNLAQISIKYQNQGKKDLLQASAEDDVKAIKDLLDQTNNLL |
| | HKLKIFHISQSEDKANILDKDEHFYLVFEECYFELANIVPLYNKIRNYITQKPY |
| | SDEKFKLNFENSTLANGWDKNKEPDNTAILFIKDDKYYLGVMNKKNNKIFD |
| | DKAIKENKGEGYKKIVYKLLPGANKMLPKVFFSAKSIKFYNPSEDILRIRNHS |
| | THTKNGSPQKGYEKFEFNIEDCRKFIDFYKQSISKHPEWKDFGFRFSDTQRYN |
| | SIDEFYREVENQGYKLTFENISESYIDSVVNQGKLYLFQIYNKDFSAYSKGRPN |
| | LHTLYWKALFDERNLQDVVYKLNGEAELFYRKQSIPKKITHPAKEAIANKNK |
| | DNPKKESVFEYDLIKDKRFTEDKFFFHCPITINFKSSGANKFNDEINLLLKEKA |
| | NDVHILSIDRGERHLAYYTLVDGKGNIIKQDTFNIIGNDRMKTNYHDKLAAIE |
| | KDRDSARKDWKKINNIKEMKEGYLSQVVHEIAKLVIEYNAIVVFEDLNFGFK |
| | RGRFKVEKQVYQKLEKMLIEKLNYLVFKDNEFDKTGGVLRAYQLTAPFETF |
| | KKMGKQTGIIYYVPAGFTSKICPVTGFVNQLYPKYESVSKSQEFFSKFDKICY |
| | NLDKGYFEFSFDYKNFGDKAAKGKWTIASFGSRLINFRNSDKNHNWDTREV |
| | YPTKELEKLLKDYSIEYGHGECIKAAICGESDKKFFAKLTSVLNTILQMRNSK |
| | TGTELDYLISPVADVNGNFFDSRQAPKNMPQDADANGAYHIGLKGLMLLGRI |
| | KNNQEGKKLNLVIKNEEYFEFVQNRNN |
| SEQ | MENYQEFTNLFQLNKTLRFELKPIGKTCELLEEGKIFASGSFLEKDKVRADNV |
| ID | SYVKKEIDKKHKIFIEETLSSFSISNDLLKQYFDCYNELKAFKKDCKSDEEEVK |
| NO: | KTALRNKCTSIQRAMREAISQAFLKSPQKKLLAIKNLIENVFKADENVQHFSE |
| 41 | FTSYFSGFETNRENFYSDEEKSTSIAYRLVHDNLPIFIKNIYIFEKLKEQFDAKT |
| | LSEIFENYKLYVAGSSLDEVFSLEYFNNTLTQKGIDNYNAVIGKIVKEDKQEIQ |
| | GLNEHINLYNQKHKDRRLPFFISLKKQILSDREALSWLPDMFKNDSEVIKALK |
| | GFYIEDGFENNVLTPLATLLSSLDKYNLNGIFIRNNEALSSLSQNVYRNFSIDE |
| | AIDANAELQTFNNYELIANALRAKIKKETKQGRKSFEKYEEYIDKKVKAIDSL |
| | SIQEINELVENYVSEFNSNSGNMPRKVEDYFSLMRKGDFGSNDLIENIKTKLS |
| | AAEKLLGTKYQETAKDIFKKDENSKLIKELLDATKQFQHFIKPLLGTGEEADR |
| | DLVFYGDFLPLYEKFEELTLLYNKVRNRLTQKPYSKDKIRLCFNKPKLMTGW |
| | VDSKTEKSDNGTQYGGYLFRKKNEIGEYDYFLGISSKAQLFRKNEAVIGDYE |
| | RLDYYQPKANTIYGSAYEGENSYKEDKKRLNKVIIAYIEQIKQTNIKKSIIESIS |
| | KYPNISDDDKVTPSSLLEKIKKVSIDSYNGILSFKSFQSVNKEVIDNLLKTISPL |
| | KNKAEFLDLINKDYQIFTEVQAVIDEICKQKTFIYFPISNVELEKEMGDKDKPL |
| | CLFQISNKDLSFAKTFSANLRKKRGAENLHTMLFKALMEGNQDNLDLGSGAI |
| | FYRAKSLDGNKPTHPANEAIKCRNVANKDKVSLFTYDIYKNRRYMENKFLF |
| | HLSIVQNYKAANDSAQLNSSATEYIRKADDLHIIGIDRGERNLLYYSVIDMKG |
| | NIVEQDSLNIIRNNDLETDYHDLLDKREKERKANRQNWEAVEGIKDLKKGYL |
| | SQAVHQIAQLMLKYNAIIALEDLGQMFVTRGQKIEKAVYQQFEKSLVDKLSY |
| | LVDKKRPYNELGGILKAYQLASSITKNNSDKQNGFLFYVPAWNTSKIDPVTG |
| | FTDLLRPKAMTIKEAQDFFGAFDNISYNDKGYFEFETNYDKFKIRMKSAQTR |
| | WTICTFGNRIKRKKDKNYWNYEEVELTEEFKKLFKDSNIDYENCNLKEEIQN |
| | KDNRKFFDDLIKLLQLTLQMRNSDDKGNDYIISPVANAEGQFFDSRNGDKKL |
| | PLDADANGAYNIARKGLWNIRQIKQTKNDKKLNLSISSTEWLDFVREKPYLK |
| SEQ | MTQFEGFTNLYQVSKTLRFELIPQGKTLKHIQEQGFIEEDKARNDHYKELKPII |
| ID | DRIYKTYADQCLQLVQLDWENLSAAIDSYRKEKTEETRNALIEEQATYRNAI |
| NO: | HDYFIGRTDNLTDAINKRHAEIYKGLFKAELFNGKVLKQLGTVTTTEHENAL |
| 42 | LRSFDKFTTYFSGFYENRKNVFSAEDISTAIPHRIVQDNFPKFKENCHIFTRLIT |
| | AVPSLREHFENVKKAIGIFVSTSIEEVFSFPFYNQLLTQTQIDLYNQLLGGISRE |
| | AGTEKIKGLNEVLNLAIQKNDETAHIIASLPHRFIPLFKQILSDRNTLSFILEEFK |
| | SDEEVIQSFCKYKTLLRNENVLETAEALFNELNSIDLTHIFISHKKLETISSALC |
| | DHWDTLRNALYERRISELTGKITKSAKEKVQRSLKHEDINLQEIISAAGKELSE |
| | AFKQKTSEILSHAHAALDQPLPTTLKKQEEKEILKSQLDSLLGLYHLLDWFAV |
| | DESNEVDPEFSARLTGIKLEMEPSLSFYNKARNYATKKPYSVEKFKLNFQMPT |
| | LASGWDVNKEKNNGAILFVKNGLYYLGIMPKQKGRYKALSFEPTEKTSEGF |
| | DKMYYDYFPDAAKMIPKCSTQLKAVTAHFQTHTTPILLSNNFIEPLEITKEIYD |
| | LNNPEKEPKKFQTAYAKKTGDQKGYREALCKWIDFTRDFLSKYTKTTSIDLS |
| | SLRPSSQYKDLGEYYAELNPLLYHISFQRIAEKEIMDAVETGKLYLFQIYNKD |
| | FAKGHHGKPNLHTLYWTGLFSPENLAKTSIKLNGQAELFYRPKSRMKRMAH |
| | RLGEKMLNKKLKDQKTPIPDTLYQELYDYVNHRLSHDLSDEARALLPNVITK |
| | EVSHEIIKDRRFTSDKFFFHVPITLNYQAANSPSKFNQRVNAYLKEHPETPIIGI |
| | DRGERNLIYITVIDSTGKILEQRSLNTIQQFDYQKKLDNREKERVAARQAWSV |
| | VGTIKDLKQGYLSQVIHEIVDLMIHYQAVVVLENLNFGFKSKRTGIAEKAVY |
| | QQFEKMLIDKLNCLVLKDYPAEKVGGVLNPYQLTDQFTSFAKMGTQSGFLF |
| | YVPAPYTSKIDPLTGFVDPFVWKTIKNHESRKHFLEGFDFLHYDVKTGDFILH |
| | FKMNRNLSFQRGLPGFMPAWDIVFEKNETQFDAKGTPFIAGKRIVPVIENHRF |
| | TGRYRDLYPANELIALLEEKGIVFRDGSNILPKLLENDDSHAIDTMVALIRSVL |
| | QMRNSNAATGEDYINSPVRDLNGVCFDSRFQNPEWPMDADANGAYHIALKG |
| | QLLLNHLKESKDLKLQNGISNQDWLAYIQELRN |
| SEQ | MNIKNFTGLYPLSKTLRFELKPIGKTKENIEKNGILTKDEQRAKDYLIVKGFID |
| ID | EYHKQFIKDRLWDFKLPLESEGEKNSLEEYQELYELTKRNDAQEADFTEIKD |
| NO: | NLRSSITEQLTKSGSAYDRIFKKEFIREDLVNFLEDEKDKNIVKQFEDFTTYFT |
| 43 | GFYENRKNMYSSEEKSTAIAYRLIHQNLPKFMDNMRSFAKIANSSVSEHFSDI |
| | YESWKEYLNVNSIEEIFQLDYFSETLTQPHIEVYNYIIGKKVLEDGTEIKGINEY |
| | VNLYNQQQKDKSKRLPFLVPLYKQILSDREKLSWIAEEFDSDKKMLSAITESY |
| | NHLHNVLMGNENESLRNLLLNIKDYNLEKINITNDLSLTEISQNLFGRYDVFT |
| | NGIKNKLRVLTPRKKKETDENFEDRINKIFKTQKSFSIAFLNKLPQPEMEDGKP |
| | RNIEDYFITQGAINTKSIQKEDIFAQIENAYEDAQVFLQIKDTDNKLSQNKTAV |
| | EKIKTLLDALKELQHFIKPLLGSGEENEKDELFYGSFLAIWDELDTITPLYNKV |
| | RNWLTRKPYSTEKIKLNFDNAQLLGGWDVNKEHDCAGILLRKNDSYYLGIIN |
| | KKTNHIFDTDITPSDGECYDKIDYKLLPGANKMLPKVFFSKSRIKEFEPSEAIIN |
| | CYKKGTHKKGKNFNLTDCHRLINFFKTSIEKHEDWSKFGFKFSDTETYEDISG |
| | FYREVEQQGYRLTSHPVSASYIHSLVKEGKLYLFQIWNKDFSQFSKGTPNLHT |
| | LYWKMLFDKRNLSDVVYKLNGQAEVFYRKSSIEHQNRIIHPAQHPITNKNEL |
| | NKKHTSTFKYDIIKDRRYTVDKFQFHVPITINFKATGQNNINPIVQEVIRQNGIT |
| | HIIGIDRGERHLLYLSLIDLKGNIIKQMTLNEIINEYKGVTYKTNYHNLLEKRE |
| | KERTEARHSWSSIESIKELKDGYMSQVIHKITDMMVKYNAIVVLEDLNGGFM |
| | RGRQKVEKQVYQKFEKKLIDKLNYLVDKKLDANEVGGVLNAYQLTNKFESF |
| | KKIGKQSGFLFYIPAWNTSKIDPITGFVNLFNTRYESIKETKVFWSKFDIIRYNK |
| | EKNWFEFVFDYNTFTTKAEGTRTKWTLCTHGTRIQTFRNPEKNAQWDNKEI |
| | NLTESFKALFEKYKIDITSNLKESIMQETEKKFFQELHNLLHLTLQMRNSVTG |
| | TDIDYLISPVADEDGNFYDSRINGKNFPENADANGAYNIARKGLMLIRQIKQA |
| | DPQKKFKFETITNKDWLKFAQDKPYLKD |

In some cases, the dCas comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 1-SEQ ID NO: 43. In some cases, the dCas comprises a sequence of any of SEQ ID NO: 1-SEQ ID NO: 43.

In some cases, the dCas comprises a mutated Cas protein. In some cases, the dCas is a truncated Cas protein. In some cases, dCas truncations are utilized to repress or activate genes. These truncations include but are not limited to, REC2 domain deletion, REC3 domain deletion, HNH deletion, and deletions of the domains of the nuclease (NUC) lobe, or any combination of the aforementioned domains.

In some cases, the dCas comprises dCas9. In some cases, the dCas9 is a truncated or mutated Cas9 protein. In some cases, the dCas comprises dCas12. In some cases, the dCas12 is a truncated or mutated Cas12 protein.

In some cases, the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteurianus, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9.

For compositions and methods described herein using a CRISPR/Cas system, the nucleic acid comprises and/or is administered with one or more guide RNA that binds to one or more target molecules. In some cases, the one or more guide RNA is 2, 3, 4, or 5 guide RNA sequences.

In some embodiments, a guide RNA sequence may target the promoter used to drive the expression of the final construct, creating a regulatory feedback loop to control the expression levels of the therapeutic. A guide RNA may bind to a target sequence and a Cas protein (e.g., a dead Cas protein) and may deliver the Cas protein to the target sequence. Examples of guide RNAs that may target Naᵥ1.7 or Naᵥ1.8 are provided in TABLE 2.

TABLE 2
Exemplary Guide RNA Sequences

| | | Gene |
|---|---|---|
| SEQ ID NO | Sequence | Target |
| SEQ ID NO: 55 | GGCTGCTAGCGGCAGGCGTCCC | SCN9A |
| SEQ ID NO: 56 | GCAGAATCTGGCTCCAGGAGAG | SCN9A |
| SEQ ID NO: 57 | GGTGGGGATGATCGGCGGGCTA | SCN9A |
| SEQ ID NO: 58 | TAATTCCTCTTCAGCTCCTCAC | SCN9A |
| SEQ ID NO: 59 | AGGGCTCTTCTGGCTGCTGGAC | SCN9A |
| SEQ ID NO: 60 | GGGACGCCTGCCGCTAGCAGCC | SCN9A |
| SEQ ID NO: 61 | TCCCCACAGAAGCAGCAAAAGA | SCN9A |
| SEQ ID NO: 62 | TTTCGCCCGTGCTCGCCTCAAC | SCN9A |
| SEQ ID NO: 63 | GTTTTTCTCCAGCTCCCCACCA | SCN9A |
| SEQ ID NO: 64 | AAGGGAAGGGCTCTTCTGGCTG | SCN9A |
| SEQ ID NO: 65 | GTTCCAGAACCGAGATGTGAAA | SCN9A |
| SEQ ID NO: 66 | TTTTCTCCAGCTCCCCACCA | SCN9A |
| SEQ ID NO: 67 | CCTCTCTTCTTTCCAGGTGG | SCN9A |
| SEQ ID NO: 68 | GGGGTCCAAGTCCTCCAGGGGC | SCN9A |
| SEQ ID NO: 69 | CCCAGCGCGCCCGGGTCCCC | SCN9A |
| SEQ ID NO: 70 | GCGCGCTGGGAGGAGTGGAG | SCN9A |
| SEQ ID NO: 71 | GCTAGCCCAGCCTCAGCCGA | SCN9A |
| SEQ ID NO: 72 | TGCACCTGCAGAATCTGGCT | SCN9A |
| SEQ ID NO: 73 | ACCCAGTGCACCTGCAGAAT | SCN9A |
| SEQ ID NO: 74 | CAGGTGCACTGGGTGGGGAT | SCN9A |
| SEQ ID NO: 75 | GCCTCTTATGTGAGGAGCTG | SCN9A |
| SEQ ID NO: 76 | CTTCAGCTCCTCACATAAGA | SCN9A |
| SEQ ID NO: 77 | TATATTTTAATTCCTCTTCA | SCN9A |
| SEQ ID NO: 78 | CCCGGCATGGTGTCAGAGCC | SCN9A |
| SEQ ID NO: 79 | GGCGGTCGCCAGCGCTCCAG | SCN9A |
| SEQ ID NO: 80 | GCCACCTGGAAAGAAGAGAG | SCN9A |
| SEQ ID NO: 81 | GGTCGCCAGCGCTCCAGCGG | SCN9A |
| SEQ ID NO: 82 | GCCAGCAATGGGAGGAAGAA | SCN9A |
| SEQ ID NO: 83 | GTTCCAGGTGGCGTAATACA | SCN9A |
| SEQ ID NO: 84 | GGCGGGGCTGCTACCTCCAC | SCN9A |
| SEQ ID NO: 85 | GGGCGCAGTCTGCTTGCAGG | SCN9A |
| SEQ ID NO: 86 | GGCGCTCCAGCGGCGGCTGT | SCN9A |
| SEQ ID NO: 87 | GACCGGGTGGTTCCAGCAAT | SCN9A |
| SEQ ID NO: 88 | GGGGTGGTTCCAGCAATGGG | SCN9A |
| SEQ ID NO: 89 | ACAGTGGGCAGGATTGAAA | SCN9A |
| SEQ ID NO: 90 | GCAGGTGCACTCACCGGGT | SCN9A |
| SEQ ID NO: 91 | GAGCTCAGGGAGCATCGAGG | SCN9A |
| SEQ ID NO: 92 | AGAGTCGCAATTGGAGCGC | SCN9A |
| SEQ ID NO: 93 | CCAGACCAGCCTGCACAGT | SCN9A |
| SEQ ID NO: 94 | GAGCGCAGGCTAGGCCTGCA | SCN9A |
| SEQ ID NO: 95 | CTAGGAGTCCGGGATACCC | SCN9A |
| SEQ ID NO: 96 | GAATCCGCAGGTGCACTCAC | SCN9A |
| SEQ ID NO: 97 | GACCAGCCTGCACAGTGGGC | SCN9A |
| SEQ ID NO: 98 | GCGACGCGGTTGGCAGCCGA | SCN9A |
| SEQ ID NO: 99 | GTTTACTCCGGAGTCACTGG | SCN10A |
| SEQ ID NO: 100 | GCTATCTCCACCAGTGACTC | SCN10A |
| SEQ ID NO: 101 | GACATCACCCAGGGCCAAGG | SCN10A |
| SEQ ID NO: 102 | GTAGTTTCGAGGGATCCAAT | SCN10A |
| SEQ ID NO: 103 | GCTCCCAGCAGAACTGATCG | SCN10A |
| SEQ ID NO: 104 | GTGACTCCGGAGTAAAGCGA | SCN10A |
| SEQ ID NO: 105 | GGGAGCTCACCATAGAACTT | SCN10A |
| SEQ ID NO: 106 | GACGGATCTAGATCCTCCAG | SCN10A |
| SEQ ID NO: 107 | GCCGGGTAAGAGCTACTAGT | SCN10A |
| SEQ ID NO: 108 | GCCCGGTGTGTGCTGTAGAA | SCN10A |
| SEQ ID NO: 268 | GGCAGGGTGGAACTCGTGAC | SCN10A |
| SEQ ID NO: 269 | GCACCATCCAGCAAGCAGGG | SCN10A |
| SEQ ID NO: 270 | GCGTCACTCAAGGATCTACA | SCN10A |
| SEQ ID NO: 271 | GATGGGAATGGCACCCACGA | SCN10A |
| SEQ ID NO: 272 | GCCTTTAGACGGAGAACAGA | SCN10A |
| SEQ ID NO: 273 | GAGATCCTTGAGTGACGGAC | SCN10A |
| SEQ ID NO: 274 | GCGGGGCTCCTCCACGAAGG | SCN10A |
| SEQ ID NO: 275 | GCAAGGAATCACGCCTTCGT | SCN10A |
| SEQ ID NO: 276 | GGCCATGCGCGAATGCTGAG | SCN10A |
| SEQ ID NO: 277 | GGCAAGCCCAGCCACCTTCG | SCN10A |

In some cases, a guide RNA targeting Naᵥ1.7 comprises a sequence having at least 50% at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 55-SEQ ID NO: 98. In some cases, the guide RNA targeting Naᵥ1.7 comprises a sequence of any of SEQ ID NO: 55-SEQ ID NO: 98. In some cases, a guide RNA targeting Naᵥ1.8 comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 99-SEQ ID NO: 108 or SEQ ID NO: 268-SEQ ID NO: 277. In some cases, the guide RNA targeting Naᵥ1.8 comprises a sequence of any of SEQ ID NO: 99-SEQ ID NO: 108 or SEQ ID NO: 268-SEQ ID NO: 277.

### Zinc Finger Proteins

Zinc finger proteins (ZFP) comprise a DNA-binding domain made up of Cys2His2 zinc fingers. ZFPs constitute the largest individual family of transcriptional modulators encoded by the genomes of higher organisms.

In some embodiments, the nucleic acid compositions comprise a sequence encoding a ZFP. The ZFP may comprise a native or modified sequence. Non-limiting examples of ZFP sequences are provided in TABLE 3.

TABLE 3
Exemplary Zinc Finger Proteins

| SEQ | | Gene |
|---|---|---|
| ID NO | Sequence | Target |
| SEQ ID | MAERPFQCRICMRNFSTSGHLSRHIRTHTGEKPFACDICGRKFAD | SCN9A |
| NO: | RSHLARHTKIHTGSQKPFQCRICMRNFSQSGDLTRHIRTHTGEKP | |
| 134 | FACDICGRKFAYRWLRNNHTKIHTGSQKPFQCRICMRNFSRSDT | |
| | LSEHIRTHTGEKPFACDICGRKFARAQHLQQHTKIHLRQKDAAR | |
| SEQ ID | MAERPFQCRICMRNFSDRSHLSRHIRTHTGEKPFACDICGRKFAD | SCN9A |
| NO: | RSALARHTKIHTGSQKPFQCRICMRNFSQSSDLSRHIRTHTGEKP | |
| 135 | FACDICGRKFARSDDLTRHTKIHTGSQKPFQCRICMRNFSRSDDL | |
| | TRHIRTHTGEKPFACDICGRKFAQRSTLSSHTKIHLRQKDAAR | |
| SEQ ID | MAERPFQCRICMRNFSQSSDLSRHIRTHTGEKPFACDICGRKFAR | SCN9A |
| NO: | SDALARHTKIHTGSQKPFQCRICMRNFSQSGDLTRHIRTHTGEKP | |
| 136 | FACDICGRKFARSWGLQVHTKIHTGSQKPFQCRICMRNFSRSDH | |
| | LSQHIRTHTGEKPFACDICGRKFADSSTRKKHTKIHLRQKDAAR | |
| SEQ ID | MAERPFQCRICMRNFSDRSHLTRHIRTHTGEKPFACDICGRKFAQ | SCN9A |
| NO: | SGDLTRHTKIHTGSQKPFQCRICMRNFSRSDDLTRHIRTHTGEKP | |
| 137 | FACDICGRKFAQRSTLSSHTKIHTGSQKPFQCRICMRNFSRSDVL | |
| | SEHIRTHTGEKPFACDICGRKFARNQHRKTHTKIHLRQKDAAR | |
| SEQ ID | MAERPFQCRICMRNFSRSDNLSEHIRTHTGEKPFACDICGRKFAE | SCN9A |
| NO: | RANRNSHTKIHTGSQKPFQCRICMRNFSRSDNLARHIRTHTGEKP | |
| 138 | FACDICGRKFAWRGDRVKHTKIHTGSQKPFQCRICMRNFSDRSD | |
| | LSRHIRTHTGEKPFACDICGRKFARRTDLRRHTKIHLRQKDAAR | |
| SEQ ID | MAERPFQCRICMRNFSDRSDLSRHIRTHTGEKPFACDICGRKFAR | SCN9A |
| NO: | RTDLRRHTKIHTGSQKPFQCRICMRNFSDRSDLSRHIRTHTGEKP | |
| 139 | FACDICGRKFARSHHLKAHTKIHTGSQKPFQCRICMRNFSRSANL | |
| | ARHIRTHTGEKPFACDICGRKFARSDNLREHTKIHLRQKDAAR | |
| SEQ ID | MAERPFQCRICMRNFSRSAHLSRHIRTHTGEKPFACDICGRKFAT | SCN9A |
| NO: | SGHLSRHTKIHTGSQKPFQCRICMRNFSRSDALSEHIRTHTGEKP | |
| 140 | FACDICGRKFAQNATRTKHTKIHTGSQKPFQCRICMRNFSTSGHL | |
| | SRHIRTHTGEKPFACDICGRKFAQSGDLTRHTKIHLRQKDAAR | |
| SEQ ID | MAERPFQCRICMRNFSQSGDLTRHIRTHTGEKPFACDICGRKFA | SCN9A |
| NO: | QSGARNIHTKIHTGSQKPFQCRICMRNFSRSANLARHIRTHTGEK | |
| 141 | PFACDICGRKFARSDALARHTKIHTGSQKPFQCRICMRNFSDRSD | |
| | LSRHIRTHTGEKPFACDICGRKFARRTDLRRHTKIHLRQKDAAR | |
| SEQ ID | MAERPFQCRICMRNFSRSDVLSRHIRTHTGEKPFACDICGRKFAD | SCN9A |
| NO: | SRDRKNHTKIHTGSQKPFQCRICMRNFSRSADLTRHIRTHTGEKP | |
| 142 | FACDICGRKFADRSHLARHTKIHTGSQKPFQCRICMRNFSRSDNL | |
| | SEHIRTHTGEKPFACDICGRKFASKQYLIKHTKIHLRQKDAAR | |
| SEQ ID | MAERPFQCRICMRNFSRSADLTRHIRTHTGEKPFACDICGRKFAQ | SCN9A |
| NO: | SGHLSRHTKIHTGSQKPFQCRICMRNFSQSSDLSRHIRTHTGEKPF | |
| 143 | ACDICGRKFAHRKSLSRHTKIHTGSQKPFQCRICMRNFSDRSDLS | |
| | RHIRTHTGEKPFACDICGRKFAQSSTRARHTKIHLRQKDAAR | |
| SEQ ID | MAERPFQCRICMRNFSDRSHLTRHIRTHTGEKPFACDICGRKFAR | SCN9A |
| NO: | SDDLTRHTKIHTGSQKPFQCRICMRNFSDRSHLTRHIRTHTGEKP | |
| 144 | FACDICGRKFARSDNLTRHTKIHTGSQKPFQCRICMRNFSQSGHL | |
| | ARHIRTHTGEKPFACDICGRKFAQKGTLGEHTKIHLRQKDAAR | |
| SEQ ID | MAERPFQCRICMRNFSRSAHLSRHIRTHTGEKPFACDICGRKFAQ | SCN9A |
| NO: | SGNLARHTKIHTGSQKPFQCRICMRNFSRSDAMSQHIRTHTGEK | |
| 145 | PFACDICGRKFARNASRTRHTKIHTGSQKPFQCRICMRNFSRSAN | |
| | LARHIRTHTGEKPFACDICGRKFADRSHLARHTKIHLRQKDAAR | |
| SEQ ID | MAERPFQCRICMRNFSRSANLARHIRTHTGEKPFACDICGRKFA | SCN9A |
| NO: | DSSDRKKHTKIHTGSQKPFQCRICMRNFSTSGSLSRHIRTHTGEK | |
| 146 | PFACDICGRKFAHSLSLKNHTKIHTGSQKPFQCRICMRNFSQSSD | |
| | LSRHIRTHTGEKPFACDICGRKFAWKWNLRAHTKIHLRQKDAA | |
| | R | |
| SEQ ID | MAERPFQCRICMRNFSRSAHLSRHIRTHTGEKPFACDICGRKFAT | SCN9A |
| NO: | SGHLSRHTKIHTGSQKPFQCRICMRNFSRSDHLSQHIRTHTGEKP | |
| 147 | FACDICGRKFAASSTRTKHTKIHTGSQKPFQCRICMRNFSQSSHL | |
| | TRHIRTHTGEKPFACDICGRKFARSDNLTRHTKIHLRQKDAAR | |
| SEQ ID | MAERPFQCRICMRNFSDRSHLTRHIRTHTGEKPFACDICGRKFAD | SCN9A |
| NO: | RSHLARHTKIHTGSQKPFQCRICMRNFSRSDNLSEHIRTHTGEKP | |
| 148 | FACDICGRKFARSAALARHTKIHTGSQKPFQCRICMRNFSRSDTL | |
| | SQHIRTHTGEKPFACDICGRKFATRDHRIKHTKIHLRQKDAAR | |
| SEQ ID | MAERPFQCRICMRNFSRSDNLARHIRTHTGEKPFACDICGRKFAT | SCN9A |
| NO: | SGSLTRHTKIHTGSQKPFQCRICMRNFSRSDDLSKHIRTHTGEKP | |
| 278 | FACDICGRKFARSDALARHTKIHTGSQKPFQCRICMRNESTSGHL | |
| | SRHIRTHTGEKPFACDICGRKFARSDALARHTKIHLRQKDAAR | |
| SEQ ID | MAERPFQCRICMRNFSRSDALSEHIRTHTGEKPFACDICGRKFAR | SCN9A |
| NO: | SADRTRHTKIHTGSQKPFQCRICMRNFSQSGHLARHIRTHTGEKP | |
| 279 | FACDICGRKFAHRSTLSRHTKIHTGSQKPFQCRICMRNFSDRSDL | |
| | SRHIRTHTGEKPFACDICGRKFADRSDLSRHTKIHLRQKDAAR | |
| SEQ ID | MAERPFQCRICMRNFSQSGDLTRHIRTHTGEKPFACDICGRKFA | SCN9A |
| NO: | QSGARNIHTKIHTGSQKPFQCRICMRNFSRSANLARHIRTHTGEK | |
| 280 | PFACDICGRKFARSDALARHTKIHTGSQKPFQCRICMRNFSDRSD | |
| | LSRHIRTHTGEKPFACDICGRKFARRTDLRRHTKIHLRQKDAAR | |
| | GS | |
| SEQ ID | MAERPFQCRICMRNFSRSDNLSRHIRTHTGEKPFACDICGRKFAH | SCN9A |
| NO: | SATRKRHTKIHTGSQKPFQCRICMRNFSRSDDLSKHIRTHTGEKP | |
| 281 | FACDICGRKFARSDALARHTKIHTGSQKPFQCRICMRNFSRSSHL | |
| | ARHIRTHTGEKPFACDICGRKFARSDALARHTKIHLRQKDAAR | |
| SEQ ID | MAERPFQCRICMRNFSRSDTLTRHIRTHTGEKPFACDICGRKFAR | SCN9A |
| NO: | SDARTNHTKIHTGSQKPFQCRICMRNFSRSSDLTRHIRTHTGEKP | |
| 282 | FACDICGRKFAQSGHLSRHTKIHTGSQKPFQCRICMRNFSRSDTL | |
| | TQHIRTHTGEKPFACDICGRKFADSSDLSRHTKIHLRQKDAAR | |
| SEQ ID | MAERPFQCRICMRNFSRSDDLTRHIRTHTGEKPFACDICGRKFAR | SCN9A |
| NO: | SDNLARHTKIHTGSQKPFQCRICMRNFSQSSALSRHIRTHTGEKP | |
| 283 | FACDICGRKFARSDHLSRHTKIHTGSQKPFQCRICMRNFSRSDSL | |
| | ARHIRTHTGEKPFACDICGRKFANRHHLTRHTKIHLRQKDAAR | |
| SEQ ID | MAERPFQCRICMRNFSDRSSLTTHIRTHTGEKPFACDICGRKFAD | SCN9A |
| NO: | RSHLARHTKIHTGSQKPFQCRICMRNFSRSDNLARHIRTHTGEKP | |
| 284 | FACDICGRKFAQSAHLARHTKIHTGSQKPFQCRICMRNFSQSGN | |
| | LARHIRTHTGEKPFACDICGRKFARSDHLSRHTKIHLRQKDAAR | |
| SEQ ID | MAERPFQCRICMRNFSRSDNLSEHIRTHTGEKPFACDICGRKFAQ | SCN9A |
| NO: | SANRNKHTKIHTGSQKPFQCRICMRNFSRSDNLARHIRTHTGEKP | |
| 285 | FACDICGRKFAWRGDRVKHTKIHTGSQKPFQCRICMRNFSDRSD | |
| | LSRHIRTHTGEKPFACDICGRKFARRTDLRRHTKIHLRQKDAAR | |
| SEQ ID | MAERPFQCRICMRNFSQSGSLSRHIRTHTGEKPFACDICGRKFAR | SCN9A |
| NO: | SDDLSRHTKIHTGSQKPFQCRICMRNFSRSDSLSQHIRTHTGEKPF | |
| 286 | ACDICGRKFARSGHLSRHTKIHTGSQKPFQCRICMRNFSDRSDLS | |
| | RHIRTHTGEKPFACDICGRKFAQSSTLARHTKIHLRQKDAAR | |
| SEQ ID | MAERPFQCRICMRNFSDQSTLRSHIRTHTGEKPFACDICGRKFAD | SCN9A |
| NO: | RSNLSRHTKIHTGSQKPFQCRICMRNFSRSDALSEHIRTHTGEKPF | |
| 287 | ACDICGRKFARSADRKRHTKIHTGSQKPFQCRICMRNFSQSGHL | |
| | ARHIRTHTGEKPFACDICGRKFATSSTLSKHTKIHLRQKDAAR | |
| SEQ ID | MAERPFQCRICMRNFSRSDHLSRHIRTHTGEKPFACDICGRKFAT | SCN9A |
| NO: | SSHLARHTKIHTGSQKPFQCRICMRNFSTSGNLARHIRTHTGEKP | |
| 288 | FACDICGRKFATSGNLVRHTKIHTGSQKPFQCRICMRNFSRSDHL | |
| | TRHIRTHTGEKPFACDICGRKFATSGHLVRHTKIHLRQKDAAR | |
| SEQ ID | MAERPFQCRICMRNFSRSDNLARHIRTHTGEKPFACDICGRKFA | SCN9A |
| NO: | RSDHRKKHTKIHTGSQKPFQCRICMRNFSQSGNLARHIRTHTGE | |
| 289 | KPFACDICGRKFARSGNLRAHTKIHTGSQKPFQCRICMRNFSDSS | |
| | ALSTHIRTHTGEKPFACDICGRKFARSDHLSQHTKIHLRQKDAAR | |
| SEQ ID | MAERPFQCRICMRNFSERSDLSVHIRTHTGEKPFACDICGRKFAD | SCN9A |
| NO: | RDDLSRHTKIHTGSQKPFQCRICMRNFSRSDDRKVHIRTHTGEKP | |
| 290 | FACDICGRKFADRSHRIRHTKIHTGSQKPFQCRICMRNFSRSDNL | |
| | ARHIRTHTGEKPFACDICGRKFAQSGHLARHTKIHLRQKDAAR | |
| SEQ ID | MAERPFQCRICMRNFSQSSALTRHIRTHTGEKPFACDICGRKFAR | SCN10A |
| NO: | SDNLRAHTKIHTGSQKPFQCRICMRNFSQSGDLTRHIRTHTGEKP | |
| 149 | FACDICGRKFAQSGDLTRHTKIHTGSQKPFQCRICMRNFSQSGHL | |
| | VTHIRTHTGEKPFACDICGRKFATSGNLVRHTKIHLRQKDAAR | |
| SEQ ID | MAERPFQCRICMRNFSERSALATHIRTHTGEKPFACDICGRKFAD | SCN10A |
| NO: | RSALARHTKIHTGSQKPFQCRICMRNFSQSAHLSRHIRTHTGEKP | |
| 150 | FACDICGRKFARSDALARHTKIHTGSQKPFQCRICMRNFSQSGDL | |
| | TRHIRTHTGEKPFACDICGRKFATSGNLSRHTKIHLRQKDAAR | |
| SEQ ID | MAERPFQCRICMRNFSDRSALTRHIRTHTGEKPFACDICGRKFAQ | SCN10A |
| NO: | SGDLTRHTKIHTGSQKPFQCRICMRNFSRSDSLTRHIRTHTGEKP | |
| 151 | FACDICGRKFATSGNLTRHTKIHTGSQKPFQCRICMRNFSTSGHL | |
| | TRHIRTHTGEKPFACDICGRKFARSDALREHTKIHLRQKDAAR | |
| SEQ ID | MAERPFQCRICMRNFSQSGDLTRHIRTHTGEKPFACDICGRKFA | SCN10A |
| NO: | QSGDLTRHTKIHTGSQKPFQCRICMRNFSQSGHLSTHIRTHTGEK | |
| 152 | PFACDICGRKFATSGNLSRHTKIHTGSQKPFQCRICMRNFSQSGS | |
| | LRAHIRTHTGEKPFACDICGRKFARSDNLSKHTKIHLRQKDAAR | |
| SEQ ID | MAERPFQCRICMRNFSDRSHLTTHIRTHTGEKPFACDICGRKFAD | SCN10A |
| NO: | RSALTRHTKIHTGSQKPFQCRICMRNFSQSGDLTRHIRTHTGEKP | |
| 153 | FACDICGRKFARSDALAAHTKIHTGSQKPFQCRICMRNFSTSGNL | |
| | TRHIRTHTGEKPFACDICGRKFATSGHLTRHTKIHLRQKDAAR | |
| SEQ ID | MAERPFQCRICMRNFSRSDSLSVHIRTHTGEKPFACDICGRKFAQ | SCN10A |
| NO: | SGDLTRHTKIHTGSQKPFQCRICMRNFSTSGNLTRHIRTHTGEKP | |
| 154 | FACDICGRKFAQSTHLTSHTKIHTGSQKPFQCRICMRNFSQSGHL | |
| | ARHIRTHTGEKPFACDICGRKFAQLTHLNSHTKIHLRQKDAAR | |
| SEQ ID | MAERPFQCRICMRNFSDRSALSRHIRTHTGEKPFACDICGRKFAR | SCN10A |
| NO: | SQHLQTHTKIHTGSQKPFQCRICMRNFSDRSALSDHIRTHTGEKP | |
| 155 | FACDICGRKFAQSGNLARHTKIHTGSQKPFQCRICMRNFSRSDNL | |
| | ARHIRTHTGEKPFACDICGRKFAQSGHLARHTKIHLRQKDAAR | |
| SEQ ID | MAERPFQCRICMRNFSQSSNLSRHIRTHTGEKPFACDICGRKFAR | SCN10A |
| NO: | SDHLSTHTKIHTGSQKPFQCRICMRNFSRSDTLSEHIRTHTGEKPF | |
| 156 | ACDICGRKFAQSATLTRHTKIHTGSQKPFQCRICMRNFSRSDNLA | |
| | RHIRTHTGEKPFACDICGRKFAQSGALSRHTKIHLRQKDAAR | |
| SEQ ID | MAERPFQCRICMRNFSERGTLARHIRTHTGEKPFACDICGRKFAR | SCN10A |
| NO: | SDHRKTHTKIHTGSQKPFQCRICMRNFSDRSNLTRHIRTHTGEKP | |
| 157 | FACDICGRKFARSDNLARHTKIHTGSQKPFQCRICMRNFSRSDNL | |
| | ARHIRTHTGEKPFACDICGRKFATSANLVRHTKIHLRQKDAAR | |
| SEQ ID | MAERPFQCRICMRNFSDRSNLSRHIRTHTGEKPFACDICGRKFAR | SCN10A |
| NO: | SDNLTRHTKIHTGSQKPFQCRICMRNFSRSDTLSVHIRTHTGEKP | |
| 158 | FACDICGRKFADNSTRIKHTKIHTGSQKPFQCRICMRNFSRSDHL | |
| | SNHIRTHTGEKPFACDICGRKFADNRDRIKHTKIHLRQKDAAR | |
| SEQ ID | MAERPFQCRICMRNFSDRSNLSRHIRTHTGEKPFACDICGRKFAQ | SCN10A |
| NO: | LASRTKHTKIHTGSQKPFQCRICMRNFSRSDNLSAHIRTHTGEKP | |
| 159 | FACDICGRKFATSQNRITHTKIHTGSQKPFQCRICMRNFSRSDNL | |
| | SRHIRTHTGEKPFACDICGRKFAQSGNLVRHTKIHLRQKDAAR | |
| SEQ ID | MAERPFQCRICMRNFSQSGSLTRHIRTHTGEKPFACDICGRKFAR | SCN10A |
| NO: | SDALARHTKIHTGSQKPFQCRICMRNFSTSSNLTRHIRTHTGEKP | |
| 160 | FACDICGRKFATSGHLTRHTKIHTGSQKPFQCRICMRNFSRSDHL | |
| | SEHIRTHTGEKPFACDICGRKFADSRDRTKHTKIHLRQKDAAR | |
| SEQ ID | MAERPFQCRICMRNFSDRSALARHIRTHTGEKPFACDICGRKFA | SCN10A |
| NO: | QSGNLARHTKIHTGSQKPFQCRICMRNFSRSDNLARHIRTHTGE | |
| 161 | KPFACDICGRKFAQSGHLARHTKIHTGSQKPFQCRICMRNFSRSD | |
| | SLTQHIRTHTGEKPFACDICGRKFAQSSHLTRHTKIHLRQKDAAR | |
| SEQ ID | MAERPFQCRICMRNFSDRSHLSEHIRTHTGEKPFACDICGRKFAR | SCN10A |
| NO: | SDARKTHTKIHTGSQKPFQCRICMRNFSRSDTLSTHIRTHTGEKP | |
| 162 | FACDICGRKFADRSNLTRHTKIHTGSQKPFQCRICMRNFSRSDNL | |
| | ARHIRTHTGEKPFACDICGRKFARKDNRITHTKIHLRQKDAAR | |
| SEQ ID | MAERPFQCRICMRNFSQSGHLARHIRTHTGEKPFACDICGRKFA | SCN10A |
| NO: | QSGNLTRHTKIHTGSQKPFQCRICMRNFSQSGDLTRHIRTHTGEK | |
| 163 | PFACDICGRKFAQSGHLARHTKIHTGSQKPFQCRICMRNFSQSGH | |
| | LSQHIRTHTGEKPFACDICGRKFAQSTSLSKHTKIHLRQKDAAR | |
| SEQ ID | MAERPFQCRICMRNESTSSNLSAHIRTHTGEKPFACDICGRKFAR | SCN10A |
| NO: | SDNRKKHTKIHTGSQKPFQCRICMRNFSQNSNLRRHIRTHTGEKP | |
| 164 | FACDICGRKFAQSGNLTRHTKIHTGSQKPFQCRICMRNFSQLGSL | |
| | SRHIRTHTGEKPFACDICGRKFALKSNLRRHTKIHLRQKDAAR | |
| SEQ ID | MAERPFQCRICMRNFSQSGNLQAHIRTHTGEKPFACDICGRKFA | SCN10A |
| NO: | QSGNLRAHTKIHTGSQKPFQCRICMRNFSQSGNLTRHIRTHTGEK | |
| 165 | PFACDICGRKFALKQHLRTHTKIHTGSQKPFQCRICMRNFSQSSA | |
| | LSRHIRTHTGEKPFACDICGRKFARSDSLRQHTKIHLRQKDAAR | |
| SEQ ID | MAERPFQCRICMRNFSRSDHLSEHIRTHTGEKPFACDICGRKFAR | SCN10A |
| NO: | SDHRKRHTKIHTGSQKPFQCRICMRNFSTSSALSRHIRTHTGEKP | |
| 166 | FACDICGRKFARSDNLAAHTKIHTGSQKPFQCRICMRNFSTSGNL | |
| | ARHIRTHTGEKPFACDICGRKFAQSGDLTRHTKIHLRQKDAAR | |
| SEQ ID | MAERPFQCRICMRNFSRSWKLQAHIRTHTGEKPFACDICGRKFA | SCN10A |
| NO: | QSGNLTRHTKIHTGSQKPFQCRICMRNFSRSGHLSRHIRTHTGEK | |
| 167 | PFACDICGRKFARSDNLRAHTKIHTGSQKPFQCRICMRNFSTSSN | |
| | LSRHIRTHTGEKPFACDICGRKFARSDNLTQHTKIHLRQKDAAR | |
| SEQ ID | MAERPFQCRICMRNFSQSGDLTRHIRTHTGEKPFACDICGRKFAL | SCN10A |
| NO: | RTSLRKHTKIHTGSQKPFQCRICMRNFSTPSYLPTHIRTHTGEKPF | |
| 168 | ACDICGRKFADRSALARHTKIHTGSQKPFQCRICMRNFSQSSHLT | |
| | RHIRTHTGEKPFACDICGRKFARSDALARHTKIHLRQKDAAR | |
| SEQ ID | MAERPFQCRICMRNFSQSGNLSRHIRTHTGEKPFACDICGRKFAR | SCN10A |
| NO: | SDALRNHTKIHTGSQKPFQCRICMRNFSRSGNLARHIRTHTGEKP | |
| 169 | FACDICGRKFARSDNRITHTKIHTGSQKPFQCRICMRNFSRSDNL | |
| | SAHIRTHTGEKPFACDICGRKFARSDNRITHTKIHLRQKDAAR | |
| SEQ ID | MAERPFQCRICMRNFSRSDNLARHIRTHTGEKPFACDICGRKFA | SCN10A |
| NO: | QSANRTKHTKIHTGSQKPFQCRICMRNFSRSDNLARHIRTHTGE | |
| 170 | KPFACDICGRKFAQWNYRGSHTKIHTGSQKPFQCRICMRNFSQS | |
| | SDLSRHIRTHTGEKPFACDICGRKFAHRSNLSKHTKIHLRQKDAA | |
| | R | |
| SEQ ID | MAERPFQCRICMRNFSQSGHLARHIRTHTGEKPFACDICGRKFA | SCN10A |
| NO: | TSGNLARHTKIHTGSQKPFQCRICMRNFSQSGNLTRHIRTHTGEK | |
| 171 | PFACDICGRKFARSDNRITHTKIHTGSQKPFQCRICMRNFSRSAN | |
| | LSRHIRTHTGEKPFACDICGRKFAQSDTRITHTKIHLRQKDAAR | |
| SEQ ID | MAERPFQCRICMRNFSQRSNLSRHIRTHTGEKPFACDICGRKFAT | SCN10A |
| NO: | SGHLSRHTKIHTGSQKPFQCRICMRNFSRSDTLSEHIRTHTGEKPF | |
| 172 | ACDICGRKFAANSNRIKHTKIHTGSQKPFQCRICMRNFSRSDNLS | |
| | VHIRTHTGEKPFACDICGRKFARRDTRNNHTKIHLRQKDAAR | |
| SEQ ID | MAERPFQCRICMRNFSRSDTLSEHIRTHTGEKPFACDICGRKFAQ | SCN10A |
| NO: | NANRIKHTKIHTGSQKPFQCRICMRNFSRSDSLSQHIRTHTGEKP | |
| 173 | FACDICGRKFARSHNRKTHTKIHTGSQKPFQCRICMRNFSRSDNL | |
| | SRHIRTHTGEKPFACDICGRKFATNQNRITHTKIHLRQKDAAR | |
| SEQ ID | MAERPFQCRICMRNFSERGTLSRHIRTHTGEKPFACDICGRKFAR | SCN10A |
| NO: | SDHLSRHTKIHTGSQKPFQCRICMRNFSQSSTRKTHIRTHTGEKP | |
| 174 | FACDICGRKFATSSTLSNHTKIHTGSQKPFQCRICMRNFSRSDHL | |
| | SQHIRTHTGEKPFACDICGRKFAQSATRKTHTKIHLRQKDAAR | |
| SEQ ID | MAERPFQCRICMRNFSTSDHLSEHIRTHTGEKPFACDICGRKFAQ | SCN10A |
| NO: | SASRTKHTKIHTGSQKPFQCRICMRNFSQSGSLTRHIRTHTGEKP | |
| 175 | FACDICGRKFAQSGNLRKHTKIHTGSQKPFQCRICMRNFSDRSDL | |
| | SRHIRTHTGEKPFACDICGRKFAQSSDLSRHTKIHLRQKDAAR | |
| SEQ ID | MAERPFQCRICMRNFSRSDNLARHIRTHTGEKPFACDICGRKFA | SCN10A |
| NO: | RSDALARHTKIHTGSQKPFQCRICMRNFSRSDALSEHIRTHTGEK | |
| 176 | PFACDICGRKFARSSDRTKHTKIHTGSQKPFQCRICMRNFSRSDH | |
| | LSRHIRTHTGEKPFACDICGRKFARNQDRTNHTKIHLRQKDAAR | |
| SEQ ID | MAERPFQCRICMRNFSRSDTLSVHIRTHTGEKPFACDICGRKFAD | SCN10A |
| NO: | RSHLSRHTKIHTGSQKPFQCRICMRNFSRSDNLSVHIRTHTGEKP | |
| 177 | FACDICGRKFADRGNLTRHTKIHTGSQKPFQCRICMRNFSRSDNL | |
| | ARHIRTHTGEKPFACDICGRKFADSHHRITHTKIHLRQKDAAR | |
| SEQ ID | MAERPFQCRICMRNFSQSGNLSRHIRTHTGEKPFACDICGRKFAR | SCN10A |
| NO: | SDALATHTKIHTGSQKPFQCRICMRNFSRSDNLSTHIRTHTGEKP | |
| 178 | FACDICGRKFARSGNLSRHTKIHTGSQKPFQCRICMRNFSRSDNL | |
| | STHIRTHTGEKPFACDICGRKFATSSNRTKHTKIHLRQKDAAR | |
| SEQ ID | MAERPFQCRICMRNFSDRSALSRHIRTHTGEKPFACDICGRKFAR | SCN10A |
| NO: | SDALARHTKIHTGSQKPFQCRICMRNFSPCRYRLDHIRTHTGEKP | |
| 179 | FACDICGRKFARSANLTRHTKIHTGSQKPFQCRICMRNFSRSDNL | |
| | STHIRTHTGEKPFACDICGRKFADNSYLPRHTKIHLRQKDAAR | |
| SEQ ID | MAERPFQCRICMRNFSNSGDLTRHIRTHTGEKPFACDICGRKFAT | SCN10A |
| NO: | SGHLSRHTKIHTGSQKPFQCRICMRNFSRSDALSEHIRTHTGEKP | |
| 180 | FACDICGRKFAQNATRTKHTKIHTGSQKPFQCRICMRNFSQSGN | |
| | LTRHIRTHTGEKPFACDICGRKFAQSGDLGEHTKIHLRQKDAAR | |
| SEQ ID | MAERPFQCRICMRNFSRSDNLSRHIRTHTGEKPFACDICGRKFAR | SCN10A |
| NO: | SDARTRHTKIHTGSQKPFQCRICMRNFSDRSDLARHIRTHTGEKP | |
| 181 | FACDICGRKFARSSDLSRHTKIHTGSQKPFQCRICMRNFSRSDHL | |
| | SRHIRTHTGEKPFACDICGRKFADSQDRKTHTKIHLRQKDAAR | |
| SEQ ID | MAERPFQCRICMRNFSQSGSLTRHIRTHTGEKPFACDICGRKFAH | SCN10A |
| NO: | RQHLQTHTKIHTGSQKPFQCRICMRNFSDRSNLSRHIRTHTGEKP | |
| 182 | FACDICGRKFALKQVLVRHTKIHTGSQKPFQCRICMRNFSRSDSL | |
| | LRHIRTHTGEKPFACDICGRKFADRSNRRKHTKIHLRQKDAAR | |
| SEQ ID | MAERPFQCRICMRNFSRSDDLTAHIRTHTGEKPFACDICGRKFA | SCN10A |
| NO: | QSAHRKRHTKIHTGSQKPFQCRICMRNFSTSAHLARHIRTHTGE | |
| 183 | KPFACDICGRKFATSASRTRHTKIHTGSQKPFQCRICMRNFSTSG | |
| | HLTRHIRTHTGEKPFACDICGRKFARSDALAQHTKIHLRQKDAA | |
| | R | |
| SEQ ID | MAERPFQCRICMRNFSRSGNLTAHIRTHTGEKPFACDICGRKFA | SCN10A |
| NO: | QNANRIKHTKIHTGSQKPFQCRICMRNFSQSGSLTQHIRTHTGEK | |
| 184 | PFACDICGRKFATSQNLTRHTKIHTGSQKPFQCRICMRNFSTSDS | |
| | LLRHIRTHTGEKPFACDICGRKFATSHHLTRHTKIHLRQKDAAR | |
| SEQ ID | MAERPFQCRICMRNFSQSSTRKTHIRTHTGEKPFACDICGRKFAT | SCN10A |
| NO: | SSTRTKHTKIHTGSQKPFQCRICMRNFSDRSALSRHIRTHTGEKPF | |
| 185 | ACDICGRKFAQSGDLTRHTKIHTGSQKPFQCRICMRNFSRSDSLA | |
| | RHIRTHTGEKPFACDICGRKFATSGNLARHTKIHLRQKDAAR | |
| SEQ ID | MAERPFQCRICMRNFSTSGHLKEHIRTHTGEKPFACDICGRKFAQ | SCN10A |
| NO: | SGHLSRHTKIHTGSQKPFQCRICMRNFSQSSDLSRHIRTHTGEKPF | |
| 186 | ACDICGRKFAQSGNLSKHTKIHTGSQKPFQCRICMRNFSQSGHL | |
| | NRHIRTHTGEKPFACDICGRKFAQSGNLARHTKIHLRQKDAAR | |
| SEQ ID | MAERPFQCRICMRNFSTSSNRKTHIRTHTGEKPFACDICGRKFAQ | SCN10A |
| NO: | SANRITHTKIHTGSQKPFQCRICMRNFSQSGSLTRHIRTHTGEKPF | |
| 187 | ACDICGRKFALKQNRIKHTKIHTGSQKPFQCRICMRNFSTPSYLP | |
| | THIRTHTGEKPFACDICGRKFADRSALARHTKIHLRQKDAAR | |
| SEQ ID | MAERPFQCRICMRNFSRSGTLTRHIRTHTGEKPFACDICGRKFAQ | SCN10A |
| NO: | SGDLTRHTKIHTGSQKPFQCRICMRNFSQSGHLARHIRTHTGEKP | |
| 188 | FACDICGRKFARRSTRKSHTKIHTGSQKPFQCRICMRNFSQSGNL | |
| | ARHIRTHTGEKPFACDICGRKFAQSGNLARHTKIHLRQKDAAR | |
| SEQ ID | MAERPFQCRICMRNFSRSDHLSQHIRTHTGEKPFACDICGRKFAR | Scn10a |
| NO: | SAVRKNHTKIHTGSQKPFQCRICMRNFSRSDHLSEHIRTHTGEKP | |
| 189 | FACDICGRKFAQSHHRKTHTKIHTGSQKPFQCRICMRNFSDRSNL | |
| | SRHIRTHTGEKPFACDICGRKFALKQHLNEHTKIHLRQKDAAR | |
| SEQ ID | MAERPFQCRICMRNFSDRSNLSRHIRTHTGEKPFACDICGRKFAR | Scn10a |
| NO: | SDDRKTHTKIHTGSQKPFQCRICMRNFSERGTLARHIRTHTGEKP | |
| 190 | FACDICGRKFAQSGHLSRHTKIHTGSQKPFQCRICMRNFSQSGHL | |
| | ARHIRTHTGEKPFACDICGRKFAVSHHLRDHTKIHLRQKDAAR | |
| SEQ ID | MAERPFQCRICMRNFSTSGHLSRHIRTHTGEKPFACDICGRKFAD | Scn10a |
| NO: | RSHLARHTKIHTGSQKPFQCRICMRNFSQSGDLTRHIRTHTGEKP | |
| 191 | FACDICGRKFAYRWLRNNHTKIHTGSQKPFQCRICMRNFSRSDT | |
| | LSEHIRTHTGEKPFACDICGRKFARAQHLQQHTKIHLRQKDAAR | |
| SEQ ID | MAERPFQCRICMRNFSQSGNLARHIRTHTGEKPFACDICGRKFA | Scn10a |
| NO: | RLDILQQHTKIHTGSQKPFQCRICMRNFSRSDVLSEHIRTHTGEK | |
| 192 | PFACDICGRKFATRNGLKYHTKIHTGSQKPFQCRICMRNFSQSSD | |
| | LSRHIRTHTGEKPFACDICGRKFARKYYLAKHTKIHLRQKDAAR | |

In some cases, a ZFP targeting Naᵥ1.7 comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 134-SEQ ID NO: 148 or SEQ ID NO: 278-SEQ ID NO: 290. In some cases, the ZFP targeting Naᵥ1.7 comprises a sequence of any of SEQ ID NO: 134-SEQ ID NO: 148 or SEQ ID NO: 278-SEQ ID NO: 290. In some cases, a ZFP targeting Naᵥ1.8 comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence ofany of SEQ ID NO: 149-SEQ ID NO: 192. In some cases, the ZFP targeting Naᵥ1.8 comprises a sequence ofany of SEQ ID NO: 149-SEQ ID NO: 192.

A zinc finger protein may bind to a target sequence. In some embodiments, the target sequence is a portion of a gene, such as a gene encoding Naᵥ1.7 or Naᵥ1.8. Examples of zinc finger protein target sequences are provided in TABLE 4.

TABLE 4
Exemplary Target Sequences

| SEQ ID NO | Sequence | Gene Target |
|---|---|---|
| SEQ ID NO: 206 | AGTCTGCTTGCAGGCGGT | SCN9A |
| SEQ ID NO: 207 | CCAGCGGCGGCTGTCGGC | SCN9A |
| SEQ ID NO: 208 | GCCTGGGTGCCAGTGGCT | SCN9A |
| SEQ ID NO: 209 | TGGCTGCTAGCGGCAGGC | SCN9A |
| SEQ ID NO: 210 | GCGTCCCCTGAGCAACAG | SCN9A |
| SEQ ID NO: 211 | AAGGAGAGGCCCGCGCCC | SCN9A |
| SEQ ID NO: 212 | GCAGGTGCACTGGGTGGG | SCN9A |
| SEQ ID NO: 213 | GCGCCCGTGGAGGTAGCA | SCN9A |
| SEQ ID NO: 214 | TGCCAGGGCGCGCCCGTG | SCN9A |
| SEQ ID NO: 215 | ACAGCCGCCGCTGGAGCG | SCN9A |
| SEQ ID NO: 216 | CCAGGAGAGGGCGCGGGC | SCN9A |
| SEQ ID NO: 217 | GGCGAGGTGATGGAAGGG | SCN9A |
| SEQ ID NO: 218 | GAGGGAGCTAGGGGTGGG | SCN9A |
| SEQ ID NO: 219 | AGTGCTAATGTTTCCGAG | SCN9A |
| SEQ ID NO: 220 | TAGACGGTGCAGGGCGGA | SCN9A |
| SEQ ID NO: 291 | GTGGGTGTGGCGGTTGAG | SCN9A |
| SEQ ID NO: 292 | GCCGCCGCTGGAGCGCTG | SCN9A |
| SEQ ID NO: 293 | GCGCCCGTGGAGGTAGCA | SCN9A |
| SEQ ID NO: 294 | GCCGCTGGAGCGCTGGCG | SCN9A |
| SEQ ID NO: 295 | GGTGTGGCGGTTGAGGCG | SCN9A |
| SEQ ID NO: 296 | GGGGAAGGAGAGGCCCGC | SCN9A |
| SEQ ID NO: 297 | GCGTCCCCTGAGCAACAG | SCN9A |
| SEQ ID NO: 298 | TCAGCCGAGCTGGCGGAA | SCN9A |
| SEQ ID NO: 299 | GCTGGAGCGCTGGCGACC | SCN9A |
| SEQ ID NO: 300 | GGTGGGGATGATCGGCGG | SCN9A |
| SEQ ID NO: 301 | TGGTTCCAGCAATGGGAG | SCN9A |
| SEQ ID NO: 302 | GGAGAGGGCGCGGGCCTC | SCN9A |
| SEQ ID NO: 221 | GATTGAGCAGCAAAGGTT | SCN10A |
| SEQ ID NO: 222 | GATGCAGTGGGAGTCTTC | SCN10A |
| SEQ ID NO: 223 | CTGGGTGATGTGGCAGTC | SCN10A |
| SEQ ID NO: 224 | AAGCAAGATTGAGCAGCA | SCN10A |
| SEQ ID NO: 225 | GGTGATGTGGCAGTCTGC | SCN10A |
| SEQ ID NO: 226 | AGAGGAAGAGATGCAGTG | SCN10A |
| SEQ ID NO: 227 | GGAGAGGAAGTCTCGGTC | SCN10A |
| SEQ ID NO: 228 | GTAGAGGCAATCTGGGAT | SCN10A |
| SEQ ID NO: 229 | GATGAGAGGGACAGGGCC | SCN10A |
| SEQ ID NO: 230 | CCCAGGGCCAAGGAGGAC | SCN10A |
| SEQ ID NO: 231 | GAAGAGTATAAACTTGAC | SCN10A |
| SEQ ID NO: 232 | GCCCTGGGTGATGTGGCA | SCN10A |
| SEQ ID NO: 233 | GCTCTTGGAGAGGAAGTC | SCN10A |
| SEQ ID NO: 234 | AAGGAGGACTTTCTGGGC | SCN10A |
| SEQ ID NO: 235 | GCATAGGGAGCAGAAGGA | SCN10A |
| SEQ ID NO: 236 | TACGTAGAAGACAAGCAA | SCN10A |
| SEQ ID NO: 237 | TTGGTTTATGAATAAAAT | SCN10A |
| SEQ ID NO: 238 | GCAGCAAAGGTTTGGCTG | SCN10A |
| SEQ ID NO: 239 | AAGGATAAGGGTGAAAAT | SCN10A |
| SEQ ID NO: 240 | GTGGGAGTCTTCTATGCA | SCN10A |
| SEQ ID NO: 241 | AAGAATGAGAAGATGGAA | SCN10A |
| SEQ ID NO: 242 | TATCCTTCAGAGGAAGAG | SCN10A |
| SEQ ID NO: 243 | GAAGAATGAGAAGATGGA | SCN10A |
| SEQ ID NO: 244 | CTGAAGGATAAGGGTGAA | SCN10A |
| SEQ ID NO: 245 | AATGAGAAGATGGAATTC | SCN10A |
| SEQ ID NO: 246 | TCAAGTCCAGCTGGGGCC | SCN10A |
| SEQ ID NO: 247 | GCTGCCCAAGTTCTATGG | SCN10A |
| SEQ ID NO: 248 | CCGGAGTCACTGGTGGAG | SCN10A |
| SEQ ID NO: 249 | TGAGAGGGACAGGGCCAG | SCN10A |
| SEQ ID NO: 250 | GACCACGAGCTCCTGGAA | SCN10A |
| SEQ ID NO: 251 | TTCCAGGAGCTCGTGGTC | SCN10A |
| SEQ ID NO: 252 | CCAAGAACTATGGGTGTT | SCN10A |
| SEQ ID NO: 253 | ATCGGGGAGCCCCTGGAG | SCN10A |
| SEQ ID NO: 254 | TCTGTGGCTAACAGTGTA | SCN10A |
| SEQ ID NO: 255 | ATGGGTGTTTGAGGAAGG | SCN10A |
| SEQ ID NO: 256 | TGTGTTGATATAAAAAGA | SCN10A |
| SEQ ID NO: 257 | GATGTGGCAGTCTGCTAA | SCN10A |
| SEQ ID NO: 258 | GAATGTTAAGCTGAACGT | SCN10A |
| SEQ ID NO: 259 | GTCTTCTACGTAAGAAAT | SCN10A |
| SEQ ID NO: 260 | GAAGCATAGGGAGCAGAA | SCN10A |
| SEQ ID NO: 261 | AGTGACGGACGGGTGAGG | SCN10A |
| SEQ ID NO: 262 | GTGGAGGAGCCCCGGAC | SCN10A |
| SEQ ID NO: 263 | GGTGCTCCAGAAAGTACA | SCN10A |

In some cases, a ZFP target sequence for modulating Naᵥ1.7 expression comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 206-SEQ ID NO: 220 or SEQ ID NO: 291-SEQ ID NO: 302. In some cases, the ZFP target sequence for modulating Naᵥ1.7 expression comprises a sequence of any of SEQ ID NO: 206-SEQ ID NO: 220 or SEQ ID NO: 291-SEQ ID NO: 302. In some cases, a ZFP target sequence for modulating Naᵥ1.8 expression comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 221-SEQ ID NO: 263. In some cases, the ZFP target sequence for modulating Naᵥ1.8 expression comprises a sequence of any of SEQ ID NO: 221-SEQ ID NO: 263.

Examples of epigenetic modulators comprising a zinc finger protein and a repressor domain are provided in TABLE 5.

TABLE 5
Exemplary Epigenetic Modulators

| SEQ | | Target | Gene |
|---|---|---|---|
| ID NO | Sequence | Sequence | Target |
| SEQ ID | RSMHDYKDHDGDYKDHDIDYKDDDDKMAPKK | GGCGAGGT | SCN9A |
| NO: | KRKVGIHGVPAAMAERPFQCRICMRNFSRSAHL | GATGGAAG | |
| 264 | SRHIRTHTGEKPFACDICGRKFAQSGNLARHTKI | GG (SEQ ID | |
| | HTGSQKPFQCRICMRNFSRSDAMSQHIRTHTGE | NO: 217) | |
| | KPFACDICGRKFARNASRTRHTKIHTGSQKPFQC | | |
| | RICMRNFSRSANLARHIRTHTGEKPFACDICGRK | | |
| | FADRSHLARHTKIHLRQKDAARGSRTLVTFKDV | | |
| | FVDFTREEWKLLDTAQQIVYRNVMLENYKNLV | | |
| | SLGYQLTKPDVILRLEKGEEPWLVDYKDDDDK | | |
| | RS | | |
| SEQ ID | RSMHDYKDHDGDYKDHDIDYKDDDDKMAPKK | GAGGGAGC | SCN9A |
| NO: | KRKVGIHGVPAAMAERPFQCRICMRNFSRSANL | TAGGGGTG | |
| 265 | ARHIRTHTGEKPFACDICGRKFADSSDRKKHTKI | GG (SEQ ID | |
| | HTGSQKPFQCRICMRNFSTSGSLSRHIRTHTGEK | NO: 218) | |
| | PFACDICGRKFAHSLSLKNHTKIHTGSQKPFQCR | | |
| | ICMRNFSQSSDLSRHIRTHTGEKPFACDICGRKF | | |
| | AWKWNLRAHTKIHLRQKDAARGSRTLVTFKDV | | |
| | FVDFTREEWKLLDTAQQIVYRNVMLENYKNLV | | |
| | SLGYQLTKPDVILRLEKGEEPWLVDYKDDDDK | | |
| | RS | | |
| SEQ ID | RSMHDYKDHDGDYKDHDIDYKDDDDKMAPKK | AGTGCTAA | SCN9A |
| NO: | KRKVGIHGVPAAMAERPFQCRICMRNFSRSAHL | TGTTTCCG | |
| 266 | SRHIRTHTGEKPFACDICGRKFATSGHLSRHTKI | AG (SEQ ID | |
| | HTGSQKPFQCRICMRNFSRSDHLSQHIRTHTGEK | NO: 219) | |
| | PFACDICGRKFAASSTRTKHTKIHTGSQKPFQCR | | |
| | ICMRNFSQSSHLTRHIRTHTGEKPFACDICGRKF | | |
| | ARSDNLTRHTKIHLRQKDAARGSRTLVTFKDVF | | |
| | VDFTREEWKLLDTAQQIVYRNVMLENYKNLVS | | |
| | LGYQLTKPDVILRLEKGEEPWLVDYKDDDDKR | | |
| | S | | |
| SEQ ID | RSMHDYKDHDGDYKDHDIDYKDDDDKMAPKK | TAGACGGT | SCN9A |
| NO: | KRKVGIHGVPAAMAERPFQCRICMRNFSDRSHL | GCAGGGCG | |
| 267 | TRHIRTHTGEKPFACDICGRKFADRSHLARHTKI | GA (SEQ ID | |
| | HTGSQKPFQCRICMRNFSRSDNLSEHIRTHTGEK | NO: 220) | |
| | PFACDICGRKFARSAALARHTKIHTGSQKPFQCR | | |
| | ICMRNFSRSDTLSQHIRTHTGEKPFACDICGRKF | | |
| | ATRDHRIKHTKIHLRQKDAARGSRTLVTFKDVF | | |
| | VDFTREEWKLLDTAQQIVYRNVMLENYKNLVS | | |
| | LGYQLTKPDVILRLEKGEEPWLVDYKDDDDKR | | |
| | S | | |

### Additional Nucleic Acid Binding Domains

Further non-limiting examples of nucleic acid binding domains include meganuclease and transcription activator-like effectors (TALEs).

### Expression Modulating Agents

An expression modulating agent may modulate expression of a target molecule. In certain embodiments, the expression modulating agent comprises an activator that activates expression. In certain embodiments, the expression modulating agent comprises a repressor that represses expression. The expression modulating agent may comprise a transcription regulatory domain that has transcription repression activity (e.g., a repressor domain) or transcription activation activity (e.g., an activator domain). In some cases, the repressor domain comprises ZIM3. In some cases, the repressor domain comprises a Krueppel-associated box (KRAB) domain (recruitment of histone methyltransferases and deacetylases). Non-limiting examples of repressor domains are provided in TABLE 6.

TABLE 6
Exemplary Repressor Domains

| | SEQ ID | |
|---|---|---|
| Repressor | NO | Sequence |
| ZIM3 | SEQ ID | VTFEDVTVNFTQGEWQRLNPEQRNLYRDVMLENYSNLVSV |
| | NO: 193 | GQGETTKPDVILRLEQGKEPWL |
| KOX1 | SEQ ID | VTFKDVFVDFTREEWKLLDTAQQIVYRNVMLENYKNLVSL |
| | NO: 194 | GYQLTKPDVILRLEKGEEPWLV |
| ZNF554 | SEQ ID | VTFEDVSMDFSQEEWELLEPAQKNLYREVMLENYRNVVSL |
| | NO: 195 | EALKNQCTDVGIKEGPLSPAQT |
| ZNF264 | SEQ ID | VTFDDVAVTFTKEEWGQLDLAQRTLYQEVMLENCGLLVSL |
| | NO: 196 | GCPVPKAELICHLEHGQEPWT |
| ZNF324 | SEQ ID | MAFEDVAVYFSQEEWGLLDTAQRALYRRVMLDNFALVASL |
| | NO: 197 | GLSTSRPRVVIQLERGEEPWV |
| MeCP2 | SEQ ID | VQVKRVLEKSPGKLLVKMPFQASPGGKGEGGGATTSAQVM |
| | NO: 198 | VIKRPGRKRKAEADPQAIPKKRGRKPGSVVAAAAAEAKKK |
| | | AVKESSIRSVQETVLPIKKRKTRETVSIEVKEVVKPLLVSTLG |
| | | EKSGKGLKTCKSPGRKSKESSPKGRSSSASSPPKKEHHHHHH |
| | | HAESPKAPMPLLPPPPPPEPQSSEDPISPPEPQDLSSSICKEEK |
| | | MPRAGSLESDGCPKEPAKTQPMVAAAATTTTTTTTTVAEKY |
| | | KHRGEGERKDIVSSSMPRPNREEPVDSRTPVTERVS |
| MBD2b | SEQ ID | ARYLGNTVDLSSFDFRTGKMMPSKLQKNKQRLRNDPLNQN |
| | NO: 199 | KGKPDLNTTLPIRQTASIFKQPVTKVTNHPSNKVKSDPQRMN |
| | | EQPRQLFWEKRLQGLSASDVTEQIIKTMELPKGLQGVGPGS |
| | | NDETLLSAVASALHTSSAPITGQVSAAVEKNPAVWLNTSQP |
| | | LCKAFIVTDEDIRKQEERVQQVRKILEDALMADILSRAADTE |
| | | EMDIEMDSGDEAEF |
| SID | SEQ ID | MNIQMLLEAADYLERREREAEHGYASMLP |
| | NO: 200 | |
| HP1a | SEQ ID | MKEGENNKPREKSEGNKRKSSFSNSADDIKSKKKREQSNDI |
| | NO: 201 | ARGFERGLEPEKIIGATDSCGDLMFLMKWKDTDEADLVLAK |
| | | EANVKCPQIVIAFYEERLTWHAYPEDAENKEKESAKSEF |
| SIRT5 | SEQ ID | SSSMADFRKFFAKAKHIVIISGAGVSAESGVPTFRGAGGYWR |
| | NO: 202 | KWQAQDLATPLAFAHNPSRVWEFYHYRREVGSKEPNAGHR |
| | | AIAECETRLGKQGRRVWITQNIDELHRKAGTKNLLEIHGSLF |
| | | KTRCTSCGWAENYKSPICPALSGKGAPEPGTQDASIPVEKLP |
| | | RCEEAGCGGLLRPHWWFGENLDPAILEEVDRELAHCDLCL |
| | | WGTSSWYPAAFAPQVAARGVPVAEFNTETTPATNRFRFHFQ |
| | | GPCGTTLPEALACHENETVS |
| SETD8 | SEQ ID | SCDSTNAALAKQALKKPIKGKQAPRKKAQGKTQQNRKLTDF |
| | NO: 203 | YPVRRSSRKSKAELQSEERKRIDELIESGKEEGKIDLIDGKGR |
| | | GVIATKQFSRGDFVVEYHGDLIEITDAKKREALYAQDPSTGC |
| | | YYYFQYLSKTYCVDATRETNRLGRLINHSKCGNCQTKLHDI |
| | | DGVPHLILIASRDIAAGEELLYDYGDRSKASIEAFPWLKHEF |
| HDT1 | SEQ ID | MEFWGIEVKSGKPVTVTPEEGILIHVSQASLGECKNKKGEFV |
| | NO: 204 | PLHVKVGNQNLVLGTLSTENIPQLFCDLVFDKEFELSHTWG |
| | | KGSVYFVGYKTPNIEPQGYSEEEEEEEEEVPAGNAAKAVAK |
| | | PKAKPAEVKPAVDDEEDESDSDGMDEDDSDGEDSEEEEPTP |
| | | KKPASSKKRANETTPKAPVSAKKAKVAVTPQKTDEKKKGG |
| | | KAANQSEF |
| SUPRD | SEQ ID | DLELRL |
| | NO: 205 | |

A composition of the present disclosure may comprise or encode a repressor domain of any of SEQ ID NO: 193-SEQ ID NO: 205, or a sequence having at least 50%, at least 60%, at least 70%, at least 75%, at least 80%, at least 85%, at least 90%, at least 92%, at least 95%, at least 97%, at least 98%, at least 99%, or 100% sequence identity to any one of SEQ ID NO: 193-SEQ ID NO: 205. The repressor domain may be a variant or combination of repressor domains of any of SEQ ID NO: 193-SEQ ID NO: 205. In some embodiments, the repressor domain comprises having at least 50%, at least 60%, at least 70%, at least 75%, at least 80%, at least 85%, at least 90%, at least 92%, at least 95%, at least 97%, at least 98%, at least 99%, or 100% sequence identity to ZIM3 (SEQ ID NO: 193). In some embodiments, the repressor domain comprises having at least 50%, at least 60%, at least 70%, at least 75%, at least 80%, at least 85%, at least 90%, at least 92%, at least 95%, at least 97%, at least 98%, at least 99%, or 100% sequence identity to KOX1 (SEQ ID NO: 194).

In certain embodiments, the expression modulating agent comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb1 self-association domain (recruits enhancer-associated endogenous Ldb1), SAM activator (VP64, p65, HSF1)(recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2)(histone methyltransferase), DNMT3a (DNA methyltransferase), or DNMT3a-DNMT3L (DNA methyltransferase), p16, p300, CD, SunTag, FOG1, DNMT3A, DNMT3L, DMT3, or a variant or combination thereof.

In certain embodiments, the expression modulating agent comprises KRAB (also referred to as KOX), SID, MBD2, MBD3, HP1a, DNMT family (including DNMT1, DNMT3A, DNMT3B, DNMT3L, DNMT2A), Sin3a, Rb, MeCP2 (methyl-CpG binding protein 2), ROM2, AtHD2A, LSD1, SUV39H1, or G9a (EHMT2), or a variant or combination thereof. Variants of KRAB domains include ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, or ZNF257, or a variant or combination thereof.

In certain embodiments, the expression modulating agent comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof.

### Simultaneous Activation and Repression

In some embodiments, two or more genes are activated, repressed, or one gene is activated and another gene is repressed. In some embodiments, the system comprises a first sequence comprising the amino-terminus of dCas9 (1-573), guide RNA for SCN9A, and a KRAB repressor domain; and second sequence comprising the carboxy-terminus of dCas9 (574-1398), guide RNA for PenK, and a VP64 activation domain. This system may be utilized to simultaneously repress SCN9A and activate PenK. The simultaneous activation and repression may be via gRNA-M2M recruiting MCP-VP64 and gRNA-Com recruiting Com-KRAB. This system may be utilized with other activation and/or repressor domains, and gRNA for simultaneous targeting of different target molecules. Although certain example components are shown, the figure should not be construed as limiting. For instance, although a CMV promoter is shown, it is understood that other promoters, e.g., a Naᵥ1.7 promoter, may be used instead.

### Linkers

In certain embodiments, the transcription modulating agent is linked to the nucleic acid-binding agent. The transcription modulating agent may be positioned N- or C-terminal of the nucleic acid-binding agent. The domains may be linked via a peptide linker. The domains may be linked via a disulfide bond.

In some embodiments, the epigenetic modulator is linked to a nucleic acid binding domain via a peptide linker. Non-limiting examples of peptide linkers include (GGS)n (SEQ ID NO: 303), (GGGS)ₙ (SEQ ID NO: 304), (GGGGS)ₙ (SEQ ID NO:305), (G)ₙ(SEQ ID NO: 306), (EAAAK)ₙ (SEQ ID NO: 307), A(EAAAK)ₙALEA(EAAAK)ₙA (SEQ ID NO: 308), PAPAP (SEQ ID NO: 309), AEAAAKEAAAKA (SEQ ID NO: 310), (Ala-Pro)ₓ(SEQ ID NO: 311), LE, GlySer-polyPro(Glyc)-polyPro(Glyc)-polyPro(Glyc), GlySer-polyPro-polyPro(Glyc)-polyPro, GlySer-polyPro-GlySer(Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-02m-GlySer-β2m-polyPro, GlySer-polyPro-Ub-GlySer, GlySer-polyPro-ZAG-polyPro, GlySer-GlySer-ZAG-GlySer-ZAG-polyPro, GlySer(Glyc)-GlySer(Glyc)-polyPro, (G₄S)₃-cTPR3-(G₄S)₃, (G₄S)₃-cTPR6-(G₄S)₃, (G₄S)₃-cTPR9-(G₄S)₃, (G₄S)₃-cTPR12-(G₄S)₃, and (G₄S)ₙ(SEQ ID NO: 305); wherein n is independently selected from 1 to 10, x is 10-34, polyPro is proline-rich hinge sequence from IgA1, polyPro(Glyc) is proline-rich hinge sequence from IgA1 with an embedded potential N-linked glycosylation site (Asn-Ser-Ser), β2m is 02-microglobulin, Ub is ubiquitin, ZAG is Zn-α2-glycoprotein, and cTPRX is consensus tetratricopeptide repeat sequence with X number of repeats.

### Polynucleotide Compositions

A composition of the present disclosure may comprise a polynucleotide encoding an epigenetic modulator or a component of an epigenetic modulator. For example, the polynucleotide may encode a nucleic acid-binding agent, an expression modulating agent (e.g., a repressor domain, an activator domain, or an epigenetic editor), or a combination thereof. In some embodiments, the nucleic acid-binding agent and the expression modulating agent may be expressed as a fusion protein. The polynucleotide may encode a component of a Cas system (e.g., a Cas protein, a dead Cas protein, a guide RNA, or combinations thereof). In some embodiments, a dead Cas protein may be expressed as a fusion protein with an expression modulating agent.

### Coding Sequences

The polynucleotide may comprise one or more coding sequences. In some embodiments, a coding sequence may encode an epigenetic modulating agent of the present disclosure or a portion of an epigenetic modulating agent. For example, a coding sequence may encode a nucleic acid-binding agent (e.g., a zinc finger protein, a guide RNA, or a TALE), a Cas protein, an expression modulating agent (e.g., a repressor domain or an activator domain), or a combination thereof.

### Promoters

The polynucleotide may further comprise a promoter operably linked to a coding sequence encoding a component of an epigenetic modulator (e.g., a nucleic acid-binding agent, an expression modulating agent, a component of a Cas system, or a combination thereof). The promoter may regulate transcription of the coding sequence. In some embodiments, the promoter may be a ubiquitous promoter that activates transcription of the coding sequence, independent of tissue type. In some embodiments, the promoter may be a cell-specific promoter (e.g., a neuronal-specific promoter) that activates transcription of the coding sequence in a cell type of interest. For example, the promoter may be specific for cells expressing Naᵥ1.7, Naᵥ1.8, TRPV 1, or TRPA1. Examples of promoters that may be included in a polynucleotide of the present disclosure are provided in TABLE 7.

TABLE 7
Exemplary Promoters

| | SEQ ID | |
|---|---|---|
| Name | NO | Sequence |
| | SEQ ID | CAGCCACTGCCCCAGACTCAGGGCTTTGCCATTGGTCCCCACCTCCTCTGCTC |
| | NO: 44 | CGGAGTTTTTCTCCAGCTCCCCACCAAGCCACACAAAGTGACTTCTCGGAAAC |
| | | ATTAGCCGATTCTGCTGAGCAGGAAGGGAGGAAAGGGATGATGGGGGCGGG |
| | | GGTGAGATAAGGGAAGGGCTCTTCTGGCTGCTGGACACACACACACACACAC |
| | | TCAAACACACACACGCCCCACCCAATGGGTGGCCGTGGATGGCAGGTCGTGC |
| | | AACCCCCTCCTCCGCCTTCTATTAGCGCATGGTGCAGAGGCTACAGCGTCGCC |
| | | ACCACCGCGCCCCTAGCTGGGTCCCCGCCCTGCGCCGCCCGCAGGAGTGGAG |
| | | AGAGGGAGGGAGGGAGGGAGCAAGGGGTGGGGACCCGGGCGCGCTGGGAG |
| | | GAGTGGAGGAGGCAAAGCGGCGCAGCTGCCCTCGGGGAGGCGGGGCTGCTA |
| | | CCTCCACGGGCGCGCCCTGGCAGGAGGGGCGCAGTCTGCTTGCAGGCGGTCG |
| | | CCAGCGCTCCAGCGGCGGCTGTCGGCTTTCCAATTCCGCCAGCTCGGCTGAG |
| | | GCTGGGCTAGCCTGGGTGCCAGTGGCTGC |
| | SEQ ID | CTTGAGCGTCACCGCCCCACTCGCGGTTGTGAGCAAAGCCCTACGGAAGAAA |
| | NO: 45 | CCAATTCCCAGCCTAGACTCTTCAGAGCCCAAGGTCGGGGAGGCGCTGGCCT |
| | | GGCGGTGTTGTCTGGCTCCCCAGCCACTGCCCCAGACTCAGGGCTTTGCCATT |
| | | GGTCCCCACCTCCTCTGCTCCGGAGTTTTTCTCCAGCTCCCCACCAAGCCACA |
| | | CAAAGTGACTTCTCGGAAACATTAGCCGATTCTGCTGAGCAGGAAGGGAGGA |
| | | AAGGGATGATGGGGGCGGGGGTGAGATAAGGGAAGGGCTCTTCTGGCTGCT |
| | | GGACACACACACACACACACTCAAACACACACACGCCCCACCCAATGGGTGG |
| | | CCGTGGATGGCAGGTCGTGCAACCCCCTCCTCCGCCTTCTATTAGCGCATGGT |
| | | GCAGAGGCTACAGCGTCGCCACCACCGCGCCCCTAGCTGGGTCCCCGCCCTG |
| | | CGCCGCCCGCAGGAGTGGAGAGAGGGAGGGAGGGAGGGAGCAAGGGGTGG |
| | | GGACCCGGGCGCGCTGGGAGGAGTGGAGGAGGCAAAGCGGCGCAGCTGCCC |
| | | TCGGGGAGGCGGGGCTGCTACCTCCACGGGCGCGCCCTGGCAGGAGGGGCG |
| | | CAGTCTGCTTGCAGGCGGTCGCCAGCGCTCCAGCGGCGGCTGTCGGCTTTCCA |
| | | ATTCCGCCAGCTCGGCTGAGGCTGGGCTAGCCTGGGTGCCAGTGGCTGC |
| | SEQ ID | GAGTGGAGAGAGGGAGGGAGGGAGGGAGCAAGGGGTGGGGACCCGGGCGC |
| | NO: 46 | GCTGGGAGGAGTGGAGGAGGCAAAGCGGCGCAGCTGCCCTCGGGGAGGCGG |
| | | GGCTGCTACCTCCACGGGCGCGCCCTGGCAGGAGGGGCGCAGTCTGCTTGCA |
| | | GGCGGTCGCCAGCGCTCCAGCGGCGGCTGTCGGCTTTCCAATTCCGCCAGCTC |
| | | GGCTGAGGCTGGGCTAGCCTGGGTGCCAGTGGCTGCTAGCGGCAGGCGTCCC |
| | | CTGAGCAACAGGAGCCCAGAGAAAAAGAAGCAGCCCTGAGAGAGCGCCGGG |
| | | GAAGGAGAGGCCCGCGCCCTCTCCTGGAGCCAGATTCTGCAGGTGCACTGGG |
| | | TGGGGATGATCGGCGGGCTAGGTTGCAAGTAAGTGCCTTTTCTTTTGCTGCTT |
| | | TCTTTATTTCTTTGTTTCCATCCAGGCCTCTTATGTGAGGAGCTGAAGAGGAA |
| | | TTAAAATATACAGGATGAAAAGGCGGCCGCCATGGTGAGCAAGGGCGAGGA |
| | | GGATAA |
| | SEQ ID | TCGAATTAACACAAGACAGAAGTCATATAAATCTTAAGAAGGTAGAAAATGT |
| | NO: 47 | TACTTCTAGAAAACTCCACCAAAAGTTGGTTGGATAATTTCAGGTCTTCTTTC |
| | | TGGAAAACGAAGAGTCACAAAGTCTATTTTTCTTTCTACTCCCAAGTTCCACT |
| | | CATTCTCCAATTAAATGTGACTCATAGGTGACTGAATGTCATGTTTTGGCCAC |
| | | TCTAAGCAAAGGGATTCCTTGGACTTGGGTACACATATTTTAAGAATATTGTA |
| | | TATATAAGAATATTGTATATATTCAACTGTCCATCTTGCTGCTTCTCTCTTACA |
| | | CCAACTTCATTTTGTAGCATCAGCTGCATAAGCTCTTGCTTTTCCCCCTATGCA |
| | | GACATCTATTCATCAGGCTCTGTGTACACTGGCAACATAGGAATAAACTGGA |
| | | TAAGGTTGCTGTTCCCAGAAGAATCTCAGAATATTGTATTTATTTAATGGCAT |
| | | CAGGAAAAACCAGTCCACCAATTTTAGAAAGAAAAAAGTCTTAAAGCTGCAC |
| | | ATTTTTATGAACTTAGACAATTACTATATTTTGACTATTATTGACTCTAATCTA |
| | | TTCCAAGAATTATACAGACAGAAAGAGCTTTGGTAACAGCCTACATATCAAT |
| | | ATTTACTGAACCAAGAACTATCACAAAACGTCTGTTGAAAATTTCCATGTTTT |
| | | AATGATGATGATGCTAATGGCAGCTATGATTTATTAAACACTTTCTATGTGCA |
| | | GGATGTAAGCTAAGTAATTTGTACACATTATCTTATTTAAACTTATTACAAGT |
| | | CTTATTTTCTTGAGGTAGATGTTATTATCCTATTTTCACAGACAGACTTATGCC |
| | | TTGAAGAATTTGGTAAAGTCACTAAGTAGTCAGTAGCCAAATCATGTATTCTT |
| | | AATCCTTATTCAATATTGTCCCCCTATAGAAGAAACCTTGAGTTTGTAACTTT |
| | | TCAGTAAGGCTATTTAGCTTGTGTCCTGAAGACACTCTCACCTATA |
| | SEQ ID | TCGAATTAACACAAGACAGAAGTCATATAAATCTTAAGAAGGTAGAAAATGT |
| | NO: 48 | TACTTCTAGAAAACTCCACCAAAAGTTGGTTGGATAATTTCAGGTCTTCTTTC |
| | | TGGAAAACGAAGAGTCACAAAGTCTATTTTTCTTTCTACTCCCAAGTTCCACT |
| | | CATTCTCCAATTAAATGTGACTCATAGGTGACTGAATGTCATGTTTTGGCCAC |
| | | TCTAAGCAAAGGGATTCCTTGGACTTGGGTACACATATTTTAAGAATATTGTA |
| | | TATATAAGAATATTGTATATATTCAACTGTCCATCTTGCTGCTTCTCTCTTACA |
| | | CCAACTTCATTTTGTAGCATCAGCTGCATAAGCTCTTGCTTTTCCCCCTATGCA |
| | | GACATCTATTCATCAGGCTCTGTGTACACTGGCAACATAGGAATAAACTGGA |
| | | TAAGGTTGCTGTTCCCAGAAGAATCTCAGAATATTGTATTTATTTAATGGCAT |
| | | CAGGAAAAACCAGTCCACCAATTTTAGAAAGAAAAAAGTCTTAAAGCTGCAC |
| | | ATTTTTATGAACTTAGACAATTACTATATTTTGACTATTATTGACTCTAATCTA |
| | | TTCCAAGAATTATACAGACAGAAAGAGCTTTGGTAACAGCCTACATATCAAT |
| | | ATTTACTGAACCAAGAACTATCACAAAACGTCTGTTGAAAATTTCCATGTTTT |
| | | AATGATGATGATGCTAATGGCAGCTATGATTTATTAAACACTTTCTATGTGCA |
| | | GGATGTAAGCTAAGTAATTTGTACACATTATCTTATTTAAACTTATTACAAGT |
| | | CTTATTTTCTTGAGGTAGATGTTATTATCCTATTTTCACAGACAGACTTATGCC |
| | | TTGAAGAATTTGGTAAAGTCACTAAGTAGTCAGTAGCCAAATCATGTATTCTT |
| | | AATCCTTATTCAATATTGTCCCCCTATAGAAGAAACCTTGAGTTTGTAACTTT |
| | | TCAGTAAGGCTATTTAGCTTGTGTCCTGAAGACACTCTCACCTATA |
| | SEQ ID | TCAATTATAACAGACATGTCTTAGTCTACCTTTTATTGCTGTAACTGAATACC |
| | NO: 49 | TGAGACTGGTTAAAGTATTCAATAAAGAAATTTATTTCTTAAAGTTCTGGAAG |
| | | CTGGGAAGTCTAAGGTGAAGGGTCCACATCTGGTGGGAACTTTCTTGCTGGT |
| | | GGGCACACCAGAGAGTCCCAAGGCAGCTCATGGCATCACATGGCGAGGGGT |
| | | GTTCACTGAGAGCCAAACTGGGTTTTATAATAAACCCACTCTTGTGATATCTA |
| | | ACTCACTCCCATGATAACCCATTAAGCCCTTACTCTATATTCCATTCATGAAG |
| | | GCAGAACCTTCATGACCTCATCACCTTTTAAAGGCTCTGCCTCTTAATACTGT |
| | | TACATTGGGAATTAAGTTCCAACATGAGTTTCAAAAGGGACAAACATTCAAA |
| | | CCACAGCAAAACATTAAAAAAAAAATTTTCTGATTAAAGAGGGAGCATACAT |
| | | ATTTTCAAGTGTTCTTAAGTGGAAAATAATGGATGTGAATGACTGGGCAAAT |
| | | TTAAGAGTGGCATGAGGAGTAATTTATTCTGCTAAATACGTAGCTTTAGGAAT |
| | | TTAAAAAAAAAAGCCATGTTACATTTAGTGAGACATCTGGAAATACTACATG |
| | | AGGATGAGCACTTTAAAACACTTCCCGCTTCATCAGTCAGTTTACCTGTCTCT |
| | | GATGGTAACAGCAATGAACTTTTTGTGAACAATGACTTTTTCTACGCCTGAAA |
| | | TAGATAAATATATAAAGTTGGCTCTCATCTGTTCCTATTCTTCCCTCGAAAAA |
| | | TTGAATCTCTCAGTCACTAACTTATTCGTAATGAAGAACTGCATTCCAGACAT |
| | | TTTTCTTAGACATCGTCTCCTGCTACACAGGATGTGAGGCAAATGAATATTTT |
| | | CCATGGAACTAATTCAGAGAGTAAACATATTTATAGATCCAGATCTAAGGTC |
| | | ATGGAGGAGCTCATTGCTTTGTAACTAAAATGTGTATTGATTGTATTGCTGTT |
| | | C |
| | SEQ ID | CAGTCTCAGTCACTGAAGTTTCCTTCCTTCAATAGCACATCTTACAAGTCAAG |
| | NO: 50 | AATGTAAGGTCCAAAGCCCCAGGACAAATCTCAGAAGCAAAGAAAGCAGCA |
| | | GGGAAAAGTAGACCCTGGGATTTGATTTCCTTCCGGTTATCTCTAAGCATCAT |
| | | TTCCATGATAGAAGGTGTGGAAGCAAATAATAAAAGTGCCCGTCACTAGTGT |
| | | TTATCCTGCAAAGTGGTCTGCCCTTTTGAGAGGCACCCTGCCCTATGGCCATC |
| | | CTTTGATTTCTTCCCTTGGTGGAAATTTCCTGTTTCTCTTTGAGAAAGATAATT |
| | | CAACCCTGTTTCCATTGTTCTTCCTCCCAGGCTGCTGTAGGAAAGCGCACGCA |
| | | CACACTCCACACAAAATGAATTTTTAAAAAATTTATTTTCACAGTCGCTCCTA |
| | | CCAGCTCTGAAATTCAGAACCCATATGACTGATGGCATATTCAGATAATCGG |
| | | GTCCCAGGTCTGGAAAAGCAGCCTTTTCCCCACGTTTCTTTCCCCACCTAGGA |
| | | CCTCCTCTGATTCTTCACTGCATCTTCGAAAGAAAATGTATTATTTGCTTGCCT |
| | | GGAAGACGCTGCAATTCAATTGATTTTATATATACATATATATAAAGAAAAC |
| | | AGAAAACATAGCCTAGATACCGGTCTTGAGCGTCACCGCCCCACTCGCGGTT |
| | | GTGAGCAAAGCCCTACGGAAGAAACCAATTCCCAGCCTAGACTCTTCAGAGC |
| | | CCAAGGTCGGGGAGGCGCTGGCCTGGCGGTGTTGTCTGGCTCCCCAGCCACT |
| | | GCCCCAGACTCAGGGCTTTGCCATTGGTCCCCACCTCCTCTGCTCCGGAGTTT |
| | | TTCTCCAGCTCCCCACCAAGCCACACAAAGTGACTTCTCGGAAACATTAGCC |
| | | GATTCTGCTGAGCAGGAAGGGAGGAAAGGGATGATGGGGGCGGGGGTGAGA |
| | | TAAGGGAAGGGCTCTTCTGGCTGCTGGACACACACACACACACACTCAAACA |
| | | CACACACGCCCCACCCAATGGGTGGCCGTGGATGGCAGGTCGTGCAACCCCC |
| | | TCCTCCGCCTTCTATTAGCGCATGGTGCAGAGGCTACAGCGTCGCCACCACCG |
| | | CGCCCCTAGCTGGGTCCCCGCCCTGCGCCGCCCGCAGGAGTGGAGAGAGGGA |
| | | GGGAGGGAGGGAGCAAGGGGTGGGGACCCGGGCGCGCTGGGAGGAGTGGA |
| | | GGAGGCAAAGCGGCGCAGCTGCCCTCGGGGAGGCGGGGCTGCTACCTCCAC |
| | | GGGCGCGCCCTGGCAGGAGGGGCGCAGTCTGCTTGCAGGCGGTCGCCAGCGC |
| | | TCCAGCGGCGGCTGTCGGCTTTCCAATTCCGCCAGCTCGGCTGAGGCTGGGCT |
| | | AGCCTGGGTGCCAGTGGCTGCTAGCGGCAGGCGTCCCCTGAGCAACAGGAGC |
| | | CCAGAGAAAAAGAAGCAGCCCTGAGAGA |
| hSyn | SEQ ID | CAGTGCAAGTGGGTTTTAGGACCAGGATGAGGCGGGGTGGGGGTGCCTACCT |
| | NO: 51 | GACGACCGACCCCGACCCACTGGACAAGCACCCAACCCCCATTCCCCAAATT |
| | | GCGCATCCCCTATCAGAGAGGGGGAGGGGAAACAGGATGCGGCGAGGCGCG |
| | | TGCGCACTGCCAGCTTCAGCACCGCGGACAGTGCCTTCGCCCCCGCCTGGCG |
| | | GCGCGCGCCACCGCCGCCTCAGCACTGAAGGCGCGCTGACGTCACTCGCCGG |
| | | TCCCCCGCAAACTCCCCTTCCCGGCCACCTTGGTCGCGTCCGCGCCGCCGCCG |
| | | GCCCAGCCGGACCGCACCACGCGAGGCGCGAGATAGGGGGGCACGGGCGCG |
| | | ACCATCTGCGCTGCGGCGCCGGCGACTCAGCGCTGCCTCAGTCTGCGGTGGG |
| | | CAGCGGAGGAGTCGTGTCGTGCCTGAGAGCGCAG |
| CMV | SEQ ID | CTCGAGGCGTTGACATTGATTATTGACTAGTTATTAATAGTAATCAATTACGG |
| | NO: 52 | GGTCATTAGTTCATAGCCCATATATGGAGTTCCGCGTTACATAACTTACGGTA |
| | | AATGGCCCGCCTGGCTGACCGCCCAACGACCCCCGCCCATTGACGTCAATAA |
| | | TGACGTATGTTCCCATAGTAACGCCAATAGGGACTTTCCATTGACGTCAATGG |
| | | GTGGAGTATTTACGGTAAACTGCCCACTTGGCAGTACATCAAGTGTATCATAT |
| | | GCCAAGTACGCCCCCTATTGACGTCAATGACGGTAAATGGCCCGCCTGGCAT |
| | | TATGCCCAGTACATGACCTTATGGGACTTTCCTACTTGGCAGTACATCTACGT |
| | | ATTAGTCATCGCTATTACCATGGTGATGCGGTTTTGGCAGTACATCAATGGGC |
| | | GTGGATAGCGGTTTGACTCACGGGGATTTCCAAGTCTCCACCCCATTGACGTC |
| | | AATGGGAGTTTGTTTTGGCACCAAAATCAACGGGACTTTCCAAAATGTCGTA |
| | | ACAACTCCGCCCCATTGACGCAAATGGGCGGTAGGCGTGTACGGTGGGAGGT |
| | | CTATATAAGCAGAGCTCTCTGGCTAACT |
| pJET | SEQ ID | GGGCGGAGTTAGGGCGGAGCCAATCAGCGTGCGCCGTTCCGAAAGTTGCCTT |
| | NO: 53 | TTATGGCTGGGCGGAGAATGGGCGGTGAACGCCGATGATTATATAAGGACGC |
| | | GCCGGGTGTGGCACAGCTAGTTCCGTCGCAGCCGGGATTTGGGTCGCGGTTC |
| | | TTGTTTGT |
| CAG | SEQ ID | GCGTTACATAACTTACGGTAAATGGCCCGCCTGGCTGACCGCCCAACGACCC |
| | NO: 54 | CCGCCCATTGACGTCAATAATGACGTATGTTCCCATAGTAACGCCAATAGGG |
| | | ACTTTCCATTGACGTCAATGGGTGGAGTATTTACGGTAAACTGCCCACTTGGC |
| | | AGTACATCAAGTGTATCATATGCCAAGTACGCCCCCTATTGACGTCAATGAC |
| | | GGTAAATGGCCCGCCTGGCATTATGCCCAGTACATGACCTTATGGGACTTTCC |
| | | TACTTGGCAGTACATCTACGTATTAGTCATCGCTATTACCATGGTCGAGGTGA |
| | | GCCCCACGTTCTGCTTCACTCTCCCCATCTCCCCCCCCTCCCCACCCCCAATTT |
| | | TGTATTTATTTATTTTTTAATTATTTTGTGCAGCGATGGGGGCGGGGGGGGGG |
| | | GGGGGGCGCGCGCCAGGCGGGGCGGGGCGGGGCGAGGGGGGGGCGGGGC |
| | | GAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGCG |
| | | CGGCGGGCG |

In some embodiments, a polynucleotide may comprise a promoter having at least 50%, at least 60%, at least 70%, at least 75%, at least 80%, at least 85%, at least 90%, at least 92%, at least 95%, at least 97%, at least 98%, at least 99%, or 100% sequence identity to any one of SEQ ID NO: 44-SEQ ID NO: 54. In some embodiments, the polynucleotide may comprise a promoter of any one of SEQ ID NO: 44-SEQ ID NO: 54. The promoter may regulate transcription of one or more coding sequences of the polynucleotide. In some embodiments, the promoter may regulate transcription of a two or more epigenetic modulators. For example, the promoter may regulate expression of a Naᵥ1.7 epigenetic modulator and a Naᵥ1.8 epigenetic modulator.

In some embodiments, the promoter is specific to a target molecule so that the nucleic acid composition is specific for, or only expressed in, those cells expressing the target for increased therapeutic selectivity. As a non-limiting example, the target molecule is SCN9A, SCN10A, or both.

In some embodiments, the promoter further increases the specificity of the AAV tropism for cells that express a target molecule. As a non-limiting example, the target molecule is SCN9A. Downregulating or upregulating a target molecule only in target molecule-expressing cells may reduce off-target effects and general toxicity. This is important to prevent the expression of the effectors (e.g., dCas or ZFP) in immune cells such as glial cells, microglia, macrophages, astrocytes, etcetera, that can mediate an immune reaction against the gene therapy proposed herein.

In some embodiments, the promoter is specific to SCN9A (Naᵥ1.7) and is utilized to drive the repression of Naᵥ1.7 or other gene products (e.g., SEQ ID NO: 44-SEQ ID NO: 50). In some embodiments, the promoter is specific to a gene target described herein. In some embodiments, the promoter is a ubiquitous promoter (e.g., SEQ ID NO: 52-SEQ ID NO: 54)

In some embodiments, some promoters can be induced with small molecules or other means. These inducible expression promoters include tetracycline responsive promoter, a glucocorticoid responsive promoter, an RU-486 responsive promoter, a peroxide inducible promoter and tamoxifen induced promoter.

Furthermore, there are promoters that can be induced when a pathology arises, such as injury or inflammation. Injury induced promoters include the galanin promoter specific to nociceptive afferent neurons. The inflammation-inducible promoter NF-κB could also be used for pain associated with inflammation.

Tandem promoters and combinations of promoters can also be used to prevent immune responses and create more cell-type specific expression.

In some embodiments, expression of the epigenetic modulator and/or transcription regulatory domain occurs upon a natural or physiological induction of the promoter.

In some embodiments, pan-neuronal gene promoters are used for modulation of gene expression in neurological diseases and for repression of pain-related genes. Non-limiting example promoters include the promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, the promoter of the NeuN gene (Fox-3, Rbfox3, or Hexaribonucleotide Binding Protein-3), the promoter of the α-calcium/calmodulin-dependent protein kinase II [CaMKIIα]), the promoter of the Rheb gene (ras homolog enriched in brain), TRKA promoter (Tyrosine Kinase A). In some embodiments, the promoter is neuronal specific, such as pol II promoters, including Thy1 and Hlxb9. Small latency-associated promoters from the herpesvirus pseudorabies virus can also be used for pan-neuronal expression of the effectors (dCas9 or ZFP) fused to a repressor domain.

Additional promoters include cytomegalovirus (CMV), SV40, elongation factor 1-alpha (EF1a) promoter, cytomegalovirus enhancer/chicken β-actin (CAG) promoter, jET promoter and herpes simplex virus (HSV).

In some embodiments, the promoter comprises a sequence at least about 90%, 91%, 92%, 93%, 94%, 95%, 96%, 97%, 98%, 99% or 100% identical to sequence of any of SEQ ID NO: 44-SEQ ID NO: 54.

In some embodiments, the promoter is a SCN9A promoter or SCN10A promoter.

In some embodiments, the promoter is naturally associated with a gene associated with a channelopathy, Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), pain (e.g., inflammatory pain, visceral pain, migraine pain, erythromelalgia pain, fibromyalgia pain, idiopathic pain, somatic pain), a neurological disease, dementia, Alzheimer's disease, Parkinson's disease, ALS, Multiple Sclerosis, a central nervous system ailment, or a combination thereof.

In some embodiments, the promoter is a promoter of a gene selected from SNCA, GBA, LRRK2, SOD1, ataxin-2, SCA2, BFD1, FUS, C9orf72, Brain-derived neurotrophic factor, Nerve growth factor, Neurotrophin, BCL11A, FMR1, DNM2, PrP, UBE3A, GYS1, and GFAP.

In some embodiments, the promoter is a pol II promoter (e.g., Thy1 and Hlxb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter.

### Other Components

A polynucleotide may further comprise an enhancer, an intron, a nuclear localization signal, an inverted terminal repeat (ITR), a terminator sequence, or combinations thereof. The polynucleotide may be included in a delivery vehicle, such as a vector.

Also provided herein are nucleic acid compositions comprising an adeno-associated virus modified with a sequence encoding a peptide specific for a protein product of a target molecule. Such compositions may be useful for targeting the nucleic acid to a cell expressing the target molecule for a targeted therapeutic approach. For example, the peptide specifically binds to the protein product of the target molecule. Non-limiting examples of specific binding include peptides that bind to the protein product of the target molecule with a high affinity, e.g., an affinity in the nanomolar range.

### Delivery Vectors

In some embodiments, a composition herein is delivered in a delivery vector. The delivery vector may be used to deliver an epigenetic modulator, a component of an epigenetic modulator, or a polynucleotide encoding an epigenetic modulator. In some embodiments, the delivery vector encapsulates a protein or a polynucleotide. In other embodiments, the composition is delivered complexed with cationic molecules.

In some embodiments, the composition is delivered to the subject via a vehicle. The vehicle may be a liposome, lipid nanoparticle, nanocapsule, or exosome.

In some embodiments, the composition is delivered via a viral vehicle. Non-limiting viral vehicles include, but are not limited to, retroviral vectors, lentiviral vectors, adenoviruses vectors, adeno-associated viral vectors (e.g., AAV1, AAV2, AAV3, AAV4, AAV5, AAV6, AAV7, AAV8, AAV9, AAV10, AAVhu68, AAVrh.10, AAVrh74, AAVDJ), and the like. In some cases, the vehicle is a recombinant adeno-associated virus (AAV). In some cases, the AAV is AAV9. AAV9 may be selected because it has the highest tropism for dorsal root ganglia (DRG) neurons where pain associated proteins such as Naᵥ1.7 are highly expressed. Further, AAV9 has been shown to be safe.

All delivery vehicles (viral vectors or non-viral) can have an improved tropism towards cells that express the protein product of a target molecule. For example, the vehicles can comprise a peptide that binds to the protein product of a target molecule. As a non-limiting example, the peptide binds to Naᵥ1.7 to target the nucleic acid to Naᵥ1.7 expressing cells.

### Targeting Moieties

In some embodiments, a composition herein is delivered via a viral vehicle, e.g., an AAV capsid described herein, with a nucleic acid sequence encoding a peptide targeting moiety.

In some embodiments, a composition herein is delivered via a non-viral delivery vehicle, such as, without limitation, a liposome, lipid nanoparticle, nanocapsule, or exosome. For some such embodiments, the vehicle may comprise and/or be connected to a targeting moiety, such as a small molecule or peptide targeting moiety.

In some embodiments, the targeting moiety comprises a peptide targeting moiety that binds to protein product of the target molecule in order to target the nucleic acid to a specific cell. In some cases, the target molecule is present on a target cell. In some cases, the target cell is associated with the disease or condition in the subject.

Non-limiting examples of peptide targeting moieties for use with viral and non-viral delivery methods include JNJ63955, m3-Huwentoxin-IV, Phlotoxin 1 (PhlTx1), Protoxin-II (ProTx-II), Ceratotoxin-1 (CcoTx1), Huwentoxin-IV (HwTx-IV), μ-TRTX-Pn3a, Jz-Tx-V, GsAFI, Tp1a (Protoxin-III), GpTx-1, HpTx1, Hm1a, and variants and combinations thereof. The peptide may originate from one or more of the following organisms: Thrixopelma prurient, Pamphobeteus nigricolor, Chilobrachys jingzhao, Grammostola spatulata, Phlogiellus genus, Thrixopelma pruriens, Grammostola porteri, Selenocosmia huwena, Ceratogyrus cornuatus, Heteropoda venatoria, and/or Heteroscodra maculate.

TABLE 8 provides examples of peptides that may be used for targeting.

TABLE 8
Exemplary Peptide Targeting Moieties

| | SEQ ID | |
|---|---|---|
| Name | NO | Sequence |
| Protoxin-II | SEQ ID | YCQKWMWTCDSERKCCEGMVCRLWCKKKLW |
| | NO: 109 | |
| Protoxin-II variant | SEQ ID | GPYCQKWMQTCDSERKCCEGMVCRLWCKKKLL |
| (JNJ63955) | NO: 110 | |
| μ-TRTX-Pn3a | SEQ ID | DCRYMFGDCEKDEDCCKHLGCKRKMKYCAWDF |
| | NO: 111 | TFT |
| Jz-Tx-V | SEQ ID | YCQKWMWTCDSKRACCEGLRCKLWCRKII |
| | NO: 112 | |
| Jz-Tx-V variant | SEQ ID | YCQKWMWTCDSKRACCEGLRCKLWCRKEI |
| | NO: 113 | |
| GsAFI | SEQ ID | YCQKWLWTCDSERKCCEDMVCRLWCKKRL |
| | NO: 114 | |
| Phlotoxin 1 | SEQ ID | ACLGQWDSCDPKASKCCPNYACEWKYPWCRYKL |
| | NO: 115 | F |
| Phlotoxin 1 variant | SEQ ID | ACLGQWASCDPKASKCCPNYACEWKYPWCRYKL |
| | NO: 116 | F |
| Tp1a (Protoxin-III) | SEQ ID | DCLKFGWKCNPRNDKCCSGLKCGSNHNWCKLHL |
| | NO: 117 | |
| GpTx-1 | SEQ ID | DCLGFMRKCIPDNDKCCRPNLVCSRTHKWCKYVF |
| | NO: 118 | |
| GpTx-1 variant | SEQ ID | DCLGFFRKCIPDNDKCCRPNLVCSRLHRWCKYVF |
| | NO: 119 | |
| GpTx-1 variant | SEQ ID | DCLGAMRKCIPDNDKCCRPNLVCSRTHKWCKYV |
| | NO: 120 | F |
| HwTx-IV | SEQ ID | ECLEIFKACNPSNDQCCKSSKLVCSRKTRWCKYQI |
| | NO: 121 | |
| m3-HwTx-IV variant | SEQ ID | GCLGIFKACNPSNDQCCKSSKLVCSRKTRWCKWQ |
| (m3-Huwentoxin-IV) | NO: 122 | I |
| CcoTx1 variant | SEQ ID | DCLGMFKSCDPENDKCCKRLVCSRSHRWCKWKL |
| | NO: 123 | |
| CcoTx1 variant | SEQ ID | ICLGMFKSCDPENDKCCKRLVCSRSHRWCKWKL |
| | NO: 124 | |
| HpTx1 | SEQ ID | DCGTIWHYCGTDQSECCEGWKCSRQLCKYVIDW |
| | NO: 125 | |
| Hm1a | SEQ ID | ECRYLFGGCSSTSDCCKHLSCRSDWKYCAWDGTF |
| | NO: 126 | S |
| AM-0422 | SEQ ID | YCQKW[Nle]WTCDSKRACC[Pra]GLRCKLWCRKEI |
| | NO: 127 | |
| OD-1 | SEQ ID | GVRDAYIADDKNCVYTCASNGYCNTECTKNGAES |
| | NO: 128 | GYCQWIGRYGNACWCIKLPDEVPIRIPGKCR |
| Penetration enhancing | SEQ ID | LLIILRRRIRKQAHAHSK |
| peptide | NO: 129 | |
| Penetration enhancing | SEQ ID | KETWWETWWTEWSQPKKKRKV |
| peptide | NO: 130 | |
| Penetration enhancing | SEQ ID | KLALKLALKALKAALKLA |
| peptide | NO: 131 | |
| Penetration enhancing | SEQ ID | LSTAADMQGVVTDGMASGLDKDYLKPDD |
| peptide | NO: 132 | |
| Penetration enhancing | SEQ ID | LLKKKCWLRCVMGECCKRESDCTQMWKQCYPG |
| peptide | NO: 133 | |

In some cases, the peptide comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 109-SEQ ID NO: 133. The peptides of TABLE 8 may bind to Naᵥ1.7 and thus may be useful to provide AAV tropism to Naᵥ1.7 expressing cells. In some cases, the peptide comprises ProTx-II, ProTx-III, the ProTx-II variant JNJ63955, HwTx-IV variant m3-HwTx-IV, CcoTx1 variant 2670, PhlTx1 variant D7A-PhlTx1, or JxTx-V variant AM-0422.

### AAV Vectors

Recombinant adeno-associated viruses (AAVs) are among the most commonly used vehicles for in vivo gene delivery. To enter a host cell, the virus 1) binds a receptor and glycan co-receptor on the cell surface, 2) is endocytosed, 3) progresses through the endosomal compartment, 4) escapes the endosome, and 5) traffics to the nucleus. Once inside the nucleus, the virus sheds its coat and its single-stranded genome is converted to a double-stranded one which the host cell can now use as a template for gene expression. The AAV receptor (AAVR, KIAA0319L) has been reported to be essential for AAV entry into cells, however, some recombinant AAVs can enter cells independent of AAVR. Glycosylation is also known to play a role in viral transduction efficiency. Changing the capsid composition of an AAV changes the ability of that AAV to enter cells. There are at least four major techniques currently used towards modifying and improving AAV tropism: rational engineering, directed evolution, evolutionary lineage analysis, and chemical conjugation.

One way to go about modifying a virus's tropism is to generate a large library of peptides to add onto the AAV surface and then characterize the resultant variants to determine their tropism. This method is labor intensive, requiring massive screening efforts as the library of peptides screened are more or less generated randomly so success is based on a numbers game. In a similar technique referred to as directed viral evolution, organs are collected from the first round of viral infection and screened to select viral variants with desired tropism (ex: brain-specific) for subsequent rounds of infection to further select even more specific tropism. However, some of these screening methods are not able to be translated between species.

Instead of using a random library screening, described herein is a rational design method to improve AAV tropism. Rational design strategies for AAV capsid engineering include peptide domain insertions and chemical biology approaches. One can add peptides which are known to specifically interact with cells of interest. By using binding peptides that bind to the protein encoding a target molecule, AAVs described herein have increased tropism towards target molecule expressing cells, generating more targeted strategies.

In some embodiments, the peptide to increase tropism binds to Naᵥ1.7. Peptides to increase Naᵥ1.7 tropism include: JNJ63955, m3-Huwentoxin-IV, Phlotoxin 1 (PhlTx1), Protoxin-II (ProTx-II), Ceratotoxin-1 (CcoTx1), Huwentoxin-IV (HwTx-IV), and those described elsewhere herein, e.g., a peptide of any of SEQ ID NO: 109-SEQ ID NO: 133 or a sequence at least about 90%, 91%, 92%, 93%, 94%, 95%, 96%, 97%, 98%, 99% or 100% identical to any of SEQ ID NO: 109-SEQ ID NO: 133.

### Target Molecules

In certain embodiments compositions described herein modulate expression of one or more target molecules associated with a disease or condition in a subject. In some cases, expression of the target molecule is activated. In some cases, expression of the target molecule is repressed.

The one or more target molecules may comprise DNA or RNA. In some embodiments, a target molecule comprises DNA. In some embodiments, a target molecule comprises a coding region of a gene. In some embodiments, a target molecule comprises DNA complementary to non-coding RNA. The non-coding RNA may be associated with a disease or condition described herein (e.g., pain). For example, the non-coding RNA is associated with neuropathic pain such as spinal nerve ligation, spared nerve injury, chronic constriction injury, or diabetic neuropathy, or a combination thereof.

In some embodiments, the non-coding RNA comprises a SCN9A natural antisense transcript (NAT), a Kcna2 antisense RNA, H19, Gm21781, MRAK009713, uc.48+, NONRATT021972, BC168687, Speer7-ps, Uc007pbc.1, XLOC_041439, Mlxipl, Rn50_X_0739.1, CCAT1, rno circ 0004058, rno_circRNA_007512, or Egr2 antisense RNA, or a combination thereof.

In some embodiments, the one or more target molecules comprises a nucleic acid associated with a disease or condition described here. Non-limiting examples of nucleic acids associated with diseases and conditions described herein are shown in Tables 1-3. In some embodiments, the one or more target molecules comprises a nucleic acid associated with pain. Pain includes neuropathic pain, inflammatory pain, visceral pain, migraine pain, erythromelalgia pain, fibromyalgia pain, idiopathic pain, and somatic pain.

In some embodiments, the one or more target molecules comprises a nucleic acid associated with a channelopathy. In some cases, the one or more target molecules comprises a nucleic acid encoding a channel. The channel may be an ion channel, e.g., sodium channel, potassium channel, calcium channel, and/or chloride channel.

In some embodiments, the one or more target molecules comprises a nucleic acid associated with a neurological disease. In some embodiments, the one or more target molecules comprises a nucleic acid associated with dementia. In some embodiments, the one or more target molecules comprises a nucleic acid associated with Alzheimer's disease. In some embodiments, the one or more target molecules comprises a nucleic acid associated with Parkinson's disease. In some embodiments, the one or more target molecules comprises a nucleic acid associated with Huntington's disease. In some embodiments, the one or more target molecules comprises a nucleic acid associated with schizophrenia. In some embodiments, the one or more target molecules comprises a nucleic acid associated with Amyotrophic lateral sclerosis (ALS). In some embodiments, the one or more target molecules comprises a nucleic acid associated with Multiple Sclerosis. In some embodiments, the one or more target molecules comprises a nucleic acid associated with a central nervous system ailment. In some embodiments, the one or more target molecules comprises a nucleic acid associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

In some embodiments, the one or more target molecules comprises one or more genes of Tables 1-3. In some embodiments, the one or more target molecules comprises SCN9A, SCN10A, or SCN11A, or a combination thereof. In some embodiments, the one or more target molecules comprises SNCA, GBA, or LRRK2, or a combination thereof. In some embodiments, the one or more target molecules comprises GPR52. In some embodiments, the one or more target molecules comprises SOD1, ataxin-2, or SCA2, or a combination thereof. In some embodiments, the one or more target molecules comprises one or more genes selected from the group comprising BFD1, FUS, C9orf72, Brain-derived neurotrophic factor, Nerve growth factor, a Neurotrophin, BCL11A, FMR1, DNM2, PrP, UBE3A, GYS1, and GFAP.

### Genes Involved in Pain

The human genome encodes genes that can confer protection to unnecessary pain. Genetic studies have correlated a hereditary loss-of-function mutation in a human-voltage gated sodium channel—Naᵥ1.7 (SCN9A)—with a rare genetic disorder, which leads to insensitivity to pain without other neurodevelopmental alterations. Thus, this sodium channel has been an attractive target for developing chronic pain therapies. However, efforts to develop selective small molecule inhibitors have been hampered due to the high sequence identity between Naᵥ subtypes, and in fact, many small-molecule drugs targeting Naᵥ1.7 have failed due to lack of specificity. Antibodies have faced a similar situation since there is a tradeoff between selectivity and potency due to the antibody binding to a specific (open or close) conformation of the channel. Indeed, even commercially available antibodies targeting the human channel are poor for a western blot. Interference RNA (RNAi) has also been utilized to target Naᵥ1.7. As an exogenous system, however, RNAi competes with endogenous machinery such as microRNA or RISC complex function. Thus, RNAi can compete with and impair fundamental homeostatic mechanisms of RNA synthesis and degradation. In addition, due to high RNA turnover, RNAi methods have poorer pharmacokinetics prospects and require higher dosage. It is mainly due to these drawbacks that none of the Naᵥ1.7-targeting treatments based on these methods have yet succeed to reach the final phase of clinical trials. In contrast, disclosed here is the use of nucleic acid binding domains (e.g., nuclease-inactivated “dead” CRISPR-Cas (dCas)—also known as CRISPR interference or CRISPRi- and Zinc-Finger proteins) and epigenetic modulators (e.g., KRAB repressor) to repress the transcription of SCN9A, SCN10A, and/or other genes associated with pain. As permanent ablation of pain is not desired, there is no permanent genome editing using such methods. Instead, these epigenomic engineering methods enable transient modulation of Naᵥ1.7 gene expression, Naᵥ1.8 gene expression, or both. Additionally, this approach may have lower risk of off-target effects than other approaches. Rather than pharmacologically targeting the protein or RNA, this approach targets Naᵥ1.7, Naᵥ1.8, or both at the DNA level. This may result in longer lasting results than methods targeting protein or RNA. With this approach, one can engineer highly specific, long-lasting, and reversible treatments for pain. Treatment duration is important because many pain states resulting from chronic inflammation and nerve injury are enduring conditions which typically require continual re-medication. This genetic approach provides ongoing, controllable regulation of the aberrant pain processing. Further, since the disclosed approach can be easily designed to target several genes, it represents a new paradigm in pain management since it provides a synergistic way of targeting single or multiple sodium channels for more potent pain relief.

In addition to SCN9A and SCN10A, TABLE 9 provides other genes involved in pain. For genes that are upregulated, the methods herein may repress expression; and for genes that are downregulated, the methods herein may activate expression. In some cases, the methods repress and activate expression of one or more genes.

TABLE 9
Genes Involved in Pain

| | | | Activate or |
|---|---|---|---|
| | | Up/downregulated in | Repress with |
| Gene name | Biological process | response to pain | Gene Therapy |
| Cxcl13 | Chemokine-mediated | Upregulated | Repress |
| | signaling pathway | | |
| C1qc | Innate immune response | Upregulated | Repress |
| Ccl2 | Regulation of sensory | Upregulated | Repress |
| | perception of pain | | |
| Fcgr3a | Regulation of sensory | Upregulated | Repress |
| | perception of pain | | |
| Ngf | Sensory perception of pain | Upregulated | Repress |
| Ltc4s | Response to axon injury | Upregulated | Repress |
| Rpe65 | Cellular response to | Upregulated | Repress |
| | electrical stimulus | | |
| Aoah | Inflammatory response | Upregulated | Repress |
| Aif1 | Response to axon injury | Upregulated | Repress |
| Capn11 | Calcium-dependent cysteine | Upregulated | Repress |
| | protease | | |
| HDAC5, | Regulates neuropathic pain | Upregulated | Repress |
| HDAC5, | through SRY-related HMG- | | |
| HDAC7 | box 10 (SOX10) | | |
| P2x3 | Purinergic receptor | Upregulated | Repress |
| P2x4 | Purinergic receptor | Upregulated | Repress |
| NMDA | Subunit of the N-methyl-D- | Upregulated | Repress |
| receptor | aspartate (NMDA) receptor | | |
| NR2B | is an example of a | | |
| | heteromeric ligand-gated | | |
| | ion channel that interacts | | |
| | with multiple intracellular | | |
| | proteins by way of different | | |
| | subunits. | | |
| NMDA | Subunit of the N-methyl-D- | Upregulated | Repress |
| receptor | aspartate (NMDA) receptor | | |
| NR1 | is an example of a | | |
| | heteromeric ligand-gated | | |
| | ion channel that interacts | | |
| | with multiple intracellular | | |
| | proteins by way of different | | |
| | subunits. | | |
| MMP-2 & | Metalloprotease-9 and -2 | Upregulated | Repress |
| MMP-9 | | | |
| K channel | Potassium channel | Downregulated | Activate |
| Kir4 | | | |
| TRPV1/2/3/4 | Capsaicin receptor | Upregulated | Both |
| EP receptor | Prostaglandin E(2) | Upregulated | Repress |
| EP4 | (PGE(2)) is both an | | |
| | inflammatory mediator | | |
| | released at the site of tissue | | |
| | inflammation and a | | |
| | neuromodulator that alters | | |
| | neuronal excitability and | | |
| | synaptic processing | | |
| Nav1.1 | Voltage-gated sodium | Upregulated | Repress |
| (SCN1A) | channel | | |
| Nav1.3 | Voltage-gated sodium | Upregulated | Repress |
| (SCN3A) | channel | | |
| Nav1.6 | Voltage-gated sodium | Upregulated | Repress |
| (SCN8A) | channel | | |
| Nav1.7 | Voltage-gated sodium | Upregulated | Repress |
| (SCN9A) | channel | | |
| Nav1.8 | Voltage-gated sodium | Upregulated | Repress |
| (SCN10A) | channel | | |
| Nav1.9 | Voltage-gated sodium | Upregulated | Both |
| (SCN11A) | channel | | |
| KCNQ | Voltage-gated potassium | Downregulated/Upregulated | Activate |
| (KCNQ2, | channels | depending on pain | |
| KCNQ3, | | phenotype (KCNQ2 up) | |
| KCNQ4, | | | |
| KCNQ5) | | | |
| TRPM8 | Transient receptor potential | Downregulated/Upregulated | Repress/Activate |
| | melastatin 8 | depending on pain | depending on |
| | | phenotype | pain type |
| TRPA1 | Transient receptor potential | Upregulated | Repress |
| | cation channel | | |
| CTNNB1 | Encodes an important | Upregulated | Repress |
| | cytoplasmic component of | | |
| | the classical cadherin | | |
| | adhesion complex, also | | |
| | involved in Wnt signaling | | |
| Wnt3a | Wnt signaling | Upregulated | Repress |
| IRS1 | Insulin receptor substrate | Downregulated | Activate |
| mTORC1 | Mammalian target of | Downregulated | Activate |
| | rapamycin complex 1 | | |
| ZFHX2 | Putative transcription factor | Downregulated | Activate |
| TAK1 | Tumor necrosis factor-α | Upregulated | Repress |
| TRPC5 | Transient receptor potential | Downregulated | Activate |
| | canonical 5 | | |
| CDK6 | Cell division protein kinase | Upregulated | Repress |
| | 6 | | |
| PKIA-AS1 | IncRNA | Upregulated | Repress |
| GSK3B | Serine/threonine kinase | Upregulated | Repress |
| OAT | Ornithine Aminotransferase | Upregulated | Repress |
| SHANK3 | Connects neurotransmitter | Upregulated | Repress |
| | receptors, ion channels, and | | |
| | other membrane proteins to | | |
| | the actin cytoskeleton and | | |
| | G-protein-coupled signaling | | |
| | pathways. | | |
| ODC1 | Ornithine decarboxylase | Downregulated | Activate |
| TRPM1 | Transient Receptor | Downregulated | Activate |
| | Potential Cation Channel | | |
| | Subfamily M Member 1) | | |
| TLR4 | Toll Like Receptor 4 | Upregulated | Repress |
| ARRB2 | Beta-arrestin-2 | Downregulated | Activate |
| PKC | Protein Kinase C | Upregulated | Repress |
| CACNA1A | Calcium Voltage-Gated | Downregulated (mutations | Activate |
| | Channel Subunit Alpha1 A | involved in familial | |
| | | migraine) | |
| AIBP | Apolipoprotein A-I binding | Downregulated | Activate |
| | protein | | |
| IL-10 | Pleiotropic cytokine | Downregulated | Activate |
| POMC | Proopiomelanocortin | Downregulated | Activate |
| Cav2.2 | Voltage-gated calcium | Upregulated | Repress |
| | channel | | |
| Cav3.1 | Voltage-gated calcium | Upregulated | Repress |
| | channel | | |
| Cav3.2 | Voltage-gated calcium | Upregulated | Repress |
| | channel | | |
| Kelch-like 1 | A structural protein that | | Repress |
| (KLHL1) | binds to Cav3.2 and actin | | |
| mTOR | member of the | Upregulated | Repress |
| | phosphatidylinositol 3- | | |
| | kinase-related kinase family | | |
| | of protein kinases | | |
| KCNA2 | voltage-gated potassium | Downregulated | Activate |
| (Kv1.2) | channel | | |
| NK1R | Neurokinin 1 receptor | Activated in pain conditions | Repress |
| | (NK1R) antagonist | as well as anxiety, | |
| | possesses antidepressant, | depression via substance P. | |
| | anxiolytic, and antiemetic | | |
| | properties. They boost the | | |
| | efficacy of 5-HT3 | | |
| | antagonists to prevent | | |
| | nausea and vomiting. | | |
| STING1 | Stimulator of Interferon | Downregulated | Activate |
| (TMEM- | Response cGAMP | | |
| 173) | Interactor-1. Stimulator of | | |
| | interferon genes. | | |
| HCN2 | Hyperpolarization-activated | Upregulated | Repress |
| | cyclic nucleotide-gated ion | | |
| | channel | | |
| HCN3 | Hyperpolarization-activated | Activated | Repress |
| | cyclic nucleotide-gated ion | | |
| | channel | | |
| ASIC3 | Acid-sensing ion channel. A | Activated | Repress |
| | sensor of acidic and primary | | |
| | inflammatory pain. | | |
| Caveolin-1 | Cholesterol binding and | Downregulated | Activate |
| | resident protein. Organizes | | |
| | and targets synaptic | | |
| | components of the | | |
| | neurotransmitter and | | |
| | neurotrophic receptor | | |
| | signaling pathways. | | |

### Genes Involved in Channelopathies

In certain embodiments, the target molecule comprises a gene involved in a channelopathy. The genes may be associated with or encode a channel such as a sodium channel, potassium channel, calcium channel, and/or chloride channel. Non-limiting examples of such genes are provided in TABLE 10.

TABLE 10
Genes Involved in Channelopathies

| Channels | Potassium | Sodium | Calcium | Chloride |
|---|---|---|---|---|
| Epileptic syndromes | KCNA1, KCNA2, | SCN1A, | CACNA1H | CLCN2, |
| | KCNQ2/3, KCNMA1, | SCN1B, | | GLIALCAM |
| | KCNT1, KCND2, | SCN2A, | | |
| | KCNH5, KCNJ10, | SCN3A, | | |
| | KCNJ11, | SCN8A, | | |
| Ataxia syndromes | KCNA1, KCNC3, | | CACNA1A | |
| | KCND3 | | | |
| Familial Hemiplegic | | SCN1A | CACNA1A | |
| Migraine | | | | |
| Pain syndromes and | KCNQ2 | SCN9A | TRPA1 | — |
| neuropathies | | | | |
| Bartter's syndrome | ROMK1 | | | CLCNKB, BSDN |
| Dent disease | | | | CLCN5 |
| EAST/SESAME | KCNJ11 | | | |
| syndrome | | | | |
| Long QT and Short | KCNQ1, KCNH2, | SCN5A | CACNAIC | |
| QT syndromes | KCNE1, KCNJ2 | | | |
| Brugada syndromes | KCNE3, HCN4 | SCN5a, | CACNAIC | |
| | | SCN1B, | | |
| | | SCN3B | | |
| Catecholaminergic | | | RYR2 | |
| polymorphic | | | | |
| ventricular | | | | |
| tachycardia | | | | |
| Familial Congenital | KCNJ11, ABCC8 | | | |
| hyperinsulinism and | | | | |
| neonatal diabetes | | | | |
| mellitus | | | | |
| Non dystrophic | | SCN4A | | CLCN1 |
| myotonias | | | | |
| Periodic paralysis | KCNJ2, KCNJ18 | SCN4A | CACNA1S | |
| Osteopetrosis | | | | CLCN7, OSTM1 |

### Genes Involved in Neurological Diseases or Conditions

In certain embodiments, the target molecule comprises a gene involved in a neurological disease or condition of the central nervous system. Non-limiting example diseases and conditions include Alzheimer's disease, Parkinson disease, Huntington's disease, Amyotrophic lateral sclerosis (ALS) and Multiple Sclerosis.

Alzheimer's disease (AD) is a progressive disease that causes brain cells to waste away (degenerate) and die. Alzheimer's disease is the most common cause of dementia a continuous decline in thinking, behavioral and social skills that disrupts a person's ability to function independently. Activation and/or repression of genes could potentially slow the progression or prevent Alzheimer's disease.

Research has shown that the binding of B-Amyloids to LilrB2 (e.g., UniProt Ref No. Q8N423) is one of the first steps leading to Alzheimer's disease. Thus, repression of LilrB2 such that it affects the B-Amyloid binding could prevent the onset of Alzheimer's disease. D1 is associated with Uniprot Ref No. P21728. D2 is associated with Uniprot Ref No. 14416. Therefore, provided herein are compositions and methods of repressing expression of LilrB2.

MS4A4A and TREM2 operate in the microglia, the brain's immune cells. They influence Alzheimer's disease risk by altering levels of TREM2, a protein that helps microglia cells clear excessive amounts of the Alzheimer's proteins amyloid and tau from the brain. Thus, activation of TREM2 in the cerebrospinal fluid (CSF) could help prevent and/or slow the progression of Alzheimer's disease. Therefore, provided herein are compositions and methods of activating expression of TREM2.

Apolipoprotein E4 (apoE4), the most prevalent genetic risk factor of Alzheimer's disease, is expressed in more than half of Alzheimer's disease patients and is thus an important possible therapeutic target. Of the three polymorphic forms of apoE, namely apoE2, apoE3, and apoE4, carriers of apoE4 are more likely to develop Alzheimer's disease. Blocking the apoE4 effect would help the 40-60% of AD patients who carry apoE4, whereas, if all apoE forms are in fact toxic, another approach would be to block all apoE action. In addition, several studies have revealed that apoE4 is also a risk factor for other diseases including cerebral amyloid angiopathy, dementia with Lewy bodies, tauopathy, cerebrovascular disease, multiple sclerosis, and vascular dementia. Accordingly, provided herein are compositions and methods for repressing APOE4 expression.

VEGF (vascular endothelial growth factor), originally described as a key angiogenic factor, has been shown to play an important role in neurogenesis and neuroprotection and to affect neuronal plasticity and repair. Animal model studies revealed that brain levels of VEGF and its receptor (VEGFR-2) were reduced in the hippocampus of apoE4 mice. Thus, upregulation of the levels of hippocampal VEGF may reverse the accumulation of A3 and hyperphosphorylated tau in hippocampal neurons. Thus, provided herein are compositions and methods for upregulating VEGF expression.

Similarly, ABCA1 (ATP-binding cassette transporter ABCA1) upregulation reverses the apoE4-driven cognitive and brain pathology, and therefore ABCA1 is a target molecule for upregulation and Alzheimer's disease treatment.

Transactive response DNA-binding protein 43 (TDP-43), an RNA-binding protein that functions in axon skipping, has recently been shown to be deposited in AD brain. TDP-43 is present in the brain of 65-80% of AD patients and was shown to be associated with progressive hippocampal atrophy. Therefore, in some embodiments, a target molecule is TDP-43, and compositions described herein repress TDP-43 expression.

Target molecules also include genes having known mutations that lead to early-onset Alzheimer's disease, such as presenilin 1 (PSEN1) and presenilin 2 (PSEN2). Thus, activation of these genes could be another potential avenue for treatment of Alzheimer's disease.

Further non-limiting target molecules involved in neurological diseases such as Alzheimer's disease are shown in TABLE 11. Such genes may be repressed or upregulated using the compositions and methods described herein. Activation or repression may be desired depending on the pathology; example modulations are provided for Alzheimer's disease.

TABLE 11
Genes Involved in Neurological Disease

| | | Activate or |
|---|---|---|
| | | Repress with |
| Gene name | Biological process | Gene Therapy |
| Triggering receptor expressed | Operate Microglia | Activate |
| on myeloid cells 2 TREM2 | | |
| Membrane-Spanning 4- | Operate Microglia | Activate |
| domains subfamily A (MS4A) | | |
| LilrB2 | | Repress |
| APOE4 | Lipid metabolism | Repress |
| Presenilins PSENI, PSEN2 | | Activate |
| Vascular endothelial growth | Neurogenesis and | Upregulation |
| factor VEGF | neuroprotection | |
| Vascular endothelial growth | Neurogenesis and | Upregulation |
| factor Receptor VEGFR | neuroprotection | |
| RANBP17 | Nuclear transport | Repress |
| | receptor (importin-beta | |
| | superfamily) | |
| LAMA3 | Human laminin, alpha 3 | Repress |
| PCDH10 | Human protocadherin 10 | Repress |
| MAPT (encodes Tau protein) | microtubule-associated | Repress |
| | protein in neuro | |
| SODI (superoxide dismutase | It is implicated in | Repress |
| 1) | apoptosis and familial | |
| | amyotrophic lateral | |
| | sclerosis. | |
| C9orf72 (chromosome 9 open | It is the most common | Repress |
| reading frame 72) | mutation affected gene | |
| | that is associated with | |
| | familial frontotemporal | |
| | dementia (FTD) and/or | |
| | amyotrophic lateral | |
| | sclerosis (ALS). | |
| TDP-43 (transactive response | Correlated with ALS. | Repress |
| DNA binding protein 43 kDa) | | |
| FUS (FUsed in Sarcoma) | RNA-binding protein. | Repress |
| | Pathological link to ALS. | |

Additional target molecules include alpha-synuclein, microtubule-associated protein tau, APP, and huntingtin (SNCA, MAPT, APP, and HTT, respectively). Precise transcriptional modulation has been performed through CRISPRa of neurodegenerative disease-related genes in human iPSC-derived neurons. TSS2-2 sgRNA and dCas9-VPR transcriptional activator mediated the activation of alpha-synuclein in normal alpha-synuclein levels (NAS) iPSC-derived neurons from healthy control patient and iPSCs derived from a patient with Parkinson's disease caused by alpha-synuclein triplication (AST). An eightfold activation of endogenous SNCA expression was achieved in the NAS iPSC-derived neurons and through dCas9-KRAB repression, a 40% reduction in alpha-synuclein mRNA levels in the AST iPSC-derived neurons. In addition, targeting the genes close to the TSS region reduced the off-target effects considerably, reducing the negative side effects of using SpCas9. Overall, these findings suggest the possibility of exploiting the tunable CRISPRa/CRISPRi platform for multiplex transcriptional repression of molecular pathological signatures in vivo in the mammalian brain and the possibility for addressing neurodegeneration in the familial and sporadic disease states.

In some embodiments, the target molecule comprises one or more of alpha synuclein (SNCA), glucocerebrosidase (GBA), and/or leucine-rich repeat kinase (LRRK2). In some cases, SNCA degradation is prevented by inhibiting tyrosine kinase c-ABL. In some cases, expression of SNCA is repressed. The target molecule may be repressed for treatment of Parkinson's or another condition of the nervous system.

In some embodiments, the target molecule comprises HTT encoding Huntington's protein. Repression of mutated HTT may be performed to treat Huntington's disease.

In some embodiments, the target molecule comprises GPR52. Modulation of GPR52 may be performed to treat Huntington's disease and/or schizophrenia. GPR52 upregulation may be used for schizophrenia, cognitive impairment, psychiatric disorders, brain malformation and hyperactivity. Repression of GPR52 may be used for the treatment of Huntington's disease, as GPR52 is associated with the abnormal expression of huntingtin that is observed in patients with this disorder.

In some embodiments, the target molecule comprises at least one of superoxide dismutase 1 (SOD1), ataxin-2, and/or transactive response DNA-binding protein 43 (TDP-43). Modulation of the target molecules may be performed to treat ALS, such as repression of SOD1, ataxin-2 or TDP43. Modulation of the target molecules may be performed to treat frontotemporal dementia (FTD) or other neurological diseases.

In some embodiments, the target molecule comprises BFD1, encoding bradyzoite-formation deficient 1, a transcription-factor protein. BFD1 can drive the expression of genes needed for the formation of bradyzoites of Toxoplasma Gondii, and therefore its repression may prevent chronification of this infection and other infections using a similar route of chronification.

A mutation in the C9orf72 gene is the most common genetic cause of two neurodegenerative diseases. Studies of mice and humans suggest a role for loss of the C9orf72 protein in some neurodegenerative disorders as with reduced C9orf72 levels, there is more inflammation mediated by the STING protein in immune and brain cells. Therefore, in some embodiments, the target molecule comprises C9orf72, and compositions herein upregulate C9orf72 expression. In some embodiments, the target molecule comprises the STING gene.

Additional genes that could be upregulated to treat neurological diseases include neurotrophic factors such as Brain-derived neurotrophic factor, Nerve growth factor and Neurotrophins. The DDC gene encoding AADC can be upregulated to treat Parkinson's disease. Genes that could be downregulated to treat the following diseases include: B-thalassemia (BCL11A), Fragile X (FMR1), centronuclear myopathy (DNM2), Prion disease (PrP), Angelman Syndrome (UBE3A), Lafora disease (GYS1), and Alexander disease (GFAP).

### Methods of Treatment

Various embodiments provide for methods of treating a disease or condition in a subject with the compositions described herein. In some embodiments, the composition comprises an epigenetic modulator or a polynucleotide encoding an epigenetic modulator. The composition may be delivered via AAV or non-viral vehicles.

In example embodiments, the disease or condition comprises pain. Pain includes neuropathic, inflammatory, visceral, migraine, erythromelalgia, fibromyalgia, idiopathic and somatic pain. In some embodiments, the pain is chemotherapy-induced (e.g., paclitaxel-induced). Inflammatory pain comprises rheumatoid arthritis pain. The disease or condition also includes those where Naᵥ1.7 or other genes involved in pain could be targeted. In some embodiments, a method of treatment comprises treating inflammation in the subject with a composition comprising an epigenetic modulator or a polynucleotide encoding an epigenetic modulator. The inflammation may be associated with a disease or condition, such as arthritis.

In some embodiments, the disease or condition comprises a channelopathy. Channelopathies include Dravet syndrome, Epilepsy syndromes, Familial hemiplegic migraine, Ohtahara syndrome, West syndrome, Lennox-Gastaut syndrome, sodium channel myotonia, autism, Long QT syndrome, Brugada syndrome, Progressive cardiac conduction disease (also called Lenègre disease), and the like. Example diseases and conditions are shown in TABLE 11.

In some embodiments, the disease or condition comprises a neurological disease or condition. Neurological diseases or conditions include dementia, Alzheimer's disease, Parkinson disease, Huntington's disease, schizophrenia, Amyotrophic lateral sclerosis (ALS) and Multiple Sclerosis. The disease or condition may comprise a central nervous system ailment.

In some embodiments, the disease or condition comprises an inflammatory disease or condition. As a non-limiting example, the inflammatory disease or condition is rheumatoid arthritis.

In some embodiments, the disease or condition comprises an infection.

In some embodiments, the disease or condition comprises Beta-thalassemia, Fragile X, centronuclear myopathy, Prion disease, Angelman Syndrome, Lafora disease, or Alexander disease, or a combination thereof.

In some embodiments, a method of treatment comprises administering a nucleic acid composition described herein and one or more additional active agents. For instance, the additional active agent may be used to complementarily treat the disease or condition.

In some embodiments, a subject refers to any animal, including, but not limited to, humans, non-human primates, rodents, and domestic and game animals. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a human.

In various embodiments, a subject can be one who has been previously diagnosed with or identified as suffering from or having a disease or condition in need of treatment. In various embodiments, the subject previously diagnosed with or identified as suffering from or having the disease or condition may or may not have undergone treatment for a condition. In other embodiments, a subject can also be one who has not been previously diagnosed as having a disease or condition and the therapeutic is used for prevention (prophylactically).

### Pharmaceutical Compositions, Administration and Dosage

In various embodiments, the compositions herein are formulated for delivery via any route of administration. “Route of administration” may refer to any administration pathway known in the art, including but not limited to intrathecal, epidural, intravenous, transdermal, intranasal, oral, mucosal, or other delivery methods, and/or via single or multiple doses. Example routes of administration for the nucleic acids described herein include lumbar intrathecal puncture, intracisterna magna administration, and intraganglionic administration.

In an example embodiment, the composition is delivered into the spinal intrathecal space using any appropriate delivery method. This approach may be particularly useful when targeting Naᵥ1.7 because the role played by Naᵥ1.7 is in the nociceptive afferents, and their cell bodies are in the respective segmental dorsal root ganglion (DRG) neurons. Therefore, delivery to the spinal intrathecal space may efficiently deliver compositions targeting Naᵥ1.7 to the DRG neurons, which can minimize the possibility of off target biodistribution and reduce viral load required for transduction.

It is appreciated that actual dosage can vary depending on the route of administration, the delivery system used (e.g., AAV or liposome, etc), the target cell, organ, or tissue, the subject, as well as the degree of effect sought. Size and weight of the tissue, organ, and/or patient can also affect dosing. Doses may further include additional agents, including but not limited to a carrier. Non-limiting examples of suitable carriers are known in the art: for example, water, saline, ethanol, glycerol, lactose, sucrose, dextran, agar, pectin, plant-derived oils, phosphate-buffered saline, and/or diluents. The pharmaceutical compositions can also contain any pharmaceutically acceptable carrier.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of nucleic acid (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. When the intrathecal route is recommended, the pharmaceutical product may be diluted ex vivo with the subject cerebrospinal fluid prior to administration to achieve an isobaric solution.

### Kits

Further provided is a kit to perform methods described herein. The kit is an assemblage of components, including at least one of the compositions described herein. Thus, in some embodiments, the kit comprises a nucleic acid encoding a nucleic acid binding domain and an epigenetic modulator. The nucleic acid may be combined with, or complexed to, another component such as a vehicle for delivery, or may be unmodified for direct delivery. In some cases, the nucleic acid is complexed with a cationic molecule. In some cases, the nucleic acid is configured for delivery via a viral delivery vehicle such as an AAV capsid protein. For certain kits where the nucleic acid binding domain comprises a dCas protein, the kit comprises one or more guide RNA sequences.

Instructions for use of the components may be included in the kit. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of a composition containing a nucleic acid herein. The packaging material generally has an external label which indicates the contents and/or purpose of the kit and/or its components.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term percent “identity,” in the context of two or more nucleic acid or polypeptide sequences, may refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity may be performed using the BLAST algorithm, which is described in Altschul et al. (J. Mol. Biol. 215:403-410 (1990)). Software for performing BLAST analyses is publicly available through the National Center for Biotechnology Information.

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the joints (intraarticular), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21st Ed., Mack Publ. Co., Easton, Pa. (2005), incorporated herein by reference in its entirety.

### EXAMPLES

The invention is further illustrated by the following non-limiting examples.

### Example 1

### Preparation of Compositions Comprising Naᵥ1.7-Binding Peptides

This example describes preparation of compositions comprising Naᵥ1.7-binding peptides. In this experiment, each of the following peptides are attached to the surface of a different AAV9 packaging mCherry reporter construct: the peptides of SEQ ID NO: 109-SEQ ID NO: 133, including ProTx-II (SEQ ID NO: 109), ProTx-III (SEQ ID NO: 117), the ProTx-II variant JNJ63955 (SEQ ID NO: 110), HwTx-IV (SEQ ID NO: 121), variant m3-HwTx-IV SEQ ID NO: 122), CcoTx1 variants (SEQ ID NO: 123 or SEQ ID NO: 124), PhlTx1 variant D7A-PhlTx1 (SEQ ID NO: 116), and JzTx-V variant AM-0422 (SEQ ID NO: 113). These peptides were chosen because they are established Naᵥ1.7-binding ligands and because they represent a varied selection of peptides with different sequences and differential interactions with the Naᵥ1.7 channel. Peptide attachment to the AAV is performed by both C-term NHS modification of the peptide and N-term Benzoyl-NH modification to bind with the nine lysine residues on the AAV9 surface. Crosslinking the peptides at the N- and C-terminals increases the chances that at least one of the peptides will keep the peptide conformation to bind Naᵥ1.7.

### Example 2

### Preparation of Compositions Encoding Naᵥ1.7-Binding Peptides

This example describes preparation of compositions encoding Naᵥ1.7-binding peptides. As attachment of peptides onto AAV9 may not be an economically feasible option for scaled-up manufacturing, sequences encoding one or more peptides of peptides of SEQ ID NO: 109-SEQ ID NO: 133 are incorporated into the viral genome to be expressed on viral protein loops. AAV viral capsids are composed of three proteins, VP1, VP2 and VP3. The N terminus of VP2 capsid protein accepts large-peptide insertions as well as proteins, thus, insertion of targeting peptides at VP2 re-targets vector tropism. Other potential sites to insert these target peptides are found in VP3 and VP1.

In a first experiment, a sequence encoding a peptide of any one of SEQ ID NO: 109-SEQ ID NO: 133 is cloned in frame within a—GS—flanking linker to be expressed in different loops of the AAV9 capsid. In this example, there are five inserting positions: 453, 447, 588, 589, and downstream of the N-terminal methionine of the VP2 start codon. Transduction efficiency is determined by mCherry reporter expression in the Naᵥ1.7-expressing cell lines HuH7 and Neuro2a.

Quality assessment tests are conducted to check that the surface modifications do not negatively affect packaging and capsid structure. This assessment focuses on viral titers since yield is usually the trait most affected after capsid modifications. Titers are measured by RT-qPCR. Ratios of viral proteins VP1, VP2, and VP3 are analyzed by protein gels.

### Example 3

### Design of Naᵥ1.7 Promoter Regions

This example describes design of Naᵥ1.7 promoter regions. Different Naᵥ1.7 promoters (e.g., SEQ ID NO: 44-SEQ ID NO: 50), a neuronal promotor (e.g., SEQ ID NO: 51), and ubiquitous promoters (e.g., SEQ ID NO: 52-SEQ ID NO: 54) are investigated for expression of mCherry in Naᵥ1.7 expressing cell lines, and non-Naᵥ1.7 expressing cell lines as negative controls, to determine specificity.

Design of minimal human Naᵥ1.7 promoter regions: The promoters tested vary between 700 bp-1.2 kbp in length. Promoters of various lengths are tested to enable their packaging into a single AAV. In addition, Naᵥ1.7 gene enhancers as described in the EPD website (https://epd.epfl.ch//index.php) are tested even if they are not part of the promoter region. These new sequences are cloned in a mCherry expressing vector with the CMV removed.

In vitro testing of human Naᵥ1.7 promoters: All cloned sequences are tested in human cell lines, and mCherry expression is compared to the vector harboring the CMV promoter. High Naᵥ1.7 expressing lines HuH7 and IMR-90 are utilized. The negative control is the non-Naᵥ1.7-expressing cell line MCF-7. RT-qPCR, FACS-sorting, and imaging analysis is performed to determine the expression (activity and intensity) levels of the transgene using these promoters.

iPCS testing of human Naᵥ1.7 promoters: The promoters that are specific (high expression in Naᵥ1.7 expressing cells and no expression in MCF-7 negative control) are tested in nociceptor-like iPCS cells. RT-qPCR, FACS-sorting, and imaging analysis is performed to determine the expression (activity and intensity) levels of mCherry using these promoters.

### Example 4

### AAV9 Vectors Encoding Naᵥ1.7 or Naᵥ1.8 Epigenetic Modulators

This example describes AAV9 vectors encoding Naᵥ1.7 or Naᵥ1.8 epigenetic modulators. A payload polynucleotide encoding an epigenetic modulator under transcriptional control of a promoter is encapsulated by an AAV9 capsid. The epigenetic modulator, when expressed from the payload polynucleotide, modulates Naᵥ1.7 or Naᵥ1.8 expression. The Naᵥ1.7 epigenetic modulator comprises a zinc figure protein targeting Naᵥ1.7 (e.g., any of SEQ ID NO: 134-SEQ ID NO: 148 or SEQ ID NO: 278-SEQ ID NO: 290) linked to a repressor domain (e.g., any of SEQ ID NO: 193-SEQ ID NO: 205) or a guide RNA targeting Naᵥ1.7 (e.g., any of SEQ ID NO: 55-SEQ ID NO: 98) and a dead Cas (e.g., any of SEQ ID NO: 1-SEQ ID NO: 43) linked to a repressor domain (e.g., any of SEQ ID NO: 193-SEQ ID NO: 205). The Naᵥ1.8 epigenetic modulator comprises a zinc figure protein targeting Naᵥ1.8 (e.g., SEQ ID NO: 149-SEQ ID NO: 192) linked to a repressor domain (e.g., any of SEQ ID NO: 193-SEQ ID NO: 205) or a guide RNA targeting Naᵥ1.8 (e.g., any of SEQ ID NO: 99-SEQ ID NO: 108 or SEQ ID NO: 268-SEQ ID NO: 277) and a dead Cas (e.g., any of SEQ ID NO: 1-SEQ ID NO: 43) linked to a repressor domain (e.g., any of SEQ ID NO: 193-SEQ ID NO: 205). The promoter is a Naᵥ1.7-specific promoter (e.g., any of SEQ ID NO: 44-SEQ ID NO: 50), a neuron-specific promoter (e.g., a human synapsin promoter of SEQ ID NO: 51), a ubiquitous promoter (e.g., any of SEQ ID NO: 52-SEQ ID NO: 54), or a Naᵥ1.8-specific promoter. Optionally, both a Naᵥ1.7 epigenetic modulator and a Naᵥ1.8 epigenetic modulator are encoded by the payload polynucleotide under transcriptional control of the promoter.

### Example 5

### Prevention of Pain and Inflammation in a Rheumatoid Arthritis Mouse Model

This example describes prevention of pain and inflammation in a rheumatoid arthritis mouse model by epigenetically modulating Naᵥ1.7 expression. Serum transfer mouse models for rheumatoid arthritis were generated as described in Christianson et al (Methods Mol Biol. 2012; 851: 249-260), which is incorporated by reference in its entirety. Briefly, K/BxN mice were generated by crossing KRN mice on a C57BL/6 genetic background with nonobese diabetic mice to produce K/BxN mice which exhibit sever arthritis. Transient inflammatory arthritis, representative of human rheumatoid arthritis, was produced in mice by transferring serum from the K/BxN into C57BL/6 genetic background mice.

A prevention mouse model for rheumatoid arthritis was generated by injecting C57BL/6 genetic background mice via intrathecal (IT) injection with AAV9 vectors carrying an experimental payload on day 0 and performing the serum transfer from K/BxN mice on day 16. Mice were injected with an AAV9 vector carrying a payload encoding a Naᵥ1.7 transcriptional modulator comprising a SCN9A-binding zinc finger protein of SEQ ID NO: 148 linked to a KOX1 repressor domain of SEQ ID NO: 194 under transcriptional control of a CMV promoter of SEQ ID NO: 52 (“K/BxN+ZF Naᵥ1.7”). A first control group of mice received the K/BxN serum transfer but no AAV9 vector (“K/BxN”). A second control group of mice received neither the K/BxN serum transfer nor the AAV9 vector (“Naïve”). Mechanical threshold tests were performed on days 17 through 45 (corresponding to days 1 through 28 following serum transfer) to evaluate pain and inflammation.

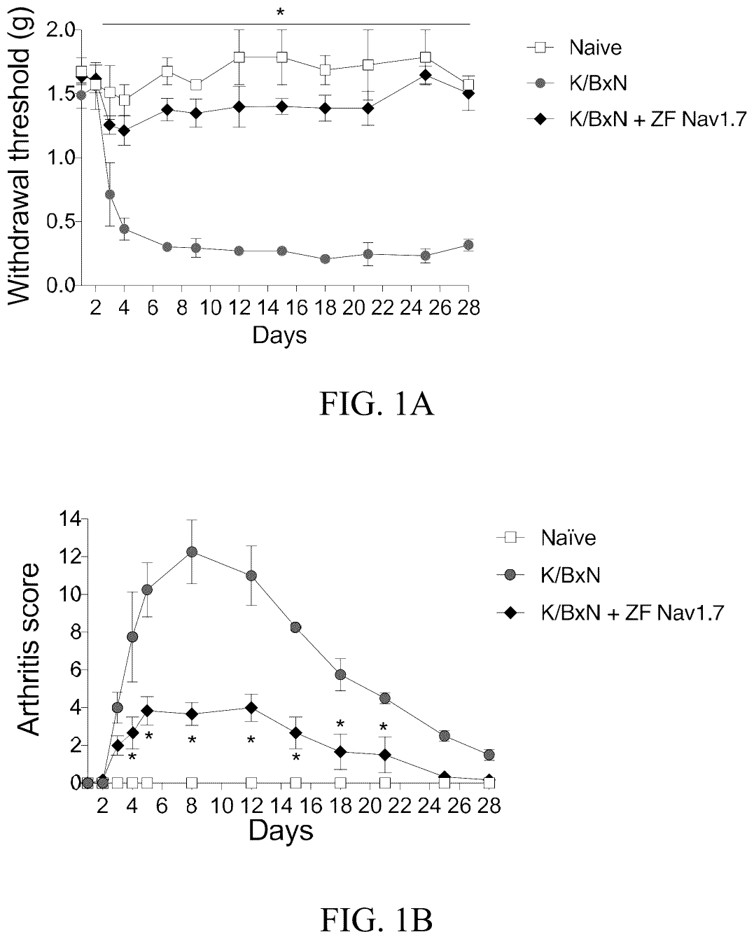

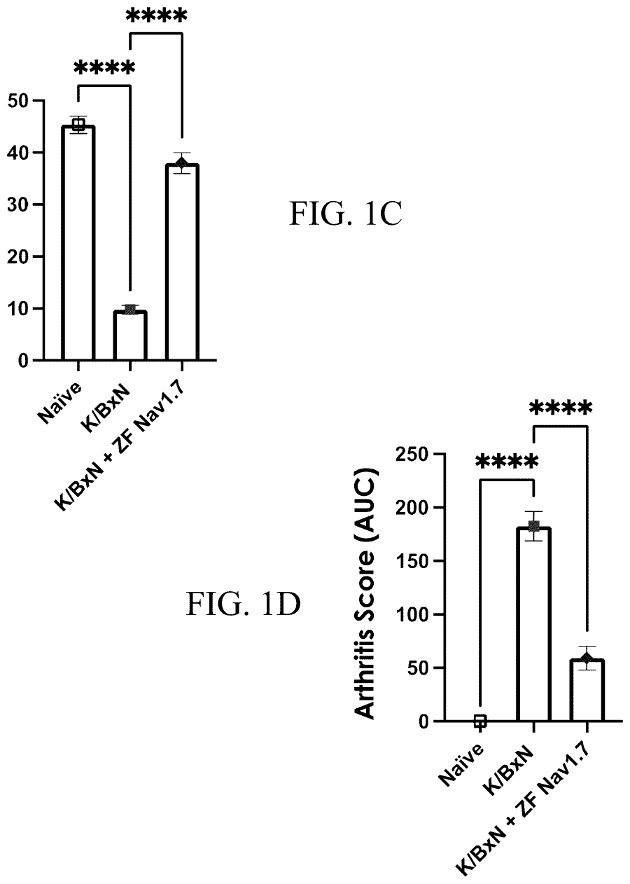

Withdrawal threshold, a measure of arthritis pain, was measured on days 1 through 28 following serum transfer. A lower withdrawal threshold corresponded to a higher pain level. As shown in FIG. 1A, mice treated with K/BxN serum that did not receive AAV9 treatment (“K/BxN”) showed a decrease in withdrawal threshold (increase in pain) on days 2 through 4 and maintained low withdrawal thresholds through day 28. Mice treated with K/BxN serum following treatment with AAV9 carrying the payload encoding the Naᵥ1.7 transcriptional modulator (“K/BxN+ZF Naᵥ1.7”) showed statistically higher withdrawal thresholds (lower pain levels) than mice not receiving AAV9 treatment. Naïve mice that received neither the K/BxN serum transfer nor the AAV9 vector (“Naïve”) also showed significantly higher withdrawal thresholds than mice treated with K/BxN serum but not AAV9 (* denotes p<0.05). The corresponding area under the curve (AUC) is provided in FIG. 1C.

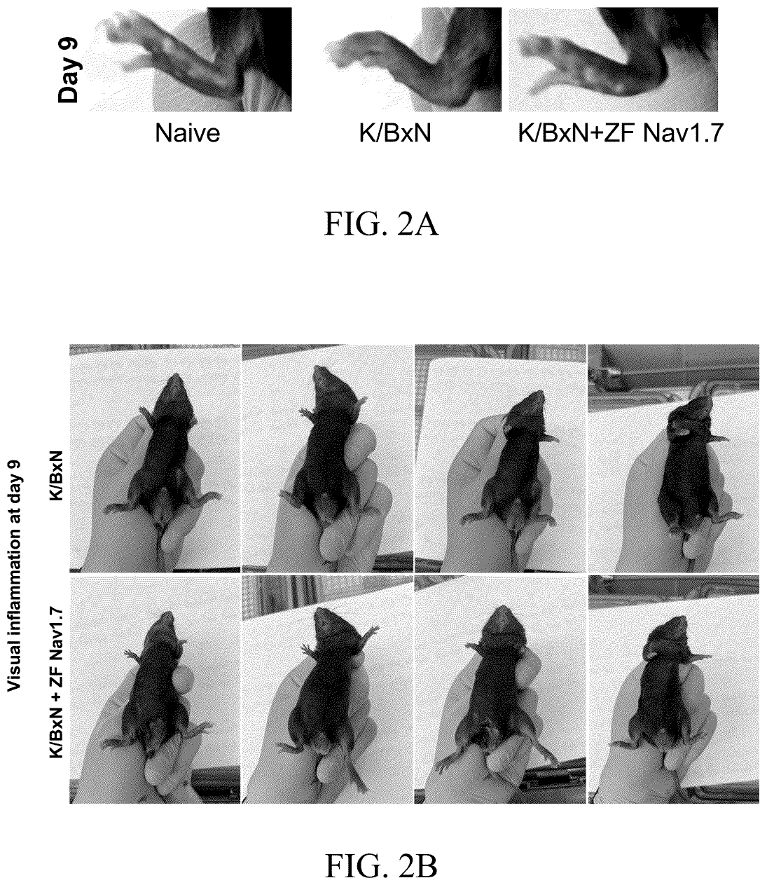

Arthritis score, a measure of inflammation, was evaluated by scoring 1 point for each inflamed toe and 2 points for each inflamed ankle, for a maximum score of 14 points. A higher arthritis score corresponded to greater inflammation. As shown in FIG. 1B, mice treated with K/BxN serum that did not receive AAV9 treatment (“K/BxN”) showed an increase in arthritis score (increase in inflammation) on days 2 through 8 followed by a slow decrease through day 28. Mice treated with K/BxN serum following treatment with AAV9 carrying the payload encoding the Naᵥ1.7 transcriptional modulator (“K/BxN+ZF Naᵥ1.7”) showed statistically lower arthritis scores (less inflammation) on days 4 through 21 than mice not receiving AAV9 treatment (* denotes p<0.05). The corresponding area under the curve (AUC) is provided in FIG. 1D. Inflammation was not observed in naïve mice that received neither the K/BxN serum transfer nor the AAV9 vector (“Naïve”). FIG. 2A shows photos taken on day 9 of hind feet from mice of the K/BxN, K/BxN+ZF Naᵥ1.7, and Naïve treatment groups, and FIG. 2B shows photos taken on day 9 of mice from the K/BxN and K/BxN+ZF Naᵥ1.7 treatment groups. Visible inflammation can be seen in the mice from the K/BxN treatment group. Together these data demonstrate that epigenetic modulation of Naᵥ1.7 using a Naᵥ1.7 transcriptional modulator can prevent pain and inflammation in a rheumatoid arthritis model.

### Example 6

### Reversion of Pain and Inflammation in a Rheumatoid Arthritis Mouse Model

This example describes reversion of pain and inflammation in a rheumatoid arthritis mouse model by epigenetically modulating Naᵥ1.7 expression. Serum transfer mouse models for rheumatoid arthritis were generated as described in Christianson et al (Methods Mol Biol. 2012; 851: 249-260), which is incorporated by reference in its entirety. Briefly, K/BxN mice were generated by crossing KRN mice on a C57BL/6 genetic background with nonobese diabetic mice to produce K/BxN mice which exhibit sever arthritis. Transient inflammatory arthritis, representative of human rheumatoid arthritis, was produced in mice by transferring serum from the K/BxN into C57BL/6 genetic background mice.

A reversion mouse model for rheumatoid arthritis was generated by performing the serum transfer from K/BxN mice into C57BL/6 genetic background mice on day 0 and injecting the mice via intrathecal (IT) injection with AAV9 vectors carrying an experimental payload on day 3. Mice were injected with AAV9 vectors carrying either a payload encoding a Naᵥ1.7 transcriptional modulator comprising a SCN9A-binding zinc finger protein of SEQ ID NO: 148 linked to a KOX1 repressor domain of SEQ ID NO: 194 under transcriptional control of a CMV promoter of SEQ ID NO: 52 (“K/BxN+ZF Naᵥ1.7”) or a payload encoding an mCherry marker under transcriptional control of a CMV promoter of SEQ ID NO: 52 (“K/BxN+mCherry”). A first control group of mice received the K/BxN serum transfer but no AAV9 vector (“K/BxN”). A second control group of mice received neither the K/BxN serum transfer nor the AAV9 vector (“Naïve”). Mechanical threshold tests were performed on days 1 through 28 to evaluate pain and inflammation.

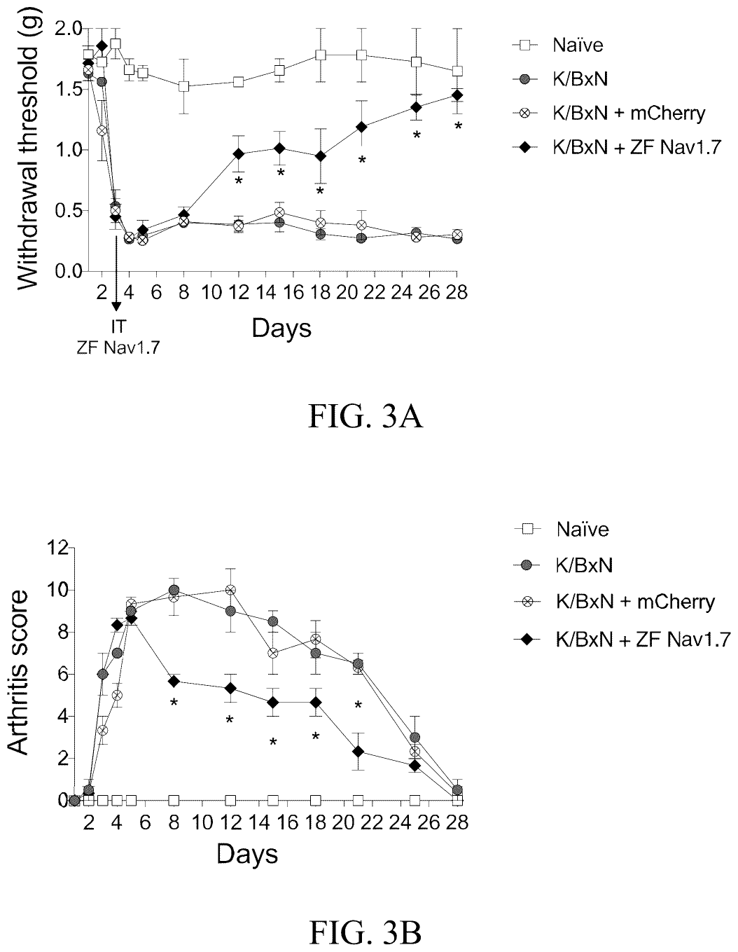

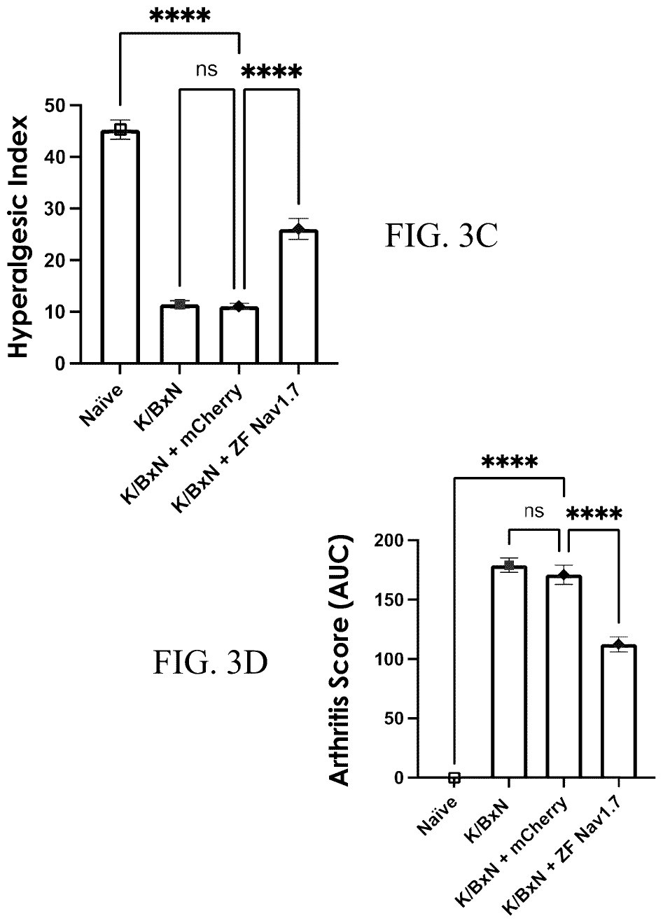

Withdrawal threshold, a measure of arthritis pain, was measured on days 1 through 28 following serum transfer. A lower withdrawal threshold corresponded to a higher pain level. As shown in FIG. 3A, mice treated with K/BxN serum that did not receive AAV9 treatment (“K/BxN”) showed a decrease in withdrawal threshold (increase in pain) on days 2 through 4 and maintained low withdrawal thresholds through day 28. Mice treated with K/BxN serum and subsequently receiving with AAV9 carrying the payload encoding the Naᵥ1.7 transcriptional modulator (“K/BxN+ZF Naᵥ1.7”) on day 3 showed a decrease in withdrawal threshold prior to receiving AAV9 treatment and then showed statistically higher withdrawal thresholds (lower pain levels) after AAV9 treatment (days 12 through 28) than mice not receiving AAV9 treatment (* denotes p<0.05). The corresponding area under the curve (AUC) is provided in FIG. 3C. Mice treated with K/BxN and AAV9 carrying the payload encoding mCherry (“K/BxN+mCherry”) exhibited withdrawal thresholds comparable to mice treated with K/BxN serum but not AAV9. Naïve mice that received neither the K/BxN serum transfer nor the AAV9 vector (“Naïve”) consistently higher withdrawal thresholds after day 2 than mice treated with K/BxN serum but not AAV9.

Arthritis score, a measure of inflammation, was evaluated by scoring 1 point for each inflamed toe and 2 points for each inflamed ankle, for a maximum score of 14 points. A higher arthritis score corresponded to greater inflammation. As shown in FIG. 3B, mice treated with K/BxN serum that did not receive AAV9 treatment (“K/BxN”) showed an increase in arthritis score (increase in inflammation) on days 2 through 8 followed by a slow decrease through day 28. Mice treated with K/BxN serum and subsequently receiving with AAV9 carrying the payload encoding the Naᵥ1.7 transcriptional modulator (“K/BxN+ZF Naᵥ1.7”) on day 3 showed an increase in arthritis score prior to receiving AAV9 treatment and then showed statistically lower arthritis scores (less inflammation) on days 8 through 18 than mice not receiving AAV9 treatment (* denotes p<0.05). The corresponding area under the curve (AUC) is provided in FIG. 3D. Mice treated with K/BxN and AAV9 carrying the payload encoding mCherry (“K/BxN+mCherry”) exhibited arthritis scores comparable to mice treated with K/BxN serum but not AAV9. Inflammation was not observed in naïve mice that received neither the K/BxN serum transfer nor the AAV9 vector (“Naïve”). Together these data demonstrate that epigenetic modulation of Naᵥ1.7 using a Naᵥ1.7 transcriptional modulator can reverse pain and inflammation in a rheumatoid arthritis model.

### Example 7

### Reduction of Nerve Sprouting in a Rheumatoid Arthritis Mouse Model

This example describes reduction of nerve sprouting in a rheumatoid arthritis mouse model. In arthritic pathologies, such as rheumatoid arthritis, joints can remain painful after successful treatment of synovial inflammation. The quality of this pain can have neuropathic features. One potential mechanism that may explain the dissociation between disease progression and pain in arthritis is an ectopic sprouting of sensory and sympathetic nerve fibers. The impact of epigenetic modulation of Naᵥ1.7 on nerve sprouting was evaluated in both a pain prevention model for rheumatoid arthritis, using mice treated as described in EXAMPLE 5, and in a pain reversion model for rheumatoid arthritis, using mice treated as described in EXAMPLE 6. Mice from EXAMPLE 5 and EXAMPLE 6 were sacrificed on day 28, and nerve sprouting was evaluated using confocal microscopy of immunostained sections of the ankles.

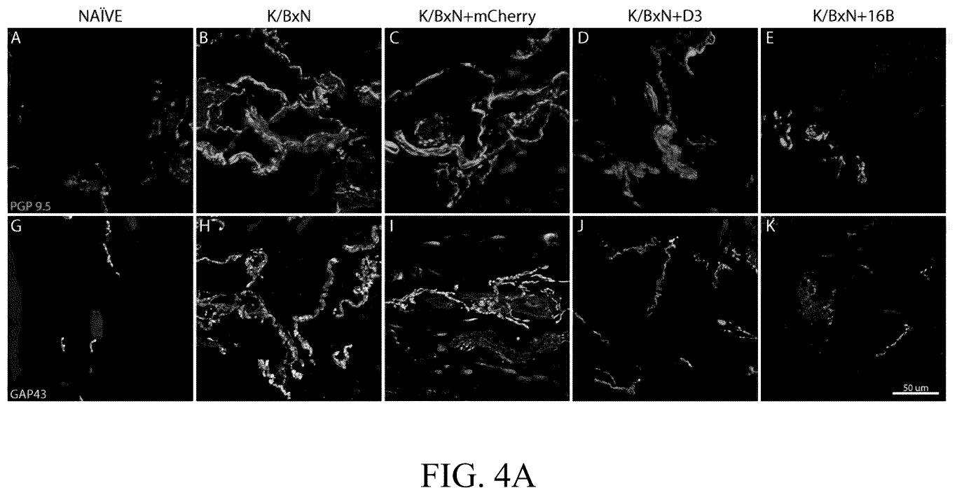

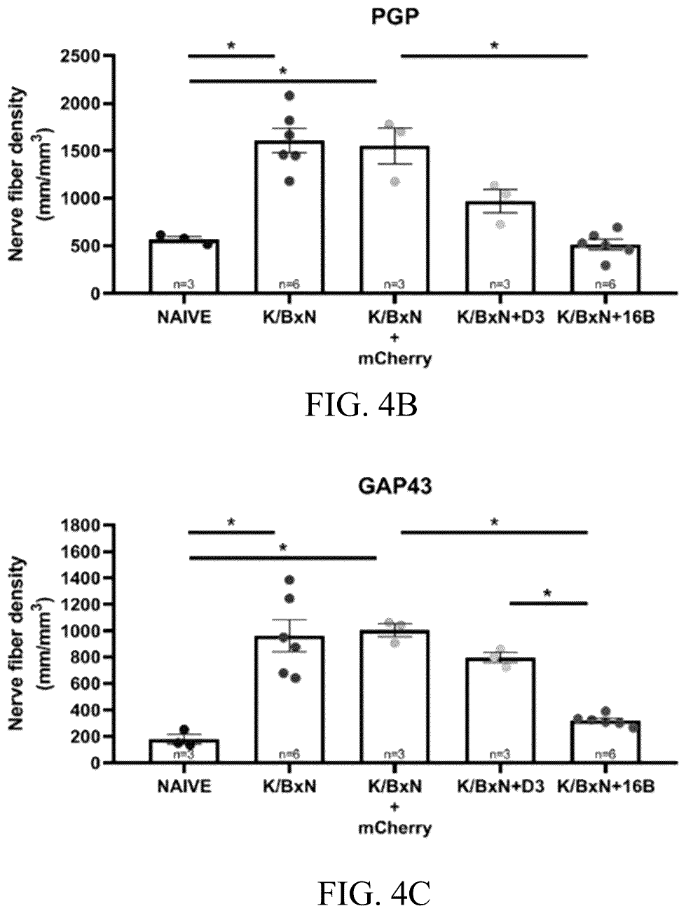

FIG. 4A shows confocal images of sections collected from naïve mice that received neither the K/BxN serum transfer nor the AAV9 vector (“Naïve”), mice treated with K/BxN serum that did not receive AAV9 treatment (“K/BxN”), treated with K/BxN and AAV9 carrying the payload encoding mCherry (“K/BxN+mCherry”), mice treated with K/BxN serum following treatment with AAV9 carrying the payload encoding the Naᵥ1.7 transcriptional modulator in a pain prevention model (“K/BxN+16B”), and mice treated with K/BxN serum and subsequently receiving with AAV9 carrying the payload encoding the Naᵥ1.7 transcriptional modulator in a pain reversion model (“K/BxN+D3”). Sections were stained for PGP9.5, a pan-neuronal marker (FIG. 4A, top) and GAP-43, a marker of nerve sprouting (FIG. 4A, bottom). The staining was quantified in FIG. 4B and FIG. 4C. As shown in FIG. 4B, mice treated with AAV9 carrying the payload encoding the Naᵥ1.7 transcriptional modulator in both the pain prevention and pain reversion models showed decreased nerve fiber density compared to the mice treated with K/BxN serum receiving either no AAV9 treatment or treatment with AAV9 carrying the payload encoding mCherry (* denotes p<0.05). Additionally, as shown in FIG. 4C, mice treated with AAV9 carrying the payload encoding the Naᵥ1.7 transcriptional modulator in the pain reversion model showed decreased density of nerve sprouting compared to the mice treated with K/BxN serum receiving either no AAV9 treatment or treatment with AAV9 carrying the payload encoding mCherry (* denotes p<0.05).

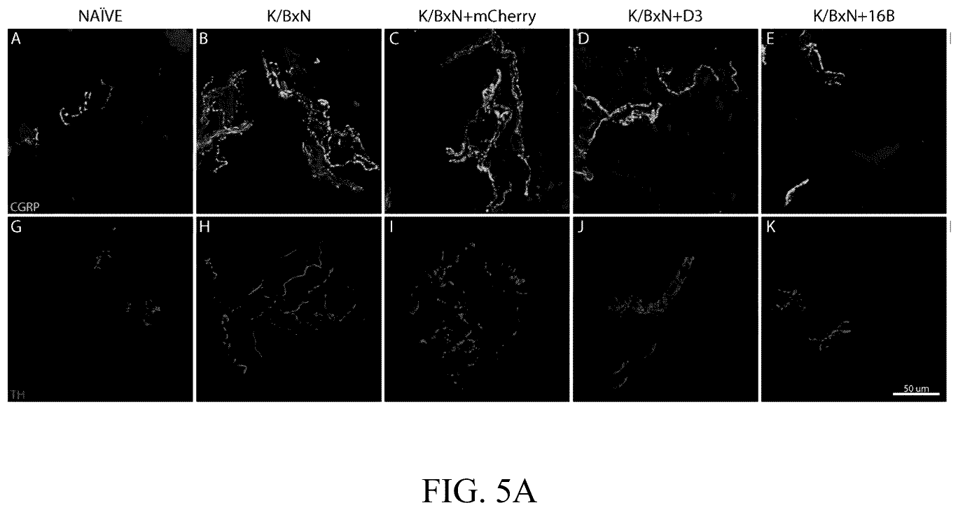

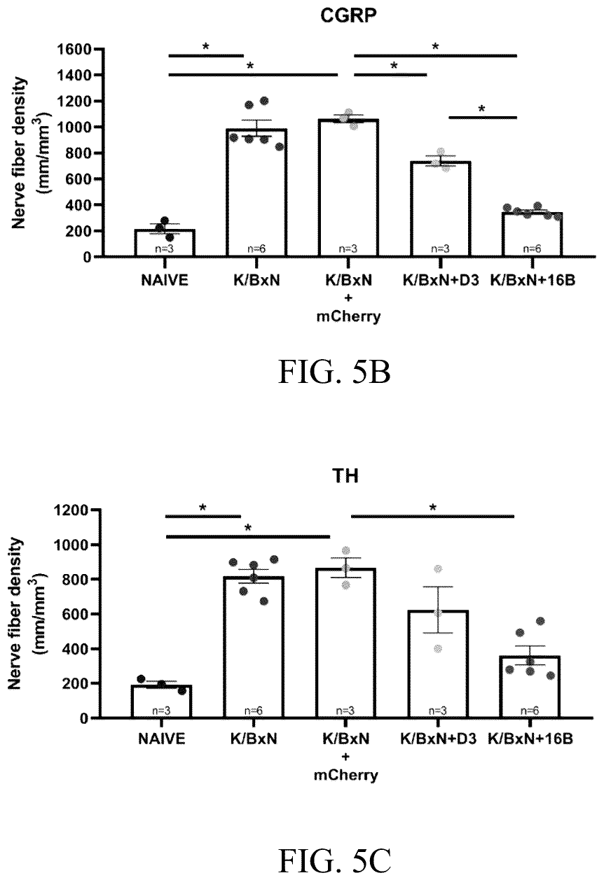

FIG. 5A shows confocal images of sections collected from naïve mice that received neither the K/BxN serum transfer nor the AAV9 vector (“Naïve”), mice treated with K/BxN serum that did not receive AAV9 treatment (“K/BxN”), treated with K/BxN and AAV9 carrying the payload encoding mCherry (“K/BxN+mCherry”), mice treated with K/BxN serum following treatment with AAV9 carrying the payload encoding the Naᵥ1.7 transcriptional modulator in a pain prevention model (“K/BxN+16B”), and mice treated with K/BxN serum and subsequently receiving with AAV9 carrying the payload encoding the Naᵥ1.7 transcriptional modulator in a pain reversion model (“K/BxN+D3”). Sections were stained for CGRP, a marker for primary afferent neurons (FIG. 5A, top) and TH, a marker for sympathetic nerve fibers (FIG. 5A, bottom). The staining was quantified in FIG. 5B and FIG. 5C. As shown in FIG. 5B, mice treated with AAV9 carrying the payload encoding the Naᵥ1.7 transcriptional modulator in both the pain prevention and pain reversion models showed decreased density of primary afferent neurons compared to the mice treated with K/BxN serum receiving either no AAV9 treatment or treatment with AAV9 carrying the payload encoding mCherry (* denotes p<0.05). Additionally, as shown in FIG. 5C, mice treated with AAV9 carrying the payload encoding the Naᵥ1.7 transcriptional modulator in both the pain prevention and pain reversion models showed decreased density of sympathetic nerve fibers compared to the mice treated with K/BxN serum receiving either no AAV9 treatment or treatment with AAV9 carrying the payload encoding mCherry (* denotes p<0.05). Together these data show that epigenetic modulation of Naᵥ1.7 using a Naᵥ1.7 transcriptional modulator can reduce nerve sprouting in a rheumatoid arthritis model.

### Example 8

### Reduction of Chronic Pain and Inflammation in a Complete Freud's Adjuvant Mouse Model by Downregulating Naᵥ1.7, Naᵥ1.8, or Both

This example describes reduction of pain and inflammation in a complete Freud's adjuvant (CFA) mouse model by downregulating Naᵥ1.7, Naᵥ1.8, or both via epigenetic modulation. CFA mouse models, a model for chronic pain and inflammation, were generated by injecting mice via intrathecal (IT) injection with AAV9 vectors carrying an experimental payload on day 0 and administering CFA ankle injections on day 16. Mice were injected with either AAV9 vectors carrying a payload encoding a Naᵥ1.7 transcriptional modulator comprising a SCN9A-binding zinc finger protein of SEQ ID NO: 148 linked to a KOX1 repressor domain of SEQ ID NO: 194 under transcriptional control of a CMV promoter of SEQ ID NO: 52 (“CFA+ZF Naᵥ1.7”); AAV9 vectors carrying a payload encoding a Naᵥ1.8 transcriptional modulator comprising a SCN10A-binding zinc finger protein of SEQ ID NO: 192 linked to a KOX1 repressor domain of SEQ ID NO: 194 under transcriptional control of a CMV promoter of SEQ ID NO: 52 (“CFA+ZF Naᵥ1.8”); AAV9 vectors carrying a payload encoding a Naᵥ1.7 transcriptional modulator comprising a SCN9A-binding zinc finger protein of SEQ ID NO: 148 linked to a KOX1 repressor domain of SEQ ID NO: 194 and a Naᵥ1.8 transcriptional modulator comprising a SCN10A-binding zinc finger protein of SEQ ID NO: 192 linked to a KOX1 repressor domain of SEQ ID NO: 194 both under transcriptional control of a CMV promoter of SEQ ID NO: 52 (“CFA+ZF Naᵥ1.7/1.8”); or AAV9 vectors carrying a payload encoding an mCherry marker under transcriptional control of a CMV promoter of SEQ ID NO: 52 (“CFA+mCherry”). A first control group of mice received the CFA ankle injections and a AAV9 vector expressing mCherry (“CFA+mCherry”). A second control group of mice received neither the CFA ankle injections nor the AAV9 vector (“Naïve”). Mechanical threshold tests were performed a 1 hour through 8 days following CFA injection to evaluate pain and inflammation.

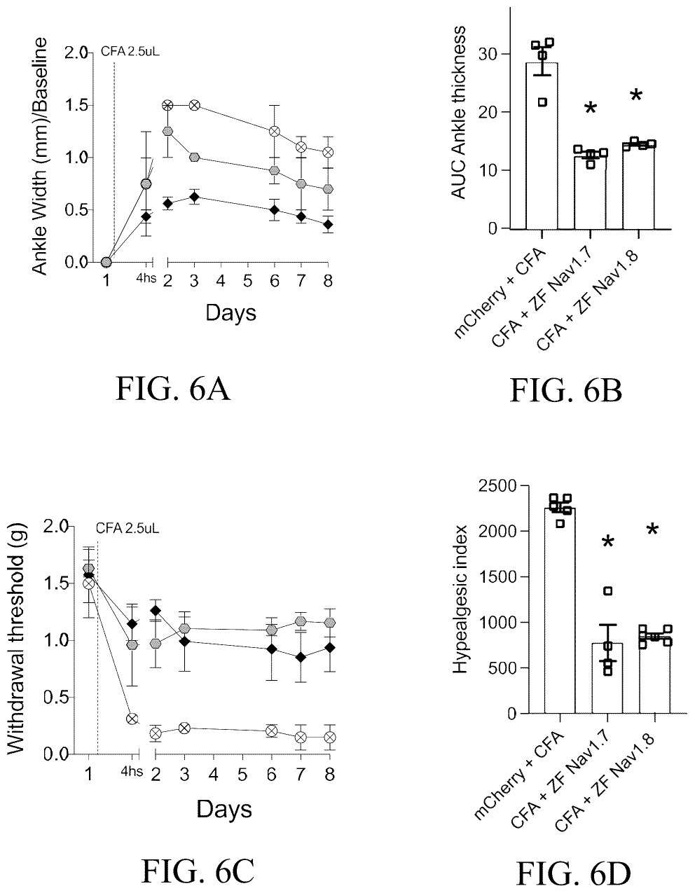

Ankle width, a measure of inflammation, was measured starting at 1 hour through 8 days following CFA injection. As shown in FIG. 6A and FIG. 6B, mice treated with CFA and AAV9 carrying the payload encoding the Naᵥ1.8 transcriptional modulator at a dose of 1×10¹² viral genomes (vg) per mouse (“CFA+ZF 1.8(1E+12)”, FIG. 6A and FIG. 6B) and mice treated with CFA and AAV9 carrying the payload encoding the Naᵥ1.7 transcriptional modulator at a dose of 1×10¹² vg/mouse (“CFA+ZF 1.7(1E+12)”, FIG. 6A and FIG. 6B) exhibited less inflammation than mice treated with CFA and AAV9 carrying the payload encoding mCherry at a dose of 1×10¹² vg/mouse (“CFA+mCherry(1E+12)”, FIG. 6A and FIG. 6B) (* denotes p<0.05).

Withdrawal threshold, a measure of arthritis pain, was measured at 1 hour through 8 days following CFA injection. As shown in FIG. 6C and FIG. 6D, mice treated with CFA and AAV9 carrying the payload encoding the Naᵥ1.8 transcriptional modulator at a dose of 1×10¹² vg/mouse (“CFA+ZF 1.8(1E+12)”, FIG. 6C and FIG. 6D) and mice treated with CFA and AAV9 carrying the payload encoding the Naᵥ1.7 transcriptional modulator at a dose of 1×10¹² vg/mouse (“CFA+ZF 1.7(1E+12)”, FIG. 6C and FIG. 6D) exhibited a higher withdrawal threshold (lower pain) than mice treated with CFA and AAV9 carrying the payload encoding mCherry at a dose of 1×10¹² vg/mouse (“CFA+mCherry(1E+12)”, FIG. 6C and FIG. 6D) (* denotes p<0.05).

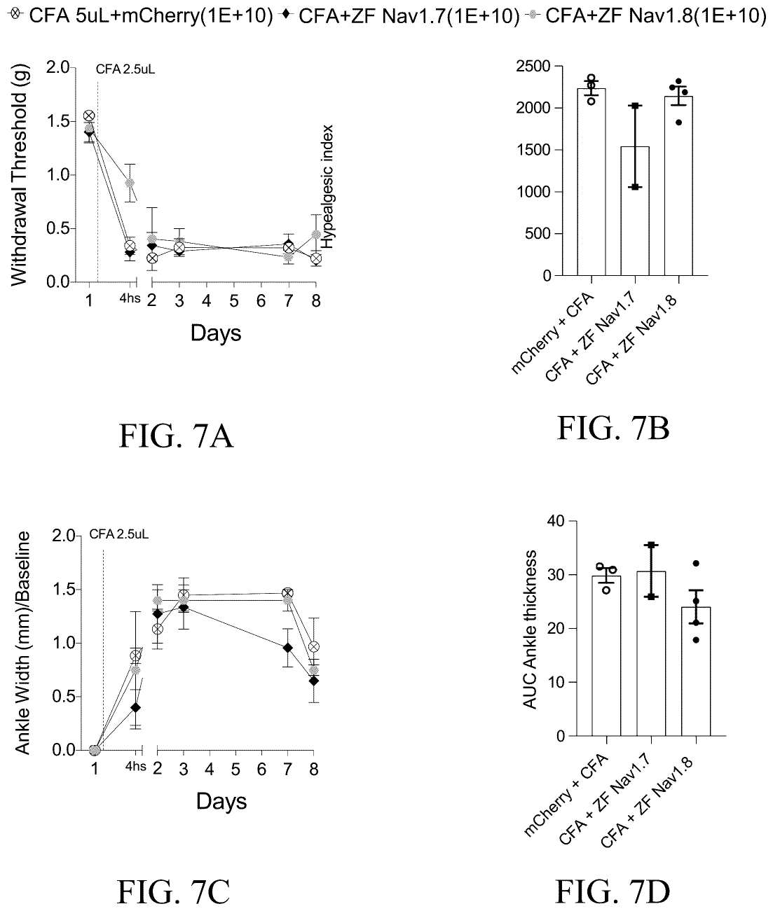

Comparable experiments were performed using 100-fold lower doses of 1×10¹⁰ AAV9 vg/mouse. As shown in FIG. 7A-FIG. 7D, mice receiving 100-fold lower doses of AAV9 carrying the payload encoding the Naᵥ1.7 transcriptional modulator or the payload encoding the Naᵥ1.8 transcriptional modulator did not exhibit a reduction in pain (FIG. 7A and FIG. 7B) or inflammation (FIG. 7C and FIG. 7D) relative control as was seen at the higher dose. This data suggests a dose-dependent effect of Naᵥ1.7 and Naᵥ1.8 transcriptional modulators on pain and inflammation.

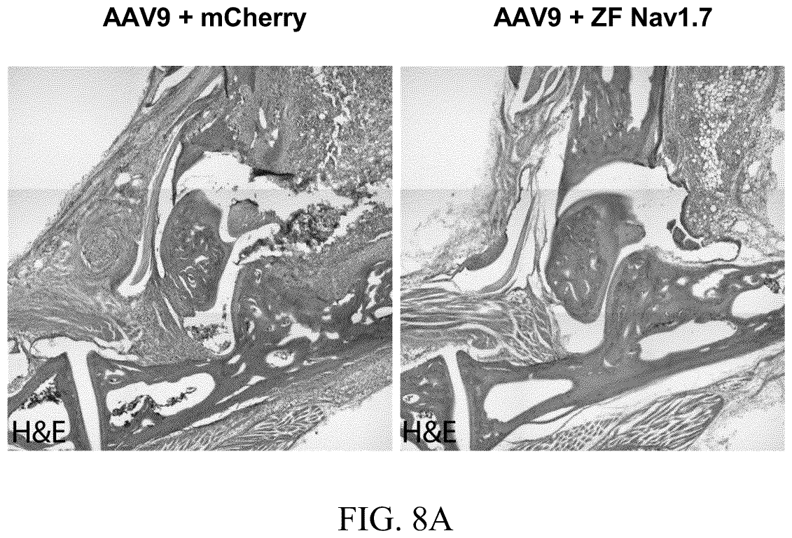

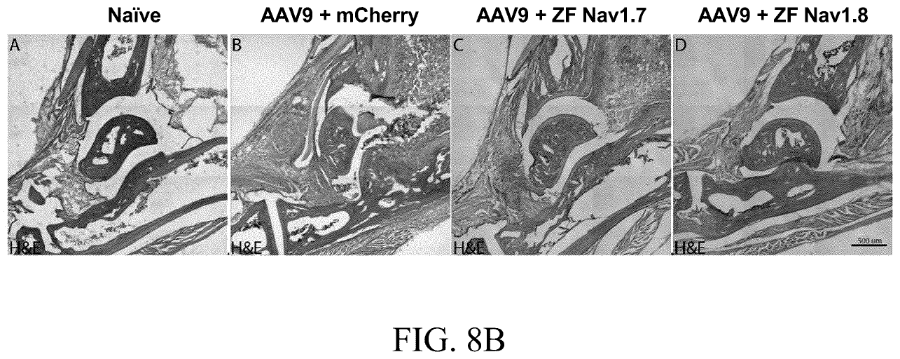

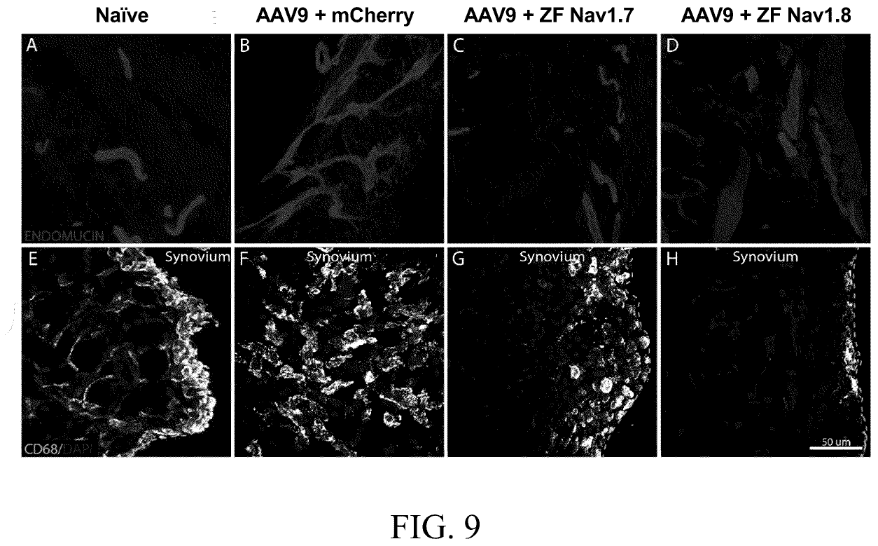

Downregulation of Naᵥ1.7 or Naᵥ1.8 also prevented joint remodeling and reduced cell infiltration in the CFA mouse model. FIG. 8A and FIG. 8B show mouse tissue sections stained with hematoxylin and eosin (“H&E”). As seen in FIG. 8A and FIG. 8B, mice from the AAV9 Naᵥ1.7 treatment group (“CFA+ZF 1.7”) and mice from the AAV9 Naᵥ1.8 treatment group (“CFA+ZF 1.8”) showed less joint remodeling and cell infiltration than the AAV9 mCherry (“CFA+mCherry”) treatment group. Additionally, mice from the AAV9 Naᵥ1.7 treatment group (“CFA+ZF 1.7”) and mice from the AAV9 Naᵥ1.8 treatment group (“CFA+ZF 1.8”) showed aberrant innervation, which increases with CFA, and reduced CD68 expression, a marker of inflammation, compared to the CFA only (“CFA”) treatment group. FIG. 9 shows confocal images of mouse tissue sections stained for endomucin (top) or CD68 and DAPI (bottom).

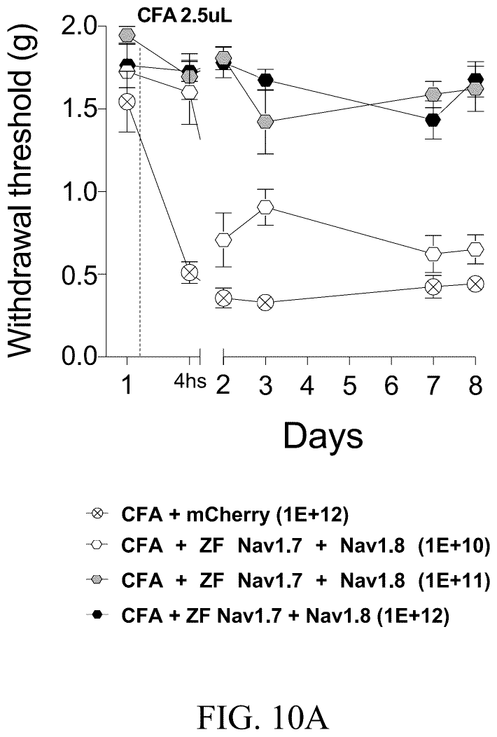

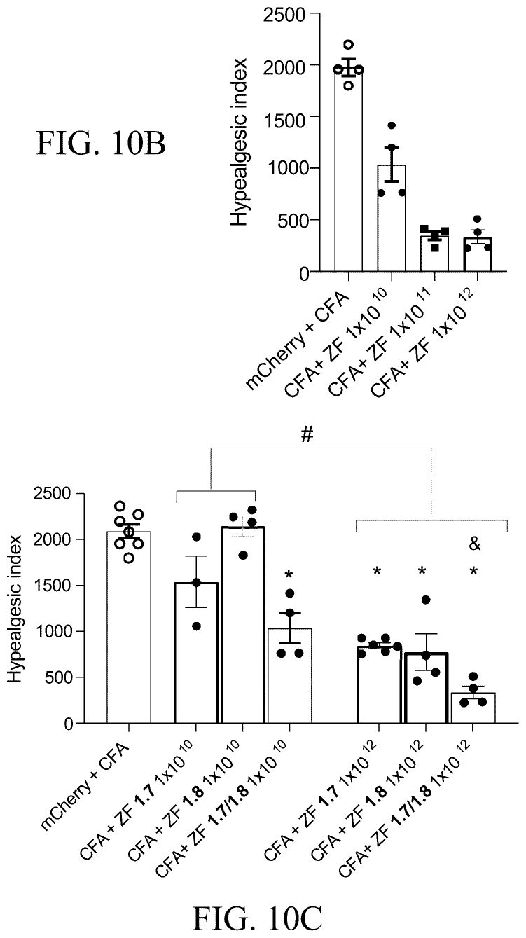

Withdrawal threshold was also measured in mice treated with CFA and AAV9 carrying the payload encoding the Naᵥ1.7 transcriptional modulator and the Naᵥ1.8 transcriptional modulator, multiplexing Naᵥ1.7 and Naᵥ1.8 repression, at different doses, as shown in FIG. 10A. Mice treated at a dose of 1×10¹² vg/mouse (“CFA+ZF Naᵥ1.7+Naᵥ1.8 (1E+12)”), at a dose of 1×10¹¹ vg/mouse (“CFA+ZF Naᵥ1.7+Naᵥ1.8 (1E+11)”), or at a dose of 1×10¹⁰ vg/mouse (“CFA+ZF Naᵥ1.7+Naᵥ1.8 (1E+10)”) exhibited higher withdrawal thresholds (lower pain) than mice treated with AAV9 carrying the payload encoding mCherry at a dose of 1×10¹² vg/mouse (“CFA+mCherry (1E+12)”). A hyperalgesic index (area under the curve for mechanical allodynia over time) was determined for the mice treated with both Naᵥ1.7 and Naᵥ1.8 AAV9 vectors at the 1×10¹⁰, 1×10¹¹, and 1×10¹² doses, as shown in FIG. 10B. The hyperalgesic index was compared for mice treated with the Naᵥ1.7 AAV9 vector at a dose of 1×10¹⁰ (“CFA+ZF 1.7×10¹⁰”) or a dose of 1×10¹² (“CFA+ZF 1.7×10¹²”), the Naᵥ1.8 AAV9 vector at a dose of 1×10¹⁰ (“CFA+ZF 1.8×10¹⁰”) or a dose of 1×10¹² (“CFA+ZF 1.8×10¹²”), or both the Naᵥ1.7 and Naᵥ1.8 AAV9 vectors at a dose of 1×10¹⁰ (“CFA+ZF 1.7/1.8×10¹⁰”) or a dose of 1×10¹² (“CFA+ZF 1.7/1.8×10¹²”), as shown in FIG. 10C.

Together these data show that epigenetic modulation of Naᵥ1.7 or Naᵥ1.8 can be used to reduce chronic pain and inflammation in a CFA model. Furthermore, simultaneous downregulation of both Naᵥ1.7 or Naᵥ1.8 via epigenetic modulation provided synergistic effects on pain and inflammation, given that a decrease in dose of 100× was just as efficacious as single targeting of Naᵥ1.7 or Naᵥ1.8.

### Example 9

### Reduction of Chemotherapy-Induced Peripheral Neuropathy (CIPN) by Downregulating Naᵥ1.7, Naᵥ1.8, or Both

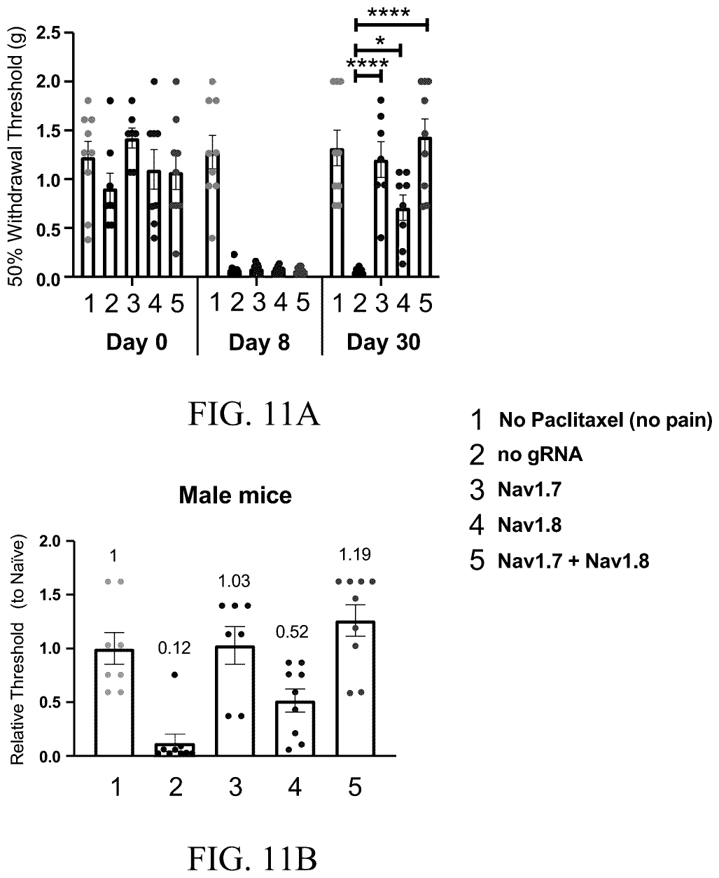

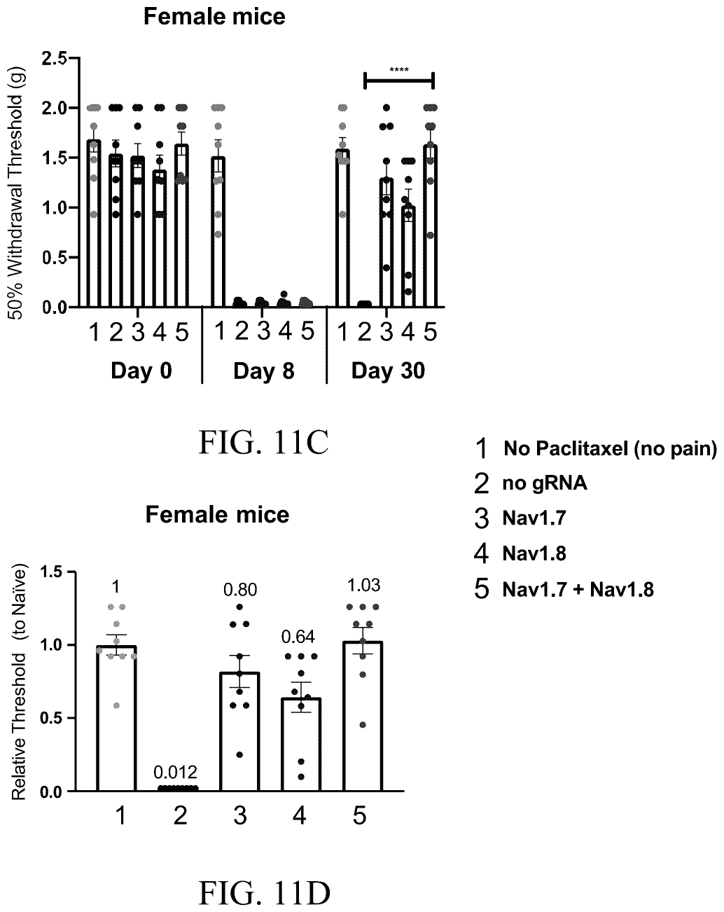

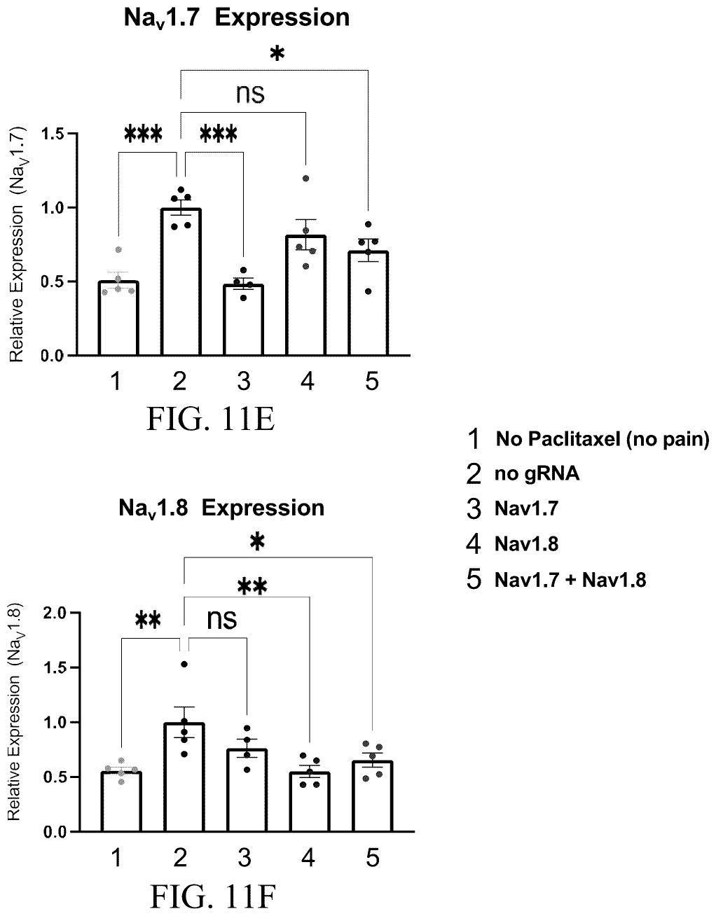

This example describes reduction of chemotherapy-induced peripheral neuropathy (CIPN) by downregulating Naᵥ1.7, Naᵥ1.8, or both. Mice were administered paclitaxel via intraperitoneal administration (IP) on days 1, 3, 5, and 7, with a dose of 8 mg/kg (total cumulative dosage of 32 mg/kg), with a group of saline injected mice not receiving any paclitaxel (n=9) to establish the tactile allodynia caused by the chemotherapeutic. After confirming tactile allodynia on day 8, mice (n=9/group) were injected with 1-10¹² vg/mouse of AAV9-KRAB-dCas9 with guide RNA (gRNA) targeting Naᵥ1.7, Naᵥ1.8, Naᵥ1.7+Naᵥ1.8, No-gRNA (non-targeting), or saline. The At day 30, mice were then tested for tactile allodynia via von Frey filaments (FIG. 11A). A 50% tactile threshold was calculated. A decrease in tactile threshold was observed in mice receiving No-gRNA, while mice that received KRAB-dCas9 targeting Naᵥ1.7, Naᵥ1.8 or Naᵥ1.7+Naᵥ1.8 had increased withdrawal thresholds, indicating that in situ Naᵥ1.7 and Naᵥ1.8 repression can prevent chemotherapy induced tactile allodynia (FIG. 11A). To determine the level of mechanical allodynia reversal, the relative threshold (compared to the no-paclitaxel group) was plotted. Mice injected with the Naᵥ1.8 repressor (comprising gRNAs of SEQ ID NO: 271 and SEQ ID NO: 273) had 48% lower threshold as compared to the no-paclitaxel control while Naᵥ1.7 and the Naᵥ1.7+Naᵥ1.8 dual repressor (comprising gRNAs of SEQ ID NO: 91 and SEQ ID NO: 271) had complete reversal of mechanical allodynia in male mice (FIG. 11B). Of note, pain was repressed, but complete ablation of pain was not observed with Naᵥ1.7, and the levels of pain relief are expected to depend on dosage.

FIG. 11A-FIG. 11F show in vivo efficacy of KRAB-dCas9 in reversing mechanical allodynia in a chemotherapy-induced neuropathic pain model. In order to establish a baseline level of sensitivity, mice were tested for tactile threshold using von Frey filaments. Mice were then injected i.p. with 8 mg/kg of paclitaxel at days 1, 3, 5, and 7. After confirming tactile allodynia via von Frey filaments, mice were IT injected at day 9 with 1·10¹² vg/mouse of AAV9-KRAB-dCas9-no-gRNA, AAV9-KRAB-dCas9-Naᵥ1.7, AAV9-KRAB-dCas9-Naᵥ1.8, or AAV9-KRAB-dCas9-Naᵥ1.7+Naᵥ1.8. 21 days later, mice were tested for tactile allodynia via von Frey filaments. FIG. 11A shows in situ repression of Naᵥ1.7, Naᵥ1.8, and of simultaneous Naᵥ1.7+Naᵥ1.8 via KRAB-dCas9 reverses paclitaxel-induced tactile allodynia in male mice (dots represent individual biological replicates; n=9; error bars are SEM; two-way ANOVA with Bonferonni post hoc test; ****p<0.0001, *p=0.0144). FIG. 11B shows Relative mechanical allodynia threshold relative to AAV9-KRAB-dCas9-no-gRNA injected male mice. FIG. 11C shows in situ repression of Naᵥ1.7, Naᵥ1.8, and of simultaneous Naᵥ1.7+Naᵥ1.8 via KRAB-dCas9 reverses paclitaxel-induced tactile allodynia in female mice (dots represent individual biological replicates; n=9; error bars are SEM; two-way ANOVA with Bonferonni post hoc test; ****<p). FIG. 11D shows relative mechanical allodynia threshold relative to AAV9-KRAB-dCas9-no-gRNA injected female mice. FIG. 11E shows in vivo Naᵥ1.7 repression levels: mice DRG (L4-L6) were harvested and Naᵥ1.7 repression efficacy was determined by qPCR (dots represent individual biological replicates; qPCR was performed in technical triplicates; n=5; error bars are SEM; values normalized to Gapdh; Student's t-test; ***p=0.0004 (Naᵥ1.7), ***p=0.0003 (no paclitaxel), *p=0.0276, ns=not significant). FIG. 11F shows in vivo Naᵥ1.8 repression levels: mice DRG (L4-L6) were harvested and Naᵥ1.8 repression efficacy was determined by qPCR (dots represent individual biological replicates; qPCR was performed in technical triplicates; n=5; error bars are SEM; values normalized to Gapdh; Student's t-test; **p=0.0038 (Naᵥ1.8), **p=0.0044 (no paclitaxel), *p=0.0269, ns=not significant).

Similar to male mice, a decrease in tactile threshold was observed in female mice receiving our gene therapy (FIG. 11C). Interestingly, Naᵥ1.7 seemed to have a lower impact on mechanical allodynia while Naᵥ1.8 seemed to have a bigger impact compared to male mice. Of note, female mice injected with the dual repressor (AAV9-KRAB-dCas9-Naᵥ1.7+Naᵥ1.8) had complete reversal of mechanical allodynia while repression of Naᵥ1.7 or Naᵥ1.8 alone only had a partial effect (FIG. 11D). This suggests a synergistic effect of Naᵥ1.7+Naᵥ1.8 in pain signal transmission as previously hypothesized by us and others. Mice DRG were extracted to test for Naᵥ1.7 and Naᵥ1.8 repression via RT-qPCR (FIG. 11E and FIG. 11F), and observed an increase in Naᵥ1.7 expression in mice injected with No-gRNA as compared to the no-paclitaxel control, as paclitaxel is known to induce Naᵥ expression. A decrease in Naᵥ1.7 levels was observed in mice injected with Naᵥ1.7 and Naᵥ1.7+Naᵥ1.8 dual repressor, as compared to No-gRNA group. A decrease in Naᵥ1.8 expression was also observed in mice injected with the Naᵥ1.8 repressor and the dual Naᵥ1.7+Naᵥ1.8 repressor, as compared to the No-gRNA group. Whether Naᵥ1.7, Naᵥ1.8, and Naᵥ1.7+Naᵥ1.8 in situ repression could lead to a decrease in chemotherapy-induced cold allodynia was also tested. Male and female mice injected were found to have decreased cold allodynia compared to the No-gRNA control. The key for success of this therapeutic approach is the duration of the effect to avoid frequent lumbar punctures and our published results for Naᵥ1.7 indicate that pain can be relieved for at least three months in a CIPN model, and for 44 weeks in an inflammatory model.

### Example 10

### Potential Novel Mechanisms of Action to Reduce Pain and Inflammation

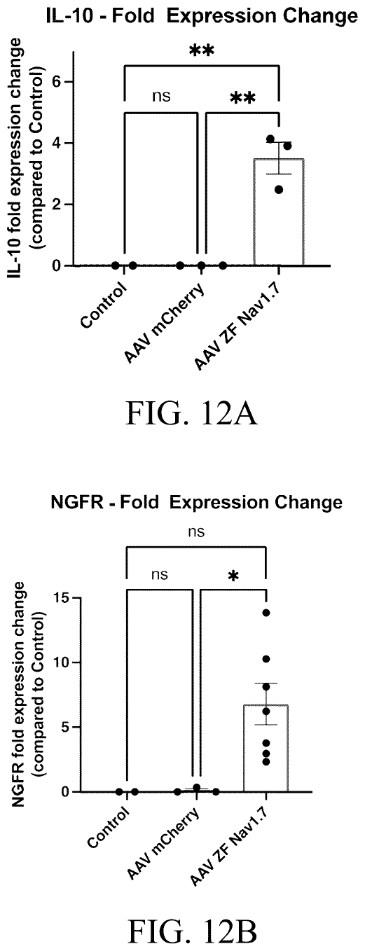

This example describes changes in gene expression levels related to pain in a complete Freund's adjuvant (CFA) mouse model, where NaV1.7 is downregulated through epigenetic modulation. FIG. 12A and FIG. 12B describe the results obtained from mice 24 days after intrathecal injections or 8 days after CFA administration. The ankles of these mice were harvested, and qPCR analysis was performed to assess gene expression. The analysis revealed a significant upregulation of IL-10 and NGFR (NGF receptor) in the treated mice compared to the control group, as shown in FIG. 12A and FIG. 12B, respectively (the error bars in the figure represent the standard error of the mean (SEM), and the statistical significance was determined using a two-way ANOVA with Dunnett's post hoc test, resulting in P-values of 0.0011 (**) for IL-10 and 0.024 (*) for NGFR.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a Nav1.7 epigenetic modulator comprising a SCN9A-binding agent and a first repressor domain, or a polynucleotide encoding the Nav1.7 epigenetic modulator; and
a Nav1.8 epigenetic modulator comprising a SCN10A-binding agent and a second repressor domain, or a polynucleotide encoding the Nav1.8 epigenetic modulator.

2. The composition of claim 1, wherein the SCN9A-binding agent comprises a SCN9A-binding protein, a SCN9A-binding polynucleotide, or a combination thereof, and wherein the SCN10A-binding agent comprises a SCN10A-binding protein, a SCN10A-binding polynucleotide, or a combination thereof.

3. The composition of claim 2, wherein the SCN9A-binding protein comprises a SCN9A-binding zinc finger protein or a SCN9A-binding transcription activator-like effector (TALE), and wherein the SCN10A-binding protein comprises a SCN10A-binding zinc finger protein or a SCN10A-binding transcription activator-like effector (TALE).

4. The composition of claim 3, wherein the SCN9A-binding zinc finger protein comprises a sequence having at least 90% sequence identity to any one of SEQ ID NO: 134-SEQ ID NO: 148 or SEQ ID NO: 278-SEQ ID NO: 290, and wherein the SCN10A-binding zinc finger protein comprises a sequence having at least 90% sequence identity to any one of SEQ ID NO: 149-SEQ ID NO: 192.

5. The composition of claim 2, wherein the SCN9A-binding polynucleotide comprises a SCN9A-binding guide RNA, and wherein the SCN10A-binding polynucleotide comprises a SCN10A-binding guide RNA.

6. The composition of claim 5, wherein the SCN9A-binding guide RNA comprises a sequence having at least 90% sequence identity to any one of SEQ ID NO: 55-SEQ ID NO: 98, and wherein the SCN10A-binding guide RNA comprises a sequence having at least 90% sequence identity to any one of SEQ ID NO: 99-SEQ ID NO: 108 or SEQ ID NO: 268-SEQ ID NO: 277.

7. The composition of claim 1, wherein the SCN9A-binding agent has affinity for a SCN9A polynucleotide sequence, and wherein the SCN10A-binding agent has affinity for a SCNA10 polynucleotide sequence.

8. The composition of claim 7, wherein the SCN9A-binding agent has affinity for a sequence having at least 90% sequence identity to any one of SEQ ID NO: 206-SEQ ID NO: 220 or SEQ ID NO: 291-SEQ ID NO: 302, and wherein the SCN10A-binding agent has affinity for a sequence having at least 90% sequence identity to any one of SEQ ID NO: 221-SEQ ID NO: 263.

9. The composition of claim 1, wherein the first repressor domain, the second repressor domain, or both, comprises ZIM3, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof.

10. The composition of claim 1, wherein the first repressor domain comprises a sequence having at least 90% sequence identity to any one of SEQ ID NO: 193-SEQ ID NO: 205.

11. The composition of claim 1, wherein the SCN9A-binding agent is linked to the first repressor domain, and wherein the SCN10A-binding agent is linked to the second repressor domain.

12. The composition of claim 1, wherein the polynucleotide Nav1.7 epigenetic modulator, the polynucleotide encoding the Nav1.8 epigenetic modulator, or both are under transcriptional control of a promoter.

13. The composition of claim 12, wherein the promoter comprises a sequence having at least 90% sequence identity to any one of SEQ ID NO: 44-SEQ ID NO: 54.

14. The composition of claim 1, wherein the Nav1.7 epigenetic modulator, the polynucleotide encoding the Nav1.7 epigenetic modulator, the Nav1.8 epigenetic modulator, the polynucleotide encoding the Nav1.8 epigenetic modulator, or a combination thereof are encapsulated in a delivery vector.

15. The composition of claim 14, wherein the delivery vector comprises a viral vector, and wherein the viral vector comprises a delivery-enhancing peptide.

16. The composition of claim 15, wherein the delivery-enhancing peptide comprises a protoxin, a jingzhaotoxina, a theraphotoxin, a phlotoxin, Grammostola porter toxin, a huwentoxin, a Ceratogyrus cornuatus toxin, a heteropodatoxin, a heteroscodratoxin, or a penetration enhancing peptide.

17. The composition of claim 15, wherein the delivery-enhancing peptide comprises a sequence having at least 90% sequence identity to any one of SEQ ID NO: 109-SEQ ID NO: 133.

18. The composition of claim 15, wherein the viral vector comprises an AAV vector or a lentiviral vector.

19. A method of downregulating a Nav1.7 and a Nav1.8 in a cell, the method comprising delivering to the cell a Nav1.7 epigenetic modulator comprising a SCN9A-binding agent and a first repressor domain and a Nav1.8 epigenetic modulator comprising a SCN10A-binding agent and a second repressor domain.

20. A method of treating inflammation in a subject in need thereof, the method comprising administering to the subject a composition comprising:
a Nav1.7 epigenetic modulator comprising a SCN9A-binding agent and a first repressor domain; a polynucleotide encoding the Nav1.7 epigenetic modulator;
a Nav1.8 epigenetic modulator comprising a SCN10A-binding agent and a second repressor domain; and
a polynucleotide encoding the Nav1.8 epigenetic modulator; or combinations thereof.


SEQUENCE LISTING (311 sequences)

SEQ ID NO: 1 - protein, 1368 aa, Streptococcus pyogenes [sequence omitted]

SEQ ID NO: 2 - protein, 1269 aa, Mycoplasma gallisepticum
MNNSIKSKPEVTIGLDLGVGSVGWAIVDNETNIIHHLGSRLFSQAKTAEDRRSFRGVRRLIRRRKYKLKRFVNLIWKYNSYFGFKNKEDILNNYQEQQKLHNTVLNLKSEALNAKIDPKALSWILHDYLKNRGHFYEDNRDFNVYPTKELAKYFDKYGYYKGIIDSKEDNDNKLEEELTKYKFSNKHWLEEVKKVLSNQTGLPEKFKEEYESLFSYVRNYSEGPGSINSVSPYGIYHLDEKEGKVVQKYNNIWDKTIGKCNIFPDEYRAPKNSPIAMIFNEINELSTIRSYSIYLTGWFINQEFKKAYLNKLLDLLIKTNGEKPIDARQFKKLREETIAESIGKETLKDVENEEKLEKEDHKWKLKGLKLNTNGKIQYNDLSSLAKFVHKLKQHLKLDFLLEDQYATLDKINFLQSLFVYLGKHLRYSNRVDSANLKEFSDSNKLFERILQKQKDGLFKLFEQTDKDDEKILAQTHSLSTKAMLLAITRMTNLDNDEDNQKNNDKGWNFEAIKNFDQKFIDITKKNNNLSLKQNKRYLDDRFINDAILSPGVKRILREATKVFNAILKQFSEEYDVTKVVIELARELSEEKELENTKNYKKLIKKNGDKISEGLKALGISEDEIKDILKSPTKSYKFLLWLQQDHIDPYSLKEIAFDDIFTKTEKFEIDHIIPYSISFDDSSSNKLLVLAESNQAKSNQTPYEFISSGNAGIKWEDYEAYCRKFKDGDSSLLDSTQRSKKFAKMMKTDTSSKYDIGFLARNLNDTRYATIVFRDALEDYANNHLVEDKPMFKVVCINGSVTSFLRKNFDDSSYAKKDRDKNIHHAVDASIISIFSNETKTLFNQLTQFADYKLFKNTDGSWKKIDPKTGVVTEVTDENWKQIRVRNQVSEIAKVIEKYIQDSNIERKARYSRKIENKTNISLFNDTVYSAKKVGYEDQIKRKNLKTLDIHESAKENKNSKVKRQFVYRKLVNVSLLNNDKLADLFAEKEDILMYRANPWVINLAEQIFNEYTENKKIKSQNVFEKYMLDLTKEFPEKFSEFLVKSMLRNKTAIIYDDKKNIVHRIKRLKMLSSELKENKLSNVIIRSKNQSGTKLSYQDTINSLALMIMRSIDPTAKKQYIRVPLNTLNLHLGDHDFDLHNMDAYLKKPKFVKYLKANEIGDEYKPWRVLTSGTLLIHKKDKKLMYISSFQNLNDVIEIKNLIETEYKENDDSDSKKKKKANRFLMTLSTILNDYILLDAKDNFDILGLSKNRIDEILNSKLGLDKIVK

SEQ ID NO: 3 - protein, 1334 aa, Staphylococcus pseudintermedius
MGRKPYILSLDIGTGSVGYACMDKGFNVLKYHDKDALGVYLFDGALTAQERRQFRTSRRRKNRRIKRLGLLQELLAPLVQNPNFYQFQRQFAWKNDNMDFKNKSLSEVLSFLGYESKKYPTIYHLQEALLLKDEKFDPELIYMALYHLVKYRGHFLFDHLKIENLTNNDNMHDFVELIETYENLNNIKLNLDYEKTKVIYEILKDNEMTKNDRAKRVKNMEKKLEQFSIMLLGLKFNEGKLFNHADNAEELKGANQSHTFADNYEENLTPFLTVEQSEFIERANKIYLSLTLQDILKGKKSMAMSKVAAYDKFRNELKQVKDIVYKADSTRTQFKKIFVSSKKSLKQYDATPNDQTFSSLCLFDQYLIRPKKQYSLLIKELKKIIPQDSELYFEAENDTLLKVLNTTDNASIPMQINLYEAETILRNQQKYHAEITDEMIEKVLSLIQFRIPYYVGPLVNDHTASKFGWMERKSNESIKPWNFDEVVDRSKSATQFIRRMTNKCSYLINEDVLPKNSLLYQEMEVLNELNATQIRLQTDPKNRKYRMMPQIKLFAVEHIFKKYKTVSHSKFLEIMLNSNHRENFMNHGEKLSIFGTQDDKKFASKLSSYQDMTKIFGDIEGKRAQIEEIIQWITIFEDKKILVQKLKECYPELTSKQINQLKKLNYSGWGRLSEKLLTHAYQGHSIIELLRHSDENFMEILTNDVYGFQNFIKEENQVQSNKIQHQDIANLTTSPALKKGIWSTIKLVRELTSIFGEPEKIIMEFATEDQQKGKKQKSRKQLWDDNIKKNKLKSVDEYKYIIDVANKLNNEQLQQEKLWLYLSQNGKCMYSGQSIDLDALLSPNATKHYEVDHIFPRSFIKDDSIDNKVLVIKKMNQTKGDQVPLQFIQQPYERIAYWKSLNKAGLISDSKLHKLMKPEFTAMDKEGFIQRQLVETRQISVHVRDFLKEEYPNTKVIPMKAKMVSEFRKKFDIPKIRQMNDAHHAIDAYLNGVVYHGAQLAYPNVDLFDFNFKWEKVREKWKALGEFNTKQKSRELFFFKKLEKMEVSQGERLISKIKLDMNHFKINYSRKLANIPQQFYNQTAVSPKTAELKYESNKSNEVVYKGLTPYQTYVVAIKSVNKKGKEKMEYQMIDHYVFDFYKFQNGNEKELALYLAQRENKDEVLDAQIVYSLNKGDLLYINNHPCYFVSRKEVINAKQFELTVEQQLSLYNVMNNKETNVEKLLIEYDFIAEKVINEYHHYLNSKLKEKRVRTFFSESNQTHEDFIKALDELFKVVTASATRSDKIGSRKNSMTHRAFLGKGKDVKIAYTSISGLKTTKPKSLFKLAESRNEL

SEQ ID NO: 4 - protein, 1084 aa, Corynebacterium diphtheriae
MKYHVGIDVGTFSVGLAAIEVDDAGMPIKTLSLVSHIHDSGLDPDKIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDEPSDAFKAIREEIKRASGQPVPETATVGQMVTLCELGTLKLRGEGGVLSARLQQSDHAREIQEICRMQEIGQELYRKIIDVVFAAESPKGSASSRVGKDPLQPGKNRALKASDAFQRYRIAALIGNLRVRVDGEKRILSVEEKNLVFDHLVNLAPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLADGVDLYTARLQEFGIEPSWTPPAPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLEFLDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHEETIGKLSKVKLGSQLSVSDIDKASSEALWCALTREPDFDPKDGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFGDTRLRLRPLQMSKEGIKKESAPELSKIIDRPGWLPAVNKLFSEGNVTVVRRDSLGRVRLESTAHLPVTWKVQ

SEQ ID NO: 5 - protein, 967 aa, unidentified
MLGSSRYLRYNLTSFEGKEPFLIMGYYKEYNKELSSKAQKEFNDQISEFNSYYKLGIDLGDKTGIAIVKGNKIILAKTLIDLHSQKLDKRREARRNRRTRLSRKKRLARLRSWVMRQKVGNQRLPDPYKIMHDNKYWSIYNKSNSANKKNWIDLLIHSNSLSADDFVRGLTIIFRKRGYLAFKYLSRLSDKEFEKYIDNLKPPISKYEYDEDLEELSSRVENGEIEEKKFEGLKNKLDKIDKESKDFQVKQREEVKKELEDLVDLFAKSVDNKIDKARWKRELNNLLDKKVRKIRFDNRFILKCKIKGCNKNTPKKEKVRDFELKMVLNNARSDYQISDEDLNSFRNEVINIFQKKENLKKGELKGVTIEDLRKQLNKTFNKAKIKKGIREQIRSIVFEKISGRSKFCKEHLKEFSEKPAPSDRINYGVNSAREQHDFRVLNFIDKKIFKDKLIDPSKLRYITIESPEPETEKLEKGQISEKSFETLKEKLAKETGGIDIYTGEKLKKDFEIEHIFPRARMGPSIRENEVASNLETNKEKADRTPWEWFGQDEKRWSEFEKRVNSLYSKKKISERKREILLNKSNEYPGLNPTELSRIPSTLSDFVESIRKMFVKYGYEEPQTLVQKGKPIIQVVRGRDTQALRWRWHALDSNIIPEKDRKSSFNHAEDAVIAACMPPYYLRQKIFREEAKIKRKVSNKEKEVTRPDMPTKKIAPNWSEFMKTRNEPVIEVIGKVKPSWKNSIMDQTFYKYLLKPFKDNLIKIPNVKNTYKWIGVNGQTDSLSLPSKVLSISNKKVDSSTVLLVHDKKGGKRNWVPKSIGGLLVYITPKDGPKRIVQVKPATQGLLIYRNEDGRVDAVREFINPVIEMYNNGKLAFVEKENEEELLKYFNLLEKGQKFERIRRYDMITYNSKFYYVTKINKNHRVTIQEESKIKAESDKVKSSSGKEYTRKETEELSLQKLAELISI

SEQ ID NO: 6 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 7 - protein, 1338 aa, Coprococcus catus
MKQEYFLGLDMGTGSLGWAVTDSTYQVMRKHGKALWGTRLFESASTAEERRMFRTARRRLDRRNWRIQVLQEIFSEEISKVDPGFFLRMKESKYYPEDKRDAEGNCPELPYALFVDDNYTDKNYHKDYPTIYHLRKMLMETTEIPDIRLVYLVLHHMMKHRGHFLLSGDISQIKEFKSTFEQLIQNIQDEELEWHISLDDAAIQFVEHVLKDRNLTRSTKKSRLIKQLNAKSACEKAILNLLSGGTVKLSDIFNNKELDESERPKVSFADSGYDDYIGIVEAELAEQYYIIASAKAVYDWSVLVEILGNSVSISEAKIKVYQKHQADLKTLKKIVRQYMTKEDYKRVFVDTEEKLNNYSAYIGMTKKNGKKVDLKSKQCTQADFYDFLKKNVIKVIDHKEITQEIESEIEKENFLPKQVTKDNGVIPYQVHDYELKKILDNLGTRMPFIKENAEKIQQLFEFRIPYYVGPLNRVDDGKDGKFTWSVRKSDARIYPWNFTEVIDVEASAEKFIRRMTNKCTYLVGEDVLPKDSLVYSKFMVLNELNNLRLNGEKISVELKQRIYEELFCKYRKVTRKKLERYLVIEGIAKKGVEITGIDGDFKASLTAYHDFKERLTDVQLSQRAKEAIVLNVVLFGDDKKLLKQRLSKMYPNLTTGQLKGICSLSYQGWGRLSKTFLEEITVPAPGTGEVWNIMTALWQTNDNLMQLLSRNYGFTNEVEEFNTLKKETDLSYKTVDELYVSPAVKRQIWQTLKVVKEIQKVMGNAPKRVFVEMAREKQEGKRSDSRKKQLVELYRACKNEERDWITELNAQSDQQLRSDKLFLYYIQKGRCMYSGETIQLDELWDNTKYDIDHIYPQSKTMDDSLNNRVLVKKNYNAIKSDTYPLSLDIQKKMMSFWKMLQQQGFITKEKYVRLVRSDELSADELAGFIERQIVETRQSTKAVATILKEALPDTEIVYVKAGNVSNFRQTYELLKVREMNDLHHAKDAYLNIVVGNAYFVKFTKNAAWFIRNNPGRSYNLKRMFEFDIERSGEIAWKAGNKGSIVTVKKVMQKNNILVTRKAYEVKGGLFDQQIMKKGKGQVPIKGNDERLADIEKYGGYNKAAGTYFMLVKSLDKKGKEIRTIEFVPLYLKNQIEINHESAIQYLAQERGLNSPEILLSKIKIDTLFKVDGFKMWLSGRTGNQLIFKGANQLILSHQEAAILKGVVKYVNRKNENKDAKLSERDGMTEEKLLQLYDTFLDKLSNTVYSIRLSAQIKTLTEKRAKFIGLSNEDQCIVLNEILHMFQCQSGSANLKLIGGPGSAGILVMNNNITACKQISVINQSPTGIYEKEIDLIKL

SEQ ID NO: 8 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 9 - protein, 1150 aa, Enterococcus faecalis
MYSIGLDLGISSVGWSVIDERTGNVIDLGVRLFSAKNSEKNLERRTNRGGRRLIRRKTNRLKDAKKILAAVGFYEDKSLKNSCPYQLRVKGLTEPLSRGEIYKVTLHILKKRGISYLDEVDTEAAKESQDYKEQVRKNAQLLTKYTPGQIQLQRLKENNRVKTGINAQGNYQLNVFKVSAYANELATILKTQQAFYPNELTDDWIALFVQPGIAEEAGLIYRKRPYYHGPGNEANNSPYGRWSDFQKTGEPATNIFDKLIGKDFQGELRASGLSLSAQQYNLLNDLTNLKIDGEVPLSSEQKEYILTELMTKEFTRFGVNDVVKLLGVKKERLSGWRLDKKGKPEIHTLKGYRNWRKIFAEAGIDLATLPTETIDCLAKVLTLNTEREGIENTLAFELPELSESVKLLVLDRYKELSQSISTQSWHRFSLKTLHLLIPELMNATSEQNTLLEQFQLKSDVRKRYSEYKKLPTKDVLAEIYNPTVNKTVSQAFKVIDALLVKYGKEQIRYITIEMPRDDNEEDEKKRIKELHAKNSQRKNDSQSYFMQKSGWSQEKFQTTIQKNRRFLAKLLYYYEQDGICAYTGLPISPELLVSDSTEIDHIIPISISLDDSINNKVLVLSKANQVKGQQTPYDAWMDGSFKKINGKFSNWDDYQKWVESRHFSHKKENNLLETRNIFDSEQVEKFLARNLNDTRYASRLVLNTLQSFFTNQETKVRVVNGSFTHTLRKKWGADLDKTRETHHHHAVDATLCAVTSFVKVSRYHYAVKEETGEKVMREIDFETGEIVNEMSYWEFKKSKKYERKTYQVKWPNFREQLKPVNLHPRIKFSHQVDRKANRKLSDATIYSVREKTEVKTLKSGKQKITTDEYTIGKIKDIYTLDGWEAFKKKQDKLLMKDLDEKTYERLLSIAETTPDFQEVEEKNGKVKRVKRSPFAVYCEENDIPAIQKYAKKNNGPLIRSLKYYDGKLNKHINITKDSQGRPVEKTKNGRKVTLQSLKPYRYDIYQDLETKAYYTVQLYYSDLRFVEGKYGITEKEYMKKVAEQTKGQVVRFCFSLQKNDGLEIEWKDSQRYDVRFYNFQSANSINFKGLEQEMMPAENQFKQKPYNNGAINLNIAKYGKEGKKLRKFNTDILGKKHYLFYEKEPKNIIK

SEQ ID NO: 10 - protein, 1233 aa, Mycoplasma canis
MEKKRKVTLGFDLGIASVGWAIVDSETNQVYKLGSRLFDAPDTNLERRTQRGTRRLLRRRKYRNQKFYNLVKRTEVFGLSSREAIENRFRELSIKYPNIIELKTKALSQEVCPDEIAWILHDYLKNRGYFYDEKETKEDFDQQTVESMPSYKLNEFYKKYGYFKGALSQPTESEMKDNKDLKEAFFFDFSNKEWLKEINYFFNVQKNILSETFIEEFKKIFSFTRDISKGPGSDNMPSPYGIFGEFGDNGQGGRYEHIWDKNIGKCSIFTNEQRAPKYLPSALIFNFLNELANIRLYSTDKKNIQPLWKLSSVDKLNILLNLFNLPISEKKKKLTSTNINDIVKKESIKSIMISVEDIDMIKDEWAGKEPNVYGVGLSGLNIEESAKENKFKFQDLKILNVLINLLDNVGIKFEFKDRNDIIKNLELLDNLYLFLIYQKESNNKDSSIDLFIAKNESLNIENLKLKLKEFLLGAGNEFENHNSKTHSLSKKAIDEILPKLLDNNEGWNLEAIKNYDEEIKSQIEDNSSLMAKQDKKYLNDNFLKDAILPPNVKVTFQQAILIFNKIIQKFSKDFEIDKVVIELAREMTQDQENDALKGIAKAQKSKKSLVEERLEANNIDKSVFNDKYEKLIYKIFLWISQDFKDPYTGAQISVNEIVNNKVEIDHIIPYSLCFDDSSANKVLVHKQSNQEKSNSLPYEYIKQGHSGWNWDEFTKYVKRVFVNNVDSILSKKERLKKSENLLTASYDGYDKLGFLARNLNDTRYATILFRDQLNNYAEHHLIDNKKMFKVIAMNGAVTSFIRKNMSYDNKLRLKDRSDFSHHAYDAAIIALFSNKTKTLYNLIDPSLNGIISKRSEGYWVIEDRYTGEIKELKKEDWTSIKNNVQARKIAKEIEEYLIDLDDEVFFSRKTKRKTNRQLYNETIYGIATKTDEDGITNYYKKEKFSILDDKDIYLRLLREREKFVINQSNPEVIDQIIEIIESYGKENNIPSRDEAINIKYTKNKINYNLYLKQYMRSLTKSLDQFSEEFINQMIANKTFVLYNPTKNTTRKIKFLRLVNDVKINDIRKNQVINKFNGKNNEPKAFYENINSLGAIVFKNSANNFKTLSINTQIAIFGDKNWDIEDFKTYNMEKIEKYKEIYGIDKTYNFHSFIFPGTILLDKQNKEFYYISSIQTVRDIIEIKFLNKIEFKDENKNQDTSKTPKRLMFGIKSIMNNYEQVDISPFGINKKIFE

SEQ ID NO: 11 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYIAIWASQRPVASKDDIEKKVGFCAFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKEKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 12 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 13 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 14 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 15 - protein, 1377 aa, Streptococcus agalactiae
MNKPYSIGLDIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATMQEEKYYHEKFPTIYHLRKELADKKEKADLRLVYLALAHIIKFRGHFLIEDDRFDVRNTDIQKQYQAFLEIFDTTFENNHLLSQNVDVEAILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHHEDLKHLKQFVKASLPENYREVFADSSKDGYAGYIEGKTNQEAFYKYLLKLLTKQEGSEYFLEKIKNEDFLRKQRTFDNGSIPHQVHLTELRAIIRRQSEYYPFLKENQDRIEKILTFRIPYYVGPLAREKSDFAWMTRKTDDSIRPWNFEDLVDKEKSAEAFIHRMTNNDLYLPEEKVLPKHSLIYEKFTVYNELTKVRFLAEGFKDFQFLNRKQKETIFNSLFKEKRKVTEKDIISFLNKVDGYEGIAIKGIEKQFNASLSTYHDLKKILGKDFLDNTDNELILEDIVQTLTLFEDREMIKKCLDIYKDFFTESQLKKLYRRHYTGWGRLSAKLINGIRNKENQKTILDYLIDDGSANRNFMQLINDDDLSFKPIIDKARTGSHSDNLKEVVGELAGSPAIKKGILQSLKIVDELVKVMGYEPEQIVVEMARENQTTAKGLSRSRQRLTTLRESLANLKSNILEEKKPKYVKDQVENHHLSDDRLFLYYLQNGRDMYTKKALDIDNLSQYDIDHIIPQAFIKDDSIDNRVLVSSAKNRGKSDDVPSIEIVKARKMFWKNLLDAKLMSQRKYDNLTKAERGGLTSDDKARFIQRQLVETRQITKHVARILDERFNNEVDNGKKICKVKIVTLKSNLVSNFRKEFGFYKIREVNDYHHAHDAYLNAVVAKAILTKYPQLEPEFVYGMYRQKKLSKIVHEDKEEKYSEATRKMFFYSNLMNMFKRVVRLADGSIVVRPVIETGRYMRKTAWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNESKGKPEEIEKKQEFVNQHVSYFDDILQLINDFSKRVILADANLEKINKLYQDNKENIPVDELANNIINLFTFTSLGAPAAFKFFDKIVDRKRYTSTKEVLNSTLIHQSITGLYETRIDLGKLGED

SEQ ID NO: 16 - protein, 1130 aa, Streptococcus sp.
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 17 - protein, 1082 aa, Neisseria lactamica
MAAFKPNPMNYILGLDIGIASVGWAMVEVDEEENPIRLIDLGVRVFERAEVPKTGDSLAMARRLARSVRRLTRRRAHRLLRARRLLKREGVLQDADFDENGLVKSLPNTPWQLRAAALDRKLTCLEWSAVLLHLVKHRGYLSQRKNEGETADKELGALLKGVADNAHALQTGDFRTPAELALNKFEKESGHIRNQRGDYSHTFSRKDLQAELNLLFEKQKEFGNPHVSDGLKEDIETLLMAQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSTELQDEIGTAFSLFKTDKDITGRLKDRVQPEILEALLKHISFDKFVQISLKALRRIVPLMEQGKRYDEACAEIYGDHYCKKNAEEKIYLPPIPADEIRNPVVLRALSQARKVINCVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHILLTGKGKRRVFASNGQITNLLRGFWGLRKVRTENDRHHALDAVVVACSTVAMQQKITRFVRYKEMNAFDGKTIDKETGEVLHQKAHFPQPWEFFAQEVMIRVFGKPDGKPEFEEADTPEKLRTLLAEKLSSRPEAVHEYVTPLFVSRAPNRKMSGQGHMETVKSAKRLDEGISVLRVPLTQLKLKGLEKMVNREREPKLYDALKAQLETHKDDPAKAFAEPFYKYDKAGSRTQQVKAVRIEQVQKTGVWVRNHNGIADNATMVRVDVFEKGGKYYLVPIYSWQVAKGILPDRAVVAFKDEEDWTVMDDSFEFRFVLYANDLIKLTAKKNEFLGYFVSLNRATGAIDIRTHDTDSTKGKNGIFQSVGVKTALSFQKNQIDELGKEIRPCRLKKRPPVR

SEQ ID NO: 18 - protein, 1125 aa, Francisella tularensis
MNVKILPIAIDLDVKNTGVFSAFYQKGTSLEKLDNKNGKVYELSKDSYTLLMNNRTARRHQRRGIDRKQLVKRLFKLVWTEQLNLEWDKDTQQAISFLFNRRGFSFITDGYSTEYLNIVPEQVKAILMDIFDDYNGEDDLDSYLKLATEQESKISEIYNKLMQKILEFKLRKLCTDIKDDKVSTKTLKEITSYEFELLADYLANYSESLKIQKFSYTDKQGNLKELSYYHHDKYNIQEFLKRHATINDEILDTLLTDDFDIWNFNFEKFDFDKNEEKLQNQEDKDHTQAHLHHFVFAVNKIKSEMASGGRHRSQYFQEITNVLDENNHQEGYLKNFCENLHNKKYSNLSVKNLVNLVGNLSNLELKPLRKYFNDKIHAKADHWDEQKFTETYCHWILGEWRVGVKDQDKKDGAKYSYKDLCNELKQKVTKAGLIDFLLELDPCRTIPPYLDNNNRKPPKCQSLILNPKFLDNQYPNWQQYLQELKKLQSIQDYLDSFETDLKDLKSSKDQPYFVEYKSSNQQMASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLDKYNNTGRFDDNNQLLTYCNHKPRQKRYQLLNDLAGVLQVSRNQLLSSVEEWFQQAQRVGEISKSQDEQIFEWLKSFKIASYCKAAVEMQKQYRGTLKNAIQTAIFRQSENINKNKNKNTGNQQQALSENSKDVKSLTADEKKLLKLIENIAKASQKIGESLGLNDKQIKKFNSIYSFAQIQQIAFAERKGNANTCAVCSADNAHRMQQIKITELVEDNKDNIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRDDNKNIFAIDTSKWFEIETPSDLRDIGVATIQYKIDNNSRPKVRVKLDYVIDDDSKINYFMNHSLLKSRYPDKVLEILKQSTIIEFESSGFNKTIKEMLGMTLAGIYNETSNN

SEQ ID NO: 19 - protein, 1064 aa, Bradyrhizobium sp.
MKRTSLRAYRLGVDLGANSLGWFVVWLDDHGQPEGLGPGGVRIFPDGRNPQSKQSNAAGRRLARSARRRRDRYLQRRGKLMGLLVKHGLMPADEPARKRLECLDPYGLRAKALDEVLPLHHVGRALFHLNQRRGLFANRAIEQGDKDASAIKAAAGRLQTSMQACGARTLGEFLNRRHQLRATVRARSPVGGDVQARYEFYPTRAMVDAEFEAIWAAQAPHHPTMTAEAHDTIREAIFSQRAMKRPSIGKCSLDPATSQDDVDGFRCAWSHPLAQRFRIWQDVRNLAVVETGPTSSRLGKEDQDKVARALLQTDQLSFDEIRGLLGLPSDARFNLESDRRDHLKGDATGAILSARRHFGPAWHDRSLDRQIDIVALLESALDEAAIIASLGTTHSLDEAAAQRALSALLPDGYCRLGLRAIKRVLPLMEAGRTYAEAASAAGYDHALLPGGKLSPTGYLPYYGQWLQNDVVGSDDERDTNERRWGRLPNPTVHIGIGQLRRVVNELIRWHGPPAEITVELTRDLKLSPRRLAELEREQAENQRKNDKRTSLLRKLGLPASTHNLLKLRLWDEQGDVASECPYTGEAIGLERLVSDDVDIDHLIPFSISWDDSAANKVVCMRYANREKGNRTPFEAFGHRQGRPYDWADIAERAARLPRGKRWRFGPGARAQFEELGDFQARLLNETSWLARVAKQYLAAVTHPHRIHVLPGRLTALLRATWELNDLLPGSDDRAAKSRKDHRHHAIDALVAALTDQALLRRMANAHDDTRRKIEVLLPWPTFRIDLETRLKAMLVSHKPDHGLQARLHEDTAYGTVEHPETEDGANLVYRKTFVDISEKEIDRIRDRRLRDLVRAHVAGERQQGKTLKAAVLSFAQRRDIAGHPNGIRHVRLTKSIKPDYLVPIRDKAGRIYKSYNAGENAFVDILQAESGRWIARATTVFQANQANESHDAPAAQPIMRVFKGDMLRIDHAGAEKFVKIVRLSPSNNLLYLVEHHQAGVFQTRHDDPEDSFRWLFASFDKLREWNAELVRIDTLGQPWRRKRGLETGSEDATRIGWTRPKKWP

SEQ ID NO: 20 - protein, 1136 aa, Streptococcus gordonii
MNGLVLGLDIGIASVGVGILEKDTGKIIHASSRLFPAATADNNVERRSNRQGRRLNRRKKHRSVRLQDLFEGYGLLTDFSKVSMNLNPYQLRVQGMENQLTNEELFVALKNIVKRRGISYLDDASEDGGTVSSDYGKAVEENRKLLAEKTPGQIQLERFEKYGQLRGDFTVEENGEKHRLINVFSTSAYRKEAERILRKQQEFNSKITDEFIEDYLIILTGKRKYYHGPGNEKSRTDYGRFRTDGTTLDNIFGILIGKCTFYTEEYRASKASYTAQEFNLLNDLNNLTVPTETKKLSEEQKKLIIEYAKSAKTLGASTLLKYIAKMIDASVDQIRGYRVDVNNKPEMHTFEVYRKMQSLETIKVEELPRKVLDELAHILTLNTEREGIEEAINSKLKDIFNRDQVLELVQFRKNNSSLFSKGWHNFSIKLMMELIPELYETSEEQMTILTRLGKQRSKETSKRTKYIDEKELTEEIYNPVVAKSVRQAIKIINEATKKYGIFDNIVIEMARENNEEDAKKDYIKRQKANQDEKNAAMEKAAFQYNGKKELPDNIFHGHKELTTKIRLWHQQGEKCLYTGKNIPISDLIHNQYKYEIDHILPLSLSFDDSLSNKVLVLATANQEKGQRTPFQALDSMDDAWSYREFKSYVKDSKLLSNKKKDYLLTEEDISKIEVKQKFIERNLVDTRYSSRVVLNALQDFYKSHQLDTTISVVRGQFTSQLRRKWGIEKSRETYHHHAVDALIIAASSQLRLWKKHSNPLIAYKEGQFVDSETGEIVSLSDEEYKELVFKAPYDHFVDTLRSKKFEDSILFSYQVDSKYNRKISDATIYATRKAKLDKEKKEYTYTLGKIKDIYALGTKTPSKTGFYKFLDLYKTDKSQFLMYQKDRKTWDEVIEKIIEQYRPFKEYDKNGKEVDFNPFEKYRIGNGPIRKYSKKGNGPEIKSLKYYDILLGKHKNITPDGSRNTVALLSLNPWRTDVYYNSETKKYEFLGLKYADLCFEEGGAYGISEVKYKKIREKEGIGKNSEFKFTLYKNDLILIKDTETNCQQFFRFWSRTGKDNPKSFEKHKIELKPYEKAKFEKGEELKVLGKVPPSSNQFQKNMQIENLSIYKVKTDILGNKHFIKKEGDEPKLKFKK

SEQ ID NO: 21 - protein, 984 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNNGDEEKSEILKAIKQNEEKLVNYQSVGEYLYKEYFQKFKENSKEFINVRNKKESYERCITQSFLKDELKLIFQKQREFGFSFSKKFEEEVLSVAFYKRALKDFSHLVGNCSFFTDEKRAPKNSPLAFMFVALTRIINILNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKILNALLKKYGKVHKINIELAREVGKNHSQRAKIEKEQNENYKAKKDAEIECEKLGLKINSKNILKLRLFKEQKEFCAYSGEKIKLSDLQDEKMLEIDHIYPYSRSFDDSYMNKVLVFTKQNQEKLNKTPFEAFGNDSTKWQKIEVLAKNLPEKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAAIIAYANNSIVKAFSDFKKEQESNSVELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPELVYFNAFTSSTVSLIVSKHDNKFETLSKNQKILFKNANEKEVIAKSIGIQNLKVFEKYIVSALGEVTKAEFRQREDFKK

SEQ ID NO: 22 - protein, 1079 aa, Dinoroseobacter shibae
MRLGLDIGTSSIGWWLYETDGAGSDARITGVVDGGVRIFSDGRDPKSGASLAVDRRAARAMRRRRDRYLRRRATLMKVLAETGLMPADPAEAKALEALDPFALRAAGLDEPLPLPHLGRALFHLNQRRGFKSNRKTDRGDNESGKIKDATARLDMEMMANGARTYGEFLHKRRQKATDPRHVPSVRTRLSIANRGGPDGKEEAGYDFYPDRRHLEEEFHKLWAAQGAHHPELTETLRDLLFEKIFFQRPLKEPEVGLCLFSGHHGVPPKDPRLPKAHPLTQRRVLYETVNQLRVTADGREARPLTREERDQVIHALDNKKPTKSLSSMVLKLPALAKVLKLRDGERFTLETGVRDAIACDPLRASPAHPDRFGPRWSILDADAQWEVISRIRRVQSDAEHAALVDWLTEAHGLDRAHAEATAHAPLPDGYGRLGLTATTRILYQLTADVVTYADAVKACGWHHSDGRTGECFDRLPYYGEVLERHVIPGSYHPDDDDITRFGRITNPTVHIGLNQLRRLVNRIIETHGKPHQIVVELARDLKKSEEQKRADIKRIRDTTEAAKKRSEKLEELEIEDNGRNRMLLRLWEDLNPDDAMRRFCPYTGTRISAAMIFDGSCDVDHILPYSRTLDDSFPNRTLCLREANRQKRNQTPWQAWGDTPHWHAIAANLKNLPENKRWRFAPDAMTRFEGENGFLDRALKDTQYLARISRSYLDTLFTKGGHVWVVPGRFTEMLRRHWGLNSLLSDAGRGAVKAKNRTDHRHHAIDAAVIAATDPGLLNRISRAAGQGEAAGQSAELIARDTPPPWEGFRDDLRVRLDRIIVSHRADHGRIDHAARKQGRDSTAGQLHQETAYSIVDDIHVASRTDLLSLKPAQLLDEPGRSGQVRDPQLRKALRVATGGKTGKDFENALRYFASKPGPYQAIRRVRIIKPLQAQARVPVPAQDPIKAYQGGSNHLFEIWRLPDGEIEAQVITSFEAHTLEGEKRPHPAAKRLLRVHKGDMVALERDGRRVVGHVQKMDIANGLFIVPHNEANADTRNNDKSDPFKWIQIGARPAIASGIRRVSVDEIGRLRDGGTRPI

SEQ ID NO: 23 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELNLLFEKQKEFGNPHISDGLKEGIETLLMTQRPALSGDAVQKMLGHCTFEPAEPKAAKNTYTAERFIWLTKLNNLRILEQGSERPLTDTERTTLIDEPYRKSKLTYAQARKLLELDNTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGKRYDEACAEIYGDHYGKKNTEEKIDLPPIPADEIRNPVVLRALSQARKVINGVVRRYGSPARIHIETAREVGKSFKDRKEIEKRQEENRKDREKAATKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHMLLTGKGKRRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 24 - protein, 1195 aa, Elusimicrobium minutum
MQKNINTKQNHIYIKQAQKIKEKLGDKPYRIGLDLGVGSIGFAIVSMEENDGNVLLPKEIIMVGSRIFKASAGAADRKLSRGQRNNHRHTRERMRYLWKVLAEQKLALPVPADLDRKENSSEGETSAKRFLGDVLQKDIYELRVKSLDERLSLQELGYVLYHIAGHRGSSAIRTFENDSEEAQKENTENKKIAGNIKRLMAKKNYRTYGEYLYKEFFENKEKHKREKISNAANNHKFSPTRDLVIKEAEAILKKQAGKDGFHKELTEEYIEKLTKAIGYESEKLIPESGFCPYLKDEKRLPASHKLNEERRLWETLNNARYSDPIVDIVTGEITGYYEKQFTKEQKQKLFDYLLTGSELTPAQTKKLLGLKNTNFEDIILQGRDKKAQKIKGYKLIKLESMPFWARLSEAQQDSFLYDWNSCPDEKLLTEKLSNEYHLTEEEIDNAFNEIVLSSSYAPLGKSAMLIILEKIKNDLSYTEAVEEALKEGKLTKEKQAIKDRLPYYGAVLQESTQKIIAKGFSPQFKDKGYKTPHTNKYELEYGRIANPVVHQTLNELRKLVNEIIDILGKKPCEIGLETARELKKSAEDRSKLSREQNDNESNRNRIYEIYIRPQQQVIITRRENPRNYILKFELLEEQKSQCPFCGGQISPNDIINNQADIEHLFPIAESEDNGRNNLVISHSACNADKAKRSPWAAFASAAKDSKYDYNRILSNVKENIPHKAWRFNQGAFEKFIENKPMAARFKTDNSYISKVAHKYLACLFEKPNIICVKGSLTAQLRMAWGLQGLMIPFAKQLITEKESESFNKDVNSNKKIRLDNRHHALDAIVIAYASRGYGNLLNKMAGKDYKINYSERNWLSKILLPPNNIVWENIDADLESFESSVKTALKNAFISVKHDHSDNGELVKGTMYKIFYSERGYTLTTYKKLSALKLTDPQKKKTPKDFLETALLKFKGRESEMKNEKIKSAIENNKRLFDVIQDNLEKAKKLLEEENEKSKAEGKKEKNINDASIYQKAISLSGDKYVQLSKKEPGKFFAISKPTPTTTGYGYDTGDSLCVDLYYDNKGKLCGEIIRKIDAQQKNPLKYKEQGFTLFERIYGGDILEVDFDIHSDKNSFRNNTGSAPENRVFIKVGTFTEITNNNIQIWFGNIIKSTGGQDDSFTINSMQQYNPRKLILSSCGFIKYRSPILKNKEG

SEQ ID NO: 25 - protein, 1101 aa, Akkermansia muciniphila
MSRSLTFSFDIGYASIGWAVIASASHDDADPSVCGCGTVLFPKDDCQAFKRREYRRLRRNIRSRRVRIERIGRLLVQAQIITPEMKETSGHPAPFYLASEALKGHRTLAPIELWHVLRWYAHNRGYDNNASWSNSLSEDGGNGEDTERVKHAQDLMDKHGTATMAETICRELKLEEGKADAPMEVSTPAYKNLNTAFPRLIVEKEVRRILELSAPLIPGLTAEIIELIAQHHPLTTEQRGVLLQHGIKLARRYRGSLLFGQLIPRFDNRIISRCPVTWAQVYEAELKKGNSEQSARERAEKLSKVPTANCPEFYEYRMARILCNIRADGEPLSAEIRRELMNQARQEGKLTKASLEKAISSRLGKETETNVSNYFTLHPDSEEALYLNPAVEVLQRSGIGQILSPSVYRIAANRLRRGKSVTPNYLLNLLKSRGESGEALEKKIEKESKKKEADYADTPLKPKYATGRAPYARTVLKKVVEEILDGEDPTRPARGEAHPDGELKAHDGCLYCLLDTDSSVNQHQKERRLDTMTNNHLVRHRMLILDRLLKDLIQDFADGQKDRISRVCVEVGKELTTFSAMDSKKIQRELTLRQKSHTDAVNRLKRKLPGKALSANLIRKCRIAMDMNWTCPFTGATYGDHELENLELEHIVPHSFRQSNALSSLVLTWPGVNRMKGQRTGYDFVEQEQENPVPDKPNLHICSLNNYRELVEKLDDKKGHEDDRRRKKKRKALLMVRGLSHKHQSQNHEAMKEIGMTEGMMTQSSHLMKLACKSIKTSLPDAHIDMIPGAVTAEVRKAWDVFGVFKELCPEAADPDSGKILKENLRSLTHLHHALDACVLGLIPYIIPAHHNGLLRRVLAMRRIPEKLIPQVRPVANQRHYVLNDDGRMMLRDLSASLKENIREQLMEQRVIQHVPADMGGALLKETMQRVLSVDGSGEDAMVSLSKKKDGKKEKNQVKASKLVGVFPEGPSKLKALKAAIEIDGNYGVALDPKPVVIRHIKVFKRIMALKEQNGGKPVRILKKGMLIHLTSSKDPKHAGVWRIESIQDSKGGVKLDLQRAHCAVPKNKTHECNWREVDLISLLKKYQMKRYPTSYTGTPR

SEQ ID NO: 26 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEIAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDLQKAQVSGQGDSLHEHIANLAGSPAIKKGILQTVKVVDELVKVMGRHKPENIVIEMARENQTTQKGQKNSRERMKRIEEGIKELGSDILKEYPVETTQLQNEKLYLYYLQNGRDMYVDQELDINRLSDYDVDHIVPQSFLKDDSIDNKVLTRSDKNRGKSDNVPSEEVVKKMKNYWKQLLNAKLITQRKFDNLTKAERGGLSELDKAGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDIRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELVGITIMERSSFEKDPVDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKCFDTTIGRNRYKSIKEVLDATLIHQSITGLYETRIDLSQLGGD

SEQ ID NO: 27 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 28 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 29 - protein, 1131 aa, Acidovorax sp.
MAQHVFGLDIGIASVGWAILGEQRIIDLGVRCFDKAETAKEGDPLNLTRRQARLLRRRLYRRAWRLTQLSRLLKRKGLIADAKLFAKAPSYGDSAWELRRQGLDRLLTPLEWARVIYHQCKHRGFHWTSKAEEAKADSDAEGGRVKQGLAHTKALMQAKNYRSAAEMVLAEFPDAQRNKRGQYDKALSRVLLGEELALLFATQRRLGNPHASDFFEKLILGDGDRKSGLFWQQKPALSGADLLKMLGKCTFEKGEYRAPKASFSVERHVWLTRLNNLRIVVDGRSRPLNEAERQAALLLPYQTETSKYKTLKNAFIKAGLWGDGVRFGGLAYPSQAQIDAEKTKDPEDQFLVKLPAWHELRKAFKAAGHEALWQQISTPALDGDPTLLDQIATVLSVYKDGAEVVQQLRQLALPEPAASIAVLEKISFDKFSSLSLKALRRIVPLMQSGLRYDEAVAQIPEYGHHSQRIEPGAAKHLYLPPFYEAQRKYAGKGDHIGSMQFRDDADIPRNPVVLRALNQARKVVNALIREYGSPIAVNIEMARDLSRPLDERNKVKRAQEEFRDRNDRARSEFERDFGYKPKAAAFEKWMLYREQLGQCAYSQQPLDIQRVLDDHNYAQVDHALPYSRSYDDSKNNKVLVLTHENQNKGNRTAFEYLTSFPDGEDGERWRTFVAWVQGNKAYRMAKRNRLLRKNYGVDESKGFIDRNLNDTRYICKFFKNYVEEHLQLAARADGDTARRCVVVNGQLTAFLRARWGLTKVRGDSDRHHALDAAVVAACTHGMVKALADYSRRKEISFLQEGFPDPETGEILNPAAFDRARQHFPEPWTHFAHELKARLFTDDLAALREDMQRLGSYTTEDLGRLRTLFVSRAPQRRSGGAVHKETIYAQPESLKQQGGVIEKILLTSLKLQDFDKLLNPESNDHFVEPHRNERLYAAIRQRLEQFGGRADKAFGPDNLFHKPDKNNQPTGPVVRSIKLVRGKQTGIPIRGGLAKNDSMLRVDIFTKAGKFHLVPVYVHHRVTGLPNRAIVAFKDEDEWTLIDESFAFLFSVYPNDYVKVTLKKEQQSGYYSGADRSTGAMNLWAHDRAASVGKDGLIRGIGVKTALSVEKFNVDVLGRIYLAPPETRSGLA

SEQ ID NO: 30 - protein, 1082 aa, Neisseria meningitidis
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 31 - protein, 1363 aa, Lactobacillus rhamnosus
MTKLNQPYGIGLDIGSNSIGFAVVDANSHLLRLKGETAIGARLFREGQSAADRRGSRTTRRRLSRTRWRLSFLRDFFAPHITKIDPDFFLRQKYSEISPKDKDRFKYEKRLFNDRTDAEFYEDYPSMYHLRLHLMTHTHKADPREIFLAIHHILKSRGHFLTPGAAKDFNTDKVDLEDIFPALTEAYAQVYPDLELTFDLAKADDFKAKLLDEQATPSDTQKALVNLLLSSDGEKEIVKKRKQVLTEFAKAITGLKTKFNLALGTEVDEADASNWQFSMGQLDDKWSNIETSMTDQGTEIFEQIQELYRARLLNGIVPAGMSLSQAKVADYGQHKEDLELFKTYLKKLNDHELAKTIRGLYDRYINGDDAKPFLREDFVKALTKEVTAHPNEVSEQLLNRMGQANFMLKQRTKANGAIPIQLQQRELDQIIANQSKYYDWLAAPNPVEAHRWKMPYQLDELLNFHIPYYVGPLITPKQQAESGENVFAWMVRKDPSGNITPYNFDEKVDREASANTFIQRMKTTDTYLIGEDVLPKQSLLYQKYEVLNELNNVRINNECLGTDQKQRLIREVFERHSSVTIKQVADNLVAHGDFARRPEIRGLADEKRFLSSLSTYHQLKEILHEAIDDPTKLLDIENIITWSTVFEDHTIFETKLAEIEWLDPKKINELSGIRYRGWGQFSRKLLDGLKLGNGHTVIQELMLSNHNLMQILADETLKETMTELNQDKLKTDDIEDVINDAYTSPSNKKALRQVLRVVEDIKHAANGQDPSWLFIETADGTGTAGKRTQSRQKQIQTVYANAAQELIDSAVRGELEDKIADKASFTDRLVLYFMQGGRDIYTGAPLNIDQLSHYDIDHILPQSLIKDDSLDNRVLVNATINREKNNVFASTLFAGKMKATWRKWHEAGLISGRKLRNLMLRPDEIDKFAKGFVARQLVETRQIIKLTEQIAAAQYPNTKIIAVKAGLSHQLREELDFPKNRDVNHYHHAFDAFLAARIGTYLLKRYPKLAPFFTYGEFAKVDVKKFREFNFIGALTHAKKNIIAKDTGEIVWDKERDIRELDRIYNFKRMLITHEVYFETADLFKQTIYAAKDSKERGGSKQLIPKKQGYPTQVYGGYTQESGSYNALVRVAEADTTAYQVIKISAQNASKIASANLKSREKGKQLLNEIVVKQLAKRRKNWKPSANSFKIVIPRFGMGTLFQNAKYGLFMVNSDTYYRNYQELWLSRENQKLLKKLFSIKYEKTQMNHDALQVYKAIIDQVEKFFKLYDINQFRAKLSDAIERFEKLPINTDGNKIGKTETLRQILIGLQANGTRSNVKNLGIKTDLGLLQVGSGIKLDKDTQIVYQSPSGLFKRRIPLADL

SEQ ID NO: 32 - protein, 1259 aa, Streptobacillus moniliformis
MKYILGLDLGVASIGWAVTEINEKGEPVGLIDANSVIFKPLDNDKGKLYNVERRDKRGSRRILRRKKHRVERTKMLLVNSSFLTNEEIDNLYVGKLDNIYEVRLKGLKEQLSKNEIARLMIYYCKNRGFKSNRKTEDLEILKSLGEKISEKDSDEKKLKPIIAKNTELIESKGLTPIEVIYMIRENDNSILGFKNKEGNYKFGFKRNQVIDEVRKILGTQQILSKEIVDEYIDILSSQRDFSDGPGGDSKYKIDYTKLAGKCKYTGEVRAVKSAPSYEIFTMLQKLNDIRYVKFTSEDKIEKKKLSKEVIHKLYDLVVEKNKTLTYDLIEKSIDEENIKLLNIPKLSKSKYVELRKSYFKERNDNENLTEEEINAFNEKLNNERMKQELIRNNLKAYKELKSKFKKNNIDENRIKELGGIYFLDLVAELLTYSKTDEKLEYFVENVERYSLFKEQRDIVEIIKTFPNYTKNGNLSLSLVRELNKLLMEGHDYESSLSSLNYYIDTDVDWEKFPTISEIEDSLSTKITNPNVKHILVILRKLYNTLLFKYGRPEKVHLELSRDFSNDFSTRNKIQKEQLENKVRREVAAFEMYGANKDIVAGKDRLSNDDFVRIKLWEEQNKVCMYSGRTIEKYQLTSAEVQIDHILPYSKSFDNSYSNKVLVFSNENQDKKERTPYQWLKGTEKWNEFKQRVRLNLNISNKKKENLLFEDEVVNNEFLERELHATSYSSRLALNIFQRLIPVSDEDKYDEHGNEKVKYLYNRNVIAFQGKMTSMLRNLYSLNKYTHSFESDTLDIDNKAFILKEFEINKDNLKMTAYNVNTGLEITSETKVEKNKSGEFKTIKDELLKKELDKKENLIEISDYFKDKVLFDLKIVDFEEIDTVEPEIISILYKMITALKEEVYSKNRENHLHHSLDAFLLTIMNRSMQMKLIKYNQLISVLKNKEIEIFNEEKGEYIDSKEFAEELQKQKIIDIDGNERGIKFNVNGKTHRLNLVKPYENFLDDIKNKIFDKRENNKLPYHVVKSKVSGALHAETILGESKGEITKRISVFDINYKNLGKIFDKDGSQKEIYETLVKWLEAKSKDYPKLKNGNIIKKVKIVDGNKDKLIKLGNKRYVEMGRTTVKILVLKKEAEEGLKFASIGRYKYNLLKSEKDVNISIWKDAIHFCKVRYNKLKENGYRLIHELIPGETIELELNKGNISKCLVVGFSGGKIEISSVIGDALDLINDKISTRIKERYYITVSTIKSIKKINKNILGD

SEQ ID NO: 33 - protein, 1024 aa, Helicobacter mustelae
MIRTLGIDIGIASIGWAVIEGEYTDKGLENKEIVASGVRVFTKAENPKNKESLALPRTLARSARRRNARKKGRIQQVKHYLSKALGLDLECFVQGEKLATLFQTSKDFLSPWELRERALYRVLDKEELARVILHIAKRRGYDDITYGVEDNDSGKIKKAIAENSKRIKEEQCKTIGEMMYKLYFQKSLNVRNKKESYNRCVGRSELREELKTIFQIQQELKSPWVNEELIYKLLGNPDAQSKQEREGLIFYQRPLKGFGDKIGKCSHIKKGENSPYRACKHAPSAEEFVALTKSINFLKNLTNRHGLCFSQEDMCVYLGKILQEAQKNEKGLTYSKLKLLLDLPSDFEFLGLDYSGKNPEKAVFLSLPSTFKLNKITQDRKTQDKIANILGANKDWEAILKELESLQLSKEQIQTIKDAKLNFSKHINLSLEALYHLLPLMREGKRYDEGVEILQERGIFSKPQPKNRQLLPPLSELAKEESYFDIPNPVLRRALSEFRKVVNALLEKYGGFHYFHIELTRDVCKAKSARMQLEKINKKNKSENDAASQLLEVLGLPNTYNNRLKCKLWKQQEEYCLYSGEKITIDHLKDQRALQIDHAFPLSRSLDDSQSNKVLCLTSSNQEKSNKTPYEWLGSDEKKWDMYVGRVYSSNFSPSKKRKLTQKNFKERNEEDFLARNLVDTGYIGRVTKEYIKHSLSFLPLPDGKKEHIRIISGSMTSTMRSFWGVQEKNRDHHLHHAQDAIIIACIEPSMIQKYTTYLKDKETHRLKSHQKAQILREGDHKLSLRWPMSNFKDKIQESIQNIIPSHHVSHKVTGELHQETVRTKEFYYQAFGGEEGVKKALKFGKIREINQGIVDNGAMVRVDIFKSKDKGKFYAVPIYTYDFAIGKLPNKAIVQGKKNGIIKDWLEMDENYEFCFSLFKNDCIKIQTKEMQEAVLAIYKSTNSAKATIELEHLSKYALKNEDEEKMFTDTDKEKNKTMTRESCGIQGLKVFQKVKLSVLGEVLEHKPRNRQNIALKTTPKHV

SEQ ID NO: 34 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 35 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSSDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 36 - protein, 987 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNNGDEEKSEILKAIKQNEEKLVNYQSVGEYLYKEYFQKFKENSKEFINVRNKKESYERCIAQSFLKDELKLIFQKQREFGFSFSKKFEEEVLSVAFYKRALKDFSHLVGNCSFFTDEKRAPKNSPLAFMFVALTRIINLLNNLKNTEGILYTKDDLNTLLNEVLKNGTLTYKQTKKLLGLSDDYEFKGEKGTYFIEFKKYKEFIKALGEHNLSQDNLNEIAKDITLIKDEIKLKKALAKYDLNQNQIDSLSKLEFKDHLNISFKALKLITPLMLEGKKYDEAYNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYPVPIYTMDFALKVLPNKAVVQGKDKKSGLIKDWILMDENYEFCFSLYKDSLILIQTKDMQEPELVYFNAFTSSTVSLIVSKHDNKFETLSKNQKILFKNANEKEVIAKSIGIQNLKVFEKYIVSALGEVTKAEFRQREDFKK

SEQ ID NO: 37 - protein, 984 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGDHSLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNYSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREGFKK

SEQ ID NO: 38 - protein, 984 aa, Campylobacter sp.
MVRILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNKTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSTKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 39 - protein, 984 aa, Campylobacter sp.
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGDHSLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKILNALLKKYGKVHKINIELAREVGKNHSQRAKIEKEQNENYKAKKDAEIECEKLGLKINSKNILKLRLFKEQKEFCAYSGEKIKLSDLQDEKMLEIDHIYPYSRSFDDSYMNKVLVFTKQNQEKLNKTPFEAFGNDSTKWQKIEVLAKNLPEKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAAIIAYANNSIVKAFSDFKKEQESNSVELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPELVYFNAFTSSTVSLIVSKHDNKFETLSKNQKILFKNANEKEVIAKSIGIQNLKVFEKYIVSALGEVTKAEFRQREDFKK

SEQ ID NO: 40 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 41 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 42 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 43 - protein, 1253 aa, Prevotella albensis
MNIKNFTGLYPLSKTLRFELKPIGKTKENIEKNGILTKDEQRAKDYLIVKGFIDEYHKQFIKDRLWDFKLPLESEGEKNSLEEYQELYELTKRNDAQEADFTEIKDNLRSSITEQLTKSGSAYDRIFKKEFIREDLVNFLEDEKDKNIVKQFEDFTTYFTGFYENRKNMYSSEEKSTAIAYRLIHQNLPKFMDNMRSFAKIANSSVSEHFSDIYESWKEYLNVNSIEEIFQLDYFSETLTQPHIEVYNYIIGKKVLEDGTEIKGINEYVNLYNQQQKDKSKRLPFLVPLYKQILSDREKLSWIAEEFDSDKKMLSAITESYNHLHNVLMGNENESLRNLLLNIKDYNLEKINITNDLSLTEISQNLFGRYDVFTNGIKNKLRVLTPRKKKETDENFEDRINKIFKTQKSFSIAFLNKLPQPEMEDGKPRNIEDYFITQGAINTKSIQKEDIFAQIENAYEDAQVFLQIKDTDNKLSQNKTAVEKIKTLLDALKELQHFIKPLLGSGEENEKDELFYGSFLAIWDELDTITPLYNKVRNWLTRKPYSTEKIKLNFDNAQLLGGWDVNKEHDCAGILLRKNDSYYLGIINKKTNHIFDTDITPSDGECYDKIDYKLLPGANKMLPKVFFSKSRIKEFEPSEAIINCYKKGTHKKGKNFNLTDCHRLINFFKTSIEKHEDWSKFGFKFSDTETYEDISGFYREVEQQGYRLTSHPVSASYIHSLVKEGKLYLFQIWNKDFSQFSKGTPNLHTLYWKMLFDKRNLSDVVYKLNGQAEVFYRKSSIEHQNRIIHPAQHPITNKNELNKKHTSTFKYDIIKDRRYTVDKFQFHVPITINFKATGQNNINPIVQEVIRQNGITHIIGIDRGERHLLYLSLIDLKGNIIKQMTLNEIINEYKGVTYKTNYHNLLEKREKERTEARHSWSSIESIKELKDGYMSQVIHKITDMMVKYNAIVVLEDLNGGFMRGRQKVEKQVYQKFEKKLIDKLNYLVDKKLDANEVGGVLNAYQLTNKFESFKKIGKQSGFLFYIPAWNTSKIDPITGFVNLFNTRYESIKETKVFWSKFDIIRYNKEKNWFEFVFDYNTFTTKAEGTRTKWTLCTHGTRIQTFRNPEKNAQWDNKEINLTESFKALFEKYKIDITSNLKESIMQETEKKFFQELHNLLHLTLQMRNSVTGTDIDYLISPVADEDGNFYDSRINGKNFPENADANGAYNIARKGLMLIRQIKQADPQKKFKFETITNKDWLKFAQDKPYLKD

SEQ ID NO: 44 - DNA, 600 bp, synthetic construct
  source 1..600: mol_type = other DNA
cagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgc

SEQ ID NO: 45 - DNA, 724 bp, synthetic construct
  source 1..724: mol_type = other DNA
cttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgc

SEQ ID NO: 46 - DNA, 524 bp, synthetic construct
  source 1..524: mol_type = other DNA
gagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagagagcgccggggaaggagaggcccgcgccctctcctggagccagattctgcaggtgcactgggtggggatgatcggcgggctaggttgcaagtaagtgccttttcttttgctgctttctttatttctttgtttccatccaggcctcttatgtgaggagctgaagaggaattaaaatatacaggatgaaaaggcggccgccatggtgagcaagggcgaggaggataa

SEQ ID NO: 47 - DNA, 1000 bp, synthetic construct
  source 1..1000: mol_type = other DNA
tcgaattaacacaagacagaagtcatataaatcttaagaaggtagaaaatgttacttctagaaaactccaccaaaagttggttggataatttcaggtcttctttctggaaaacgaagagtcacaaagtctatttttctttctactcccaagttccactcattctccaattaaatgtgactcataggtgactgaatgtcatgttttggccactctaagcaaagggattccttggacttgggtacacatattttaagaatattgtatatataagaatattgtatatattcaactgtccatcttgctgcttctctcttacaccaacttcattttgtagcatcagctgcataagctcttgcttttccccctatgcagacatctattcatcaggctctgtgtacactggcaacataggaataaactggataaggttgctgttcccagaagaatctcagaatattgtatttatttaatggcatcaggaaaaaccagtccaccaattttagaaagaaaaaagtcttaaagctgcacatttttatgaacttagacaattactatattttgactattattgactctaatctattccaagaattatacagacagaaagagctttggtaacagcctacatatcaatatttactgaaccaagaactatcacaaaacgtctgttgaaaatttccatgttttaatgatgatgatgctaatggcagctatgatttattaaacactttctatgtgcaggatgtaagctaagtaatttgtacacattatcttatttaaacttattacaagtcttattttcttgaggtagatgttattatcctattttcacagacagacttatgccttgaagaatttggtaaagtcactaagtagtcagtagccaaatcatgtattcttaatccttattcaatattgtccccctatagaagaaaccttgagtttgtaacttttcagtaaggctatttagcttgtgtcctgaagacactctcacctata

SEQ ID NO: 48 - DNA, 1000 bp, synthetic construct; identical to SEQ ID NO: 47
  source 1..1000: mol_type = other DNA

SEQ ID NO: 49 - DNA, 1000 bp, synthetic construct
  source 1..1000: mol_type = other DNA
tcaattataacagacatgtcttagtctaccttttattgctgtaactgaatacctgagactggttaaagtattcaataaagaaatttatttcttaaagttctggaagctgggaagtctaaggtgaagggtccacatctggtgggaactttcttgctggtgggcacaccagagagtcccaaggcagctcatggcatcacatggcgaggggtgttcactgagagccaaactgggttttataataaacccactcttgtgatatctaactcactcccatgataacccattaagcccttactctatattccattcatgaaggcagaaccttcatgacctcatcaccttttaaaggctctgcctcttaatactgttacattgggaattaagttccaacatgagtttcaaaagggacaaacattcaaaccacagcaaaacattaaaaaaaaaattttctgattaaagagggagcatacatattttcaagtgttcttaagtggaaaataatggatgtgaatgactgggcaaatttaagagtggcatgaggagtaatttattctgctaaatacgtagctttaggaatttaaaaaaaaaagccatgttacatttagtgagacatctggaaatactacatgaggatgagcactttaaaacacttcccgcttcatcagtcagtttacctgtctctgatggtaacagcaatgaactttttgtgaacaatgactttttctacgcctgaaatagataaatatataaagttggctctcatctgttcctattcttccctcgaaaaattgaatctctcagtcactaacttattcgtaatgaagaactgcattccagacatttttcttagacatcgtctcctgctacacaggatgtgaggcaaatgaatattttccatggaactaattcagagagtaaacatatttatagatccagatctaaggtcatggaggagctcattgctttgtaactaaaatgtgtattgattgtattgctgttc

SEQ ID NO: 50 - DNA, 1440 bp, synthetic construct
  source 1..1440: mol_type = other DNA
cagtctcagtcactgaagtttccttccttcaatagcacatcttacaagtcaagaatgtaaggtccaaagccccaggacaaatctcagaagcaaagaaagcagcagggaaaagtagaccctgggatttgatttccttccggttatctctaagcatcatttccatgatagaaggtgtggaagcaaataataaaagtgcccgtcactagtgtttatcctgcaaagtggtctgcccttttgagaggcaccctgccctatggccatcctttgatttcttcccttggtggaaatttcctgtttctctttgagaaagataattcaaccctgtttccattgttcttcctcccaggctgctgtaggaaagcgcacgcacacactccacacaaaatgaatttttaaaaaatttattttcacagtcgctcctaccagctctgaaattcagaacccatatgactgatggcatattcagataatcgggtcccaggtctggaaaagcagccttttccccacgtttctttccccacctaggacctcctctgattcttcactgcatcttcgaaagaaaatgtattatttgcttgcctggaagacgctgcaattcaattgattttatatatacatatatataaagaaaacagaaaacatagcctagataccggtcttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagaga

SEQ ID NO: 51 - DNA, 449 bp, synthetic construct
  source 1..449: mol_type = other DNA
cagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 52 - DNA, 607 bp, synthetic construct
  source 1..607: mol_type = other DNA
ctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaact

SEQ ID NO: 53 - DNA, 164 bp, synthetic construct
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 54 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 55 - DNA, 22 bp, synthetic construct
  source 1..22: mol_type = other DNA
ggctgctagcggcaggcgtccc

SEQ ID NO: 56 - DNA, 22 bp, synthetic construct
  source 1..22: mol_type = other DNA
gcagaatctggctccaggagag

SEQ ID NO: 57 - DNA, 22 bp, synthetic construct
  source 1..22: mol_type = other DNA
ggtggggatgatcggcgggcta

SEQ ID NO: 58 - DNA, 22 bp, synthetic construct
  source 1..22: mol_type = other DNA
taattcctcttcagctcctcac

SEQ ID NO: 59 - DNA, 22 bp, synthetic construct
  source 1..22: mol_type = other DNA
agggctcttctggctgctggac

SEQ ID NO: 60 - DNA, 22 bp, synthetic construct
  source 1..22: mol_type = other DNA
gggacgcctgccgctagcagcc

SEQ ID NO: 61 - DNA, 22 bp, synthetic construct
  source 1..22: mol_type = other DNA
tccccacagaagcagcaaaaga

SEQ ID NO: 62 - DNA, 22 bp, synthetic construct
  source 1..22: mol_type = other DNA
tttcgcccgtgctcgcctcaac

SEQ ID NO: 63 - DNA, 22 bp, synthetic construct
  source 1..22: mol_type = other DNA
gtttttctccagctccccacca

SEQ ID NO: 64 - DNA, 22 bp, synthetic construct
  source 1..22: mol_type = other DNA
aagggaagggctcttctggctg

SEQ ID NO: 65 - DNA, 22 bp, synthetic construct
  source 1..22: mol_type = other DNA
gttccagaaccgagatgtgaaa

SEQ ID NO: 66 - DNA, 20 bp, synthetic construct
  source 1..20: mol_type = other DNA
ttttctccagctccccacca

SEQ ID NO: 67 - DNA, 20 bp, synthetic construct
  source 1..20: mol_type = other DNA
cctctcttctttccaggtgg

SEQ ID NO: 68 - DNA, 22 bp, synthetic construct
  source 1..22: mol_type = other DNA
ggggtccaagtcctccaggggc

SEQ ID NO: 69 - DNA, 20 bp, synthetic construct
  source 1..20: mol_type = other DNA
cccagcgcgcccgggtcccc

SEQ ID NO: 70 - DNA, 20 bp, synthetic construct
  source 1..20: mol_type = other DNA
gcgcgctgggaggagtggag

SEQ ID NO: 71 - DNA, 20 bp, synthetic construct
  source 1..20: mol_type = other DNA
gctagcccagcctcagccga

SEQ ID NO: 72 - DNA, 20 bp, synthetic construct
  source 1..20: mol_type = other DNA
tgcacctgcagaatctggct

SEQ ID NO: 73 - DNA, 20 bp, synthetic construct
  source 1..20: mol_type = other DNA
acccagtgcacctgcagaat

SEQ ID NO: 74 - DNA, 20 bp, synthetic construct
  source 1..20: mol_type = other DNA
caggtgcactgggtggggat

SEQ ID NO: 75 - DNA, 20 bp, synthetic construct
  source 1..20: mol_type = other DNA
gcctcttatgtgaggagctg

SEQ ID NO: 76 - DNA, 20 bp, synthetic construct
  source 1..20: mol_type = other DNA
cttcagctcctcacataaga

SEQ ID NO: 77 - DNA, 20 bp, synthetic construct
  source 1..20: mol_type = other DNA
tatattttaattcctcttca

SEQ ID NO: 78 - DNA, 20 bp, synthetic construct
  source 1..20: mol_type = other DNA
cccggcatggtgtcagagcc

SEQ ID NO: 79 - DNA, 20 bp, synthetic construct
  source 1..20: mol_type = other DNA
ggcggtcgccagcgctccag

SEQ ID NO: 80 - DNA, 20 bp, synthetic construct
  source 1..20: mol_type = other DNA
gccacctggaaagaagagag

SEQ ID NO: 81 - DNA, 20 bp, synthetic construct
  source 1..20: mol_type = other DNA
ggtcgccagcgctccagcgg

SEQ ID NO: 82 - DNA, 20 bp, synthetic construct
  source 1..20: mol_type = other DNA
gccagcaatgggaggaagaa

SEQ ID NO: 83 - DNA, 20 bp, synthetic construct
  source 1..20: mol_type = other DNA
gttccaggtggcgtaataca

SEQ ID NO: 84 - DNA, 20 bp, synthetic construct
  source 1..20: mol_type = other DNA
ggcggggctgctacctccac

SEQ ID NO: 85 - DNA, 20 bp, synthetic construct
  source 1..20: mol_type = other DNA
gggcgcagtctgcttgcagg

SEQ ID NO: 86 - DNA, 20 bp, synthetic construct
  source 1..20: mol_type = other DNA
ggcgctccagcggcggctgt

SEQ ID NO: 87 - DNA, 20 bp, synthetic construct
  source 1..20: mol_type = other DNA
gaccgggtggttccagcaat

SEQ ID NO: 88 - DNA, 20 bp, synthetic construct
  source 1..20: mol_type = other DNA
ggggtggttccagcaatggg

SEQ ID NO: 89 - DNA, 19 bp, synthetic construct
  source 1..19: mol_type = other DNA
acagtgggcaggattgaaa

SEQ ID NO: 90 - DNA, 19 bp, synthetic construct
  source 1..19: mol_type = other DNA
gcaggtgcactcaccgggt

SEQ ID NO: 91 - DNA, 20 bp, synthetic construct
  source 1..20: mol_type = other DNA
gagctcagggagcatcgagg

SEQ ID NO: 92 - DNA, 19 bp, synthetic construct
  source 1..19: mol_type = other DNA
agagtcgcaattggagcgc

SEQ ID NO: 93 - DNA, 19 bp, synthetic construct
  source 1..19: mol_type = other DNA
ccagaccagcctgcacagt

SEQ ID NO: 94 - DNA, 20 bp, synthetic construct
  source 1..20: mol_type = other DNA
gagcgcaggctaggcctgca

SEQ ID NO: 95 - DNA, 19 bp, synthetic construct
  source 1..19: mol_type = other DNA
ctaggagtccgggataccc

SEQ ID NO: 96 - DNA, 20 bp, synthetic construct
  source 1..20: mol_type = other DNA
gaatccgcaggtgcactcac

SEQ ID NO: 97 - DNA, 20 bp, synthetic construct
  source 1..20: mol_type = other DNA
gaccagcctgcacagtgggc

SEQ ID NO: 98 - DNA, 20 bp, synthetic construct
  source 1..20: mol_type = other DNA
gcgacgcggttggcagccga

SEQ ID NO: 99 - DNA, 20 bp, synthetic construct
  source 1..20: mol_type = other DNA
gtttactccggagtcactgg

SEQ ID NO: 100 - DNA, 20 bp, synthetic construct
  source 1..20: mol_type = other DNA
gctatctccaccagtgactc

SEQ ID NO: 101 - DNA, 20 bp, synthetic construct
  source 1..20: mol_type = other DNA
gacatcacccagggccaagg

SEQ ID NO: 102 - DNA, 20 bp, synthetic construct
  source 1..20: mol_type = other DNA
gtagtttcgagggatccaat

SEQ ID NO: 103 - DNA, 20 bp, synthetic construct
  source 1..20: mol_type = other DNA
gctcccagcagaactgatcg

SEQ ID NO: 104 - DNA, 20 bp, synthetic construct
  source 1..20: mol_type = other DNA
gtgactccggagtaaagcga

SEQ ID NO: 105 - DNA, 20 bp, synthetic construct
  source 1..20: mol_type = other DNA
gggagctcaccatagaactt

SEQ ID NO: 106 - DNA, 20 bp, synthetic construct
  source 1..20: mol_type = other DNA
gacggatctagatcctccag

SEQ ID NO: 107 - DNA, 20 bp, synthetic construct
  source 1..20: mol_type = other DNA
gccgggtaagagctactagt

SEQ ID NO: 108 - DNA, 20 bp, synthetic construct
  source 1..20: mol_type = other DNA
gcccggtgtgtgctgtagaa

SEQ ID NO: 109 - protein, 30 aa, synthetic construct
YCQKWMWTCDSERKCCEGMVCRLWCKKKLW

SEQ ID NO: 110 - protein, 32 aa, synthetic construct
GPYCQKWMQTCDSERKCCEGMVCRLWCKKKLL

SEQ ID NO: 111 - protein, 35 aa, synthetic construct
DCRYMFGDCEKDEDCCKHLGCKRKMKYCAWDFTFT

SEQ ID NO: 112 - protein, 29 aa, synthetic construct
YCQKWMWTCDSKRACCEGLRCKLWCRKII

SEQ ID NO: 113 - protein, 29 aa, synthetic construct
YCQKWMWTCDSKRACCEGLRCKLWCRKEI

SEQ ID NO: 114 - protein, 29 aa, synthetic construct
YCQKWLWTCDSERKCCEDMVCRLWCKKRL

SEQ ID NO: 115 - protein, 34 aa, synthetic construct
ACLGQWDSCDPKASKCCPNYACEWKYPWCRYKLF

SEQ ID NO: 116 - protein, 34 aa, synthetic construct
ACLGQWASCDPKASKCCPNYACEWKYPWCRYKLF

SEQ ID NO: 117 - protein, 33 aa, synthetic construct
DCLKFGWKCNPRNDKCCSGLKCGSNHNWCKLHL

SEQ ID NO: 118 - protein, 34 aa, synthetic construct
DCLGFMRKCIPDNDKCCRPNLVCSRTHKWCKYVF

SEQ ID NO: 119 - protein, 34 aa, synthetic construct
DCLGFFRKCIPDNDKCCRPNLVCSRLHRWCKYVF

SEQ ID NO: 120 - protein, 34 aa, synthetic construct
DCLGAMRKCIPDNDKCCRPNLVCSRTHKWCKYVF

SEQ ID NO: 121 - protein, 35 aa, synthetic construct
ECLEIFKACNPSNDQCCKSSKLVCSRKTRWCKYQI

SEQ ID NO: 122 - protein, 35 aa, synthetic construct
GCLGIFKACNPSNDQCCKSSKLVCSRKTRWCKWQI

SEQ ID NO: 123 - protein, 33 aa, synthetic construct
DCLGMFKSCDPENDKCCKRLVCSRSHRWCKWKL

SEQ ID NO: 124 - protein, 33 aa, synthetic construct
ICLGMFKSCDPENDKCCKRLVCSRSHRWCKWKL

SEQ ID NO: 125 - protein, 33 aa, synthetic construct
DCGTIWHYCGTDQSECCEGWKCSRQLCKYVIDW

SEQ ID NO: 126 - protein, 35 aa, synthetic construct
ECRYLFGGCSSTSDCCKHLSCRSDWKYCAWDGTFS

SEQ ID NO: 127 - protein, 29 aa, synthetic construct
  SITE 6: note = Norleucine
  SITE 17: note = Propargylglycine
YCQKWXWTCDSKRACCXGLRCKLWCRKEI

SEQ ID NO: 128 - protein, 65 aa, synthetic construct
GVRDAYIADDKNCVYTCASNGYCNTECTKNGAESGYCQWIGRYGNACWCIKLPDEVPIRIPGKCR

SEQ ID NO: 129 - protein, 18 aa, synthetic construct
LLIILRRRIRKQAHAHSK

SEQ ID NO: 130 - protein, 21 aa, synthetic construct
KETWWETWWTEWSQPKKKRKV

SEQ ID NO: 131 - protein, 18 aa, synthetic construct
KLALKLALKALKAALKLA

SEQ ID NO: 132 - protein, 28 aa, synthetic construct
LSTAADMQGVVTDGMASGLDKDYLKPDD

SEQ ID NO: 133 - protein, 32 aa, synthetic construct
LLKKKCWLRCVMGECCKRESDCTQMWKQCYPG

SEQ ID NO: 134 - protein, 178 aa, synthetic construct
MAERPFQCRICMRNFSTSGHLSRHIRTHTGEKPFACDICGRKFADRSHLARHTKIHTGSQKPFQCRICMRNFSQSGDLTRHIRTHTGEKPFACDICGRKFAYRWLRNNHTKIHTGSQKPFQCRICMRNFSRSDTLSEHIRTHTGEKPFACDICGRKFARAQHLQQHTKIHLRQKDAAR

SEQ ID NO: 135 - protein, 178 aa, synthetic construct
MAERPFQCRICMRNFSDRSHLSRHIRTHTGEKPFACDICGRKFADRSALARHTKIHTGSQKPFQCRICMRNFSQSSDLSRHIRTHTGEKPFACDICGRKFARSDDLTRHTKIHTGSQKPFQCRICMRNFSRSDDLTRHIRTHTGEKPFACDICGRKFAQRSTLSSHTKIHLRQKDAAR

SEQ ID NO: 136 - protein, 178 aa, synthetic construct
MAERPFQCRICMRNFSQSSDLSRHIRTHTGEKPFACDICGRKFARSDALARHTKIHTGSQKPFQCRICMRNFSQSGDLTRHIRTHTGEKPFACDICGRKFARSWGLQVHTKIHTGSQKPFQCRICMRNFSRSDHLSQHIRTHTGEKPFACDICGRKFADSSTRKKHTKIHLRQKDAAR

SEQ ID NO: 137 - protein, 178 aa, synthetic construct
MAERPFQCRICMRNFSDRSHLTRHIRTHTGEKPFACDICGRKFAQSGDLTRHTKIHTGSQKPFQCRICMRNFSRSDDLTRHIRTHTGEKPFACDICGRKFAQRSTLSSHTKIHTGSQKPFQCRICMRNFSRSDVLSEHIRTHTGEKPFACDICGRKFARNQHRKTHTKIHLRQKDAAR

SEQ ID NO: 138 - protein, 178 aa, synthetic construct
MAERPFQCRICMRNFSRSDNLSEHIRTHTGEKPFACDICGRKFAERANRNSHTKIHTGSQKPFQCRICMRNFSRSDNLARHIRTHTGEKPFACDICGRKFAWRGDRVKHTKIHTGSQKPFQCRICMRNFSDRSDLSRHIRTHTGEKPFACDICGRKFARRTDLRRHTKIHLRQKDAAR

SEQ ID NO: 139 - protein, 178 aa, synthetic construct
MAERPFQCRICMRNFSDRSDLSRHIRTHTGEKPFACDICGRKFARRTDLRRHTKIHTGSQKPFQCRICMRNFSDRSDLSRHIRTHTGEKPFACDICGRKFARSHHLKAHTKIHTGSQKPFQCRICMRNFSRSANLARHIRTHTGEKPFACDICGRKFARSDNLREHTKIHLRQKDAAR

SEQ ID NO: 140 - protein, 178 aa, synthetic construct
MAERPFQCRICMRNFSRSAHLSRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAAR

SEQ ID NO: 141 - protein, 178 aa, synthetic construct
MAERPFQCRICMRNFSQSGDLTRHIRTHTGEKPFACDICGRKFAQSGARNIHTKIHTGSQKPFQCRICMRNFSRSANLARHIRTHTGEKPFACDICGRKFARSDALARHTKIHTGSQKPFQCRICMRNFSDRSDLSRHIRTHTGEKPFACDICGRKFARRTDLRRHTKIHLRQKDAAR

SEQ ID NO: 142 - protein, 178 aa, synthetic construct
MAERPFQCRICMRNFSRSDVLSRHIRTHTGEKPFACDICGRKFADSRDRKNHTKIHTGSQKPFQCRICMRNFSRSADLTRHIRTHTGEKPFACDICGRKFADRSHLARHTKIHTGSQKPFQCRICMRNFSRSDNLSEHIRTHTGEKPFACDICGRKFASKQYLIKHTKIHLRQKDAAR

SEQ ID NO: 143 - protein, 178 aa, synthetic construct
MAERPFQCRICMRNFSRSADLTRHIRTHTGEKPFACDICGRKFAQSGHLSRHTKIHTGSQKPFQCRICMRNFSQSSDLSRHIRTHTGEKPFACDICGRKFAHRKSLSRHTKIHTGSQKPFQCRICMRNFSDRSDLSRHIRTHTGEKPFACDICGRKFAQSSTRARHTKIHLRQKDAAR

SEQ ID NO: 144 - protein, 178 aa, synthetic construct
MAERPFQCRICMRNFSDRSHLTRHIRTHTGEKPFACDICGRKFARSDDLTRHTKIHTGSQKPFQCRICMRNFSDRSHLTRHIRTHTGEKPFACDICGRKFARSDNLTRHTKIHTGSQKPFQCRICMRNFSQSGHLARHIRTHTGEKPFACDICGRKFAQKGTLGEHTKIHLRQKDAAR

SEQ ID NO: 145 - protein, 178 aa, synthetic construct
MAERPFQCRICMRNFSRSAHLSRHIRTHTGEKPFACDICGRKFAQSGNLARHTKIHTGSQKPFQCRICMRNFSRSDAMSQHIRTHTGEKPFACDICGRKFARNASRTRHTKIHTGSQKPFQCRICMRNFSRSANLARHIRTHTGEKPFACDICGRKFADRSHLARHTKIHLRQKDAAR

SEQ ID NO: 146 - protein, 178 aa, synthetic construct
MAERPFQCRICMRNFSRSANLARHIRTHTGEKPFACDICGRKFADSSDRKKHTKIHTGSQKPFQCRICMRNFSTSGSLSRHIRTHTGEKPFACDICGRKFAHSLSLKNHTKIHTGSQKPFQCRICMRNFSQSSDLSRHIRTHTGEKPFACDICGRKFAWKWNLRAHTKIHLRQKDAAR

SEQ ID NO: 147 - protein, 178 aa, synthetic construct
MAERPFQCRICMRNFSRSAHLSRHIRTHTGEKPFACDICGRKFATSGHLSRHTKIHTGSQKPFQCRICMRNFSRSDHLSQHIRTHTGEKPFACDICGRKFAASSTRTKHTKIHTGSQKPFQCRICMRNFSQSSHLTRHIRTHTGEKPFACDICGRKFARSDNLTRHTKIHLRQKDAAR

SEQ ID NO: 148 - protein, 178 aa, synthetic construct
MAERPFQCRICMRNFSDRSHLTRHIRTHTGEKPFACDICGRKFADRSHLARHTKIHTGSQKPFQCRICMRNFSRSDNLSEHIRTHTGEKPFACDICGRKFARSAALARHTKIHTGSQKPFQCRICMRNFSRSDTLSQHIRTHTGEKPFACDICGRKFATRDHRIKHTKIHLRQKDAAR

SEQ ID NO: 149 - protein, 178 aa, synthetic construct
MAERPFQCRICMRNFSQSSALTRHIRTHTGEKPFACDICGRKFARSDNLRAHTKIHTGSQKPFQCRICMRNFSQSGDLTRHIRTHTGEKPFACDICGRKFAQSGDLTRHTKIHTGSQKPFQCRICMRNFSQSGHLVTHIRTHTGEKPFACDICGRKFATSGNLVRHTKIHLRQKDAAR

SEQ ID NO: 150 - protein, 178 aa, synthetic construct
MAERPFQCRICMRNFSERSALATHIRTHTGEKPFACDICGRKFADRSALARHTKIHTGSQKPFQCRICMRNFSQSAHLSRHIRTHTGEKPFACDICGRKFARSDALARHTKIHTGSQKPFQCRICMRNFSQSGDLTRHIRTHTGEKPFACDICGRKFATSGNLSRHTKIHLRQKDAAR

SEQ ID NO: 151 - protein, 178 aa, synthetic construct
MAERPFQCRICMRNFSDRSALTRHIRTHTGEKPFACDICGRKFAQSGDLTRHTKIHTGSQKPFQCRICMRNFSRSDSLTRHIRTHTGEKPFACDICGRKFATSGNLTRHTKIHTGSQKPFQCRICMRNFSTSGHLTRHIRTHTGEKPFACDICGRKFARSDALREHTKIHLRQKDAAR

SEQ ID NO: 152 - protein, 178 aa, synthetic construct
MAERPFQCRICMRNFSQSGDLTRHIRTHTGEKPFACDICGRKFAQSGDLTRHTKIHTGSQKPFQCRICMRNFSQSGHLSTHIRTHTGEKPFACDICGRKFATSGNLSRHTKIHTGSQKPFQCRICMRNFSQSGSLRAHIRTHTGEKPFACDICGRKFARSDNLSKHTKIHLRQKDAAR

SEQ ID NO: 153 - protein, 178 aa, synthetic construct
MAERPFQCRICMRNFSDRSHLTTHIRTHTGEKPFACDICGRKFADRSALTRHTKIHTGSQKPFQCRICMRNFSQSGDLTRHIRTHTGEKPFACDICGRKFARSDALAAHTKIHTGSQKPFQCRICMRNFSTSGNLTRHIRTHTGEKPFACDICGRKFATSGHLTRHTKIHLRQKDAAR

SEQ ID NO: 154 - protein, 178 aa, synthetic construct
MAERPFQCRICMRNFSRSDSLSVHIRTHTGEKPFACDICGRKFAQSGDLTRHTKIHTGSQKPFQCRICMRNFSTSGNLTRHIRTHTGEKPFACDICGRKFAQSTHLTSHTKIHTGSQKPFQCRICMRNFSQSGHLARHIRTHTGEKPFACDICGRKFAQLTHLNSHTKIHLRQKDAAR

SEQ ID NO: 155 - protein, 178 aa, synthetic construct
MAERPFQCRICMRNFSDRSALSRHIRTHTGEKPFACDICGRKFARSQHLQTHTKIHTGSQKPFQCRICMRNFSDRSALSDHIRTHTGEKPFACDICGRKFAQSGNLARHTKIHTGSQKPFQCRICMRNFSRSDNLARHIRTHTGEKPFACDICGRKFAQSGHLARHTKIHLRQKDAAR

SEQ ID NO: 156 - protein, 178 aa, synthetic construct
MAERPFQCRICMRNFSQSSNLSRHIRTHTGEKPFACDICGRKFARSDHLSTHTKIHTGSQKPFQCRICMRNFSRSDTLSEHIRTHTGEKPFACDICGRKFAQSATLTRHTKIHTGSQKPFQCRICMRNFSRSDNLARHIRTHTGEKPFACDICGRKFAQSGALSRHTKIHLRQKDAAR

SEQ ID NO: 157 - protein, 178 aa, synthetic construct
MAERPFQCRICMRNFSERGTLARHIRTHTGEKPFACDICGRKFARSDHRKTHTKIHTGSQKPFQCRICMRNFSDRSNLTRHIRTHTGEKPFACDICGRKFARSDNLARHTKIHTGSQKPFQCRICMRNFSRSDNLARHIRTHTGEKPFACDICGRKFATSANLVRHTKIHLRQKDAAR

SEQ ID NO: 158 - protein, 178 aa, synthetic construct
MAERPFQCRICMRNFSDRSNLSRHIRTHTGEKPFACDICGRKFARSDNLTRHTKIHTGSQKPFQCRICMRNFSRSDTLSVHIRTHTGEKPFACDICGRKFADNSTRIKHTKIHTGSQKPFQCRICMRNFSRSDHLSNHIRTHTGEKPFACDICGRKFADNRDRIKHTKIHLRQKDAAR

SEQ ID NO: 159 - protein, 178 aa, synthetic construct
MAERPFQCRICMRNFSDRSNLSRHIRTHTGEKPFACDICGRKFAQLASRTKHTKIHTGSQKPFQCRICMRNFSRSDNLSAHIRTHTGEKPFACDICGRKFATSQNRITHTKIHTGSQKPFQCRICMRNFSRSDNLSRHIRTHTGEKPFACDICGRKFAQSGNLVRHTKIHLRQKDAAR

SEQ ID NO: 160 - protein, 178 aa, synthetic construct
MAERPFQCRICMRNFSQSGSLTRHIRTHTGEKPFACDICGRKFARSDALARHTKIHTGSQKPFQCRICMRNFSTSSNLTRHIRTHTGEKPFACDICGRKFATSGHLTRHTKIHTGSQKPFQCRICMRNFSRSDHLSEHIRTHTGEKPFACDICGRKFADSRDRTKHTKIHLRQKDAAR

SEQ ID NO: 161 - protein, 178 aa, synthetic construct
MAERPFQCRICMRNFSDRSALARHIRTHTGEKPFACDICGRKFAQSGNLARHTKIHTGSQKPFQCRICMRNFSRSDNLARHIRTHTGEKPFACDICGRKFAQSGHLARHTKIHTGSQKPFQCRICMRNFSRSDSLTQHIRTHTGEKPFACDICGRKFAQSSHLTRHTKIHLRQKDAAR

SEQ ID NO: 162 - protein, 178 aa, synthetic construct
MAERPFQCRICMRNFSDRSHLSEHIRTHTGEKPFACDICGRKFARSDARKTHTKIHTGSQKPFQCRICMRNFSRSDTLSTHIRTHTGEKPFACDICGRKFADRSNLTRHTKIHTGSQKPFQCRICMRNFSRSDNLARHIRTHTGEKPFACDICGRKFARKDNRITHTKIHLRQKDAAR

SEQ ID NO: 163 - protein, 178 aa, synthetic construct
MAERPFQCRICMRNFSQSGHLARHIRTHTGEKPFACDICGRKFAQSGNLTRHTKIHTGSQKPFQCRICMRNFSQSGDLTRHIRTHTGEKPFACDICGRKFAQSGHLARHTKIHTGSQKPFQCRICMRNFSQSGHLSQHIRTHTGEKPFACDICGRKFAQSTSLSKHTKIHLRQKDAAR

SEQ ID NO: 164 - protein, 178 aa, synthetic construct
MAERPFQCRICMRNFSTSSNLSAHIRTHTGEKPFACDICGRKFARSDNRKKHTKIHTGSQKPFQCRICMRNFSQNSNLRRHIRTHTGEKPFACDICGRKFAQSGNLTRHTKIHTGSQKPFQCRICMRNFSQLGSLSRHIRTHTGEKPFACDICGRKFALKSNLRRHTKIHLRQKDAAR

SEQ ID NO: 165 - protein, 178 aa, synthetic construct
MAERPFQCRICMRNFSQSGNLQAHIRTHTGEKPFACDICGRKFAQSGNLRAHTKIHTGSQKPFQCRICMRNFSQSGNLTRHIRTHTGEKPFACDICGRKFALKQHLRTHTKIHTGSQKPFQCRICMRNFSQSSALSRHIRTHTGEKPFACDICGRKFARSDSLRQHTKIHLRQKDAAR

SEQ ID NO: 166 - protein, 178 aa, synthetic construct
MAERPFQCRICMRNFSRSDHLSEHIRTHTGEKPFACDICGRKFARSDHRKRHTKIHTGSQKPFQCRICMRNFSTSSALSRHIRTHTGEKPFACDICGRKFARSDNLAAHTKIHTGSQKPFQCRICMRNFSTSGNLARHIRTHTGEKPFACDICGRKFAQSGDLTRHTKIHLRQKDAAR

SEQ ID NO: 167 - protein, 178 aa, synthetic construct
MAERPFQCRICMRNFSRSWKLQAHIRTHTGEKPFACDICGRKFAQSGNLTRHTKIHTGSQKPFQCRICMRNFSRSGHLSRHIRTHTGEKPFACDICGRKFARSDNLRAHTKIHTGSQKPFQCRICMRNFSTSSNLSRHIRTHTGEKPFACDICGRKFARSDNLTQHTKIHLRQKDAAR

SEQ ID NO: 168 - protein, 178 aa, synthetic construct
MAERPFQCRICMRNFSQSGDLTRHIRTHTGEKPFACDICGRKFALRTSLRKHTKIHTGSQKPFQCRICMRNFSTPSYLPTHIRTHTGEKPFACDICGRKFADRSALARHTKIHTGSQKPFQCRICMRNFSQSSHLTRHIRTHTGEKPFACDICGRKFARSDALARHTKIHLRQKDAAR

SEQ ID NO: 169 - protein, 178 aa, synthetic construct
MAERPFQCRICMRNFSQSGNLSRHIRTHTGEKPFACDICGRKFARSDALRNHTKIHTGSQKPFQCRICMRNFSRSGNLARHIRTHTGEKPFACDICGRKFARSDNRITHTKIHTGSQKPFQCRICMRNFSRSDNLSAHIRTHTGEKPFACDICGRKFARSDNRITHTKIHLRQKDAAR

SEQ ID NO: 170 - protein, 178 aa, synthetic construct
MAERPFQCRICMRNFSRSDNLARHIRTHTGEKPFACDICGRKFAQSANRTKHTKIHTGSQKPFQCRICMRNFSRSDNLARHIRTHTGEKPFACDICGRKFAQWNYRGSHTKIHTGSQKPFQCRICMRNFSQSSDLSRHIRTHTGEKPFACDICGRKFAHRSNLSKHTKIHLRQKDAAR

SEQ ID NO: 171 - protein, 178 aa, synthetic construct
MAERPFQCRICMRNFSQSGHLARHIRTHTGEKPFACDICGRKFATSGNLARHTKIHTGSQKPFQCRICMRNFSQSGNLTRHIRTHTGEKPFACDICGRKFARSDNRITHTKIHTGSQKPFQCRICMRNFSRSANLSRHIRTHTGEKPFACDICGRKFAQSDTRITHTKIHLRQKDAAR

SEQ ID NO: 172 - protein, 178 aa, synthetic construct
MAERPFQCRICMRNFSQRSNLSRHIRTHTGEKPFACDICGRKFATSGHLSRHTKIHTGSQKPFQCRICMRNFSRSDTLSEHIRTHTGEKPFACDICGRKFAANSNRIKHTKIHTGSQKPFQCRICMRNFSRSDNLSVHIRTHTGEKPFACDICGRKFARRDTRNNHTKIHLRQKDAAR

SEQ ID NO: 173 - protein, 178 aa, synthetic construct
MAERPFQCRICMRNFSRSDTLSEHIRTHTGEKPFACDICGRKFAQNANRIKHTKIHTGSQKPFQCRICMRNFSRSDSLSQHIRTHTGEKPFACDICGRKFARSHNRKTHTKIHTGSQKPFQCRICMRNFSRSDNLSRHIRTHTGEKPFACDICGRKFATNQNRITHTKIHLRQKDAAR

SEQ ID NO: 174 - protein, 178 aa, synthetic construct
MAERPFQCRICMRNFSERGTLSRHIRTHTGEKPFACDICGRKFARSDHLSRHTKIHTGSQKPFQCRICMRNFSQSSTRKTHIRTHTGEKPFACDICGRKFATSSTLSNHTKIHTGSQKPFQCRICMRNFSRSDHLSQHIRTHTGEKPFACDICGRKFAQSATRKTHTKIHLRQKDAAR

SEQ ID NO: 175 - protein, 178 aa, synthetic construct
MAERPFQCRICMRNFSTSDHLSEHIRTHTGEKPFACDICGRKFAQSASRTKHTKIHTGSQKPFQCRICMRNFSQSGSLTRHIRTHTGEKPFACDICGRKFAQSGNLRKHTKIHTGSQKPFQCRICMRNFSDRSDLSRHIRTHTGEKPFACDICGRKFAQSSDLSRHTKIHLRQKDAAR

SEQ ID NO: 176 - protein, 178 aa, synthetic construct
MAERPFQCRICMRNFSRSDNLARHIRTHTGEKPFACDICGRKFARSDALARHTKIHTGSQKPFQCRICMRNFSRSDALSEHIRTHTGEKPFACDICGRKFARSSDRTKHTKIHTGSQKPFQCRICMRNFSRSDHLSRHIRTHTGEKPFACDICGRKFARNQDRTNHTKIHLRQKDAAR

SEQ ID NO: 177 - protein, 178 aa, synthetic construct
MAERPFQCRICMRNFSRSDTLSVHIRTHTGEKPFACDICGRKFADRSHLSRHTKIHTGSQKPFQCRICMRNFSRSDNLSVHIRTHTGEKPFACDICGRKFADRGNLTRHTKIHTGSQKPFQCRICMRNFSRSDNLARHIRTHTGEKPFACDICGRKFADSHHRITHTKIHLRQKDAAR

SEQ ID NO: 178 - protein, 178 aa, synthetic construct
MAERPFQCRICMRNFSQSGNLSRHIRTHTGEKPFACDICGRKFARSDALATHTKIHTGSQKPFQCRICMRNFSRSDNLSTHIRTHTGEKPFACDICGRKFARSGNLSRHTKIHTGSQKPFQCRICMRNFSRSDNLSTHIRTHTGEKPFACDICGRKFATSSNRTKHTKIHLRQKDAAR

SEQ ID NO: 179 - protein, 178 aa, synthetic construct
MAERPFQCRICMRNFSDRSALSRHIRTHTGEKPFACDICGRKFARSDALARHTKIHTGSQKPFQCRICMRNFSPCRYRLDHIRTHTGEKPFACDICGRKFARSANLTRHTKIHTGSQKPFQCRICMRNFSRSDNLSTHIRTHTGEKPFACDICGRKFADNSYLPRHTKIHLRQKDAAR

SEQ ID NO: 180 - protein, 178 aa, synthetic construct
MAERPFQCRICMRNFSNSGDLTRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSQSGNLTRHIRTHTGEKPFACDICGRKFAQSGDLGEHTKIHLRQKDAAR

SEQ ID NO: 181 - protein, 178 aa, synthetic construct
MAERPFQCRICMRNFSRSDNLSRHIRTHTGEKPFACDICGRKFARSDARTRHTKIHTGSQKPFQCRICMRNFSDRSDLARHIRTHTGEKPFACDICGRKFARSSDLSRHTKIHTGSQKPFQCRICMRNFSRSDHLSRHIRTHTGEKPFACDICGRKFADSQDRKTHTKIHLRQKDAAR

SEQ ID NO: 182 - protein, 178 aa, synthetic construct
MAERPFQCRICMRNFSQSGSLTRHIRTHTGEKPFACDICGRKFAHRQHLQTHTKIHTGSQKPFQCRICMRNFSDRSNLSRHIRTHTGEKPFACDICGRKFALKQVLVRHTKIHTGSQKPFQCRICMRNFSRSDSLLRHIRTHTGEKPFACDICGRKFADRSNRRKHTKIHLRQKDAAR

SEQ ID NO: 183 - protein, 178 aa, synthetic construct
MAERPFQCRICMRNFSRSDDLTAHIRTHTGEKPFACDICGRKFAQSAHRKRHTKIHTGSQKPFQCRICMRNFSTSAHLARHIRTHTGEKPFACDICGRKFATSASRTRHTKIHTGSQKPFQCRICMRNFSTSGHLTRHIRTHTGEKPFACDICGRKFARSDALAQHTKIHLRQKDAAR

SEQ ID NO: 184 - protein, 178 aa, synthetic construct
MAERPFQCRICMRNFSRSGNLTAHIRTHTGEKPFACDICGRKFAQNANRIKHTKIHTGSQKPFQCRICMRNFSQSGSLTQHIRTHTGEKPFACDICGRKFATSQNLTRHTKIHTGSQKPFQCRICMRNFSTSDSLLRHIRTHTGEKPFACDICGRKFATSHHLTRHTKIHLRQKDAAR

SEQ ID NO: 185 - protein, 178 aa, synthetic construct
MAERPFQCRICMRNFSQSSTRKTHIRTHTGEKPFACDICGRKFATSSTRTKHTKIHTGSQKPFQCRICMRNFSDRSALSRHIRTHTGEKPFACDICGRKFAQSGDLTRHTKIHTGSQKPFQCRICMRNFSRSDSLARHIRTHTGEKPFACDICGRKFATSGNLARHTKIHLRQKDAAR

SEQ ID NO: 186 - protein, 178 aa, synthetic construct
MAERPFQCRICMRNFSTSGHLKEHIRTHTGEKPFACDICGRKFAQSGHLSRHTKIHTGSQKPFQCRICMRNFSQSSDLSRHIRTHTGEKPFACDICGRKFAQSGNLSKHTKIHTGSQKPFQCRICMRNFSQSGHLNRHIRTHTGEKPFACDICGRKFAQSGNLARHTKIHLRQKDAAR

SEQ ID NO: 187 - protein, 178 aa, synthetic construct
MAERPFQCRICMRNFSTSSNRKTHIRTHTGEKPFACDICGRKFAQSANRITHTKIHTGSQKPFQCRICMRNFSQSGSLTRHIRTHTGEKPFACDICGRKFALKQNRIKHTKIHTGSQKPFQCRICMRNFSTPSYLPTHIRTHTGEKPFACDICGRKFADRSALARHTKIHLRQKDAAR

SEQ ID NO: 188 - protein, 178 aa, synthetic construct
MAERPFQCRICMRNFSRSGTLTRHIRTHTGEKPFACDICGRKFAQSGDLTRHTKIHTGSQKPFQCRICMRNFSQSGHLARHIRTHTGEKPFACDICGRKFARRSTRKSHTKIHTGSQKPFQCRICMRNFSQSGNLARHIRTHTGEKPFACDICGRKFAQSGNLARHTKIHLRQKDAAR

SEQ ID NO: 189 - protein, 178 aa, synthetic construct
MAERPFQCRICMRNFSRSDHLSQHIRTHTGEKPFACDICGRKFARSAVRKNHTKIHTGSQKPFQCRICMRNFSRSDHLSEHIRTHTGEKPFACDICGRKFAQSHHRKTHTKIHTGSQKPFQCRICMRNFSDRSNLSRHIRTHTGEKPFACDICGRKFALKQHLNEHTKIHLRQKDAAR

SEQ ID NO: 190 - protein, 178 aa, synthetic construct
MAERPFQCRICMRNFSDRSNLSRHIRTHTGEKPFACDICGRKFARSDDRKTHTKIHTGSQKPFQCRICMRNFSERGTLARHIRTHTGEKPFACDICGRKFAQSGHLSRHTKIHTGSQKPFQCRICMRNFSQSGHLARHIRTHTGEKPFACDICGRKFAVSHHLRDHTKIHLRQKDAAR

SEQ ID NO: 191 - protein, 178 aa, synthetic construct; identical to SEQ ID NO: 134

SEQ ID NO: 192 - protein, 178 aa, synthetic construct
MAERPFQCRICMRNFSQSGNLARHIRTHTGEKPFACDICGRKFARLDILQQHTKIHTGSQKPFQCRICMRNFSRSDVLSEHIRTHTGEKPFACDICGRKFATRNGLKYHTKIHTGSQKPFQCRICMRNFSQSSDLSRHIRTHTGEKPFACDICGRKFARKYYLAKHTKIHLRQKDAAR

SEQ ID NO: 193 - protein, 62 aa, synthetic construct
VTFEDVTVNFTQGEWQRLNPEQRNLYRDVMLENYSNLVSVGQGETTKPDVILRLEQGKEPWL

SEQ ID NO: 194 - protein, 62 aa, synthetic construct
VTFKDVFVDFTREEWKLLDTAQQIVYRNVMLENYKNLVSLGYQLTKPDVILRLEKGEEPWLV

SEQ ID NO: 195 - protein, 62 aa, synthetic construct
VTFEDVSMDFSQEEWELLEPAQKNLYREVMLENYRNVVSLEALKNQCTDVGIKEGPLSPAQT

SEQ ID NO: 196 - protein, 61 aa, synthetic construct
VTFDDVAVTFTKEEWGQLDLAQRTLYQEVMLENCGLLVSLGCPVPKAELICHLEHGQEPWT

SEQ ID NO: 197 - protein, 61 aa, synthetic construct
MAFEDVAVYFSQEEWGLLDTAQRALYRRVMLDNFALVASLGLSTSRPRVVIQLERGEEPWV

SEQ ID NO: 198 - protein, 286 aa, synthetic construct
VQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 199 - protein, 220 aa, synthetic construct
ARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKILEDALMADILSRAADTEEMDIEMDSGDEAEF

SEQ ID NO: 200 - protein, 29 aa, synthetic construct
MNIQMLLEAADYLERREREAEHGYASMLP

SEQ ID NO: 201 - protein, 121 aa, synthetic construct
MKEGENNKPREKSEGNKRKSSFSNSADDIKSKKKREQSNDIARGFERGLEPEKIIGATDSCGDLMFLMKWKDTDEADLVLAKEANVKCPQIVIAFYEERLTWHAYPEDAENKEKESAKSEF

SEQ ID NO: 202 - protein, 267 aa, synthetic construct
SSSMADFRKFFAKAKHIVIISGAGVSAESGVPTFRGAGGYWRKWQAQDLATPLAFAHNPSRVWEFYHYRREVGSKEPNAGHRAIAECETRLGKQGRRVWITQNIDELHRKAGTKNLLEIHGSLFKTRCTSCGWAENYKSPICPALSGKGAPEPGTQDASIPVEKLPRCEEAGCGGLLRPHWWFGENLDPAILEEVDRELAHCDLCLWGTSSWYPAAFAPQVAARGVPVAEFNTETTPATNRFRFHFQGPCGTTLPEALACHENETVS

SEQ ID NO: 203 - protein, 209 aa, synthetic construct
SCDSTNAAIAKQALKKPIKGKQAPRKKAQGKTQQNRKLTDFYPVRRSSRKSKAELQSEERKRIDELIESGKEEGKIDLIDGKGRGVIATKQFSRGDFVVEYHGDLIEITDAKKREALYAQDPSTGCYYYFQYLSKTYCVDATRETNRLGRLINHSKCGNCQTKLHDIDGVPHLILIASRDIAAGEELLYDYGDRSKASIEAFPWLKHEF

SEQ ID NO: 204 - protein, 213 aa, synthetic construct
MEFWGIEVKSGKPVTVTPEEGILIHVSQASLGECKNKKGEFVPLHVKVGNQNLVLGTLSTENIPQLFCDLVFDKEFELSHTWGKGSVYFVGYKTPNIEPQGYSEEEEEEEEEVPAGNAAKAVAKPKAKPAEVKPAVDDEEDESDSDGMDEDDSDGEDSEEEEPTPKKPASSKKRANETTPKAPVSAKKAKVAVTPQKTDEKKKGGKAANQSEF

SEQ ID NO: 205 - protein, 6 aa, synthetic construct
DLELRL

SEQ ID NO: 206 - DNA, 18 bp, Homo sapiens
  source 1..18: mol_type = genomic DNA
agtctgcttgcaggcggt

SEQ ID NO: 207 - DNA, 18 bp, Homo sapiens
  source 1..18: mol_type = genomic DNA
ccagcggcggctgtcggc

SEQ ID NO: 208 - DNA, 18 bp, Homo sapiens
  source 1..18: mol_type = genomic DNA
gcctgggtgccagtggct

SEQ ID NO: 209 - DNA, 18 bp, Homo sapiens
  source 1..18: mol_type = genomic DNA
tggctgctagcggcaggc

SEQ ID NO: 210 - DNA, 18 bp, Homo sapiens
  source 1..18: mol_type = genomic DNA
gcgtcccctgagcaacag

SEQ ID NO: 211 - DNA, 18 bp, Homo sapiens
  source 1..18: mol_type = genomic DNA
aaggagaggcccgcgccc

SEQ ID NO: 212 - DNA, 18 bp, Homo sapiens
  source 1..18: mol_type = genomic DNA
gcaggtgcactgggtggg

SEQ ID NO: 213 - DNA, 18 bp, Homo sapiens
  source 1..18: mol_type = genomic DNA
gcgcccgtggaggtagca

SEQ ID NO: 214 - DNA, 18 bp, Homo sapiens
  source 1..18: mol_type = genomic DNA
tgccagggcgcgcccgtg

SEQ ID NO: 215 - DNA, 18 bp, Homo sapiens
  source 1..18: mol_type = genomic DNA
acagccgccgctggagcg

SEQ ID NO: 216 - DNA, 18 bp, Homo sapiens
  source 1..18: mol_type = genomic DNA
ccaggagagggcgcgggc

SEQ ID NO: 217 - DNA, 18 bp, Mus musculus
  source 1..18: mol_type = genomic DNA
ggcgaggtgatggaaggg

SEQ ID NO: 218 - DNA, 18 bp, Mus musculus
  source 1..18: mol_type = genomic DNA
gagggagctaggggtggg

SEQ ID NO: 219 - DNA, 18 bp, Mus musculus
  source 1..18: mol_type = genomic DNA
agtgctaatgtttccgag

SEQ ID NO: 220 - DNA, 18 bp, Mus musculus
  source 1..18: mol_type = genomic DNA
tagacggtgcagggcgga

SEQ ID NO: 221 - DNA, 18 bp, Homo sapiens
  source 1..18: mol_type = genomic DNA
gattgagcagcaaaggtt

SEQ ID NO: 222 - DNA, 18 bp, Homo sapiens
  source 1..18: mol_type = genomic DNA
gatgcagtgggagtcttc

SEQ ID NO: 223 - DNA, 18 bp, Homo sapiens
  source 1..18: mol_type = genomic DNA
ctgggtgatgtggcagtc

SEQ ID NO: 224 - DNA, 18 bp, Homo sapiens
  source 1..18: mol_type = genomic DNA
aagcaagattgagcagca

SEQ ID NO: 225 - DNA, 18 bp, Homo sapiens
  source 1..18: mol_type = genomic DNA
ggtgatgtggcagtctgc

SEQ ID NO: 226 - DNA, 18 bp, Homo sapiens
  source 1..18: mol_type = genomic DNA
agaggaagagatgcagtg

SEQ ID NO: 227 - DNA, 18 bp, Homo sapiens
  source 1..18: mol_type = genomic DNA
ggagaggaagtctcggtc

SEQ ID NO: 228 - DNA, 18 bp, Homo sapiens
  source 1..18: mol_type = genomic DNA
gtagaggcaatctgggat

SEQ ID NO: 229 - DNA, 18 bp, Homo sapiens
  source 1..18: mol_type = genomic DNA
gatgagagggacagggcc

SEQ ID NO: 230 - DNA, 18 bp, Homo sapiens
  source 1..18: mol_type = genomic DNA
cccagggccaaggaggac

SEQ ID NO: 231 - DNA, 18 bp, Homo sapiens
  source 1..18: mol_type = genomic DNA
gaagagtataaacttgac

SEQ ID NO: 232 - DNA, 18 bp, Homo sapiens
  source 1..18: mol_type = genomic DNA
gccctgggtgatgtggca

SEQ ID NO: 233 - DNA, 18 bp, Homo sapiens
  source 1..18: mol_type = genomic DNA
gctcttggagaggaagtc

SEQ ID NO: 234 - DNA, 18 bp, Homo sapiens
  source 1..18: mol_type = genomic DNA
aaggaggactttctgggc

SEQ ID NO: 235 - DNA, 18 bp, Homo sapiens
  source 1..18: mol_type = genomic DNA
gcatagggagcagaagga

SEQ ID NO: 236 - DNA, 18 bp, Homo sapiens
  source 1..18: mol_type = genomic DNA
tacgtagaagacaagcaa

SEQ ID NO: 237 - DNA, 18 bp, Homo sapiens
  source 1..18: mol_type = genomic DNA
ttggtttatgaataaaat

SEQ ID NO: 238 - DNA, 18 bp, Homo sapiens
  source 1..18: mol_type = genomic DNA
gcagcaaaggtttggctg

SEQ ID NO: 239 - DNA, 18 bp, Homo sapiens
  source 1..18: mol_type = genomic DNA
aaggataagggtgaaaat

SEQ ID NO: 240 - DNA, 18 bp, Homo sapiens
  source 1..18: mol_type = genomic DNA
gtgggagtcttctatgca

SEQ ID NO: 241 - DNA, 18 bp, Homo sapiens
  source 1..18: mol_type = genomic DNA
aagaatgagaagatggaa

SEQ ID NO: 242 - DNA, 18 bp, Homo sapiens
  source 1..18: mol_type = genomic DNA
tatccttcagaggaagag

SEQ ID NO: 243 - DNA, 18 bp, Homo sapiens
  source 1..18: mol_type = genomic DNA
gaagaatgagaagatgga

SEQ ID NO: 244 - DNA, 18 bp, Homo sapiens
  source 1..18: mol_type = genomic DNA
ctgaaggataagggtgaa

SEQ ID NO: 245 - DNA, 18 bp, Homo sapiens
  source 1..18: mol_type = genomic DNA
aatgagaagatggaattc

SEQ ID NO: 246 - DNA, 18 bp, Homo sapiens
  source 1..18: mol_type = genomic DNA
tcaagtccagctggggcc

SEQ ID NO: 247 - DNA, 18 bp, Homo sapiens
  source 1..18: mol_type = genomic DNA
gctgcccaagttctatgg

SEQ ID NO: 248 - DNA, 18 bp, Homo sapiens
  source 1..18: mol_type = genomic DNA
ccggagtcactggtggag

SEQ ID NO: 249 - DNA, 18 bp, Homo sapiens
  source 1..18: mol_type = genomic DNA
tgagagggacagggccag

SEQ ID NO: 250 - DNA, 18 bp, Homo sapiens
  source 1..18: mol_type = genomic DNA
gaccacgagctcctggaa

SEQ ID NO: 251 - DNA, 18 bp, Homo sapiens
  source 1..18: mol_type = genomic DNA
ttccaggagctcgtggtc

SEQ ID NO: 252 - DNA, 18 bp, Homo sapiens
  source 1..18: mol_type = genomic DNA
ccaagaactatgggtgtt

SEQ ID NO: 253 - DNA, 18 bp, Homo sapiens
  source 1..18: mol_type = genomic DNA
atcggggagcccctggag

SEQ ID NO: 254 - DNA, 18 bp, Homo sapiens
  source 1..18: mol_type = genomic DNA
tctgtggctaacagtgta

SEQ ID NO: 255 - DNA, 18 bp, Homo sapiens
  source 1..18: mol_type = genomic DNA
atgggtgtttgaggaagg

SEQ ID NO: 256 - DNA, 18 bp, Homo sapiens
  source 1..18: mol_type = genomic DNA
tgtgttgatataaaaaga

SEQ ID NO: 257 - DNA, 18 bp, Homo sapiens
  source 1..18: mol_type = genomic DNA
gatgtggcagtctgctaa

SEQ ID NO: 258 - DNA, 18 bp, Homo sapiens
  source 1..18: mol_type = genomic DNA
gaatgttaagctgaacgt

SEQ ID NO: 259 - DNA, 18 bp, Homo sapiens
  source 1..18: mol_type = genomic DNA
gtcttctacgtaagaaat

SEQ ID NO: 260 - DNA, 18 bp, Homo sapiens
  source 1..18: mol_type = genomic DNA
gaagcatagggagcagaa

SEQ ID NO: 261 - DNA, 18 bp, Mus musculus
  source 1..18: mol_type = genomic DNA
agtgacggacgggtgagg

SEQ ID NO: 262 - DNA, 17 bp, Mus musculus
  source 1..17: mol_type = genomic DNA
gtggaggagccccggac

SEQ ID NO: 263 - DNA, 18 bp, Mus musculus
  source 1..18: mol_type = genomic DNA
ggtgctccagaaagtaca

SEQ ID NO: 264 - protein, 298 aa, synthetic construct
RSMHDYKDHDGDYKDHDIDYKDDDDKMAPKKKRKVGIHGVPAAMAERPFQCRICMRNFSRSAHLSRHIRTHTGEKPFACDICGRKFAQSGNLARHTKIHTGSQKPFQCRICMRNFSRSDAMSQHIRTHTGEKPFACDICGRKFARNASRTRHTKIHTGSQKPFQCRICMRNFSRSANLARHIRTHTGEKPFACDICGRKFADRSHLARHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 265 - protein, 298 aa, synthetic construct
RSMHDYKDHDGDYKDHDIDYKDDDDKMAPKKKRKVGIHGVPAAMAERPFQCRICMRNFSRSANLARHIRTHTGEKPFACDICGRKFADSSDRKKHTKIHTGSQKPFQCRICMRNFSTSGSLSRHIRTHTGEKPFACDICGRKFAHSLSLKNHTKIHTGSQKPFQCRICMRNFSQSSDLSRHIRTHTGEKPFACDICGRKFAWKWNLRAHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 266 - protein, 298 aa, synthetic construct
RSMHDYKDHDGDYKDHDIDYKDDDDKMAPKKKRKVGIHGVPAAMAERPFQCRICMRNFSRSAHLSRHIRTHTGEKPFACDICGRKFATSGHLSRHTKIHTGSQKPFQCRICMRNFSRSDHLSQHIRTHTGEKPFACDICGRKFAASSTRTKHTKIHTGSQKPFQCRICMRNFSQSSHLTRHIRTHTGEKPFACDICGRKFARSDNLTRHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 267 - protein, 298 aa, synthetic construct
RSMHDYKDHDGDYKDHDIDYKDDDDKMAPKKKRKVGIHGVPAAMAERPFQCRICMRNFSDRSHLTRHIRTHTGEKPFACDICGRKFADRSHLARHTKIHTGSQKPFQCRICMRNFSRSDNLSEHIRTHTGEKPFACDICGRKFARSAALARHTKIHTGSQKPFQCRICMRNFSRSDTLSQHIRTHTGEKPFACDICGRKFATRDHRIKHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 268 - DNA, 20 bp, synthetic construct
  source 1..20: mol_type = other DNA
ggcagggtggaactcgtgac

SEQ ID NO: 269 - DNA, 20 bp, synthetic construct
  source 1..20: mol_type = other DNA
gcaccatccagcaagcaggg

SEQ ID NO: 270 - DNA, 20 bp, synthetic construct
  source 1..20: mol_type = other DNA
gcgtcactcaaggatctaca

SEQ ID NO: 271 - DNA, 20 bp, synthetic construct
  source 1..20: mol_type = other DNA
gatgggaatggcacccacga

SEQ ID NO: 272 - DNA, 20 bp, synthetic construct
  source 1..20: mol_type = other DNA
gcctttagacggagaacaga

SEQ ID NO: 273 - DNA, 20 bp, synthetic construct
  source 1..20: mol_type = other DNA
gagatccttgagtgacggac

SEQ ID NO: 274 - DNA, 20 bp, synthetic construct
  source 1..20: mol_type = other DNA
gcggggctcctccacgaagg

SEQ ID NO: 275 - DNA, 20 bp, synthetic construct
  source 1..20: mol_type = other DNA
gcaaggaatcacgccttcgt

SEQ ID NO: 276 - DNA, 20 bp, synthetic construct
  source 1..20: mol_type = other DNA
ggccatgcgcgaatgctgag

SEQ ID NO: 277 - DNA, 20 bp, synthetic construct
  source 1..20: mol_type = other DNA
ggcaagcccagccaccttcg

SEQ ID NO: 278 - protein, 178 aa, synthetic construct
MAERPFQCRICMRNFSRSDNLARHIRTHTGEKPFACDICGRKFATSGSLTRHTKIHTGSQKPFQCRICMRNFSRSDDLSKHIRTHTGEKPFACDICGRKFARSDALARHTKIHTGSQKPFQCRICMRNFSTSGHLSRHIRTHTGEKPFACDICGRKFARSDALARHTKIHLRQKDAAR

SEQ ID NO: 279 - protein, 178 aa, synthetic construct
MAERPFQCRICMRNFSRSDALSEHIRTHTGEKPFACDICGRKFARSADRTRHTKIHTGSQKPFQCRICMRNFSQSGHLARHIRTHTGEKPFACDICGRKFAHRSTLSRHTKIHTGSQKPFQCRICMRNFSDRSDLSRHIRTHTGEKPFACDICGRKFADRSDLSRHTKIHLRQKDAAR

SEQ ID NO: 280 - protein, 180 aa, synthetic construct
MAERPFQCRICMRNFSQSGDLTRHIRTHTGEKPFACDICGRKFAQSGARNIHTKIHTGSQKPFQCRICMRNFSRSANLARHIRTHTGEKPFACDICGRKFARSDALARHTKIHTGSQKPFQCRICMRNFSDRSDLSRHIRTHTGEKPFACDICGRKFARRTDLRRHTKIHLRQKDAARGS

SEQ ID NO: 281 - protein, 178 aa, synthetic construct
MAERPFQCRICMRNFSRSDNLSRHIRTHTGEKPFACDICGRKFAHSATRKRHTKIHTGSQKPFQCRICMRNFSRSDDLSKHIRTHTGEKPFACDICGRKFARSDALARHTKIHTGSQKPFQCRICMRNFSRSSHLARHIRTHTGEKPFACDICGRKFARSDALARHTKIHLRQKDAAR

SEQ ID NO: 282 - protein, 178 aa, synthetic construct
MAERPFQCRICMRNFSRSDTLTRHIRTHTGEKPFACDICGRKFARSDARTNHTKIHTGSQKPFQCRICMRNFSRSSDLTRHIRTHTGEKPFACDICGRKFAQSGHLSRHTKIHTGSQKPFQCRICMRNFSRSDTLTQHIRTHTGEKPFACDICGRKFADSSDLSRHTKIHLRQKDAAR

SEQ ID NO: 283 - protein, 178 aa, synthetic construct
MAERPFQCRICMRNFSRSDDLTRHIRTHTGEKPFACDICGRKFARSDNLARHTKIHTGSQKPFQCRICMRNFSQSSALSRHIRTHTGEKPFACDICGRKFARSDHLSRHTKIHTGSQKPFQCRICMRNFSRSDSLARHIRTHTGEKPFACDICGRKFANRHHLTRHTKIHLRQKDAAR

SEQ ID NO: 284 - protein, 178 aa, synthetic construct
MAERPFQCRICMRNFSDRSSLTTHIRTHTGEKPFACDICGRKFADRSHLARHTKIHTGSQKPFQCRICMRNFSRSDNLARHIRTHTGEKPFACDICGRKFAQSAHLARHTKIHTGSQKPFQCRICMRNFSQSGNLARHIRTHTGEKPFACDICGRKFARSDHLSRHTKIHLRQKDAAR

SEQ ID NO: 285 - protein, 178 aa, synthetic construct
MAERPFQCRICMRNFSRSDNLSEHIRTHTGEKPFACDICGRKFAQSANRNKHTKIHTGSQKPFQCRICMRNFSRSDNLARHIRTHTGEKPFACDICGRKFAWRGDRVKHTKIHTGSQKPFQCRICMRNFSDRSDLSRHIRTHTGEKPFACDICGRKFARRTDLRRHTKIHLRQKDAAR

SEQ ID NO: 286 - protein, 178 aa, synthetic construct
MAERPFQCRICMRNFSQSGSLSRHIRTHTGEKPFACDICGRKFARSDDLSRHTKIHTGSQKPFQCRICMRNFSRSDSLSQHIRTHTGEKPFACDICGRKFARSGHLSRHTKIHTGSQKPFQCRICMRNFSDRSDLSRHIRTHTGEKPFACDICGRKFAQSSTLARHTKIHLRQKDAAR

SEQ ID NO: 287 - protein, 178 aa, synthetic construct
MAERPFQCRICMRNFSDQSTLRSHIRTHTGEKPFACDICGRKFADRSNLSRHTKIHTGSQKPFQCRICMRNFSRSDALSEHIRTHTGEKPFACDICGRKFARSADRKRHTKIHTGSQKPFQCRICMRNFSQSGHLARHIRTHTGEKPFACDICGRKFATSSTLSKHTKIHLRQKDAAR

SEQ ID NO: 288 - protein, 178 aa, synthetic construct
MAERPFQCRICMRNFSRSDHLSRHIRTHTGEKPFACDICGRKFATSSHLARHTKIHTGSQKPFQCRICMRNFSTSGNLARHIRTHTGEKPFACDICGRKFATSGNLVRHTKIHTGSQKPFQCRICMRNFSRSDHLTRHIRTHTGEKPFACDICGRKFATSGHLVRHTKIHLRQKDAAR

SEQ ID NO: 289 - protein, 178 aa, synthetic construct
MAERPFQCRICMRNFSRSDNLARHIRTHTGEKPFACDICGRKFARSDHRKKHTKIHTGSQKPFQCRICMRNFSQSGNLARHIRTHTGEKPFACDICGRKFARSGNLRAHTKIHTGSQKPFQCRICMRNFSDSSALSTHIRTHTGEKPFACDICGRKFARSDHLSQHTKIHLRQKDAAR

SEQ ID NO: 290 - protein, 178 aa, synthetic construct
MAERPFQCRICMRNFSERSDLSVHIRTHTGEKPFACDICGRKFADRDDLSRHTKIHTGSQKPFQCRICMRNFSRSDDRKVHIRTHTGEKPFACDICGRKFADRSHRIRHTKIHTGSQKPFQCRICMRNFSRSDNLARHIRTHTGEKPFACDICGRKFAQSGHLARHTKIHLRQKDAAR

SEQ ID NO: 291 - DNA, 18 bp, Homo sapiens
  source 1..18: mol_type = genomic DNA
gtgggtgtggcggttgag

SEQ ID NO: 292 - DNA, 18 bp, Homo sapiens
  source 1..18: mol_type = genomic DNA
gccgccgctggagcgctg

SEQ ID NO: 293 - DNA, 18 bp, Homo sapiens
  source 1..18: mol_type = genomic DNA
gcgcccgtggaggtagca

SEQ ID NO: 294 - DNA, 18 bp, Homo sapiens
  source 1..18: mol_type = genomic DNA
gccgctggagcgctggcg

SEQ ID NO: 295 - DNA, 18 bp, Homo sapiens
  source 1..18: mol_type = genomic DNA
ggtgtggcggttgaggcg

SEQ ID NO: 296 - DNA, 18 bp, Homo sapiens
  source 1..18: mol_type = genomic DNA
ggggaaggagaggcccgc

SEQ ID NO: 297 - DNA, 18 bp, Homo sapiens
  source 1..18: mol_type = genomic DNA
gcgtcccctgagcaacag

SEQ ID NO: 298 - DNA, 18 bp, Homo sapiens
  source 1..18: mol_type = genomic DNA
tcagccgagctggcggaa

SEQ ID NO: 299 - DNA, 18 bp, Homo sapiens
  source 1..18: mol_type = genomic DNA
gctggagcgctggcgacc

SEQ ID NO: 300 - DNA, 18 bp, Homo sapiens
  source 1..18: mol_type = genomic DNA
ggtggggatgatcggcgg

SEQ ID NO: 301 - DNA, 18 bp, Homo sapiens
  source 1..18: mol_type = genomic DNA
tggttccagcaatgggag

SEQ ID NO: 302 - DNA, 18 bp, Homo sapiens
  source 1..18: mol_type = genomic DNA
ggagagggcgcgggcctc

SEQ ID NO: 303 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 304 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 305 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 306 - protein, 10 aa, synthetic construct
  VARIANT 1..10: note = This sequence may encompass 1-10 residues
GGGGGGGGGG

SEQ ID NO: 307 - protein, 50 aa, synthetic construct
  VARIANT 1..50: note = This sequence may encompass 1-10 EAAAK repeating units
EAAAKEAAAKEAAAKEAAAKEAAAKEAAAKEAAAKEAAAKEAAAKEAAAK

SEQ ID NO: 308 - protein, 106 aa, synthetic construct
  VARIANT 2..51: note = This region may encompass 1-10 EAAAK repeating units
  VARIANT 56..105: note = This region may encompass 1-10 EAAAK repeating units
AEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKA

SEQ ID NO: 309 - protein, 5 aa, synthetic construct
PAPAP

SEQ ID NO: 310 - protein, 12 aa, synthetic construct
AEAAAKEAAAKA

SEQ ID NO: 311 - protein, 68 aa, synthetic construct
  VARIANT 1..68: note = This sequence may encompass 10-34 AP repeating units
APAPAPAPAPAPAPAPAPAPAPAPAPAPAPAPAPAPAPAPAPAPAPAPAPAPAPAPAPAPAPAPAPAP